(19) United States
(12) Patent Application Publication
(10) Pub. No.: US 2019/0194728 A1
(43) Pub. Date: Jun. 27, 2019

(54) Systemic inflammatory and pathogen biomarkers and uses therefor

(71) Applicant: ImmuneXpress Pty Ltd, Boonash, Queensland (AU)

(72) Inventors: Richard Bruce BRANDON, Boonah (AU); Dayle Lorand SAMPSON, Shoreline, WA (US); Leo Charles MCHUGH, Seattle, WA (US)

(21) Appl. No.: 16/327,687

(22) PCT Filed: Aug. 24, 2017
(86) PCT No.: PCT/AU2017/050894

§ 371 Date: Feb. 22, 2019

(30) Foreign Application Priority Data
Aug. 24, 2016 (AU) 2016903370

(51) Int. Cl. C12Q 1/689; C12Q 1/6883; C12Q 1/6893; C12Q 1/70

(52) U.S. Cl. CPC C12Q 1/689; C12Q 1/6883; C12Q 2600/158; C12Q 1/701; C12Q 1/6893

(57) ABSTRACT

Disclosed are compositions, methods and apparatus for diagnosing and/or monitoring an infection by a bacterium, virus or protozoan by measurement of pathogen-associated and non-infectious systemic inflammation and optionally in combination with detection of a pathogen specific molecule. The invention can be used for diagnosis, including early diagnosis, ruling-out, ruling-in, monitoring, making treatment decisions, or management of subjects suspected of, or having, systemic inflammation. More particularly, the present disclosure relates to host peripheral blood RNA and protein biomarkers, which are used in combination, and optionally with peripheral blood broad-range pathogen-specific detection assays, that are useful for distinguishing between bacterial, viral, protozoal and non-infectious causes of systemic inflammation.

### FIELD OF THE INVENTION

This application claims priority to Australian Provisional Application No. 2016903370 entitled “Systemic inflammatory and pathogen biomarkers and uses therefor” filed 24 Aug. 2016, the contents of which are incorporated herein by reference in their entirety.

This invention relates generally to compositions, methods and apparatus for diagnosing and/or monitoring an infection by a bacterium, virus or protozoan by measurement of pathogen-associated and non-infectious systemic inflammation and optionally in combination with detection of a pathogen specific molecule. The invention can be used for diagnosis, including early diagnosis, ruling-out, ruling-in, monitoring, making treatment decisions, or management of subjects suspected of, or having, systemic inflammation. More particularly, the present invention relates to host peripheral blood RNA and protein biomarkers, which are used in combination, and optionally with peripheral blood broad-range pathogen-specific detection assays, that are useful for distinguishing between bacterial, viral, protozoal and non-infectious causes of systemic inflammation.

### BACKGROUND OF THE INVENTION

Fever and clinical signs of systemic inflammation (or SIRS) are commonly seen in patients presenting to medical services; either in general practice clinics, outpatient clinics, emergency rooms, hospital wards or intensive care units (Rangel-Frausto et al. (1995). The natural history of the systemic inflammatory response syndrome (SIRS). A prospective study. JAMA: the Journal of the American Medical Association, 273(2), 117-123; McGowan et al. (1987). Fever in hospitalized patients. With special reference to the medical service. The American Journal of Medicine, 82(3 Spec No), 580-586; Bor et al. (1988). Fever in hospitalized medical patients: characteristics and significance. Journal of General Internal Medicine, 3(2), 119-125; Finkelstein et al. (2000). Fever in pediatric primary care: occurrence, management, and outcomes. Pediatrics, 105(1 Pt 3), 260-266).

When SIRS is the result of a confirmed infectious process it is called infection-positive SIRS (ipSIRS), otherwise known as sepsis. Within this definition lies the following assumptions; the infectious process could be local or generalized; the infection could be bacterial, viral or parasitic; the infectious process could be in an otherwise sterile body compartment. Such a definition has been updated in Levy et al. 2003 (“2001 SCCM/ESICM/ACCP/ATS/SIS International Sepsis Definitions Conference,” Critical Care Medicine 31, no. 4: 1250-1256) to accommodate clinical and research use of the definition. The revised definition allowed that the infection be in a sterile or non-sterile site (e.g., overgrowth of a pathogen/commensal in the intestine) and that the infection can be either confirmed or suspected. More recently, the definition of sepsis has been updated to be a “life-threatening organ dysfunction caused by a dysregulated host response to infection” (Singer, M., Deutschman, C. S., Seymour, C. W., Shankar-Hari, M., Annane, D., Bauer, M., et al. (2016). The Third International Consensus Definitions for Sepsis and Septic Shock (Sepsis-3). JAMA: the Journal of the American Medical Association, 315(8), 801-10).

In many instances the use of the terms SIRS and sepsis, their changing definitions, and what clinical conditions they do or do not include, are confusing in clinical situations. Such confusion leads to difficulties in clinical diagnosis and in making decisions on subsequent patient treatment and management. Difficulties in clinical diagnosis are based on the following questions: 1) what constitutes a “suspected” infection given that many body organs/sites are naturally colonized by microbes (e.g., Escherichia coli in the intestines, Staphylococcus epidermidis in skin), viruses (e.g., latent viruses such as herpes, resident human rhinovirus in otherwise healthy children) or parasites (e.g., Toxoplasma, Giardia); 2) what constitutes a pathological growth of an organism in a normally non-sterile body site?; 3) what contributions to SIRS are made by a bacterial/viral/parasitic co-infection in a non-sterile body site (e.g., upper respiratory tract), and if such an infection is suspected then should the patient be put on antibiotics, anti-viral or anti-parasitic compounds?

Patients with fever and other clinical signs of SIRS need to be carefully assessed, and tested, to determine the cause of the presenting clinical signs as there are many possible differential diagnoses (Munro, N. (2014). Fever in acute and critical care: a diagnostic approach. AACN Adv Crit Care 25: 237-248). Possible, non-limiting, differential diagnoses include infection (bacterial, viral, parasitic), trauma, allergy, drug reaction, autoimmunity, surgery, neutropenia, cancer, metabolic disorders, clotting disorders.

Patients with fever and SIRS caused by bacterial infection often require immediate medical attention and it is therefore important to quickly and accurately differentiate such patients.

Patients with fever and SIRS caused by viral infection need to be further assessed to determine 1) the degree of systemic inflammation due to viral infection, 2) the degree of involvement of microbes (commensals, microbiome, pathogens) to systemic inflammation 3) contributions that each of viruses, microbes and sterile injury are making to systemic inflammation 4) likelihood of the patient rapidly deteriorating.

Patients with fever and SIRS caused by a protozoal infection (e.g., malaria) also need to be further assessed to determine 1) the degree of systemic inflammation due to protozoal infection, 2) the degree of involvement of other microbes (commensals, microbiome, bacterial or viral pathogens) to systemic inflammation 3) contributions that each of protozoans, viruses, microbes and sterile injury are making to systemic inflammation 4) likelihood of the patient rapidly deteriorating.

The results of such an assessments aids clinicians in making appropriate management and treatment decisions. Appropriate patient management and treatment decisions leads to lower mortality, shorter hospital stays, less use of medical resources and better patient outcomes.

For the purposes of the present disclosure the following definitions are used: Bacterial associated SIRS (BaSIRS) is a condition of a patient with systemic inflammation due to bacterial infection; Viral associated SIRS (VaSIRS) is a condition of a patient with systemic inflammation due to a viral infection; Protozoal associated SIRS (PaSIRS) is a condition of a patient with systemic inflammation due to a protozoal infection; infection-negative SIRS (InSIRS) is a condition of a patient with systemic inflammation due to non-infectious causes. Patients with the conditions BaSIRS, VaSIRS, PaSIRS or InSIRS all have systemic inflammation or SIRS. BaSIRS, VaSIRS, PaSIRS and InSIRS biomarkers refer to specific host response biomarkers associated with the conditions of BaSIRS, VaSIRS, PaSIRS and InSIRS, respectively. Bacterial Infection Positive (BIP), Viral Infection Positive (VIP) and Protozoal Infection Positive (PIP) conditions are conditions of patients with detectable bacterial, viral or parasitic molecules respectively. Bacterial Infection Negative (BIN), Viral Infection Negative (VIN) and Protozoal Infection Negative (PIN) conditions are conditions of patients with non-detectable bacterial, viral or parasitic molecules respectively. BIP, VIP and PIP biomarkers refers to biomarkers that are specific to pathogen molecules as determined by the use of bacterial, viral or protozoal molecule detection assays. Collectively, BaSIRS, VaSIRS, PaSIRS and InSIRS biomarkers are referred to as “host response specific biomarkers.” BIP, VIP and PIP biomarkers are referred to as “pathogen specific biomarkers”. Patients that present with clinical signs of SIRS can be pathogen specific biomarker positive or negative. Thus, patients can be: BaSIRS/BIP, BaSIRS/BIN, VaSIRS/VIP, VaSIRS/VIN, PaSIRS/PIP, PaSIRS/PIN, InSIRS/BIP, InSIRS/BIN, InSIRS/VIP, InSIRS/VIN, InSIRS/PIP, InSIRS/PIN. Suitably, various biomarkers for each of the conditions can found in higher or lower amounts or be detected or not. The results of host response specific biomarker assays and pathogen specific biomarker assays can be combined creating a BaSIRS, VaSIRS, PaSIRS or InSIRS “indicator”.

Whether or not a host responds to a pathogen infection or insult through a SIRS depends largely upon the extent and type of exposure to antigen(s) (PAMPs) or damage associated molecular patterns (DAMPs) (Klimpel G R. Immune Defenses. In: Baron S, editor. Medical Microbiology. 4th edition. Galveston (Tex.): University of Texas Medical Branch at Galveston; 1996. Chapter 50). Factors that affect host immune system exposure to PAMPs and DAMPs include; 1) Host immune status, including vaccination, 2). Primary or secondary exposure to the same antigen(s) or antigen class or DAMPs, 3). Stage of infection or insult (early, late, re-activation, recurrence), 4). Infection type (intracellular, cytolytic, persistent, latent, integrated), 5). Mechanism of infection spread within the host (primary hematogenous, secondary hematogenous, local, nervous), 6). Pathogen or insult location (systemic or restricted to mucosal surface or a tissue/organ).

There are a limited number of microorganisms (bacteria, yeast, viruses, protozoans) that cause disease in humans and an even fewer number cause the majority of infectious diseases. TABLE 1 lists common bacterial, viral and protozoal pathogens associated with human BaSIRS, VaSIRS and PaSIRS, respectively. Such pathogens have multiple methods of interacting with the host and its cells and if a host mounts a systemic inflammatory response to an infection it means that the immune system has been exposed to sufficient levels of novel pathogen molecules. Representative types of pathogen molecules that can elicit a systemic inflammatory response include proteins, nucleic acids (RNA and/or DNA), lipoproteins, lipoteichoic acid and lipopolysaccharides, many of which can be detected (and typed) circulating in blood at some stage during the disease pathogenesis.

Many pathogen molecules are specific to a particular type of pathogen and the host immune system will respond in a specific, adaptive, and usually delayed, manner. However, it is known that there are host receptors, called pattern recognition receptors (PRR), for foreign (microbial, viral, protozoal) antigens (Perry, A. K., Chen, G., Zheng, D., Tang, H., & Cheng, G. (2005). The host type I interferon response to viral and bacterial infections. Cell Research, 15(6), 407-422; Gazzinelli R T, Kalantari P, Fitzgerald K A, Golenbock D T. Innate sensing of malaria parasites. Nat Rev Immunol. 2014 November; 14(11):744-57). PRRs recognise, in a non-specific manner, conserved molecular motifs called Pathogen Associated Molecular Patterns, or PAMPs. The cellular pathways and conserved response to PRR stimulation are well documented and includes the production of Type I interferons (Type I IFNs), tumor necrosis factor (TNF) and interleukins. Whilst different pathogens may use different initial receptors they activate common downstream molecules which ultimately leads to the production of Type I IFNs, IFN and interleukins. The variable downstream effects of these cytokine molecules are dependent upon a number of factors including cell source, concentration, receptor density, receptor avidity and affinity, cell type (Hall, J. C., & Rosen, A. (2010). Type I interferons: crucial participants in disease amplification in autoimmunity. Nature Reviews Rheumatology, 6(1), 40-49; Wajant, H., Pfizenmaier, K., & Scheurich, P. (2003). Tumor necrosis factor signaling. Cell Death and Differentiation, 10(1), 45-65). Accordingly, the host immune system responds to a pathogenic infection in both a generalized (often innate) and specific (often adaptive) manner.

The purported “gold standard” of diagnosis for bacterial infection is culture (growth of an organism and partial or complete identification by staining or biochemical or serological assays). Thus, confirmation of a diagnosis of BaSIRS requires isolation and identification of live bacteria from blood or tissue or body fluid samples using culture, but this technique has its limitations (Thierry Calandra and Jonathan Cohen, “The International Sepsis Forum Consensus Conference on Definitions of Infection in the Intensive Care Unit,” Critical Care Medicine 33, no. 7 (July 2005): 1538-1548; R Phillip Dellinger et al., “Surviving Sepsis Campaign: International Guidelines for Management of Severe Sepsis and Septic Shock: 2008.,” vol. 36, 2008, 296-327, doi:10.1097/01.CCM.0000298158.12101.41). Bacterial culture usually takes a number of days to obtain a positive result and over five days (up to a month) to confirm a negative result. A positive result confirms bacteremia if the sample used was whole blood. However, blood culture is insufficiently reliable with respect to sensitivity, specificity and predictive value, failing to detect a clinically determined ‘bacterial’ cause of fever in 60-80% of patients with suspected primary or secondary bloodstream infection, and in many instances the organism grown is a contaminant (Müller, B., Schuetz, P. & Trampuz, A. Circulating biomarkers as surrogates for bloodstream infections. International Journal of Antimicrobial Agents 30, 16-23 (2007); Jean-Louis Vincent et al., Sepsis in European Intensive Care Units: Results of the SOAP Study, Critical Care Medicine 34, no. 2 (February 2006): 344-353; Brigitte Lamy et al., What Is the Relevance of Obtaining Multiple Blood Samples for Culture? A Comprehensive Model to Optimize the Strategy for Diagnosing Bacteremia, Clinical Infectious Diseases: an Official Publication of the Infectious Diseases Society of America 35, no. 7 (Oct. 1, 2002): 842-850; M D Aronson and D H Bor, Blood Cultures”, Annals of Internal Medicine 106, no. 2 (February 1987): 246-253); Bates, D. W., Goldman, L. & Lee, T. H. Contaminant blood cultures and resource utilization. The true consequences of false-positive results. JAMA 265, 365-369 (1991)). Potential consequences of the diagnostic limitations of bacterial culture in patients suspected of having BaSIRS include; the overuse and misuse of broad-spectrum antibiotics, the development of antimicrobial resistance and Clostridium difficile infection, adverse drug reactions, and increased treatment and testing costs. Antimicrobial resistance is becoming a significant problem in critical care patient management, particularly with Gram-negative bacilli (Hotchkiss and Donaldson. 2006, Nature Reviews Immunology 6:813-822; Eber et al., 2010, Arch Intern Med. 170(4):374-353). Recent evidence suggests that indiscriminate use of antibiotics has contributed to resistance and hence guidance on antibiotic treatment duration is now imperative in order to reduce consumption in tertiary care ICU settings (Hanberger et al., 1999, JAMA. 281:61-71). Molecular nucleic acid-based tests have been developed to detect the major sepsis-causing bacterial pathogens in whole blood from patients with suspected sepsis (e.g., SeptiFast® from Roche, Iridica® from Abbott, Sepsis Panel from Biofire (Biomerieux), Prove-it® Sepsis from Mobidiag). Whilst sensitive and specific, such assays have limitations, especially with respect to clinical interpretation of assay results for suspected sepsis patients that are 1) PCR or assay positive and blood culture negative, and 2) PCR or assay negative (Bauer M, Reinhart K (2010) Molecular diagnostics of sepsis—Where are we today? International Journal of Medical Microbiology 300: 411-413). Thus, blood culture, at least in the minds of clinicians, remains the gold standard for diagnosis of sepsis (BaSIRS) because the results of molecular pathogen detection assays are difficult to interpret in isolation.

Currently, diagnosis of viral conditions is challenging. In general, the conventional method for diagnosing viral infection is cell culture and isolation (growth of virus in cell culture, observation of cytopathic effect (CPE) or hemadsorption (HAD), and partial or complete identification by staining or biochemical or immunoassay (e.g., immunofluorescence)) (Hsiung, G. D. 1984. Diagnostic virology: from animals to automation. Yale J. Biol. Med. 57:727-733; Leland D S, Ginocchio C C (2007) Role of Cell Culture for Virus Detection in the Age of Technology. Clinical Microbiology Reviews 20: 49-78). This method has limitations in that it requires; appropriate transport of the clinical sample in an appropriate virus-preservation medium, an initial strong suspicion of what the infecting virus might be (to select a suitable cell line that will grow the suspected virus), a laboratory having suitable expertise, equipment and cell lines, and, once these conditions are all in place, a lengthy incubation period (days to weeks) to grow the virus. The process is laborious and expensive.

With respect to improving the diagnosis of viral conditions, and more recently, sensitive and specific assays such as those using monoclonal antibodies or nucleic acid amplification have become available and are now widely available and used in diagnostic laboratories. Amplification of viral DNA and RNA (e.g., PCR) and viral antigen detection are fast and do not require the lengthy incubation period needed for viral isolation in cell cultures, may involve less technical expertise, and are sensitive enough to be useful for viruses that do not proliferate in standard cell cultures. Molecular detection of viral DNA and RNA also has its limitations in that an initial strong suspicion of what the infecting virus might be is also required (to use specific PCR primers and probes, for example), the method detects both live and dead virus, and most molecular tests are designed to detect only one type of virus and, as such, will only detect one type of virus. By way of example, it has been shown that mixed respiratory infections occur in up to 15% of immunocompetent children and that such mixed infections lead to an increase in disease severity (Waner, J. L. 1994. Mixed viral infections: detection and management. Clin. Microbiol. Rev. 7:143-151). A PCR designed to only one type of virus will not detect a mixed infection if the primers and probes are not specific to all viruses present in the clinical specimen. To cover the possibility of a mixed infection, as well as to cover multiple possible viral causes or strains, there are some commercially available assays capable of detecting more than one virus and/or strain at a time (e.g., BioMerieux, BioFire, FilmArray®, Respiratory Panel; Luminex, xTAG® Respiratory Viral Panel). Such an approach is especially useful in confirming an infective agent if clinical signs are pathognomonic or if a particular body system is affected (e.g., respiratory tract or gastrointestinal tract). Further, there are techniques that allow for amplification of viral DNA of unknown sequence which could be useful in situations where the clinical signs are generalized, for viruses with high mutation rates, for new and emerging viruses, or for detecting biological weapons of man-made nature (Clem et al. (2007) Virus detection and identification using random multiplex (RT)-PCR with 3′-locked random primers. Virol J 4: 65; Liang et al. (1992) Differential display of eukaryotic messenger RNA by means of the polymerase chain reaction. Science 257(5072):967-971; Nie X et al. (2001) A novel usage of random primers for multiplex RT-PCR detection of virus and viroid in aphids, leaves, and tubers. J Virol Methods 91(1):37-49; Ralph et al. (1993) RNA fingerprinting using arbitrarily primed PCR identifies differentially regulated RNAs in mink lung (Mv1Lu) cells growth arrested by transforming growth factor beta 1. Proc Natl Acad Sci USA 90(22):10710-10714.). Further, a microarray has been designed to detect every known virus for which there is DNA sequence information in GenBank (called “Virochip”) (Greninger, A. L., Chen, E. C., Sittler, T., Scheinerman, A., Roubinian, N., Yu, G., et al. (2010). A metagenomic analysis of pandemic influenza A (2009 H1N1) infection in patients from North America. PLoS ONE, 5(10), e13381; Chiu C Y, Greninger A L, Kanada K, Kwok T, Fischer K F, et al. (2008) Identification of cardioviruses related to Theiler's murine encephalomyelitis virus in human infections. Proc Natl Acad Sci USA 105: 14124-14129). The use of such a microarray for diagnostic purposes in human patients presenting with clinical signs of SIRS is perhaps superfluous since there is only a limited number of human viruses that are known to cause SIRS (see TABLES 1 and 2). However, a more directed microarray using just those human viruses that are known to cause SIRS could be used for the purpose outlined in this patent.

It has been shown that the use of molecular detection methods, compared to conventional detection methods, in patients with lower respiratory tract infections did not significantly change the treatment regimen but led to an overall increase in cost of patient management (Oosterheert J J, van Loon A M, Schuurman R, Hoepelman A I M, Hak E, et al. (2005) Impact of rapid detection of viral and atypical bacterial pathogens by real-time polymerase chain reaction for patients with lower respiratory tract infection. Clinical Infectious Diseases 41: 1438-1444). Thus, the availability of faster and more sensitive molecular detection assays for pathogens does not necessarily positively impact clinical decision making, patient outcome, antibiotic use, adoption or hospital econometrics. Further, pathogen detection assays for viruses have limitations in that the results are often difficult to interpret in a clinical context when used in isolation. Thus, the diagnosis of a viral infection, and if a virus is isolated or identified whether it is pathogenic or not, cannot always be made simply by determining the presence of such an organism in a host sample.

In some instances, detection of host antibodies to an infecting virus remains the diagnostic gold standard, because either the virus cannot be grown, or the presence of virus in a biological fluid is transient (e.g., arboviral infections) and therefore cannot be detected at times when the patient is symptomatic. Antibody detection also has limitations including: it usually takes at least 10 days for a host to generate detectable and specific immunoglobulin G antibodies in a primary infection, by which time the clinical signs have often abated; anti-viral antibodies following a primary infection can persist for a long period making it difficult to interpret the timing of an infection relapse for viruses that show latency; a specific test must be ordered to detect a specific virus. These limitations make it difficult to determine when the host was infected, whether high antibody titers to a particular virus means that a particular virus is the causative agent of the presenting clinical signs, and which test to order. In some instances the ratio of IgM to IgG antibodies can be used to determine the recency of virus infection. IgM is usually produced early in the immune response and is non-specific, whereas IgG is produced later in the immune response and is specific. Examples of the use of this approach include the diagnosis of hepatitis E (Tripathy et al. (2012). Cytokine Profiles, CTL Response and T Cell Frequencies in the Peripheral Blood of Acute Patients and Individuals Recovered from Hepatitis E Infection. PLoS ONE, 7(2), e31822), dengue (SA-Ngasang et al. (2005). Specific IgM and IgG responses in primary and secondary dengue virus infections determined by enzyme-linked immunosorbent assay. Epidemiology and Infection, 134(04), 820), and Epstein-Barr Virus (Hess, R. D. (2004). Routine Epstein-Barr Virus Diagnostics from the Laboratory Perspective: Still Challenging after 35 Years. Journal of Clinical Microbiology, 42(8), 3381-3387). The IgM/IgG ratio approach also suffers from the limitation that the clinician must know which specific test to order a priori.

Parasitic diseases place a heavy burden on human health worldwide with the majority of people affected living in developing countries. However, protozoan parasites are the most common parasitic infection and affect humans irrespective of whether they live in a first or third world country as more and more people become immunocompromised as a result of human immunodeficiency virus (HIV) infection, organ transplant or chemotherapy (Stark D, Barratt J L N, van Hal S, Marriott D, Harkness J, et al. (2009) Clinical Significance of Enteric Protozoa in the Immunosuppressed Human Population. Clinical Microbiology Reviews 22: 634-650). Common and well-known protozoan human pathogens include Plasmodium (malaria), Leishmania (leishmaniasis), Trypanosoma (sleeping sickness and Chagas disease), Cryptosporidium, Giardia, Toxoplasma, Babesia, Balantidium and Entamoeba. Common and well-known protozoan human pathogens that can be found in peripheral blood (causing a parasitemia) include Plasmodium falciparum, Plasmodium ovale, Plasmodium malariae, Plasmodium vivax, Leishmania donovani, Trypanosoma brucei, Trypanosoma cruzi, Toxoplasma gondii and Babesia microti. Diagnosis of protozoal infections is achieved by pathogen detection using a variety of methods including light microscopy, or antigen or nucleic acid detection using different techniques such as tissue biopsy and histology, fecal or blood smears and staining, ELISA, lateral flow immunochromatography, and nucleic acid amplification. These methods of diagnosis have limitations including the fact that they often require special stains and skilled personnel, the sample taken has to have the parasite present, and often the parasite is opportunistic, meaning that many people are carriers of such parasites and do not show clinical signs until their immune system is compromised. As a result, such pathogen detection assays for protozoan parasites are difficult to interpret in a clinical context when used in isolation.

Diagnosis of non-infectious SIRS is often by default—that is, elimination of an infection as a cause of SIRS.

Thus, the diagnosis of a bacterial, viral or parasitic infection, and if an organism is isolated or identified, whether it is pathogenic or not, cannot always be made simply by determining the presence of such an organism in a host sample.

In the absence of a gold standard assay for diagnosis of a condition a combination of tests or parameters, or the use of a group of experts, can be used (Hui, S. L. and X. H. Zhou (1998). Evaluation of diagnostic tests without gold standards. Statistical Methods in Medical Research 7(4), 354-370; Zhang, B., Chen, Z. & Albert, P. S. Estimating diagnostic accuracy of raters without a gold standard by exploiting a group of experts. Biometrics 68, 1294-1302 (2012); Reitsma, J. B., Rutjes, A. W. S., Khan, K. S., Coomarasamy, A. & Bossuyt, P. M. A review of solutions for diagnostic accuracy studies with an imperfect or missing reference standard. J Clin Epidemiol 62, 797-806 (2009)). In the absence of a gold standard test for BaSIRS a clinical diagnosis is provided by the physician(s) at the time the patient presents and in the absence of any results from diagnostic tests. This is done in the interests of rapid treatment and positive patient outcomes. Such an approach has proven to be reasonably reliable (AUC ˜0.88) in children but only with respect to differentiating between patients ultimately shown to be blood culture positive and those that were judged to be unlikely to have an infection at the time antibiotics were administered (Fischer, J. E. et al. Quantifying uncertainty: physicians' estimates of infection in critically ill neonates and children. Clin. Infect. Dis. 38, 1383-1390 (2004)). In Fischer et al., (2004), 54% of critically ill children were put on antibiotics during their hospital stay, of which only 14% and 16% had proven systemic bacterial infection or localized infection respectively. In this study, 53% of antibiotic treatment courses for critically ill children were for those that had an unlikely infection and 38% were antibiotic treatment courses for critically ill children as a rule-out treatment episode. Clearly, pediatric physicians err on the side of caution with respect to treating critically ill patients by placing all suspected BaSIRS patients on antibiotics—38% of all antibiotics used in critically ill children are used on the basis of ruling out BaSIRS, that is, are used as a precaution. The risks of not correctly diagnosing BaSIRS are profound (Dellinger, R. P. et al. Surviving Sepsis Campaign: international guidelines for management of severe sepsis and septic shock: 2008. in Crit. Care Med. 36, 296-327 (2008)). Thus, making a diagnosis of BaSIRS (ruling in) carries much less clinical risk than making a diagnosis of InSIRS (ruling out BaSIRS and VaSIRS and PaSIRS).

Therefore, with respect to correctly diagnosing BaSIRS, blood culture has unacceptably low negative predictive value (NPV), or unacceptably high false negative levels. With respect to correctly diagnosing BaSIRS, clinical diagnosis has unacceptably low positive predictive value (PPV), or unacceptably high false positive levels. In the latter instance the consequence is that many patients are unnecessarily prescribed antibiotics because of 1) the clinical risk of misdiagnosing BaSIRS, 2) the lack of a gold standard diagnostic test, and 3) the fact that blood culture results take too long to provide results that are clinically actionable.

Diagnosis of a viral infection, including VaSIRS, is often done based on presenting clinical signs only. The reasons for this are; most viral infections are not life-threatening, there are few therapeutic interventions available, many viral infections cause the same clinical signs, and most diagnostic assays take too long and are too expensive. The consequence is that many VaSIRS patients are unnecessarily prescribed antibiotics because of the clinical risk of misdiagnosing BaSIRS.

Diagnosis of a parasitic infection, including PaSIRS, is based on presenting clinical signs, detection of the parasite and, in areas with low parasite prevalence, exclusion of more common bacterial and viral causes. The consequence is that many PaSIRS patients are misdiagnosed, diagnosed late in the course of disease progression, or unnecessarily prescribed antibiotics because of the clinical risk of misdiagnosing BaSIRS.

Alternative diagnostic approaches to BaSIRS have been investigated including determination of host response using biomarkers (Michael Bauer and Konrad Reinhart, “Molecular Diagnostics of Sepsis—Where Are We Today?” International Journal of Medical Microbiology 300, no. 6 (Aug. 1, 2010): 411-413, doi:10.1016/j.ijmm.2010.04.006; John C Marshall and Konrad Reinhart, “Biomarkers of Sepsis,” Critical Care Medicine 37, no. 7 (July 2009): 2290-2298, doi:10.1097/CCM.0b013e3181a02afc.). A systematic literature search identified nearly 180 molecules as potential biomarkers of sepsis of which 20% have been assessed in appropriately designed sepsis studies including C-reactive protein (CRP), procalcitonin (PCT), and IL6 (Reinhart, K., Bauer, M., Riedemann, N. C. & Hartog, C. S. New Approaches to Sepsis: Molecular Diagnostics and Biomarkers. Clinical Microbiology Reviews 25, 609-634 (2012)).

Alternative diagnostic approaches to VaSIRS have been investigated including determination of host response using biomarkers to specific viruses (Huang Y, Zaas A K, Rao A, Dobigeon N, Woolf P J, et al. (2011) Temporal Dynamics of Host Molecular Responses Differentiate Symptomatic and Asymptomatic Influenza A Infection. PLoS Genet 7: e1002234; Wang Y, Dennehy P H, Keyserling H L, Tang K, Gentsch J R, et al. (2007) Rotavirus Infection Alters Peripheral T-Cell Homeostasis in Children with Acute Diarrhea. Journal of Virology 81: 3904-3912), and in one instance a common signature to a number of respiratory viruses has been published in two separate scientific papers (Zaas A K, Chen M, Varkey J, Veldman T, Hero A O III, et al. (2009) Gene Expression Signatures Diagnose Influenza and Other Symptomatic Respiratory Viral Infections in Humans. Cell Host & Microbe 6: 207-217; Tsalik, E. L., Henao, R., Nichols, M., Burke, T., Ko, E. R., McClain, M. T., et al. (2016). Host gene expression classifiers diagnose acute respiratory illness etiology. Science Translational Medicine, 8(322), 322ra11-322ra11).

Alternative diagnostic approaches to PaSIRS have been investigated including determination of host response using biomarkers (Ockenhouse C F, Hu W C, Kester K E, Cummings J F, Stewart A, et al. (2006) Common and Divergent Immune Response Signaling Pathways Discovered in Peripheral Blood Mononuclear Cell Gene Expression Patterns in Presymptomatic and Clinically Apparent Malaria. Infection and Immunity 74: 5561-5573; Chaussabel D, Semnani R T, McDowell M A, Sacks D et al. Unique gene expression profiles of human macrophages and dendritic cells to phylogenetically distinct parasites. Blood 2003 Jul. 15; 102(2):672-81).

The acute management plans for patients with BaSIRS, VaSIRS, PaSIRS and InSIRS are different. For best patient outcomes, it is important that those patients who have a suspected infection, or are at high risk of infection, are identified early and graded and monitored in order to initiate evidence-based and goal-orientated medical therapy, including early use of antibiotics, anti-viral or anti-parasitic therapies. An assay that is reliable, fast, and able to determine the presence or absence of a pathogen infection in patients with systemic inflammation will assist clinicians in making appropriate patient management and treatment decisions. In a background of high prevalence of systemic inflammation and unreliable pathogen detection assays, what is needed is a diagnostic assay that combines specific detection of systemic inflammation biomarkers with broad-range pathogen detection assays so that patients presenting with clinical signs of systemic inflammation can be confidently categorized into InSIRS, BaSIRS, VaSIRS and PaSIRS. Patients negative for both pathogen associated SIRS and pathogen detection assays can be “ruled out” as having an infection. Such an assay would have high negative predictive value for systemic pathogen infection which would have high clinical utility by allowing clinicians to confidently withhold therapies, in particular antibiotics. Patients positive for both pathogen associated SIRS and pathogen detection assays can be “ruled in” as having a particular type of infection (or mixed infection). Such an assay would have high positive predictive value for systemic pathogen infection allowing clinicians to confidently manage and treat patients.

Testing for microbes, viruses and parasites requires that clinical samples be taken from patients. Examples of clinical samples include; blood, plasma, serum, cerebrospinal fluid (CSF), stool, urine, tissue, pus, saliva, semen, skin, other body fluids. Examples of clinical sampling methods include; venipuncture, biopsy, scrapings, aspirate, lavage, collection of body fluids and stools into sterile containers. Most clinical sampling methods are invasive (physically or on privacy), or painful, or laborious, or require multiple samplings, or, in some instances, dangerous (e.g., large CSF volumes in neonates). The taking of blood via venipuncture is perhaps the least invasive method of clinical sampling and, in the case of BaSIRS, VaSIRS, PaSIRS and InSIRS, the most relevant. As such, in a background of high prevalence of SIRS, what is needed is a diagnostic assay, based on the use of a peripheral blood sample, with a high predictive value for BaSIRS so that clinicians can confidently rule out, or rule in, a bacterial cause of SIRS.

Therefore, a need exists for better ways of differentiating patients presenting with systemic inflammation to permit early diagnosis, ruling out or ruling in infection, monitoring, and making better treatment and management decisions.

### SUMMARY OF THE INVENTION

In work leading up to the present invention, it was determined that derived biomarker values that are indicative of a ratio of measured biomarkers values (e.g., biomarker levels) provide significantly more diagnostic power than measured biomarker values alone for assessing the likelihood that a particular condition, or degree thereof, is present or absent in a subject (see, WO 2015/117204). The present inventors have now determined that the vast majority of derived biomarker values in peripheral blood cells are shared between patients within different SIRS subgroups (e.g., BaSIRS, VaSIRS, PaSIRS and InSIRS), which suggests, therefore, that there are numerous biochemical pathways that are common to SIRS conditions of different etiology. Accordingly, it was reasoned that it would be necessary to subtract biomarker combinations corresponding to these derived biomarker values (also referred to herein as “derived biomarkers”) from the pool of biomarker combinations to identify derived biomarkers with improved specificity to a particular SIRS condition. Of note, it was also found that exclusion of derived biomarkers belonging to any one particular SIRS subgroup (e.g., PaSIRS) from the pool of derived biomarkers markedly changed the biomarker combinations resulting from the analysis and undermined their specificity for diagnosing individual SIRS conditions.

The present inventors have also determined that derived biomarker values in peripheral blood cells can vary between subjects with different non-SIRS inflammatory conditions including autoimmunity, asthma, stress, anaphylaxis, trauma and obesity, and between subjects of different age, gender and race. This suggests, therefore, that the corresponding derived biomarkers also need to be subtracted from the pool of derived biomarkers to identify biomarker combinations with improved specificity to a SIRS condition of specified etiology.

The present invention is also predicated in part on the identification of derived biomarkers with remarkable specificity to systemic inflammations caused by a range of different viral infections across different mammals (humans, macaques, chimpanzees, pigs, rats, mice). Because such derived biomarkers are specific to systemic inflammations associated with a variety of different types of viruses covering examples from each of the Baltimore classification groups (I-VII), they are considered to be “pan-viral” inflammatory derived biomarkers. To ensure that the derived biomarkers described herein are truly pan-viral and also specific to a viral infection, the following procedures and methods were deliberately performed: 1). A mixture of both DNA and RNA viruses were included in the “discovery” core datasets—only those derived biomarkers with strong performance across all of these datasets were selected for further analysis, 2). A wide range of virus families, including both DNA and RNA viruses, were included in the various “validation” datasets, 3). A wide range of virus families causing a variety of clinical signs were included in the various datasets, 4). Viruses covering all of the Baltimore Classification categories were included in the various datasets, 5). Viruses and samples covering a variety of stage of infection, infection type, mechanism of spread and location were included in the various datasets, 6). Controlled and time-course datasets were selected to cover more than one species of mammal (humans, macaques, chimpanzees, pigs, mice), 7). In time-course studies samples early in the infection process were chosen, prior to peak clinical signs, to limit the possibility of a bacterial co-infection, 8). Derived biomarkers shared with other inflammatory conditions were subtracted (e.g., derived biomarkers for BaSIRS, PaSIRS and InSIRS, as well as derived biomarkers for autoimmunity, asthma, bacterial infections, sarcoidosis, stress, anaphylaxis, trauma, age, obesity, gender and race), 9). Validation was performed in both adults and children with a variety of viral conditions. Following the stringent selection process only those derived biomarkers with an AUC greater than existing virus assays and clinical judgment were selected to ensure clinical utility.

The present inventors further propose that the host response specific derived biomarkers for BaSIRS, VaSIRS, PaSIRS and InSIRS disclosed herein can be used advantageously with pathogen specific biomarkers to augment the diagnosis of the etiological basis of systemic inflammation including determining whether systemic inflammation in a patient is due to a bacterial, viral, or protozoal infection, or due to some other non-infectious cause. The use of a combination of host response derived biomarkers and pathogen-specific biomarkers provides a more definitive diagnosis, especially the ability to either rule out or rule in a particular condition in patients with systemic inflammation, especially in situations where pathogen detection assay results are suspected of being either falsely positive or negative.

Based on the above determinations, the present inventors have developed various methods, apparatus, compositions, and kits, which take advantage of derived biomarkers, and optionally in combination with pathogen-specific detection assays, to determine the etiology, presence, absence or degree of a SIRS condition of a specified etiology (e.g., BaSIRS, VaSIRS, PaSIRS or InSIRS) in subjects presenting with fever or clinical signs of systemic inflammation. In certain embodiments, these methods, apparatus, compositions, and kits represent a significant advance over prior art processes and products, which have not been able to: 1) distinguish the various etiologies of systemic inflammation; and/or 2) determine the contribution of a particular type of infection (if any) to the presenting clinical signs and pathology; and/or 3) determine if an isolated or detected microorganism is a true pathogen, a commensal, a normal component of the microbiome, a contaminant, or an incidental finding. Such a combination of information provides strong positive and negative predictive power, which in turn provides clinicians with the ability to make better informed management and treatment decisions.

Accordingly, in one aspect, the present invention provides methods for determining an indicator used in assessing a likelihood of a subject having a presence, absence or degree of BaSIRS or VaSIRS. These methods generally comprise, consist or consist essentially of: (1) determining a plurality of host response specific biomarker values including a plurality of BaSIRS biomarker values and a plurality of VaSIRS biomarker values, the plurality of BaSIRS biomarker values being indicative of values measured for a corresponding plurality of BaSIRS biomarkers in a sample taken from the subject, the plurality of VaSIRS biomarker values being indicative of values measured for a corresponding plurality of VaSIRS biomarkers in the sample; (2) determining a plurality of host response specific derived biomarker values including at least one BaSIRS derived biomarker value and at least one VaSIRS derived biomarker value, each derived BaSIRS biomarker value being determined using at least a subset of the plurality of BaSIRS biomarker values, and being indicative of a ratio of levels of a corresponding at least a subset of the plurality of BaSIRS biomarkers, and each derived VaSIRS biomarker value being determined using at least a subset of the plurality of VaSIRS biomarker values, and being indicative of a ratio of levels of a corresponding at least a subset of the plurality of VaSIRS biomarkers; and (3) determining the indicator using the plurality of host response specific derived biomarker values, wherein the at least a subset of BaSIRS biomarkers forms a BaSIRS derived biomarker combination which is not a derived biomarker combination for VaSIRS, PaSIRS or InSIRS, and wherein the at least a subset of VaSIRS biomarkers forms a VaSIRS derived biomarker combination which is not a derived biomarker combination for BaSIRS, PaSIRS or InSIRS. Typically, in any of the aspects or embodiments described herein, the subject has at least one clinical sign (e.g., 1, 2, 3, 4, 5 or more) of SIRS.

Suitably, in any aspect or embodiments disclosed herein, the BaSIRS derived biomarker combination and the VaSIRS derived biomarker combination are not derived biomarker combinations for any one or more inflammatory conditions selected from autoimmunity, asthma, stress, anaphylaxis, trauma and obesity. Alternatively, or in addition, the derived BaSIRS biomarkers and derived VaSIRS biomarkers are not derived biomarkers for any one or more of age, gender and race.

In any of the aspects or embodiments disclosed herein, the methods may further comprise: (a) determining a plurality of pathogen specific biomarker values including at least one bacterial biomarker value and at least one viral biomarker value, the least one bacterial biomarker value being indicative of a value measured for a corresponding bacterial biomarker in the sample, the least one viral biomarker value being indicative of a value measured for a corresponding viral biomarker in the sample; and (b) determining the indicator using the host response specific derived biomarker values in combination with the pathogen specific biomarker values. Suitably, in some of these aspects or embodiments, the indicator is also used to rule in or rule out a SIRS condition of a particular etiology. For example, if the plurality of host response specific derived biomarker values indicates the likely presence of a pathogen-associated SIRS condition (e.g., BaSIRS, VaSIRS or InSIRS) in the subject and the pathogen specific biomarker value(s) indicate(s) the likely presence of a pathogen (e.g., bacterium, virus, protozoan) associated with the pathogen-associated SIRS condition in the subject, then the indicator determined using the combination of host response specific derived biomarker values and pathogen specific biomarker value(s) can be used to rule in the pathogen-associated SIRS condition. Alternatively, if the plurality of host response specific derived biomarker values indicates the likely absence of a pathogen-associated SIRS condition (e.g., BaSIRS, VaSIRS or InSIRS) in the subject and the pathogen specific biomarker value(s) indicate(s) the likely absence of a pathogen (e.g., bacterium, virus, protozoan) associated with the pathogen-associated SIRS condition in the subject, then the indicator determined using the combination of host response specific derived biomarker values and pathogen specific biomarker value(s) can be used to rule out the pathogen-associated SIRS condition.

Suitably, in any of the aspects or embodiments disclosed herein, each BaSIRS derived biomarker value is determined using a pair of the BaSIRS biomarker values, and is indicative of a ratio of levels of a corresponding pair of BaSIRS biomarkers. Alternatively, or in addition, each VaSIRS derived biomarker value is determined using a pair of the VaSIRS biomarker values, and is indicative of a ratio of levels of a corresponding pair of VaSIRS biomarkers.

In some embodiments, the plurality of host response specific biomarker values further includes a plurality of PaSIRS biomarker values, the plurality of PaSIRS biomarker values being indicative of values measured for a corresponding plurality of PaSIRS biomarkers in the sample, and the plurality of host response specific derived biomarker values further includes at least one PaSIRS derived biomarker value, and the methods further comprise: determining each PaSIRS derived biomarker value using at least a subset of the plurality of PaSIRS biomarker values, the PaSIRS derived biomarker value being indicative of a ratio of levels of a corresponding at least a subset of the plurality of PaSIRS biomarkers; and determining the indicator using the plurality of host response specific derived biomarker values, wherein the at least a subset of PaSIRS biomarkers forms a PaSIRS derived biomarker combination which is not a derived biomarker combination for BaSIRS, VaSIRS or InSIRS.

Suitably, in any of the aspects or embodiments disclosed herein, each PaSIRS derived biomarker value is determined using a pair of the PaSIRS biomarker values, and is indicative of a ratio of levels of a corresponding pair of PaSIRS biomarkers.

In a related aspect, the present invention provides methods for determining an indicator used in assessing a likelihood of a subject having a presence, absence or degree of BaSIRS, VaSIRS or PaSIRS. These methods generally comprise, consist or consist essentially of: (1) determining a plurality of host response specific biomarker values including a plurality of BaSIRS biomarker values, a plurality of VaSIRS biomarker values, and a plurality of PaSIRS biomarker values, the plurality of BaSIRS biomarker values being indicative of values measured for a corresponding plurality of BaSIRS biomarkers in a sample taken from the subject, the plurality of VaSIRS biomarker values being indicative of values measured for a corresponding plurality of VaSIRS biomarkers in the sample, the plurality of PaSIRS biomarker values being indicative of values measured for a corresponding plurality of PaSIRS biomarkers in the sample; (2) determining a plurality of host response specific derived biomarker values including at least one BaSIRS derived biomarker value, at least one VaSIRS derived biomarker value, and at least one PaSIRS derived biomarker value, each derived BaSIRS biomarker value being determined using at least a subset of the plurality of BaSIRS biomarker values, and being indicative of a ratio of levels of a corresponding at least a subset of the plurality of BaSIRS biomarkers, each derived VaSIRS biomarker value being determined using at least a subset of the plurality of VaSIRS biomarker values, and being indicative of a ratio of levels of a corresponding at least a subset of the plurality of VaSIRS biomarkers, and each derived PaSIRS biomarker value being determined using at least a subset of the plurality of PaSIRS biomarker values, and being indicative of a ratio of levels of a corresponding at least a subset of the plurality of PaSIRS biomarkers; and (3) determining the indicator using the plurality of host response specific derived biomarker values, wherein the at least a subset of BaSIRS biomarkers forms a BaSIRS derived biomarker combination which is not a derived biomarker combination for VaSIRS, PaSIRS or InSIRS, wherein the at least a subset of VaSIRS biomarkers forms a VaSIRS derived biomarker combination which is not a derived biomarker combination for BaSIRS, PaSIRS or InSIRS, and wherein the at least a subset of PaSIRS biomarkers forms a PaSIRS derived biomarker combination which is not a derived biomarker combination for BaSIRS, VaSIRS or InSIRS.

In some embodiments, the methods further comprise: (a) determining a plurality of pathogen specific biomarker values including at least one bacterial biomarker value, at least one viral biomarker value and at least one protozoal biomarker value, the at least one bacterial biomarker value being indicative of a value measured for a corresponding bacterial biomarker in the sample, the least one viral biomarker value being indicative of a value measured for a corresponding viral biomarker in the sample, and the least one protozoal biomarker value being indicative of a value measured for a corresponding protozoal biomarker in the sample; and (b) determining the indicator using the host response specific derived biomarker values in combination with the pathogen specific biomarker values.

In some embodiments of any of the aspects disclosed herein, the plurality of host response specific biomarker values further includes a plurality of InSIRS biomarker values, the plurality of InSIRS biomarker values being indicative of values measured for a corresponding plurality of InSIRS biomarkers in the sample, and the plurality of host response specific derived biomarker values further includes at least one InSIRS derived biomarker value, and the methods further comprise: determining each InSIRS derived biomarker value using at least a subset of the plurality of InSIRS biomarker values, the InSIRS derived biomarker value being indicative of a ratio of levels of a corresponding at least a subset of the plurality of InSIRS biomarkers; and determining the indicator using the plurality of host response specific derived biomarker values, wherein the at least a subset of InSIRS biomarkers forms a InSIRS derived biomarker combination which is not a derived marker combination for BaSIRS, VaSIRS or PaSIRS.

Accordingly, in a related aspect, the present invention provides methods for determining an indicator used in assessing a likelihood of a subject having a presence, absence or degree of BaSIRS, VaSIRS or InSIRS. These methods generally comprise, consist or consist essentially of: (1) determining a plurality of host response specific biomarker values including a plurality of BaSIRS biomarker values, a plurality of VaSIRS biomarker values, and a plurality of InSIRS biomarker values, the plurality of BaSIRS biomarker values being indicative of values measured for a corresponding plurality of BaSIRS biomarkers in a sample taken from the subject, the plurality of VaSIRS biomarker values being indicative of values measured for a corresponding plurality of VaSIRS biomarkers in the sample, the plurality of InSIRS biomarker values being indicative of values measured for a corresponding plurality of InSIRS biomarkers in the sample; (2) determining a plurality of host response specific derived biomarker values including at least one BaSIRS derived biomarker value, at least one VaSIRS derived biomarker value, and at least one InSIRS derived biomarker value, each derived BaSIRS biomarker value being determined using at least a subset of the plurality of BaSIRS biomarker values, and being indicative of a ratio of levels of a corresponding at least a subset of the plurality of BaSIRS biomarkers, each derived VaSIRS biomarker value being determined using at least a subset of the plurality of VaSIRS biomarker values, and being indicative of a ratio of levels of a corresponding at least a subset of the plurality of VaSIRS biomarkers, and each derived InSIRS biomarker value being determined using at least a subset of the plurality of InSIRS biomarker values, and being indicative of a ratio of levels of a corresponding at least a subset of the plurality of InSIRS biomarkers; and (3) determining the indicator using the plurality of host response specific derived biomarker values, wherein the at least a subset of BaSIRS biomarkers forms a BaSIRS derived biomarker combination which is not a derived biomarker combination for VaSIRS, PaSIRS or InSIRS, wherein the at least a subset of VaSIRS biomarkers forms a VaSIRS derived biomarker combination which is not a derived biomarker combination for BaSIRS, PaSIRS or InSIRS, and wherein the at least a subset of InSIRS biomarkers forms an InSIRS derived biomarker combination which is not a derived biomarker combination for BaSIRS, VaSIRS or PaSIRS.

In still another related aspect, the present invention provides methods for determining an indicator used in assessing a likelihood of a subject having a presence, absence or degree of BaSIRS, VaSIRS, PaSIRS or InSIRS. These methods generally comprise, consist or consist essentially of: (1) determining a plurality of host response specific biomarker values including a plurality of BaSIRS biomarker values, a plurality of VaSIRS biomarker values, a plurality of PaSIRS biomarker values, and a plurality of InSIRS biomarker values, the plurality of BaSIRS biomarker values being indicative of values measured for a corresponding plurality of BaSIRS biomarkers in a sample taken from the subject, the plurality of VaSIRS biomarker values being indicative of values measured for a corresponding plurality of VaSIRS biomarkers in the sample, the plurality of PaSIRS biomarker values being indicative of values measured for a corresponding plurality of PaSIRS biomarkers in the sample, the plurality of InSIRS biomarker values being indicative of values measured for a corresponding plurality of InSIRS biomarkers in the sample; (2) determining a plurality of host response specific derived biomarker values including at least one BaSIRS derived biomarker value, at least one VaSIRS derived biomarker value, at least one PaSIRS derived biomarker value, and at least one InSIRS derived biomarker value, each derived BaSIRS biomarker value being determined using at least a subset of the plurality of BaSIRS biomarker values, and being indicative of a ratio of levels of a corresponding at least a subset of the plurality of BaSIRS biomarkers, each derived VaSIRS biomarker value being determined using at least a subset of the plurality of VaSIRS biomarker values, and being indicative of a ratio of levels of a corresponding at least a subset of the plurality of VaSIRS biomarkers, each derived PaSIRS biomarker value being determined using at least a subset of the plurality of PaSIRS biomarker values, and being indicative of a ratio of levels of a corresponding at least a subset of the plurality of PaSIRS biomarkers, and each derived InSIRS biomarker value being determined using at least a subset of the plurality of InSIRS biomarker values, and being indicative of a ratio of levels of a corresponding at least a subset of the plurality of InSIRS biomarkers; and (3) determining the indicator using the plurality of host response specific derived biomarker values, wherein the at least a subset of BaSIRS biomarkers forms a BaSIRS derived biomarker combination which is not a derived biomarker combination for VaSIRS, PaSIRS or InSIRS, wherein the at least a subset of VaSIRS biomarkers forms a VaSIRS derived biomarker combination which is not a derived biomarker combination for BaSIRS, PaSIRS or InSIRS, wherein the at least a subset of PaSIRS biomarkers forms a PaSIRS derived biomarker combination which is not a derived biomarker combination for BaSIRS, VaSIRS or InSIRS, and wherein the at least a subset of InSIRS biomarkers forms an InSIRS derived biomarker combination which is not a derived biomarker combination for BaSIRS, VaSIRS or PaSIRS.

Suitably, in any of the embodiments or aspects disclosed herein, the indicator is determined by combining a plurality (e.g., 2, 3, 4, 5, 6, 7, 8, etc.) of derived biomarker values. For example, the methods may comprise combining the derived biomarker values using a combining function, wherein the combining function is at least one of: an additive model; a linear model; a support vector machine; a neural network model; a random forest model; a regression model; a genetic algorithm; an annealing algorithm; a weighted sum; a nearest neighbor model; and a probabilistic model.

Exemplary BaSIRS derived biomarker combinations can be selected from TABLE A.

TABLE A
BaSIRS Derived Biomarkers

| | | | |
|---|---|---|---|
| PDGFC:KLRF1 | GAS7:GAB2 | PDGFC:LPIN2 | GALNT2:IK |
| TMEM165:PARP8 | PDGFC:INPP5D | TSPO:NLRP1 | CD82:JARID2 |
| ITGA7:KLRF1 | ST3GAL2:PRKD2 | PCOLCE2:NMUR1 | PDGFC:ICK |
| CR1:GAB2 | HK3:INPP5D | FAM129A:GAB2 | GALNT2:SAP130 |
| PCOLCE2:KLRF1 | ENTPD7:KLRD1 | ALPL:NLRP1 | PDGFC:FBXO28 |
| ITGA7:INPP5D | PDGFC:SIDT1 | TSPO:ZFP36L2 | TSPO:GAB2 |
| GALNT2:CCNK | PDGFC:SPIN1 | ALPL:ZFP36L2 | COX15:INPP5D |
| PDGFC:KLRD1 | PCOLCE2:YPEL1 | PCOLCE2:FOXJ3 | ITGA7:LAG3 |
| PDGFC:CCNK | PDGFC:SYTL2 | PDGFC:KIAA0355 | TSPO:CAMK1D |
| CR1:ADAM19 | PDGFC:TGFBR3 | PDGFC:KIAA0907 | OPLAH:POGZ |
| ITGA7:CCNK | IGFBP7:KLRF1 | GAS7:DOCK5 | ALPL:RNASE6 |
| PCOLCE2:PRSS23 | PCOLCE2:RUNX2 | CD82:CNNM3 | RAB32:NLRP1 |
| TMEM165:PRPF38B | SMPDL3A:KLRD1 | GAS7:EXTL3 | TLR5:SEMA4D |
| PDGFC:PHF3 | GALNT2:KLRF1 | TSPO:RNASE6 | IMPDH1:NLRP1 |
| GAS7:NLRP1 | PDGFC:YPEL1 | ALPL:MME | ALPL:CAMK1D |
| PCOLCE2:KLRD1 | HK3:DENND3 | HK3:TLE3 | TSPO:NFIC |
| GALNT2:KLRD1 | PDGFC:CBLL1 | MCTP1:PARP8 | GAS7:HAL |
| KIAA0101:IL2RB | OPLAH:KLRD1 | TSPO:HCLS1 | PDGFC:NCOA6 |
| CR1:HAL | OPLAH:ZHX2 | TSPO:CASS4 | PDGFC:PIK3C2A |
| PDGFC:RFC1 | PDGFC:RYK | GAS7:RBM23 | TSPO:ADAM19 |
| ENTPD7:KLRF1 | PDGFC:IKZF5 | GAS7:EPHB4 | CD82:NOV |
| PDGFC:GRK5 | GALNT2:INPP5D | PDGFC:RBM15 | PDGFC:PDS5B |
| PCOLCE2:PYHIN1 | PDGFC:GCC2 | ADM:CLEC7A | FIG4:INPP5D |
| GAS7:PRKDC | PDGFC:MBIP | PDGFC:LEPROTL1 | TSPO:NOV |
| GAS7:CAMK1D | COX15:UTRN | PDGFC:NPAT | |
| MGAM:MME | SMPDL3A:QRICH1 | TSPO:PLA2G7 | |

In specific embodiments, a single BaSIRS derived biomarker combination (e.g., any one from TABLE A) is used for determining the indicator. In other embodiments, two BaSIRS derived biomarker combinations (e.g., any two from TABLE A) are used for determining the indicator. In still other embodiments, three BaSIRS derived biomarker combinations (e.g., any three from TABLE A) are used for determining the indicator. In still other embodiments, four BaSIRS derived biomarker combinations (e.g., any four from TABLE A) are used for determining the indicator.

In representative examples of this type, the methods comprise: (a) determining a single BaSIRS derived biomarker value using a pair of BaSIRS biomarker values, the single BaSIRS derived biomarker value being indicative of a ratio of levels of first and second BaSIRS biomarkers; and (b) determining the indicator using the single derived BaSIRS biomarker value.

In other representative examples of this type, the methods comprise: (a) determining a first BaSIRS derived biomarker value using a first pair of BaSIRS biomarker values, the first BaSIRS derived biomarker value being indicative of a ratio of levels of first and second BaSIRS biomarkers; (b) determining a second BaSIRS derived biomarker value using a second pair of BaSIRS biomarker values, the second BaSIRS derived biomarker value being indicative of a ratio of levels of third and fourth BaSIRS biomarkers; and (c) determining the indicator by combining the first and second derived BaSIRS biomarker values, using for example a combining function as disclosed herein.

In still other representative examples of this type, the methods comprise: (a) determining a first BaSIRS derived biomarker value using a first pair of BaSIRS biomarker values, the first BaSIRS derived biomarker value being indicative of a ratio of levels of first and second BaSIRS biomarkers; (b) determining a second BaSIRS derived biomarker value using a second pair of BaSIRS biomarker values, the second BaSIRS derived biomarker value being indicative of a ratio of levels of third and fourth BaSIRS biomarkers; (c) determining a third BaSIRS derived biomarker value using a third pair of BaSIRS biomarker values, the third BaSIRS derived biomarker value being indicative of a ratio of levels of fifth and fourth BaSIRS biomarkers; and (d) determining the indicator by combining the first and sixth derived BaSIRS biomarker values, using for example a combining function as disclosed herein.

In certain embodiments, individual BaSIRS derived biomarker combinations are selected from TSPO:HCLS1, OPLAH:ZHX2, TSPO:RNASE6; GAS7:CAMK1D, ST3GAL2:PRKD2, PCOLCE2:NMUR1 and CR1:HAL. In preferred embodiments, individual BaSIRS derived biomarker combinations are selected from OPLAH:ZHX2 and TSPO:HCLS1.

The bacterium associated with the BaSIRS is suitably selected from any Gram positive or Gram negative bacterial species which is capable of inducing at least one of the clinical signs of SIRS.

Typical VaSIRS derived biomarker combinations are suitably selected from TABLE B.

TABLE B
VaSIRS Derived Biomarker

| | | | |
|---|---|---|---|
| IFI6:IL16 | OASL:SERTAD2 | OASL:KIAA0247 | OASL:TOPORS |
| OASL:NR3C1 | OASL:LPAR2 | OASL:ARHGAP26 | EIF2AK2:IL16 |
| OASL:EMR2 | OASL:ITGAX | OASL:LYN | OASL:NCOA1 |
| OASL:SORL1 | OASL:TGFBR2 | OASL:PCBP2 | OASL:PTGER4 |
| OASL:TLR2 | OASL:ARHGAP25 | OASL:XPO6 | OASL:GNAQ |
| OASL:PACSIN2 | OASL:GNA12 | OASL:ATP6V1B2 | OASL:GSK3B |
| OASL:LILRA2 | OASL:NUMB | OASL:CSF2RB | OASL:IL6R |
| OASL:PTPRE | OASL:CREBBP | OASL:GYPC | OASL:MAPK14 |
| OASL:RPS6KA1 | OASL:PINK1 | OASL:IL4R | USP18:TGFBR2 |
| OASL:CASC3 | OASL:PITPNA | OASL:MMP25 | ISG15:LTB |
| OASL:VEZF1 | OASL:SEMA4D | OASL:PSEN1 | OASL:INPP5D |
| OASL:CRLF3 | OASL:TGFBI | OASL:SH2B3 | OASL:MED13 |
| OASL:NDEL1 | OASL:APLP2 | OASL:STAT5A | OASL:MORC3 |
| OASL:RASSF2 | OASL:CCNG2 | ISG15:IL16 | OASL:PTAFR |
| OASL:TLE4 | OASL:MKRN1 | MX1:LEF1 | OASL:RBM23 |
| OASL:CD97 | OASL:RGS14 | OASL:CAMK2G | OASL:SNN |
| OASL:CEP68 | OASL:LYST | OASL:ETS2 | OASL:ST13 |
| OASL:RXRA | OASL:TNRC6B | OASL:POLB | OASL:TFEB |
| OASL:SP3 | OASL:TYROBP | OASL:STK38L | OASL:ZFYVE16 |
| OASL:ABLIM1 | OASL:WDR37 | OASL:TFE3 | EIF2AK2:SATB1 |
| OASL:AOAH | OASL:WDR47 | OASL:ICAM3 | OASL:ABAT |
| OASL:MBP | UBE2L6:IL16 | OASL:ITGB2 | OASL:ABI1 |
| OASL:NLRP1 | OASL:BTG1 | OASL:PISD | OASL:ACVR1B |
| OASL:PBX3 | OASL:CD93 | OASL:PLXNC1 | OASL:GPSM3 |
| OASL:PTPN6 | OASL:DCP2 | OASL:SNX27 | OASL:MPPE1 |
| OASL:RYBP | OASL:FYB | OASL:TNIP1 | OASL:PTEN |
| OASL:IL13RA1 | OASL:MAML1 | OASL:ZMIZ1 | OASL:SEC62 |
| OASL:LCP2 | OASL:SNRK | OASL:FOXO3 | IFI6:MYC |
| OASL:LRP10 | OASL:USP4 | OASL:IL10RB | IFI6:PCF11 |
| OASL:SYPL1 | OASL:YTHDF3 | OASL:MAP3K5 | OASL:AIF1 |
| OASL:VAMP3 | OASL:CEP170 | OASL:POLD4 | OASL:CSNK1D |
| IFI44:LTB | OASL:PLEKHO2 | OASL:ARAP1 | OASL:GABARAP |
| OASL:ARHGEF2 | OASL:SMAD4 | OASL:CTBP2 | OASL:HAL |
| OASL:CTDSP2 | OASL:ST3GAL1 | OASL:DGKA | OASL:LAPTM5 |
| OASL:LST1 | OASL:ZNF292 | OASL:NFYA | OASL:XPC |
| OASL:MAPK1 | IFI44:IL4R | OASL:PCNX | USP18:NFKB1 |
| OASL:N4BP1 | OASL:HPCAL1 | OASL:PFDN5 | OASL:ACAP2 |
| OASL:STAT5B | OASL:IGSF6 | OASL:R3HDM2 | OASL:CLEC4A |
| IFI44:ABLIM1 | OASL:MTMR3 | OASL:STX6 | OASL:HIP1 |
| IFI44:IL6ST | OASL:PHF20 | EIF2AK2:SYPL1 | OASL:PIAS1 |
| OASL:BACH1 | OASL:PPARD | ISG15:ABLIM1 | OASL:PPP3R1 |
| OASL:KLF7 | OASL:PPP4R1 | OASL:FOXJ2 | OASL:RALB |
| OASL:PRMT2 | OASL:RBMS1 | OASL:IQSEC1 | OASL:RGS19 |
| OASL:HCK | OASL:RHOG | OASL:LRMP | OASL:TRIOBP |
| OASL:ITPKB | OASL:TIAM1 | OASL:NAB1 | EIF2AK2:PDE3B |
| OASL:MAP4K4 | USP18:IL16 | OASL:RAB31 | OASL:NCOA4 |
| OASL:PPM1F | OASL:CBX7 | OASL:WASF2 | OASL:RARA |
| OASL:RAB14 | OASL:RAF1 | OASL:ZNF274 | OASL:RPS6KA3 |
| IFI6:ABLIM1 | OASL:SERINC5 | OAS2:LEF1 | OASL:SIRPA |
| OAS2:FAIM3 | OASL:UBQLN2 | OASL:BRD1 | OASL:TLE3 |
| OASL:TNFRSF1A | USP18:CHMP7 | DHX58:IL16 | OASL:SLCO3A1 |
| DDX60:TGFBR2 | USP18:NECAP2 | ISG15:IL4R | OASL:ZDHHC17 |
| OASL:FLOT2 | OASL:CAP1 | OASL:BRD4 | USP18:FOXO1 |
| OASL:FNBP1 | OASL:HPS1 | OASL:CCNT2 | OASL:ASAP1 |
| OASL:MAP3K3 | OASL:IL1RAP | OASL:FGR | OASL:BAZ2B |
| OASL:STX10 | OASL:MEF2A | OASL:ITSN2 | OASL:FAM65B |
| OASL:ZDHHC18 | OASL:RNF19B | OASL:LYL1 | OASL:HHEX |
| OASL:ZNF143 | OASL:TMEM127 | OASL:PHF3 | OASL:MAX |
| TAP1:TGFBR2 | USP18:IL27RA | OASL:PSAP | OASL:PHF2 |
| OAS2:ABLIM1 | OASL:CDIPT | OASL:STX3 | OASL:RNF130 |
| OASL:ARRB2 | OASL:CREB1 | OASL:TNK2 | OASL:SOS2 |
| OASL:IKBKB | OASL:GPS2 | EIF2AK2:ZNF274 | OASL:STAM2 |
| OASL:KBTBD2 | OASL:NDE1 | OASL:ACAA1 | OASL:ZFC3H1 |
| OASL:PHC2 | OASL:RAB11FIP1 | OASL:CHD3 | IFI44:CYLD |
| OASL:PUM2 | USP18:ABLIM1 | OASL:FRY | IFIH1:CRLF3 |
| OASL:SSFA2 | EIF2AK2:TNRC6B | OASL:GRB2 | OASL:BANP |
| IFI44:MYC | OASL:FAM134A | OASL:MAP3K11 | OASL:CCND3 |
| OASL:ABHD2 | OASL:FCGRT | OASL:NEK7 | OASL:DGCR2 |
| OASL:CYLD | OASL:LPIN2 | OASL:PPP2R5A | OASL:USP15 |
| OASL:MAST3 | OASL:PECAM1 | USP18:ST13 | USP18:EIF3H |
| OASL:UBN1 | OASL:WBP2 | XAF1:LEF1 | OASL:LAT2 |
| IFI6:IL6ST | OASL:ZNF148 | OASL:CASP8 | OASL:ZYX |
| IFIH1:TGFBR2 | OASL:RTN3 | OASL:PCF11 | USP18:CAMK1D |
| OASL:CNPY3 | OASL:TYK2 | OASL:PRKCD | ZBP1:NDE1 |
| OASL:KIAA0232 | USP18:LTB | OASL:PSTPIP1 | |

In specific embodiments, a single VaSIRS derived biomarker combination (e.g., any one from TABLE B) is used for determining the indicator. In other embodiments, two VaSIRS derived biomarker combinations (e.g., any two from TABLE B) are used for determining the indicator. In still other embodiments, three VaSIRS derived biomarker combinations (e.g., any three from TABLE B) are used for determining the indicator. In still other embodiments, four VaSIRS derived biomarker combinations (e.g., any four from TABLE B) are used for determining the indicator.

In non-limiting examples of this type, the methods comprise: (a) determining a single VaSIRS derived biomarker value using a pair of VaSIRS biomarker values, the single VaSIRS derived biomarker value being indicative of a ratio of levels of first and second VaSIRS biomarkers; and (b) determining the indicator using the single derived VaSIRS biomarker value.

In other non-limiting examples of this type, the methods comprise: (a) determining a first VaSIRS derived biomarker value using a first pair of VaSIRS biomarker values, the first VaSIRS derived biomarker value being indicative of a ratio of levels of first and second VaSIRS biomarkers; (b) determining a second VaSIRS derived biomarker value using a second pair of VaSIRS biomarker values, the second VaSIRS derived biomarker value being indicative of a ratio of levels of third and fourth VaSIRS biomarkers; and (c) determining the indicator by combining the first and second derived VaSIRS biomarker values, using for example a combining function as disclosed herein.

In still other non-limiting examples of this type, the methods comprise: (a) determining a first VaSIRS derived biomarker value using a first pair of VaSIRS biomarker values, the first VaSIRS derived biomarker value being indicative of a ratio of levels of first and second VaSIRS biomarkers; (b) determining a second VaSIRS derived biomarker value using a second pair of VaSIRS biomarker values, the second VaSIRS derived biomarker value being indicative of a ratio of levels of third and fourth VaSIRS biomarkers; (c) determining a third VaSIRS derived biomarker value using a third pair of VaSIRS biomarker values, the third VaSIRS derived biomarker value being indicative of a ratio of levels of fifth and fourth VaSIRS biomarkers; and (d) determining the indicator by combining the first and sixth derived VaSIRS biomarker values, using for example a combining function as disclosed herein.

In certain embodiments, individual VaSIRS derived biomarker combinations are selected from ISG15:IL16, OASL:ADGRE5, TAP1:TGFBR2, IFIH1:CRLF3, IFI44:IL4R, EIF2AK2:SYPL1, OAS2:LEF1, STAT1:PCBP2 and IFI6:IL6ST. In preferred embodiments, individual VaSIRS derived biomarker combinations are selected from ISG15:IL16 and OASL:ADGRE5.

The virus associated with the VaSIRS is suitably selected from any one of Baltimore virus classification Groups I, II, III, IV, V, VI and VII, which is capable of inducing at least one of the clinical signs of SIRS.

Exemplary PaSIRS derived biomarker combinations are suitably selected from TABLE C.

TABLE C
PaSIRS Derived Biomarker

| | | | |
|---|---|---|---|
| RPL9:WARS | PREPL:WARS | SEH1L:WARS | EXOSC10:MYD88 |
| RPL9:CSTB | TCF4:LAP3 | EXOSC10:UBE2L6 | LY9:WARS |
| NUP160:WARS | ZBED5:WARS | TTC17:LAP3 | IMP3:CSTB |
| IMP3:ATOX1 | TCF4:POMP | SUCLG2:CEBPB | RPL15:CEBPB |
| RPS4X:WARS | NUP160:SQRDL | EXOSC10:G6PD | ARHGAP17:ATOX1 |
| TCF4:CEBPB | TRIT1:WARS | CEP192:WARS | TTC17:MYD88 |
| IMP3:LAP3 | ZBED5:CEBPB | NUP160:CD63 | EXOSC10:TCIRG1 |
| EXOSC10:WARS | IMP3:WARS | TMEM50B:WARS | ZMYND11:CEBPB |
| TTC17:WARS | RPS4X:SQRDL | EXOSC10:LDHA | CEP192:TANK |
| TCF4:WARS | NUP160:POMP | ARID1A:CSTB | IMP3:UBE2L6 |
| METAP1:WARS | EXOSC10:LAP3 | SUCLG2:WARS | RPS4X:CD63 |
| FNTA:POMP | RPS4X:GNG5 | ARID1A:CEBPB | RPL9:CD63 |
| TCF4:TANK | TOP2B:WARS | FBXO11:TANK | ARID1A:UBE2L6 |
| TOP2B:CEBPB | RPL9:POMP | SUCLG2:SH3GLB1 | TCF4:UBE2L6 |
| AHCTF1:CEBPB | EXOSC10:ATOX1 | TTC17:G6PD | ARID1A:WARS |
| RPS4X:MYD88 | TTC17:TANK | IMP3:PCMT1 | CAMK2G:G6PD |
| IMP3:CEBPB | EXOSC10:CEBPB | ARID1A:LAP3 | RPS4X:SH3GLB1 |
| RPL9:CEBPB | NOSIP:CEBPB | IMP3:SQRDL | RPL9:TANK |
| RPS4X:CEBPB | RPL22:CEBPB | TCF4:ATOX1 | IMP3:TANK |
| TTC17:CEBPB | TTC17:ATP2A2 | IMP3:SH3GLB1 | ZBED5:SH3GLB1 |
| TMEM50B:CEBPB | ZMYND11:CSTB | RPS4X:SERPINB1 | ZMYND11:SH3GLB1 |
| RPS4X:POMP | FNTA:SH3GLB1 | FBXO11:RALB | RPS14:CD63 |
| TOP2B:POMP | ARID1A:TAP1 | TMEM50B:SQRDL | CAMK2G:SQRDL |
| METAP1:POMP | NOSIP:WARS | CSNK1G2:CEBPB | ARIH2:CEBPB |
| EXOSC10:CSTB | RPS4X:UPP1 | RPL15:SH3GLB1 | ARID1A:NFIL3 |
| ZNF266:CEBPB | CNOT7:CEBPB | BCL11A:G6PD | IMP3:POMP |
| TTC17:ATOX1 | ARHGAP17:WARS | ZBED5:SQRDL | EXOSC10:ENO1 |
| CSNK1G2:G6PD | UFM1:WARS | ARID1A:SERPINB1 | PREPL:SH3GLB1 |
| SETX:CEBPB | PREPL:SQRDL | RPS14:SH3GLB1 | TTC17:BCL6 |
| ARHGAP17:CEBPB | IMP3:TAP1 | EXOSC10:TAP1 | ZMYND11:POMP |
| ZMYND11:WARS | ARID1A:PCMT1 | BCL11A:CEBPB | IMP3:RIT1 |
| IMP3:UPP1 | SUCLG2:SQRDL | ADSL:ATOX1 | CAMK2G:CD63 |
| EXOSC10:IRF1 | RPL22:SH3GLB1 | TCF4:FCER1G | IL10RA:CEBPB |
| UFM1:CEBPB | BCL11A:WARS | LY9:SH3GLB1 | FNTA:TCIRG1 |
| ARID1A:LDHA | CNOT7:WARS | IMP3:GNG5 | CAMK2G:TCIRG1 |
| RPL9:ATOX1 | ZBED5:TCIRG1 | SERTAD2:CEBPB | EXOSC10:PCMT1 |
| TTC17:GNG5 | EXOSC10:SQRDL | AHCTF1:MYD88 | RPS14:SQRDL |
| EXOSC10:POMP | AHCTF1:GNG5 | ARID1A:ENO1 | IMP3:PGD |
| ARID1A:ATOX1 | ZMYND11:FCER1G | EXOSC10:UPP1 | ZBED5:TNIP1 |
| RPL9:SH3GLB1 | TOP2B:ENO1 | CEP192:CSTB | CHN2:WARS |
| LY9:CEBPB | IMP3:IRF1 | LY9:SQRDL | IMP3:TCIRG1 |
| RPS14:WARS | CEP192:TAP1 | LY9:TNIP1 | AHCTF1:SQRDL |
| FNTA:SQRDL | RPL9:MYD88 | CNOT7:G6PD | CLIP4:WARS |
| APEX1:CD63 | RPL22:GNG5 | ARID1A:PLSCR1 | NOSIP:POMP |
| SETX:WARS | FNTA:MYD88 | CEP192:ATOX1 | RPL22:SQRDL |
| IMP3:TNIP1 | TCF4:GNG5 | IMP3:ENO1 | IMP3:VAMP3 |
| FNTA:CD63 | EXOSC10:TANK | ARID1A:IRF1 | TTC17:TIMP2 |
| TTC17:TCIRG1 | MLLT10:WARS | EXOSC10:GNG5 | TTC17:SQRDL |
| EXOSC10:SH3GLB1 | TTC17:POMP | LY9:ATOX1 | ARID1A:CD63 |
| RPS4X:FCER1G | TCF4:MYD88 | FBXO11:CEBPB | FNTA:LAP3 |
| RPS4X:PGD | IMP3:MYD88 | RPL9:SLAMF7 | BCL11A:LAP3 |
| CAMK2G:CEBPB | TOP2B:CD63 | RPL9:TNIP1 | IMP3:FCER1G |
| ZMYND11:G6PD | CEP192:RALB | PREPL:CD63 | CEP192:TNIP1 |
| FNTA:CEBPB | NUP160:PGD | ARHGAP17:SQRDL | ZMYND11:SQRDL |
| ZMYND11:CD63 | RPL9:SQRDL | ZBED5:POMP | ZMYND11:GNG5 |
| TCF4:RALB | CEP192:PCMT1 | RPS4X:TSPO | ARID1A:SLAMF7 |
| ARHGAP17:LAP3 | TCF4:SQRDL | IMP3:G6PD | ARID1A:TCIRG1 |
| IMP3:CD63 | RPL9:GNG5 | CEP192:POMP | ARID1A:TNIP1 |
| ZMYND11:C3AR1 | EXOSC10:CD63 | TMEM50B:CD63 | ZMYND11:PGD |
| AHCTF1:WARS | TCF4:SH3GLB1 | ZMYND11:ENO1 | CSNK1G2:TCIRG1 |
| RPS4X:ENO1 | ADSL:WARS | CEP192:LAP3 | TTC17:CD63 |
| CEP192:PLSCR1 | TTC17:SH3GLB1 | RPL9:UPP1 | NUP160:RTN4 |
| EXOSC9:POMP | ARID1A:SQRDL | TCF4:SERPINB1 | RPL15:SQRDL |
| FNTA:GNG5 | ARID1A:G6PD | AHCTF1:PLAUR | TTC17:UPP1 |
| CEP192:IRF1 | AHCTF1:TANK | RPL22:WARS | CAMK2G:FCER1G |
| CEP192:CEBPB | EXOSC2:CEBPB | EXOSC2:POMP | CEP192:TCIRG1 |
| IRF8:CEBPB | CNOT7:CSTB | AHCTF1:UPP1 | TTC17:SERPINB1 |
| CEP192:G6PD | ARID1A:PGD | IMP3:RALB | EXOSC2:UPP1 |
| FBXO11:UPP1 | ARID1A:STAT3 | ADK:SH3GLB1 | IMP3:TSPO |
| ARIH2:TCIRG1 | NOSIP:TCIRG1 | SUCLG2:CD63 | BCL11A:TNIP1 |
| PCID2:WARS | RPL9:FCER1G | FNTA:WARS | ADSL:ENO1 |
| CAMK2G:PGD | ARID1A:TRPC4AP | EXOSC10:TUBA1B | NOSIP:SQRDL |
| EXOSC10:FLII | ARID1A:SH3GLB1 | IMP3:PCBP1 | SERBP1:SH3GLB1 |
| RPL15:CD63 | CEP192:RAB27A | ARID1A:GRINA | ARID1A:NFKBIA |
| RPL22:CD63 | EXOSC10:FCER1G | TTC17:PGD | RPL9:ENO1 |
| CNOT7:SQRDL | SETX:SQRDL | ARID1A:TANK | ARID1A:RAB27A |
| FBXO11:SQRDL | CEP192:MYD88 | CSNK1G2:FLII | RPL15:WARS |
| TCF4:UPP1 | ARID1A:BCL6 | CEP192:STAT3 | BCL11A:CSTB |
| PCID2:CEBPB | EXOSC2:CD63 | AHCTF1:SH3GLB1 | |

In specific embodiments, a single PaSIRS derived biomarker combination (e.g., any one from TABLE C) is used for determining the indicator. In other embodiments, two PaSIRS derived biomarker combinations (e.g., any two from TABLE C) are used for determining the indicator. In still other embodiments, three PaSIRS derived biomarker combinations (e.g., any three from TABLE C) are used for determining the indicator. In still other embodiments, four PaSIRS derived biomarker combinations (e.g., any four from TABLE C) are used for determining the indicator.

In illustrative examples of this type, the methods comprise: (a) determining a single PaSIRS derived biomarker value using a pair of PaSIRS biomarker values, the single PaSIRS derived biomarker value being indicative of a ratio of levels of first and second PaSIRS biomarkers; and (b) determining the indicator using the single derived PaSIRS biomarker value.

In other illustrative examples of this type, the methods comprise: (a) determining a first PaSIRS derived biomarker value using a first pair of PaSIRS biomarker values, the first PaSIRS derived biomarker value being indicative of a ratio of levels of first and second PaSIRS biomarkers; (b) determining a second PaSIRS derived biomarker value using a second pair of PaSIRS biomarker values, the second PaSIRS derived biomarker value being indicative of a ratio of levels of third and fourth PaSIRS biomarkers; and (c) determining the indicator by combining the first and second derived PaSIRS biomarker values, using for example a combining function as disclosed herein.

In still other illustrative examples of this type, the methods comprise: (a) determining a first PaSIRS derived biomarker value using a first pair of PaSIRS biomarker values, the first PaSIRS derived biomarker value being indicative of a ratio of levels of first and second PaSIRS biomarkers; (b) determining a second PaSIRS derived biomarker value using a second pair of PaSIRS biomarker values, the second PaSIRS derived biomarker value being indicative of a ratio of levels of third and fourth PaSIRS biomarkers; (c) determining a third PaSIRS derived biomarker value using a third pair of PaSIRS biomarker values, the third PaSIRS derived biomarker value being indicative of a ratio of levels of fifth and fourth PaSIRS biomarkers; and (d) determining the indicator by combining the first and sixth derived PaSIRS biomarker values, using for example a combining function as disclosed herein.

In certain embodiments, individual PaSIRS derived biomarker combinations are suitably selected from TTC17:G6PD, HERC6:LAP3 and NUP160:TPP1.

The protozoan associated with the PaSIRS is suitably selected from any of the following protozoal genera, which are capable of inducing at least one of the clinical signs of SIRS; for example, Toxoplasma, Babesia, Plasmodium, Trypanosoma, Giardia, Entamoeba, Cryptosporidium, Balantidium and Leishmania.

Typical InSIRS derived biomarker combinations can be selected from TABLE D.

TABLE D
InSIRS Derived Biomarker

| | | | |
|---|---|---|---|
| TNFSF8:VEZT | TNFSF8:CDK6 | TNFSF8:SLC35A3 | TNFSF8:YEATS4 |
| TNFSF8:HEATR1 | TNFSF8:MANEA | ADAM19:TMEM87A | TNFSF8:CLUAP1 |
| TNFSF8:THOC2 | TNFSF8:CKAP2 | TNFSF8:LANCL1 | TNFSF8:LARP4 |
| TNFSF8:NIP7 | TNFSF8:ZNF507 | ADAM19:ERCC4 | TNFSF8:SLC35D1 |
| TNFSF8:MLLT10 | TNFSF8:GGPS1 | TNFSF8:CD28 | SYNE2:RBM26 |
| TNFSF8:EIF5B | TNFSF8:XPO4 | ADAM19:MLLT10 | TNFSF8:CD40LG |
| TNFSF8:LRRC8D | TNFSF8:PHC3 | TNFSF8:IQCB1 | VNN3:CYSLTR1 |
| TNFSF8:RNMT | TNFSF8:ASCC3 | TNFSF8:FASTKD2 | TNFSF8:SYT11 |
| STK17B:ARL6IP5 | TNFSF8:NOL10 | TNFSF8:RDX | TNFSF8:RIOK2 |
| ENTPD1:ARL6IP5 | TNFSF8:ANK3 | TNFSF8:MTO1 | TNFSF8:BZW2 |
| TNFSF8:CD84 | TNFSF8:SMC3 | IQSEC1:MACF1 | TNFSF8:LARP1 |
| TNFSF8:PWP1 | TNFSF8:REPS1 | TNFSF8:SMC6 | ADAM19:SYT11 |
| TNFSF8:IPO7 | TNFSF8:C14orf1 | TNFSF8:NEK1 | TNFSF8:NCBP1 |
| ADAM19:EXOC7 | TNFSF8:FUT8 | TNFSF8:ZNF562 | ADAM19:MACF1 |
| TNFSF8:ARHGAP5 | TNFSF8:VPS13A | TNFSF8:PEX1 | TNFSF8:NOL8 |
| TNFSF8:RMND1 | TNFSF8:RAD50 | ADAM19:SIDT2 | TNFSF8:KIAA0391 |
| TNFSF8:IDE | TNFSF8:ESF1 | TNFSF8:METTL5 | |
| TNFSF8:TBCE | TNFSF8:MRPS10 | CYP4F3:TRAPPC2 | |
| TNFSF8:G3BP1 | CDA:EFHD2 | TNFSF8:KRIT1 | |

In specific embodiments, a single InSIRS derived biomarker combination (e.g., any one from TABLE D) is used for determining the indicator. In other embodiments, two InSIRS derived biomarker combinations (e.g., any two from TABLE D) are used for determining the indicator. In still other embodiments, three InSIRS derived biomarker combinations (e.g., any three from TABLE D) are used for determining the indicator. In still other embodiments, four InSIRS derived biomarker combinations (e.g., any four from TABLE D) are used for determining the indicator.

In representative examples of this type, the methods comprise: (a) determining a single InSIRS derived biomarker value using a pair of InSIRS biomarker values, the single InSIRS derived biomarker value being indicative of a ratio of levels of first and second InSIRS biomarkers; and (b) determining the indicator using the single derived InSIRS biomarker value.

In other representative examples of this type, the methods comprise: (a) determining a first InSIRS derived biomarker value using a first pair of InSIRS biomarker values, the first InSIRS derived biomarker value being indicative of a ratio of levels of first and second InSIRS biomarkers; (b) determining a second InSIRS derived biomarker value using a second pair of InSIRS biomarker values, the second InSIRS derived biomarker value being indicative of a ratio of levels of third and fourth InSIRS biomarkers; and (c) determining the indicator by combining the first and second derived InSIRS biomarker values, using for example a combining function as disclosed herein.

In still other representative examples of this type, the methods comprise: (a) determining a first InSIRS derived biomarker value using a first pair of InSIRS biomarker values, the first InSIRS derived biomarker value being indicative of a ratio of levels of first and second InSIRS biomarkers; (b) determining a second InSIRS derived biomarker value using a second pair of InSIRS biomarker values, the second InSIRS derived biomarker value being indicative of a ratio of levels of third and fourth InSIRS biomarkers; (c) determining a third InSIRS derived biomarker value using a third pair of InSIRS biomarker values, the third InSIRS derived biomarker value being indicative of a ratio of levels of fifth and fourth InSIRS biomarkers; and (d) determining the indicator by combining the first and sixth derived InSIRS biomarker values, using for example a combining function as disclosed herein.

In certain embodiments, individual InSIRS derived biomarker combinations are suitably selected from ENTPD1:ARL6IP5, TNFSF8:HEATR1, ADAM19:POLR2A, SYNE2:VPS13C, TNFSF8:NIP7, CDA:EFHD2, ADAM19:MLLT10, PTGS1:ENTPD1, ADAM19:EXOC7 and CDA:PTGS1. In preferred embodiments, individual InSIRS derived biomarker combinations are suitably selected from ENTPD1:ARL6IP5 and TNFSF8:HEATR1.

Numerous non-infectious conditions are capable of inducing at least one of the clinical signs of SIRS, non-limiting examples of which include cancer, pancreatitis, surgery, embolism, aneurysm, autoimmune disease, sarcoidosis, trauma, asthma, allergic reaction, burn, haemorrhage, ischaemia/reperfusion, adverse drug response, stress, tissue damage/inflammation, foreign body response, obesity, coronary artery disease, anxiety, age.

Another aspect of the present invention provides apparatus for determining an indicator used in assessing a likelihood of a subject having a presence, absence or degree of BaSIRS or VaSIRS. This apparatus generally comprises at least one electronic processing device that:

determines a plurality of host response specific biomarker values including a plurality of BaSIRS biomarker values and a plurality of VaSIRS biomarker values, the plurality of BaSIRS biomarker values being indicative of values measured for a corresponding plurality of BaSIRS biomarkers in a sample taken from the subject, the plurality of VaSIRS biomarker values being indicative of values measured for a corresponding plurality of VaSIRS biomarkers in the sample;

determines a plurality of host response specific derived biomarker values including at least one BaSIRS derived biomarker value and at least one VaSIRS derived biomarker value, each derived BaSIRS biomarker value being determined using at least a subset of the plurality of BaSIRS biomarker values, and being indicative of a ratio of levels of a corresponding at least a subset of the plurality of BaSIRS biomarkers, and each derived VaSIRS biomarker value being determined using at least a subset of the plurality of VaSIRS biomarker values, and being indicative of a ratio of levels of a corresponding at least a subset of the plurality of VaSIRS biomarkers; and

determines the indicator using the plurality of host response specific derived biomarker values, wherein the at least a subset of BaSIRS biomarkers forms a BaSIRS derived biomarker combination which is not a derived biomarker combination for VaSIRS, PaSIRS or InSIRS, and wherein the at least a subset of VaSIRS biomarkers forms a VaSIRS derived biomarker combination which is not a derived biomarker combination for BaSIRS, PaSIRS or InSIRS.

In some embodiments, the at least one processing device:

(a) determines a plurality of pathogen specific biomarker values including at least one bacterial biomarker value and at least one viral biomarker value, the least one bacterial biomarker value being indicative of a value measured for a corresponding bacterial biomarker in the sample, the least one viral biomarker value being indicative of a value measured for a corresponding viral biomarker in the sample; and

(b) determines the indicator using the host response specific derived biomarker values in combination with the pathogen specific biomarker values.

In some embodiments, the plurality of host response specific biomarker values determined by the least one electronic processing device further include a plurality of PaSIRS biomarker values, the plurality of PaSIRS biomarker values being indicative of values measured for a corresponding plurality of PaSIRS biomarkers in the sample, and the plurality of host response specific derived biomarker values further includes at least one PaSIRS derived biomarker value, and the least one electronic processing device further:

determines each PaSIRS derived biomarker value using at least a subset of the plurality of PaSIRS biomarker values, the PaSIRS derived biomarker value being indicative of a ratio of levels of a corresponding at least a subset of the plurality of PaSIRS biomarkers; and

determines the indicator using the plurality of host response specific derived biomarker values, wherein the at least a subset of PaSIRS biomarkers forms a PaSIRS derived biomarker combination which is not a derived biomarker combination for BaSIRS, VaSIRS or InSIRS.

In some embodiments, the least one electronic processing device:

(a) determines a plurality of pathogen specific biomarker values including at least one bacterial biomarker value, at least one viral biomarker value and at least one protozoal biomarker value, the at least one bacterial biomarker value being indicative of a value measured for a corresponding bacterial biomarker in the sample, the least one viral biomarker value being indicative of a value measured for a corresponding viral biomarker in the sample, and the least one protozoal biomarker value being indicative of a value measured for a corresponding protozoal biomarker in the sample; and

(b) determines the indicator using the host response specific derived biomarker values in combination with the pathogen specific biomarker values.

In some embodiments, the plurality of host response specific biomarker values determined by the least one electronic processing device further include a plurality of InSIRS biomarker values, the plurality of InSIRS biomarker values being indicative of values measured for a corresponding plurality of InSIRS biomarkers in the sample, and the plurality of host response specific derived biomarker values further includes at least one InSIRS derived biomarker value, and the least one electronic processing device further:

determines each InSIRS derived biomarker value using at least a subset of the plurality of InSIRS biomarker values, the InSIRS derived biomarker value being indicative of a ratio of levels of a corresponding at least a subset of the plurality of InSIRS biomarkers; and

determines the indicator using the plurality of host response specific derived biomarker values, wherein the at least a subset of InSIRS biomarkers forms a InSIRS derived biomarker combination which is not a derived biomarker combination for BaSIRS, VaSIRS or PaSIRS.

In yet another aspect, the present invention provides compositions for determining an indicator used in assessing a likelihood of a subject having a presence, absence or degree of BaSIRS or VaSIRS. These compositions generally comprise, consist or consist essentially of: (1) a pair of BaSIRS biomarker cDNAs, and for each BaSIRS biomarker cDNA at least one oligonucleotide primer that hybridizes to the BaSIRS biomarker cDNA, and/or at least one oligonucleotide probe that hybridizes to the BaSIRS biomarker cDNA, wherein the at least one oligonucleotide primer and/or the at least one oligonucleotide probe comprises a heterologous label, and (2) a pair of VaSIRS biomarker cDNAs, and for each VaSIRS biomarker cDNA at least one oligonucleotide primer that hybridizes to the VaSIRS biomarker cDNA, and/or at least one oligonucleotide probe that hybridizes to the VaSIRS biomarker cDNA, wherein the at least one oligonucleotide primer and/or the at least one oligonucleotide probe comprises a heterologous label, wherein the pair of BaSIRS biomarker cDNAs forms a BaSIRS derived biomarker combination which is not a derived biomarker combination for VaSIRS, PaSIRS or InSIRS, wherein the pair of VaSIRS biomarker cDNAs forms a VaSIRS derived biomarker combination which is not a derived biomarker combination for BaSIRS, PaSIRS or InSIRS, wherein the BaSIRS derived biomarker combination is selected from the BaSIRS derived biomarker combinations set out in TABLE A, and wherein the VaSIRS derived biomarker combination is selected from the VaSIRS derived biomarker combinations set out in TABLE B.

In some embodiments, the compositions further comprise (a) a pair of PaSIRS biomarker cDNAs, and for each PaSIRS biomarker cDNA at least one oligonucleotide primer that hybridizes to the PaSIRS biomarker cDNA, and/or at least one oligonucleotide probe that hybridizes to the PaSIRS biomarker cDNA, wherein the at least one oligonucleotide primer and/or the at least one oligonucleotide probe comprises a heterologous label, wherein the pair of PaSIRS biomarker cDNAs forms a PaSIRS derived biomarker combination which is not a derived biomarker combination for BaSIRS, VaSIRS or InSIRS, and wherein the PaSIRS derived biomarker combination is selected from the PaSIRS derived biomarker combinations set out in TABLE C.

Alternatively, or in addition, the compositions may further comprise (b) a pair of InSIRS biomarker cDNAs, and for each InSIRS biomarker cDNA at least one oligonucleotide primer that hybridizes to the InSIRS biomarker cDNA, and/or at least one oligonucleotide probe that hybridizes to the InSIRS biomarker cDNA, wherein the at least one oligonucleotide primer and/or the at least one oligonucleotide probe comprises a heterologous label, wherein the pair of InSIRS biomarker cDNAs forms an InSIRS derived biomarker combination which is not a derived biomarker combination for BaSIRS, VaSIRS or PaSIRS, and wherein the InSIRS derived biomarker combination is selected from the InSIRS derived biomarker combinations set out in TABLE D.

Suitably, in any of the embodiments or aspects disclosed herein, the compositions further comprise a DNA polymerase. The DNA polymerase may be a thermostable DNA polymerase.

In any of the embodiments or aspects disclosed herein, the compositions suitably comprise for each cDNA a pair of forward and reverse oligonucleotide primers that hybridize to opposite complementary strands of the cDNA and that permit nucleic acid amplification of at least a portion of the cDNA to produce an amplicon. In representative examples of these embodiments, the compositions may further comprise for each cDNA an oligonucleotide probe that comprises a heterologous label and hybridizes to the amplicon.

In certain embodiments, the components of an individual composition are comprised in a mixture.

Suitably, the compositions comprise a population of cDNAs corresponding to mRNA derived from a cell or cell population from a patient sample. In preferred embodiments, the population of cDNAs represents whole leukocyte cDNA (e.g., whole peripheral blood leukocyte cDNA) with a cDNA expression profile characteristic of a subject with a SIRS condition selected from BaSIRS, VaSIRS, PaSIRS and InSIRS, wherein the cDNA expression profile comprises at least one pair of biomarkers (e.g., 1, 2, 3, 4, 5, 6, 7, 8, 9, 10, 11, 12, 13, 14, 15, 16, 17, 18, 19, 20, 30, 40, 50 or more pairs of biomarkers), wherein a respective pair of biomarkers comprises a first biomarker and a second biomarker, wherein the first biomarker is expressed at a higher level in leukocytes (e.g., whole peripheral blood leukocytes) from a subject with the SIRS condition than in leukocytes (e.g., whole peripheral blood leukocytes) from a healthy subject or from a subject without the SIRS condition (e.g., the first biomarker is expressed in leukocytes from a subject with the SIRS condition at a level that is at least 110%, 120%, 130%, 140%, 150%, 160%, 170%, 180%, 190%, 200%, 250%, 300%, 350%, 400%, 450%, 500%, 600%, 700%, 800%, 900%, 1000%, 2000%, 3000%, 4000%, or 5000% of the level of the first biomarker in leukocytes from a healthy subject or from a subject without the SIRS condition), wherein the second biomarker is expressed at about the same or at a lower level in leukocytes (e.g., whole peripheral blood leukocytes) from a subject with the SIRS condition than in leukocytes (e.g., whole peripheral blood leukocytes) from a healthy subject or from a subject without the SIRS condition (e.g., the second biomarker is expressed in leukocytes from a subject with the SIRS condition at a level that is no more than 105%, 104%, 103%, 102%, 100%, 99%, 98%, 97%, 96%, 95%, 90%, 85%, 80%, 75%, 70%, 65%, 60%, 55%, 50%, 45%, 40%, 35%, 30%, 25%, 20%, 15%, 10%, 5%, 1%, 0.5%, 0.1%, 0.05%, 0.01%, 0.005%, 0.001% of the level of the second biomarker in leukocytes from a healthy subject or from a subject without the SIRS condition) and wherein the first biomarker is a first mentioned or ‘numerator’ biomarker of a respective pair of biomarkers in any one of TABLES A, B, C or D, and the second biomarker represents a second mentioned or ‘denominator’ biomarker of the respective pair of biomarkers.

In some embodiments, the sample is a body fluid, including blood, urine, plasma, serum, urine, secretion or excretion. In some embodiments, the cell population is from blood, suitably peripheral blood. In specific embodiments, the sample comprises blood, suitably peripheral blood. Suitably, the cell or cell population is a cell or cell population of the immune system, suitably a leukocyte or leukocyte population.

Suitably, in any of the embodiments or aspects disclosed herein, the compositions may further comprise a pathogen nucleic acid and at least one oligonucleotide primer that hybridizes to the pathogen nucleic acid, and/or at least one oligonucleotide probe that hybridizes to the pathogen nucleic acid, wherein the at least one oligonucleotide primer and/or the at least one oligonucleotide probe comprises a heterologous label. Suitably the pathogen from which the pathogen nucleic acid is selected is from a bacterium, a virus and a protozoan. The pathogen nucleic acid is suitably derived from a patient sample, suitably a body fluid, illustrative examples of which include blood, urine, plasma, serum, urine, secretion or excretion. In specific embodiments, the sample comprises blood, suitably peripheral blood.

Still another aspect of the present invention provides kits for determining an indicator used in assessing a likelihood of a subject having a presence, absence or degree of BaSIRS or VaSIRS. The kits generally comprise, consist or consist essentially of: (1) for each of a pair of BaSIRS biomarker cDNAs at least one oligonucleotide primer and/or at least one oligonucleotide probe that hybridizes to the BaSIRS biomarker cDNA, wherein the at least one oligonucleotide primer and/or the at least one oligonucleotide probe comprises a heterologous label; and (2) for each of a pair of VaSIRS biomarker cDNA at least one oligonucleotide primer and/or at least one oligonucleotide probe that hybridizes to the VaSIRS biomarker cDNA, wherein the at least one oligonucleotide primer and/or the at least one oligonucleotide probe comprise(s) a heterologous label, wherein the pair of BaSIRS biomarker cDNAs forms a BaSIRS derived biomarker combination which is not a derived biomarker combination for VaSIRS, PaSIRS or InSIRS, wherein the pair of VaSIRS biomarker cDNAs forms a VaSIRS derived biomarker combination which is not a derived biomarker combination for BaSIRS, PaSIRS or InSIRS, wherein the BaSIRS derived biomarker combination is selected from the BaSIRS derived biomarker combinations set out in TABLE A, and wherein the VaSIRS derived biomarker combination is selected from the VaSIRS derived biomarker combinations set out in TABLE B.

In some embodiments, the kits further comprise (a) for each of a pair of PaSIRS biomarker cDNAs at least one oligonucleotide primer and/or at least one oligonucleotide probe that hybridizes to the PaSIRS biomarker cDNA, wherein the at least one oligonucleotide primer and/or the at least one oligonucleotide probe comprises a heterologous label, wherein the at least one oligonucleotide primer and/or the at least one oligonucleotide probe comprises a heterologous label, wherein the pair of PaSIRS biomarker cDNAs forms a PaSIRS derived biomarker combination which is not a derived biomarker combination for BaSIRS, VaSIRS or InSIRS, and wherein the PaSIRS derived biomarker combination is selected from the PaSIRS derived biomarker combinations set out in TABLE C.

Alternatively, or in addition, the kits may further comprise (b) for each of a pair of InSIRS biomarker cDNAs at least one oligonucleotide primer and/or at least one oligonucleotide probe that hybridizes to the InSIRS biomarker cDNA, wherein the at least one oligonucleotide primer and/or the at least one oligonucleotide probe comprises a heterologous label, wherein the pair of InSIRS biomarker cDNAs forms an InSIRS derived biomarker combination which is not a derived biomarker combination for BaSIRS, VaSIRS or PaSIRS, and wherein the InSIRS derived biomarker combination is selected from the InSIRS derived biomarker combinations set out in TABLE D.

Suitably, in any of the embodiments or aspects disclosed herein, the kits may further comprise at least one oligonucleotide primer that hybridizes to a pathogen nucleic acid, and/or at least one oligonucleotide probe that hybridizes to the pathogen nucleic acid, wherein the at least one oligonucleotide primer and/or the at least one oligonucleotide probe comprises a heterologous label.

In any of the embodiments or aspects disclosed herein, the kits may further comprise a DNA polymerase. Suitably, the DNA polymerase is a thermostable DNA polymerase.

In any of the embodiments or aspects disclosed herein, the kits suitably comprise for each cDNA a pair of forward and reverse oligonucleotide primers that permit nucleic acid amplification of at least a portion of the cDNA to produce an amplicon. In representative examples of these embodiments, the kits may further comprise for each cDNA an oligonucleotide probe that comprises a heterologous label and hybridizes to the amplicon.

In specific embodiments, the components of the kits when used to determine the indicator are combined to form a mixture.

The kits may further comprise one or more reagents for preparing mRNA from a cell or cell population from a patient sample (e.g., a body fluid such as blood, urine, plasma, serum, urine, secretion or excretion). In representative examples of this type, the kits comprise a reagent for preparing cDNA from the mRNA.

In a further aspect, the present invention provides methods for treating a subject with a SIRS condition selected from BaSIRS and VaSIRS and optionally one of PaSIRS or InSIRS. These methods generally comprise, consist or consist essentially of: exposing the subject to a treatment regimen for treating the SIRS condition based on an indicator obtained from an indicator-determining method, wherein the indicator is indicative of the presence, absence or degree of the SIRS condition in the subject, and wherein the indicator-determining method is as broadly described above and elsewhere herein. In some embodiments, the methods further comprise taking a sample from the subject and determining an indicator indicative of the likelihood of the presence, absence or degree of the SIRS condition using the indicator-determining method. In other embodiments, the methods further comprise sending a sample taken from the subject to a laboratory at which the indicator is determined according to the indicator-determining method. In these embodiments, the methods suitably further comprise receiving the indicator from the laboratory.

In a related aspect, the present invention provides methods for managing a subject with a specific SIRS condition selected from BaSIRS and VaSIRS and optionally one of PaSIRS or InSIRS. These methods generally comprise, consist or consist essentially of: exposing the subject to a treatment regimen for the specific SIRS condition and avoiding exposing the subject to a treatment regimen for a SIRS condition other than the specific SIRS condition, based on an indicator obtained from an indicator-determining method, wherein the indicator is indicative of the presence, absence or degree of the SIRS condition in the subject, and wherein the indicator-determining method is an indicator-determining method as broadly described above and elsewhere herein. In some embodiments, the methods further comprise taking a sample from the subject and determining an indicator indicative of the likelihood of the presence, absence or degree of BaSIRS, VaSIRS, PaSIRS, or InSIRS using the indicator-determining method. In other embodiments, the methods further comprise sending a sample taken from the subject to a laboratory at which the indicator is determined according to the indicator-determining method. In these embodiments, the methods suitably further comprise receiving the indicator from the laboratory.

In a further aspect, the present invention provides methods of monitoring the efficacy of a treatment regimen in a subject with a SIRS condition selected from BaSIRS and VaSIRS and optionally one of PaSIRS or InSIRS, wherein the treatment regimen is monitored for efficacy towards a desired health state (e.g., absence of the SIRS condition). These methods generally comprise, consist or consist essentially of: (1) obtaining a biomarker profile of a sample taken from the subject after treatment of the subject with the treatment regimen, wherein the sample biomarker profile comprises (a) for each of a plurality of derived biomarkers as broadly defined above and elsewhere herein a plurality of host response specific derived biomarker values, and optionally (b) if the SIRS condition is an infection positive SIRS condition (“IpSIRS”), a pathogen specific biomarker value as broadly defined above and elsewhere herein for a pathogen biomarker associated with the SIRS condition; and (2) comparing the sample biomarker profile to a reference biomarker profile that is correlated with a presence, absence or degree of the SIRS condition to thereby determine whether the treatment regimen is effective for changing the health status of the subject to the desired health state.

In a related aspect, the present invention provides methods of monitoring the efficacy of a treatment regimen in a subject towards a desired health state (e.g., absence of BaSIRS, VaSIRS, PaSIRS, or InSIRS). These methods generally comprise, consist or consist essentially of: (1) determining an indicator according to an indicator-determining method as broadly described above and elsewhere herein based on a sample taken from the subject after treatment of the subject with the treatment regimen; and (2) assessing the likelihood of the subject having a presence, absence or degree of a SIRS condition selected from BaSIRS and VaSIRS and optionally one of PaSIRS or InSIRS using the indicator to thereby determine whether the treatment regimen is effective for changing the health status of the subject to the desired health state. In some embodiments, the indicator is determined using a plurality of host response specific derived biomarker values. In other embodiments, the indicator is determined using a plurality of host response specific derived biomarker values and a plurality of pathogen specific biomarker values.

Another aspect of the present invention provides methods of correlating a biomarker profile with an effective treatment regimen for a SIRS condition selected from BaSIRS and VaSIRS and optionally one of PaSIRS or InSIRS. These methods generally comprise, consist or consist essentially of: (1) determining a biomarker profile of a sample taken from a subject with the SIRS condition and for whom an effective treatment has been identified, wherein the biomarker profile comprises: (a) for each of a plurality of derived biomarkers as broadly defined above and elsewhere herein a plurality of host response specific derived biomarker values, and optionally (b) if the SIRS condition is an IpSIRS, a pathogen specific biomarker value as broadly defined above and elsewhere herein for a pathogen biomarker associated with the SIRS condition; and (2) correlating the biomarker profile so determined with an effective treatment regimen for the SIRS condition.

In yet another aspect, the present invention provides methods of determining whether a treatment regimen is effective for treating a subject with a SIRS condition selected from BaSIRS and VaSIRS and optionally one of PaSIRS or InSIRS. These methods generally comprise, consist or consist essentially of: (1) determining a post-treatment biomarker profile of a sample taken from the subject after treatment with a treatment regimen, wherein the biomarker profile comprises: (a) for each of a plurality of derived biomarkers as broadly defined above and elsewhere herein a plurality of host response specific derived biomarker values, and optionally (b) if the SIRS condition is an IpSIRS, a pathogen specific biomarker value as broadly defined above and elsewhere herein for a pathogen biomarker associated with the SIRS condition; and (2) determining a post-treatment indicator using the post-treatment biomarker profile, wherein the post-treatment indicator is at least partially indicative of the presence, absence or degree of the SIRS condition, wherein the post-treatment indicator indicates whether the treatment regimen is effective for treating the SIRS condition in the subject on the basis that post-treatment indicator indicates the presence of a healthy condition or the presence of the SIRS condition of a lower degree relative to the degree of the SIRS condition in the subject before treatment with the treatment regimen.

A further aspect of the present invention provides methods of correlating a biomarker profile with a positive or negative response to a treatment regimen for treating a SIRS condition selected from BaSIRS and VaSIRS and optionally one of PaSIRS or InSIRS. These methods generally comprise, consist or consist essentially of: (1) determining a biomarker profile of a sample taken from a subject with the SIRS condition following commencement of the treatment regimen, wherein the reference biomarker profile comprises: (a) for each of a plurality of derived biomarkers as broadly defined above and elsewhere herein a plurality of host response specific derived biomarker values, and optionally (b) if the SIRS condition is an IpSIRS, a pathogen specific biomarker value as broadly defined above and elsewhere herein for a pathogen biomarker associated with the SIRS condition; and (2) correlating the sample biomarker profile with a positive or negative response to the treatment regimen.

Another aspect of the present invention provides methods of determining a positive or negative response to a treatment regimen by a subject with a SIRS condition selected from BaSIRS and VaSIRS and optionally one of PaSIRS or InSIRS. These methods generally comprise, consist or consist essentially of: (1) correlating a reference biomarker profile with a positive or negative response to the treatment regimen, wherein the biomarker profile comprises: (a) for each of a plurality of derived biomarkers as broadly defined above and elsewhere herein a plurality of host response specific derived biomarker values, and optionally (b) if the SIRS condition is an IpSIRS, a pathogen specific biomarker value as broadly defined above and elsewhere herein for a pathogen biomarker associated with the SIRS condition; (2) detecting a biomarker profile of a sample taken from the subject, wherein the sample biomarker profile comprises (i) a plurality of host response specific derived biomarker values for each of the plurality of derived biomarkers in the reference biomarker profile, and optionally (ii) a pathogen specific biomarker value for the pathogen biomarker in the reference biomarker profile, wherein the sample biomarker profile indicates whether the subject is responding positively or negatively to the treatment regimen.

Still another aspect of the present invention provides methods of determining a positive or negative response to a treatment regimen by a subject with a SIRS condition selected from BaSIRS and VaSIRS and optionally one of PaSIRS or InSIRS. These methods generally comprise, consist or consist essentially of: (1) obtaining a biomarker profile of a sample taken from the subject following commencement of the treatment regimen, wherein the biomarker profile comprises: (a) for each of a plurality of derived biomarkers as broadly defined above and elsewhere herein a plurality of host response specific derived biomarker values, and optionally (b) if the SIRS condition is an IpSIRS, a pathogen specific biomarker value as broadly defined above and elsewhere herein for a pathogen biomarker associated with the SIRS condition, wherein the sample biomarker profile is correlated with a positive or negative response to the treatment regimen; and (2) and determining whether the subject is responding positively or negatively to the treatment regimen.

Yet other aspects of the present invention contemplate the use of the indicator-determining methods as broadly described above and elsewhere herein in methods for correlating a biomarker profile with an effective treatment regimen for a SIRS condition selected from BaSIRS and VaSIRS and optionally one of PaSIRS or InSIRS, or for determining whether a treatment regimen is effective for treating a subject with the SIRS condition, or for correlating a biomarker profile with a positive or negative response to a treatment regimen, or for determining a positive or negative response to a treatment regimen by a subject with the SIRS condition.

### BRIEF DESCRIPTION OF THE DRAWINGS

FIG. 1: Plot of the performance (AUC) of the best BaSIRS derived biomarkers following a greedy search. The best derived biomarker identified was TSPO:HCLS1 with an AUC of 0.84. The addition of further derived biomarkers adds incrementally to the overall AUC. The addition of further derived biomarkers beyond the first two was considered to add noise and difficulty in translating to a commercial format.

FIG. 2: Performance (AUC) of the final BaSIRS signature, represented as bar graphs, in the various datasets used, including in the “discovery” (training), “validation” and “control” datasets. The signature was developed to provide strong AUC in BaSIRS datasets and weak AUC in datasets containing samples derived from subjects with SIRS unrelated to bacterial infection.

FIG. 3: Performance of the final BaSIRS signature (OPLAH:ZHX2 and TSPO:HCLS1), represented as box and whisker plots, in the discovery datasets. Good separation in all datasets can be seen between Control (non-BaSIRS) and Case (BaSIRS) subjects.

FIG. 4: Performance of the final BaSIRS signature (OPLAH:ZHX2 and TSPO:HCLS1) represented as box and whisker plots, in the validation datasets. Good separation in all datasets can be seen between Control (non-BaSIRS) and Case (BaSIRS) subjects.

FIG. 5: Performance of the final BaSIRS signature (OPLAH:ZHX2 and TSPO:HCLS1) represented as box and whisker plots, in the control datasets. Poor separation in all datasets can be seen between Control (healthy or SIRS other than BaSIRS) and Case (SIRS other than BaSIRS) subjects.

FIG. 6: Plot of the performance (AUC) of the best VaSIRS derived biomarkers following a greedy search. The best derived biomarker identified was ISG15:IL16 with an AUC of 0.92. The addition of further derived biomarkers adds incrementally to the overall AUC. The addition of further derived biomarkers beyond the first two was considered to add noise and difficulty in translating to a commercial format.

FIG. 7: Box and whisker plots demonstrating performance (AUC=0.962) of the final VaSIRS signature (ISG15:IL16 and OASL:ADGRE5 in the right hand plot) in pediatric patients in intensive care with systemic inflammation. This figure shows the performance of the components of the pan-viral signature, and in combination (ISG15:IL16 and OASL:ADGRE5), in three pediatric patient cohorts from a study consisting of 12 sterile systemic inflammation (InSIRS, “control”), 28 bacterial systemic inflammation (“sepsis”), 6 viral systemic inflammation (“viral”). The study was called GAPPSS. ADGRE5 is also called CD97.

FIG. 8: Box and whisker plots showing the performance of the final VaSIRS signature (ISG15:IL16 and OASL:ADGRE5) for 624 patients admitted to intensive care with suspected sepsis (MARS clinical trial). Patients are grouped based on retrospective physician diagnosis and whether a pathogenic organism was isolated (bacteria, mixed condition, virus) or not (healthy, SIRS). Good separation of those patients retrospectively diagnosed with a viral condition, and for which a virus was isolated, can be seen when using the final VaSIRS signature in this large patient cohort.

FIG. 9: Box and whisker plots showing the performance of the final VaSIRS signature (ISG15:IL16 and OASL:ADGRE5) for patients presenting to a clinic with acute clinical signs associated with Human Immunodeficiency Virus (HIV) (GSE29429). Comparison was made between two groups of subjects, including 17 healthy controls and 30 patients infected with HIV. The Area Under Curve (AUC) was 0.91.

FIG. 10: Box and whisker plots using the final VaSIRS signature in a time course study in a limited number of piglets deliberately infected (Day 0) with porcine circovirus and followed for 29 days. Blood samples were taken prior to inoculation (Day 0) and on Days 7, 14, 21 and 29 (GSE14790). The alternate and correlated biomarker N4BP1 was substituted for OASL because this latter biomarker is not found in pigs. Areas Under Curve (AUCs) were 0.812, 1.00, 1.00 and 1.00 for Days 0 vs 7, 0 vs 14, 0 vs 21 and 0 vs 29, respectively.

FIG. 11: Use of the final VaSIRS signature in children with acute mild (n=9), moderate (n=9) or severe (n=8) Respiratory Syncytial Virus (RSV) infection, and upon 4-6 weeks of recovery for those children that had acute moderate and severe infection shows good separation between those with acute infection versus those in recovery. Little difference was found between patients with RSV infection of varying severity.

FIG. 12: Time course study of the use of the final VaSIRS signature in cynomologus macaques (n=15) infected with aerosolized Marburg virus (Filoviridae, Group V). In this study 15 Marburg virus-infected macaques (1000 pfu) were studied over a nine-day period with three animals sacrificed at each two-day interval. Cytokine and gene expression analyzes revealed similar peaks by Day 7 to that of SeptiCyte VIRUS score. The first major elevation in VaSIRS signature can be seen on Day 3 post-exposure which correlates to the first detectable presence of viral antigen in regional lymph nodes and precedes first detectable viremia (Day 4) and elevated body temperature (Day 5). (original study published by Lin, K. L., Twenhafel, N. A., Connor, J. H., Cashman, K. A., Shamblin, J. D., Donnelly, G. C., et al. (2015). Temporal Characterization of Marburg Virus Angola Infection following Aerosol Challenge in Rhesus Macaques. Journal of Virology, 89(19), 9875-9885.)

FIG. 13: Use of VaSIRS signature over time using liver biopsies from chimpanzees intravenously inoculated (Week 0) with either Hepatitis C Virus (HCV, n=3) or Hepatitis E Virus (HEV, n=4). Samples were grouped based on the independent detection of viremia, including; first positive week (and the second positive week for HCV), the peak positive week, the last positive week, the first negative week and the fourth negative week. The temporal gene expression responses for each virus (each Baltimore Group IV viruses) is different. The VaSIRS signature using liver tissue largely reflected viremia detected in plasma using virus-specific RT-PCR assays, the peak of which preceded both the antibody response and peak liver histological activity index (HAI, Ishtak activity) by 1-4 weeks for both viruses. (original study published by Yu, C., Boon, D., McDonald, S. L., Myers, T. G., Tomioka, K., Nguyen, H., et al. (2010). Pathogenesis of Hepatitis E Virus and Hepatitis C Virus in Chimpanzees: Similarities and Differences. Journal of Virology, 84(21), 11264-11278.)

FIG. 14: Plot of the performance (AUC) of the best PaSIRS derived biomarkers following a greedy search. The performance of these same derived biomarkers is also shown in a merged control dataset (lower line). The best derived biomarker identified was TTC17:G6PD with an AUC of 0.96. The addition of further derived biomarkers adds incrementally to the overall AUC. The addition of further derived biomarkers beyond the first three was considered to add noise and difficulty in translating to a commercial format.

FIG. 15: Box and whisker plots of the performance of the combination of the derived biomarkers TTC17/G6PD, HERC6/LAP3 and NUP160/TPP1 for sixteen non-protozoal datasets (top two rows) and four protozoal datasets. The overall AUC across these datasets for this single derived biomarker was 0.99.

FIG. 16: Box and whisker plots of the performance of the derived biomarkers TTC17/G6PD and HERC6/LAP3 and NUP160/TPP1 for Clinical (protozoal) and Control (non-protozoal) datasets. The Clinical dataset consists of five merged datasets (GSE34404, 64610, 33811, 15221 and 5418), and the Control dataset consists of 16 merged datasets, including four viral (GSE40366, 41752, 51808, 52428), eight SIRS (GSE19301, 38485, 46743, 64813, 17755, 47655, 29532, 61672), three Triage (GSE11908, 33341, 25504) and one healthy (GSE35846). Each merged dataset contains those subjects (or patients) with the condition under study (Case) and those subjects without the condition (Control). Good separation can be observed between the Case and Control in the Clinical (protozoal) dataset whilst there is poor separation between Case and Control in the Control dataset. Such performance indicates specificity of the derived biomarkers.

FIG. 17: Box and whisker plots demonstrating the performance of the final PaSIRS signature TTC17/G6PD, HERC6/LAP3 and NUP160/TPP1 in the dataset GSE43661. Macrophages from three donors were cultured and either infected with Leishmania major (Case) or mock infected (Control). Samples were taken at time point 0 and at 3, 6, 12 and 24 hours. The value of the derived biomarkers changes over time in both infected and mock-infected samples and the largest difference between these two cohorts can be seen at time points 3 and 6 hours post-infection.

FIG. 18: Box and whisker plot showing the performance of the final PaSIRS signature TTC17/G6PD, HERC6/LAP3 and NUP160/TPP1 in the dataset GSE23750. Intestinal biopsies were taken from eight patients with Entamoeba histolytica infection on Day 1 and on Day 60 following treatment. A difference between the two time points can be observed but it is not large, perhaps because the sample was an intestinal biopsy rather than peripheral blood.

FIG. 19: Box and whisker plot showing the performance of the final PaSIRS signature TTC17/G6PD, HERC6/LAP3 and NUP160/TPP1 in dataset GSE7047. Cultured (in vitro) HeLa cells were either infected or not with Trypanosoma cruzi. Three replicates were performed. A large difference can be observed in the value obtained for this combination of derived biomarkers between infected and uninfected HeLa cells.

FIG. 20: Box and whisker plot showing the performance of the final PaSIRS signature TTC17/G6PD, HERC6/LAP3 and NUP160/TPP1 in dataset GSE50957. Five people on malaria prophylaxis were infected with Plasmodium falciparum through the bites of infected mosquitos and blood samples were taken pre- and post-infection. Blood samples from two healthy controls were also included in the study. Despite the subjects being on malaria prophylaxis a large difference can be observed between samples taken pre- and post-infection.

FIG. 21: Box and whisker plot showing the performance of the final PaSIRS signature TTC17/G6PD, HERC6/LAP3 and NUP160/TPP1 in dataset GSE52166 which is a larger study of the same design as GSE50957 but involving more patients (n=54, samples taken pre- and post-infection). Despite the subjects being on malaria prophylaxis a difference, albeit less dramatic than for GSE50957, can be observed between samples taken pre- and post-infection.

FIG. 22: Plot of the performance (AUC) of the best inSIRS derived biomarkers following a greedy search. The best derived biomarker identified was ENTPD1:ARL6IP5 with an AUC of 0.898. The addition of further derived biomarkers adds incrementally to the overall AUC. The addition of further derived biomarkers beyond the first two was considered to add noise and difficulty in translating to a commercial format.

FIG. 23: Box and whisker plots showing the performance of the inSIRS signature (ENTPD1/ARL6IP5; TNFSF8/HEATR1) using controls datasets (infectious SIRS; GSE datasets 11909 (mixed conditions including autoimmunity vs infection positive), 19301 (asthma exacerbation vs quiescent), 38485 (schizophrenia vs healthy), 41752 (Lassa virus infection vs healthy), 42834 (tuberculosis vs healthy), 51808 (Dengue virus infection vs healthy), 52428 (influenza virus infection vs healthy), 61672 (anxiety vs not) and 64813 (post-traumatic stress syndrome vs pre-stress).

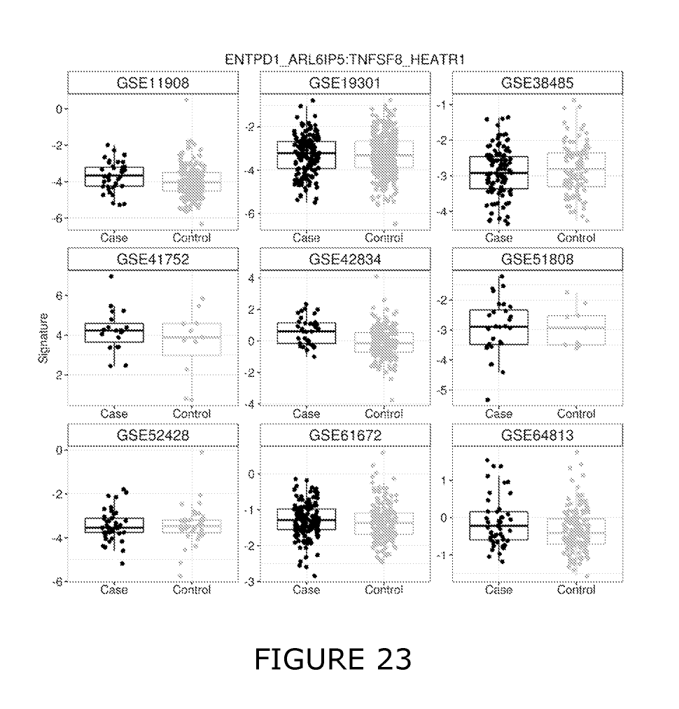

FIG. 24: Box and whisker plots showing the performance of the inSIRS signature (ENTPD1/ARL6IP5; TNFSF8/HEATR1) using discovery datasets, including GAPPSS (sepsis and surgical SIRS in children), GSE17755 (autoimmune disease vs infected), GSE36809 (trauma with and without sepsis), GSE47655 (anaphylaxis), GSE63990 (acute respiratory infection) and 74224 (sepsis and SIRS in adults).

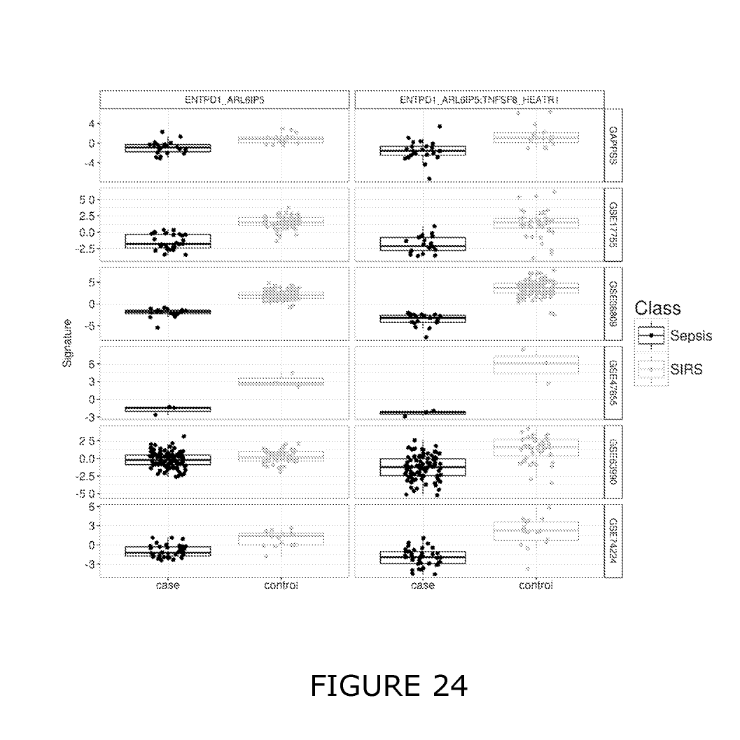

FIG. 25: Box and whisker plots showing the performance of the inSIRS signature (ENTPD1/ARL6IP5; TNFSF8/HEATR1) using a separate set of samples (validation) from the datasets, including GAPPSS (sepsis and surgical SIRS in children), GSE17755 (autoimmune disease vs infected), GSE36809 (trauma, with or without sepsis), GSE47655 (anaphylaxis), GSE63990 (acute respiratory infection) and 74224 (sepsis and SIRS in adults).

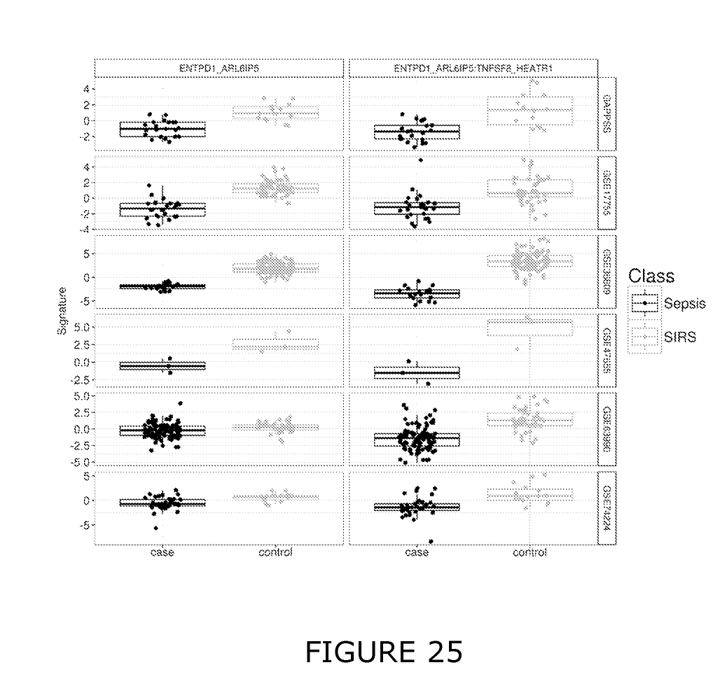

FIG. 26: Multi-dimensional scaling plot using random forest and BaSIRS and VaSIRS derived biomarkers on data associated with GSE63990. Good separation of patients with acute respiratory inflammation into those patients with bacterial and viral infections and non-infectious illness can be observed when using BaSIRS and VaSIRS derived biomarkers. It can be seen that some patients with acute respiratory inflammation due to a bacterial infection (as diagnosed by a clinician) cluster with those patients with a viral infection (as determined using multi-dimensional scaling) and vice versa.

FIG. 27: Example patient report for the host response specific biomarkers for a bacterial infection (alone)—called SeptiCyte MICROBE.

FIG. 28: Example patient report for the host response specific biomarkers for a viral infection (alone)—called SeptiCyte VIRUS.

FIG. 29: Example patient report for the host response specific biomarkers for a protozoal infection (alone)—called SeptiCyte PROTOZOAN.

FIG. 30: Example patient report for the host response specific biomarkers for bacterial, viral, protozoal and infection negative systemic inflammation combined—called SeptiCyte SPECTRUM. In this instance the patient has a predominant bacterial host response.

FIG. 31: Example patient report for the host response specific biomarkers for bacterial, viral, protozoal and infection negative systemic inflammation combined—called SeptiCyte SPECTRUM. In this instance the patient has a predominant viral host response.

FIG. 32: Example patient report for the host response specific biomarkers for bacterial, viral, protozoal and infection negative systemic inflammation combined—called SeptiCyte SPECTRUM. In this instance the patient has a predominant protozoal host response.

FIG. 33: Example patient report for the host response specific biomarkers for bacterial, viral, protozoal and infection negative systemic inflammation combined—called SeptiCyte SPECTRUM. In this instance the patient has a predominant non-infectious host response.

FIG. 34: Plot of BaSIRS signature results (Y axis, host response) versus bacterial pathogen detection results (X axis, pathogen molecule) for intensive care patients with retrospectively diagnosed “sepsis” (ipSIRS), “SIRS” (InSIRS) or “indeterminate” (three clinicians could not decide on a diagnosis). The Y axis is designated as “SeptiScore”, which is a probability of BaSIRS, and the X axis is in RT-PCR cycle time (Ct), which is a measurement of bacterial DNA in whole blood. Each dot represents a patient blood sample that has been tested and those that are circled (on the right hand side) are the only samples that were found to be blood culture positive. Such samples also have low Ct values, indicating that bacterial DNA could be detected at high levels, and high SeptiScores, indicating a strong specific host response to bacterial infection.

FIG. 35: Plot of VaSIRS signature and viral pathogen results for intensive care patients included in the MARS study. Those patients that were viral pathogen positive are circled (with varying sized circles for different virus types). In particular, those patients positive for influenza and RSV virus antigens are also strongly positive for VaSIRS signature.

FIG. 36. A plot of scores obtained for SeptiCyte™ VIRUS and SeptiCyte™ MICROBE for pediatric patients participating in a clinical trial that presented with clinical signs of SIRS. Some patients (n=28) were retrospectively diagnosed as having sepsis (nine were also positive on PCR for a viral infection), some (n=6) were retrospectively diagnosed as having a viral infection (three were also diagnosed as having confirmed or suspected sepsis), and some were retrospectively diagnosed as having systemic inflammation but no infection (n=12). Good separation can be seen between those patients having InSIRS (“Control”) compared to other causes of SIRS. However, separation between those patients with BaSIRS and VaSIRS is less clear, suggesting that, for at least some patients, inflammation due to multiple pathogen types can exist at the same time. Further, viral infection may lead to bacterial infection, or bacterial infection may lead to viral infection.

FIG. 37: Box and whisker plots demonstrating the performance, as measured by probability (Y axis), of each of the PaSIRS (“Protozoal”), BaSIRS (“Bacterial”), VaSIRS (“Viral”) and InSIRS (“SIRS”) final signatures in eight individual and independent GEO datasets covering a range of conditions including patients with sepsis, influenza, malaria, non-infectious systemic inflammation, and healthy subjects. The probabilities demonstrate that each systemic inflammatory signature is specific for its intended target condition. Combined probabilities were determined by mapping each score onto a sigmoidal curve via the logit function. Probabilities were then calculated using a LOO-CV approach.

### BRIEF DESCRIPTION OF THE TABLES

TABLE 1: Representative key human pathogens that are known to cause systemic inflammation and bacteremia, fungemia, viremia or protozoan parasitemia.

TABLE 2: Common human viruses that cause SIRS as part of their pathogenesis and for which there are specific anti-viral treatments.

TABLES 3: BaSIRS biomarker details including; Sequence identification number, gene symbol and Ensembl transcript ID.

TABLE 4: BaSIRS biomarker details including; Sequence identification number, gene symbol and GenBank accession.

TABLE 5: VaSIRS biomarker details including; Sequence identification number, gene symbol and Ensembl transcript ID.

TABLE 6: VaSIRS biomarker details including; Sequence identification number, gene symbol and GenBank accession.

TABLE 7: PaSIRS biomarker details including; Sequence identification number, gene symbol and Ensembl transcript ID.

TABLE 8: PaSIRS biomarker details including; Sequence identification number, gene symbol and GenBank accession.

TABLE 9: PaSIRS biomarker details including; Sequence identification number, gene symbol and Ensembl transcript ID.

TABLE 10: PaSIRS biomarker details including; Sequence identification number, gene symbol and GenBank accession.

TABLE 11: Exemplary Escherichia coli DNA sequence including Single Nucleotide Polymorphisms (SNPs) at positions 396 and 398 (bolded).

TABLE 12: Description of datasets and number of samples used as part of discovery of derived biomarkers for BaSIRS. The total number of genes that were able to be used across all of these datasets was 3698. All useable samples in these datasets were randomly divided into BaSIRS discovery and validation (see TABLE 10) sets.

TABLE 13: Description of datasets and number of samples used as part of validation of derived biomarkers for BaSIRS.

TABLE 14: Description of control datasets and number of samples used for subtraction from the derived biomarkers for BaSIRS. The subtraction process ensured that the BaSIRS derived biomarkers were specific.

TABLE 15: Performance (as measured by AUC) of the final BaSIRS signature in each of the Discovery, Validation and Control datasets.

TABLE 16: Performance (as meassured by AUC) of the top 102 BaSIRS derived biomarkers in each of the BaSIRS validation datasets. Only those derived biomarkers with a mean AUC>0.85 were used in a greedy search to identify the best combination of derived biomarkers.

TABLE 17: Details of Gene Expression Omnibus (GEO) datasets used for discovery of viral derived biomarkers.

TABLE 18: Details of Gene Expression Omnibus (GEO) datasets used for validation of viral derived biomarkers.

TABLE 19: Description of control datasets used for subtraction from the derived biomarkers for VaSIRS. The subtraction process ensured that the VaSIRS derived biomarkers were specific.

TABLE 20: List of derived VaSIRS biomarkers with an of AUC>0.8 in at least 11 of 14 viral datasets.

TABLE 21: Details of Gene Expression Omnibus (GEO) datasets used for discovery of protozoal derived biomarkers.

TABLE 22: Description of the GEO datasets used for validation of the protozoal derived biomarkers.

TABLE 23: Description of control datasets used for subtraction from the derived biomarkers for PaSIRS. The subtraction process ensured that the PaSIRS derived biomarkers were specific.

TABLE 24: Description of datasets used for discovery, validation and subtraction from the derived biomarkers for InSIRS. The subtraction process ensured that the InSIRS derived biomarkers were specific.

TABLE 25: Derived biomarkers grouped (A, B, C, D) based on correlation to each of the biomarkers in the final BaSIRS signature (OPLAH, ZHX2, TSPO, HCLS1).

TABLE 26: Derived biomarkers grouped (A, B, C, D) based on correlation to each of the biomarkers in the final VaSIRS signature (ISG15, IL16, OASL, ADGRE5).

TABLE 27: Derived biomarkers grouped (A, B, C, D) based on correlation to each of the biomarkers in the final PaSIRS signature (TTC17, G6PD, HERC6, LAP3, NUP160, TPP1).

TABLE 28: Derived biomarkers grouped (A, B, C, D) based on correlation to each of the biomarkers in the final inSIRS signature (ARL6IP5, ENTPD1, HEATR1, TNFSF8).

TABLE 29: Top performing (based on AUC) BaSIRS derived biomarkers following a greedy search on a combined dataset. The top derived biomarker was TSPO:HCLS1 with an AUC of 0.838. Incremental AUC increases can be made with the addition of further derived biomarkers as indicated.

TABLE 30: BaSIRS numerators and denominators appearing more than once in derived biomarkers with a mean AUC>0.85 in the validation datasets.

TABLE 31: Top performing (based on AUC) VaSIRS derived biomarkers following a greedy search on a combined dataset. The top derived biomarker was ISG15:IL16 with an AUC of 0.92. Incremental AUC increases can be made with the addition of further derived biomarkers as indicated.

TABLE 32: VaSIRS numerators and denominators appearing more than twice in the 473 derived biomarkers with a mean AUC>0.80 in at least 11 of 14 viral datasets.

TABLE 33: Top performing (based on AUC) PaSIRS derived biomarkers following a greedy search on a combined dataset. The top derived biomarker was TTC17:G6PD with an AUC of 0.96. Incremental AUC increases can be made with the addition of further derived biomarkers as indicated.

TABLE 34: PaSIRS numerators and denominators appearing more than twice in the 523 derived biomarkers with a mean AUC>0.75 in the validation datasets.

TABLE 35: TABLE of individual performance, in descending AUC, of the 523 PaSIRS derived biomarkers with an average AUC>0.75 across each of five protozoal datasets.

TABLE 36: Top performing (based on AUC) InSIRS derived biomarkers following a greedy search on a combined dataset. The top derived biomarker was ENTPD1:ARL6IP5 with an AUC of 0.898. Incremental AUC increases can be made with the addition of further derived biomarkers as indicated.

TABLE 37: inSIRS numerators and denominators appearing more than twice in the 164 derived biomarkers with a mean AUC>0.82 in the validation datasets.

TABLE 38: TABLE of individual performance, in descending AUC, of 164 inSIRS derived biomarkers with an average AUC>0.82 across each of six non-infectious systemic inflammation datasets.

TABLE 39: Interpretation of results obtained when using a combination of BaSIRS and bacterial detection.

TABLE 40: Interpretation of results obtained when using a combination of VaSIRS and virus detection.

TABLE 41: Interpretation of results obtained when using a combination of PaSIRS and protozoan detection.

### DETAILED DESCRIPTION OF THE INVENTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e., to at least one) of the grammatical object of the article. By way of example, “an element” means one element or more than one element.

As used herein, “and/or” refers to and encompasses any and all possible combinations of one or more of the associated listed items, as well as the lack of combinations when interpreted in the alternative (or).

The term “biomarker” broadly refers to any detectable compound, such as a protein, a peptide, a proteoglycan, a glycoprotein, a lipoprotein, a carbohydrate, a lipid, a nucleic acid (e.g., DNA, such as cDNA or amplified DNA, or RNA, such as mRNA), an organic or inorganic chemical, a natural or synthetic polymer, a small molecule (e.g., a metabolite), or a discriminating molecule or discriminating fragment of any of the foregoing, that is present in or derived from a sample, typically a biological sample. “Derived from” as used in this context refers to a compound that, when detected, is indicative of a particular molecule being present in the sample. For example, detection of a particular cDNA can be indicative of the presence of a particular RNA transcript in the sample. As another example, detection of or binding to a particular antibody can be indicative of the presence of a particular antigen (e.g., protein) in the sample. Here, a discriminating molecule or fragment is a molecule or fragment that, when detected, indicates presence or abundance of an above-identified compound. A biomarker can, for example, be isolated from a sample, directly measured in a sample, or detected in or determined to be in a sample. A biomarker can, for example, be functional, partially functional, or non-functional. In specific embodiments, the “biomarkers” include “host response biomarkers”, and “pathogen biomarkers”, which are described in more detail below. A biomarker is considered to be informative for a SIRS condition as disclosed herein if a measurable aspect of the biomarker is associated with the presence of the SIRS condition in a subject in comparison to a predetermined value or a reference profile from a control population. Such a measurable aspect may include, for example, the presence, absence, or level of the biomarker in the sample, and/or its presence or level as a part of a profile of more than one biomarker, for example as part of a combination with one or more other biomarkers, including as part of a derived biomarker combination as described herein.

The term “biomarker value” refers to a value measured or derived for at least one corresponding biomarker of a subject and which is typically at least partially indicative of a level of a biomarker in a sample taken from the subject. Thus, the biomarker values could be measured biomarker values, which are values of biomarkers measured for the subject. These values may be quantitative or qualitative. Fo example, a measured biomarker value may refer to the presence or absence of a biomarker or may refer to a level of a biomarker, in a sample. The measured biomarker values can be values relating to raw or normalized biomarker levels (e.g., a raw, non-normalized biomarker level, or a normalized biomarker levels that is determined relative to an internal or external control biomarker level) and to mathematically transformed biomarker levels (e.g., a logarithmic representation of a biomarker level such as amplification amount, cycle time, etc.). Alternatively, the biomarker values could be derived biomarker values, which are values that have been derived from one or more measured biomarker values, for example by applying a function to the one or more measured biomarker values. Biomarker values can be of any appropriate form depending on the manner in which the values are determined. For example, the biomarker values could be determined using high-throughput technologies such as mass spectrometry, sequencing platforms, array and hybridization platforms, immunoassays, flow cytometry, or any combination of such technologies and in one preferred example, the biomarker values relate to a level of activity or abundance of an expression product or other measurable molecule, quantified using a technique such as PCR, sequencing or the like. In this case, the biomarker values can be in the form of amplification amounts, or cycle times, which are a logarithmic representation of the levels of the biomarker within a sample and which thus correspond to mathematical transformations of raw or normalized biomarker levels, as will be appreciated by persons skilled in the art and as will be described in more detail below. Thus, in situations in which mathematically transformed biomarker values are used as measured biomarker values, the expression “derived biomarker value being indicative of a ratio of levels of a plurality of biomarkers” and the like does not necessarily mean that the derived biomarker value is one that results from a division of one measured biomarker value by another measured biomarker value. Instead, the measured biomarker values can be combined using any suitable function, whereby the resulting derived biomarker value is one that corresponds to or reflects a ratio of non-normalized (e.g., raw) or normalized biomarker levels.

The term “biomarker profile” refers to one or a plurality of one or more types of biomarkers (e.g., an mRNA molecule, a cDNA molecule and/or a protein, lipopolysaccharide, etc.), or an indication thereof, together with a feature, such as a measurable aspect (e.g., biomarker value that is measured or derived), of the biomarker(s). A biomarker profile may comprise a single biomarker level that correlates with the presence, absence or degree of a condition (e.g., BaSIRS or VaSIRS, or PaSIRS or InSIRS). Alternatively, a biomarker profile may comprise at least two such biomarkers or indications thereof, where the biomarkers can be in the same or different classes, such as, for example, a nucleic acid and a polypeptide. Thus, a biomarker profile may comprise at least 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, or 100 or more biomarkers or indications thereof. In some embodiments, a biomarker profile comprises hundreds, or even thousands, of biomarkers or indications thereof. A biomarker profile can further comprise one or more controls or internal standards. In certain embodiments, the biomarker profile comprises at least one biomarker, or indication thereof, that serves as an internal standard. In other embodiments, a biomarker profile comprises an indication of one or more types of biomarkers. The term “indication” as used herein in this context merely refers to a situation where the biomarker profile contains symbols, data, abbreviations or other similar indicia for a biomarker, rather than the biomarker molecular entity itself. The term “biomarker profile” is also used herein to refer to a biomarker value or combination of at least two biomarker values, wherein individual biomarker values correspond to values of biomarkers that can be measured or derived from one or more subjects, which combination is characteristic of a discrete condition, stage of condition, subtype of condition. The term “profile biomarkers” is used to refer to a subset of the biomarkers that have been identified for use in a biomarker profile that can be used in performing a clinical assessment, such as to rule in or rule out a specific condition, different stages or severity of conditions, or subtypes of different conditions. The number of profile biomarkers will vary, but is typically of the order of 10 or less.

The terms “complementary” and “complementarity” refer to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands.

Throughout this specification, unless the context requires otherwise, the words “comprise,” “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The term “correlating” refers to determining a relationship between one type of data with another or with a state.

The term “degree” of BaSIRS, VaSIRS, PaSIRS, or InSIRS, as used herein, refers to the seriousness, severity, stage or state of a BaSIRS, VaSIRS, PaSIRS, or InSIRS. For example, a BaSIRS, VaSIRS, PaSIRS, or InSIRS may be characterized as mild, moderate or severe. A person of skill in the art would be able to determine or assess the degree of a particular BaSIRS, VaSIRS, PaSIRS, or InSIRS. For example, the degree of a BaSIRS, VaSIRS, PaSIRS, or InSIRS may be determined by comparing the likelihood or length of survival of a subject having a BaSIRS, VaSIRS, PaSIRS, or InSIRS with the likelihood or length of survival in other subjects having BaSIRS, VaSIRS, PaSIRS, or InSIRS. In other embodiments, the degree of a BaSIRS, VaSIRS, PaSIRS, or InSIRS may be determined by comparing the clinical signs of a subject having a condition with the degree of the clinical signs in other subjects having BaSIRS, VaSIRS, PaSIRS, or InSIRS.

As used herein, the terms “diagnosis”, “diagnosing” and the like are used interchangeably herein to encompass determining the likelihood that a subject will develop a condition, or the existence or nature of a condition in a subject. These terms also encompass determining the severity of disease or episode of disease, as well as in the context of rational therapy, in which the diagnosis guides therapy, including initial selection of therapy, modification of therapy (e.g., adjustment of dose or dosage regimen), and the like. By “likelihood” is meant a measure of whether a subject with particular measured or derived biomarker values actually has a condition (or not) based on a given mathematical model. An increased likelihood for example may be relative or absolute and may be expressed qualitatively or quantitatively. For instance, an increased likelihood may be determined simply by determining the subject's measured, derived or indicator biomarker values for at least two BaSIRS, VaSIRS, PaSIRS, or InSIRS biomarkers in combination with at least one pathogen specific biomarker and placing the subject in an “increased likelihood” category, based upon previous population studies. The term “likelihood” is also used interchangeably herein with the term “probability”. The term “risk” relates to the possibility or probability of a particular event occurring at some point in the future. “Risk stratification” refers to an arraying of known clinical risk factors to allow physicians to classify patients into a low, moderate, high or highest risk of developing a particular disease or condition.

The term “gene”, as used herein, refers to a stretch of nucleic acid that codes for a polypeptide or for an RNA chain that has a function. While it is the exon region of a gene that is transcribed to form mRNA, the term “gene” also includes regulatory regions such as promoters and enhancers that govern expression of the exon region.

By “high density acid arrays” and the like is meant those arrays that contain at least 400 different features (e.g., probes) per cm².

The term “indicator” as used herein refers to a result or representation of a result, including any information, number, ratio, signal, sign, mark, or note by which a skilled artisan can estimate and/or determine a likelihood or risk of whether or not a subject is suffering from a given disease or condition. In the case of the present invention, the “indicator” may optionally be used together with other clinical characteristics, to arrive at a diagnosis (that is, the occurrence or nonoccurrence) of BaSIRS, VaSIRS, PaSIRS, or InSIRS in a subject. That such an indicator is “determined” is not meant to imply that the indicator is 100% accurate. The skilled clinician may use the indicator together with other clinical indicia to arrive at a diagnosis.

The term “immobilized” means that a molecular species of interest is fixed to a solid support, suitably by covalent linkage. This covalent linkage can be achieved by different means depending on the molecular nature of the molecular species. Moreover, the molecular species may be also fixed on the solid support by electrostatic forces, hydrophobic or hydrophilic interactions or Van-der-Waals forces. The above described physico-chemical interactions typically occur in interactions between molecules. In particular embodiments, all that is required is that the molecules (e.g., nucleic acids or polypeptides) remain immobilized or attached to a support under conditions in which it is intended to use the support, for example in applications requiring nucleic acid amplification and/or sequencing or in in antibody-binding assays. For example, oligonucleotides or primers are immobilized such that a 3′ end is available for enzymatic extension and/or at least a portion of the sequence is capable of hybridizing to a complementary sequence. In some embodiments, immobilization can occur via hybridization to a surface attached primer, in which case the immobilized primer or oligonucleotide may be in the 3′-5′ orientation. In other embodiments, immobilization can occur by means other than base-pairing hybridization, such as the covalent attachment.

The term “immune system”, as used herein, refers to cells, molecular components and mechanisms, including antigen-specific and non-specific categories of the adaptive and innate immune systems, respectively, that provide a defense against damage and insults resulting from a viral infection. The term “innate immune system” refers to a host's non-specific reaction to insult to include antigen-nonspecific defense cells, molecular components and mechanisms that come into action immediately or within several hours after exposure to almost any insult or antigen. Elements of the innate immunity include for example phagocytic cells (monocytes, macrophages, dendritic cells, polymorphonuclear leukocytes such as neutrophils, reticuloendothelial cells such as Kupffer cells, and microglia), cells that release inflammatory mediators (basophils, mast cells and eosinophils), natural killer cells (NK cells) and physical barriers and molecules such as keratin, mucous, secretions, complement proteins, immunoglobulin M (IgM), acute phase proteins, fibrinogen and molecules of the clotting cascade, and cytokines. Effector compounds of the innate immune system include chemicals such as lysozymes, IgM, mucous and chemoattractants (e.g., cytokines or histamine), complement and clotting proteins. The term “adaptive immune system” refers to antigen-specific cells, molecular components and mechanisms that emerge over several days, and react with and remove a specific antigen. The adaptive immune system develops throughout a host's lifetime. The adaptive immune system is based on leukocytes, and is divided into two major sections: the humoral immune system, which acts mainly via immunoglobulins produced by B cells, and the cell-mediated immune system, which functions mainly via T cells.

Reference herein to “immuno-interactive” includes reference to any interaction, reaction, or other form of association between molecules and in particular where one of the molecules is, or mimics, a component of the immune system.

The term “level” as used herein encompasses the absolute amount of a biomarker as referred to herein, the relative amount or concentration of the biomarker as well as any value or parameter which correlates thereto or can be derived therefrom. For example, the level can be a copy number, weight, moles, abundance, concentration such as μg/L or a relative amount such as 1.0, 1.5, 2.0, 2.5, 3, 5, 10, 15, 20, 25, 30, 40, 60, 80 or 100 times a reference or control level. Optionally, the term level includes the level of a biomarker normalized to an internal normalization control, such as the expression of a housekeeping gene.

The term “microarray” refers to an arrangement of hybridizable array elements, e.g., probes (including primers), ligands, biomarker nucleic acid sequence or protein sequences on a substrate.

By monitoring the “progression” of a SIRS condition over time, is meant that changes in the severity (e.g., worsening or improvement) of the SIRS condition or particular aspects of the SIRS condition are monitored over time.

The term “nucleic acid” or “polynucleotide” as used herein includes RNA, mRNA, miRNA, cRNA, cDNA mtDNA, or DNA. The term typically refers to a polymeric form of nucleotides of at least 10 bases in length, either ribonucleotides or deoxynucleotides or a modified form of either type of nucleotide. The term includes single and double stranded forms of DNA or RNA.

By “obtained” is meant to come into possession. Samples so obtained include, for example, nucleic acid extracts or polypeptide extracts isolated or derived from a particular source. For instance, the extract may be isolated directly from a biological fluid or tissue of a subject.

The term “pathogen biomarker” refers to any bacterial, viral or protozoan molecule. The pathogen molecules can be nucleic acid, protein, carbohydrate, lipid, metabolite or combinations of such molecules.

As used herein, the term “positive response” means that the result of a treatment regimen includes some clinically significant benefit, such as the prevention, or reduction of severity, of symptoms, or a slowing of the progression of the condition. By contrast, the term “negative response” means that a treatment regimen provides no clinically significant benefit, such as the prevention, or reduction of severity, of symptoms, or increases the rate of progression of the condition.

“Protein”, “polypeptide” and “peptide” are used interchangeably herein to refer to a polymer of amino acid residues and to variants and synthetic analogues of the same.

By “primer” is meant an oligonucleotide which, when paired with a strand of DNA, is capable of initiating the synthesis of a primer extension product in the presence of a suitable polymerizing agent. The primer is preferably single-stranded for maximum efficiency in amplification but can alternatively be double-stranded. A primer must be sufficiently long to prime the synthesis of extension products in the presence of the polymerization agent. The length of the primer depends on many factors, including application, temperature to be employed, template reaction conditions, other reagents, and source of primers. For example, depending on the complexity of the target sequence, the primer may be at least about 5, 6, 7, 8, 9, 10, 11, 12, 13, 14, 15, 16, 17, 18, 19, 20, 21, 22, 23, 24, 25, 26, 27, 28, 29, 30, 35, 40, 50, 75, 100, 150, 200, 300, 400, 500, to one base shorter in length than the template sequence at the 3′ end of the primer to allow extension of a nucleic acid chain, though the 5′ end of the primer may extend in length beyond the 3′ end of the template sequence. In certain embodiments, primers can be large polynucleotides, such as from about 35 nucleotides to several kilobases or more. Primers can be selected to be “substantially complementary” to the sequence on the template to which it is designed to hybridize and serve as a site for the initiation of synthesis. By “substantially complementary”, it is meant that the primer is sufficiently complementary to hybridize with a target polynucleotide. Desirably, the primer contains no mismatches with the template to which it is designed to hybridize but this is not essential. For example, non-complementary nucleotide residues can be attached to the 5′ end of the primer, with the remainder of the primer sequence being complementary to the template. Alternatively, non-complementary nucleotide residues or a stretch of non-complementary nucleotide residues can be interspersed into a primer, provided that the primer sequence has sufficient complementarity with the sequence of the template to hybridize therewith and thereby form a template for synthesis of the extension product of the primer.

As used herein, the term “probe” refers to a molecule that binds to a specific sequence or sub-sequence or other moiety of another molecule. Unless otherwise indicated, the term “probe” typically refers to a nucleic acid probe that binds to another nucleic acid, also referred to herein as a “target polynucleotide”, through complementary base pairing. Probes can bind target polynucleotides lacking complete sequence complementarity with the probe, depending on the stringency of the hybridization conditions. Probes can be labeled directly or indirectly and include primers within their scope.

The term “sample” as used herein includes any biological specimen that may be extracted, untreated, treated, diluted or concentrated from a subject. Samples may include, without limitation, biological fluids, exudates such as whole blood, serum, red blood cells, white blood cells, plasma, saliva, urine, stool (i.e., faeces), tears, sweat, phlegm, sebum, nipple aspirate, ductal lavage, bronchial, pharyngeal or nasal lavage or swab, tumor exudates, synovial fluid, ascitic fluid, peritoneal fluid, amniotic fluid, cerebrospinal fluid, lymph, fine needle aspirate, amniotic fluid, any other bodily fluid, cell lysates, cellular secretion products, inflammation fluid, semen and vaginal secretions. Samples may include tissue samples and biopsies, tissue homogenates, washes, swabs and the like. Advantageous samples may include ones comprising any one or more biomarkers as taught herein in detectable quantities. Suitably, the sample is readily obtainable by minimally invasive methods, allowing the removal or isolation of the sample from the subject. In certain embodiments, the sample contains blood, especially peripheral blood, or a fraction or extract thereof. Typically, the sample comprises blood cells such as mature, immature or developing leukocytes, including lymphocytes, polymorphonuclear leukocytes, neutrophils, monocytes, reticulocytes, basophils, coelomocytes, hemocytes, eosinophils, megakaryocytes, macrophages, dendritic cells natural killer cells, or fraction of such cells (e.g., a nucleic acid or protein fraction). In specific embodiments, the sample comprises leukocytes including peripheral blood mononuclear cells (PBMC).

The term “solid support” as used herein refers to a solid inert surface or body to which a molecular species, such as a nucleic acid and polypeptides can be immobilized. Non-limiting examples of solid supports include glass surfaces, plastic surfaces, latex, dextran, polystyrene surfaces, polypropylene surfaces, polyacrylamide gels, gold surfaces, and silicon wafers. In some embodiments, the solid supports are in the form of membranes, chips or particles. For example, the solid support may be a glass surface (e.g., a planar surface of a flow cell channel). In some embodiments, the solid support may comprise an inert substrate or matrix which has been “functionalized”, such as by applying a layer or coating of an intermediate material comprising reactive groups which permit covalent attachment to molecules such as polynucleotides. By way of non-limiting example, such supports can include polyacrylamide hydrogels supported on an inert substrate such as glass. The molecules (e.g., polynucleotides) can be directly covalently attached to the intermediate material (e.g., a hydrogel) but the intermediate material can itself be non-covalently attached to the substrate or matrix (e.g., a glass substrate). The support can include a plurality of particles or beads each having a different attached molecular species.

As used herein, the term SIRS (“systemic inflammatory response syndrome”) refers to a clinical response arising from a non-specific insult with two or more of the following measureable clinical characteristics; a body temperature greater than 38° C. or less than 36° C., a heart rate greater than 90 beats per minute, a respiratory rate greater than 20 per minute, a white blood cell count (total leukocytes) greater than 12,000 per mm³ or less than 4,000 per mm³, or a band neutrophil percentage greater than 10%. From an immunological perspective, it may be seen as representing a systemic response to insult (e.g., major surgery) or systemic inflammation. As used herein, “VaSIRS” includes any one or more (e.g., 1, 2, 3, 4, 5) of the clinical responses noted above but with underlying viral infection etiology. Confirmation of infection can be determined using any suitable procedure known in the art, illustrative examples of which include nucleic acid detection (e.g., polymerase chain reaction (PCR), immunological detection (e.g., ELISA), isolation of virus from infected cells, cell lysis and imaging techniques such as electron microscopy. From an immunological perspective, VaSIRS may be seen as a systemic response to viral infection, whether it is a local, peripheral or systemic infection.

The terms “subject”, “individual” and “patient” are used interchangeably herein to refer to an animal subject, particularly a vertebrate subject, and even more particularly a mammalian subject. Suitable vertebrate animals that fall within the scope of the invention include, but are not restricted to, any member of the phylum Chordata, subphylum vertebrata including primates, rodents (e.g., mice rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars etc.), marine mammals (e.g., dolphins, whales), reptiles (snakes, frogs, lizards, etc.), and fish. A preferred subject is a primate (e.g., a human, ape, monkey, chimpanzee). The subject suitably has at least one (e.g., 1, 2, 3, 4, 5 or more) clinical sign of SIRS.

As used herein, the term “treatment regimen” refers to prophylactic and/or therapeutic (i.e., after onset of a specified condition) treatments, unless the context specifically indicates otherwise. The term “treatment regimen” encompasses natural substances and pharmaceutical agents (i.e., “drugs”) as well as any other treatment regimen including but not limited to dietary treatments, physical therapy or exercise regimens, surgical interventions, and combinations thereof.

It will be appreciated that the terms used herein and associated definitions are used for the purpose of explanation only and are not intended to be limiting.

### 2. Pan-Bacterial, Pan-Viral, Pan-Protozoal and Infection-Negative SIRS Biomarkers and their Use for Identifying Subjects with BaSIRS, VaSIRS, PaSIRS or InSIRS

The present invention concerns methods, apparatus, compositions and kits for identifying subjects with BaSIRS, VaSIRS, PaSIRS or InSIRS. In particular, BaSIRS, VaSIRS, PaSIRS, or InSIRS biomarkers and BIP, VIP and PIP biomarkers are disclosed for use alone or in combination in these modalities to assess the likelihood of the presence, absence or degree of BaSIRS, VaSIRS, PaSIRS or InSIRS in subjects. The methods, apparatus, compositions and kits of the invention are useful for early detection of BaSIRS, VaSIRS, PaSIRS or InSIRS, thus allowing better treatment interventions for subjects with symptoms of SIRS that stem at least in part from a bacterial, viral, protozoal infection or non-infectious causes.

The present inventors have determined that certain expression products are commonly, specifically and differentially expressed in humans, including cells of the immune system, during systemic inflammations with a range of bacterial etiologies underscoring the conserved nature of the host response to a BaSIRS. The results presented herein provide clear evidence that a unique biologically-relevant biomarker profile predicts BaSIRS with a remarkable degree of accuracy. This “pan-bacterial” systemic inflammation biomarker profile was validated in independently derived external datasets and publicly available datasets (see, TABLES 11 and 12 for the BaSIRS datasets used) and used to distinguish BaSIRS from other SIRS conditions including VaSIRS, PaSIRS and InSIRS (including autoimmune disease associated SIRS (ADaSIRS), cancer associated SIRS (CaSIRS) and trauma associated SIRS (TaSIRS)).

The present inventors have also determined that certain expression products are commonly, specifically and differentially expressed in humans, macaques, chimpanzees, mice, rats and pigs during systemic inflammations with a range of viral etiologies (e.g., Baltimore virus classification Groups I, II, III, IV, V, VI and VII), underscoring the conserved nature of the host response to a VaSIRS. The results presented herein provide clear evidence that a unique biologically-relevant biomarker profile predicts VaSIRS with a remarkable degree of accuracy. This “pan-viral” systemic inflammation biomarker profile was validated in independently derived external datasets and publicly available datasets (see, TABLES 16 and 17 for the VaSIRS datasets used) and used to distinguish VaSIRS from other SIRS conditions including BaSIRS, PaSIRS and InSIRS (including autoimmune disease associated SIRS (ADaSIRS), cancer associated SIRS (CaSIRS) and trauma associated SIRS (TaSIRS)).

It has also been determined that certain expression products are commonly, specifically and differentially expressed in humans during systemic inflammations with a range of protozoan etiologies (Plasmodium, Leishmania, Trypanosoma, Entamoeba) underscoring the conserved nature of the host response to a PaSIRS. The results presented herein provide clear evidence that a unique biologically-relevant biomarker profile predicts PaSIRS with a remarkable degree of accuracy. This “pan-protozoal” systemic inflammation biomarker profile was validated in publicly available datasets (see, TABLES 20 and 21 for the PaSIRS datasets used) and used to distinguish PaSIRS from other SIRS conditions including BaSIRS, VaSIRS and InSIRS (including autoimmune disease associated SIRS (ADaSIRS), cancer associated SIRS (CaSIRS) and trauma associated SIRS (TaSIRS)).

Additionally, it has been determined that certain expression products are commonly, specifically and differentially expressed in humans during systemic inflammations with a range of non-infectious etiologies underscoring the conserved nature of the host response of InSIRS. The results presented herein provide clear evidence that a unique biologically-relevant biomarker profile predicts InSIRS with a remarkable degree of accuracy. This infection-negative systemic inflammation biomarker profile was validated in publicly available datasets (see, TABLE 23 for the InSIRS datasets used) and used to distinguish InSIRS from other SIRS conditions including bacterial associated SIRS (BaSIRS), virus associated SIRS (VaSIRS) and protozoal associated SIRS (PaSIRS).

Overall, these findings provide compelling evidence that the expression products disclosed herein can function as biomarkers, respectively, for BaSIRS, VaSIRS, PaSIRS and InSIRS and may serve as useful diagnostic tools for triaging treatment decisions for SIRS-affected subjects. In this regard, it is proposed that the methods, apparatus, compositions and kits disclosed herein that are based on these biomarkers may serve in point-of-care diagnostics that allow for rapid and inexpensive screening for, and differentiation of, BaSIRS, VaSIRS, PaSIRS and InSIRS, which may result in significant cost savings to the medical system as SIRS-affected subjects can be exposed to therapeutic agents that are suitable for treating the etiology (e.g., bacterial, viral, protozoan or non-infectious) of their SIRS condition as opposed to therapeutic agents for SIRS conditions with other etiologies.

The present inventors have also identified, and designed assays for, common nucleic acid molecules in bacteria and protozoans and identified assays for detection of viruses at the genus level. For bacteria, the invention arises from the discovery that limited numbers of bacterial DNA Single Nucleotide Polymorphisms (SNPs) (SNP biomarkers) can be used to sensitively detect, quantify and broadly categorize bacterial DNA in the presence of host mammalian DNA. Further, the inventors have designed a simple, multiplexed nucleic acid amplification assay that can detect a limited number of human key protozoal pathogens that cause parasitemia. Further, multiplex assays that simultaneously detect the presence of a number of different, but limited, important human pathogenic virus genera are commercially available or have been reported in the scientific literature.

Thus, specific expression products are disclosed herein as host response specific biomarkers that provide a means for identifying BaSIRS, VaSIRS, PaSIRS or InSIRS and/or for distinguishing these systemic inflammatory conditions from each other for a subject with BaSIRS, VaSIRS, PaSIRS or InSIRS. Evaluation of these BaSIRS, VaSIRS, PaSIRS or InSIRS biomarkers through analysis of their levels in a subject or in a sample taken from a subject provides a measured or derived biomarker value for determinating an indicator that can be used for assessing the presence, absence or degree of BaSIRS, VaSIRS, PaSIRS or InSIRS in a subject.

Further, specific nucleic acids are disclosed herein as pathogen specific biomarkers, including bacterial SNP biomarkers, or conserved protozoal DNA sequence biomarkers, or conserved viral DNA sequence biomarkers, that provide a means for identifying bacterial infection positive (BIP), viral infection positive (VIP) or protozoal infection positive (PIP) samples and/or for distinguishing these three infection-positive conditions from each other and other infection-negative conditions. Evaluation of these nucleic acid biomarkers through analysis of their levels in a subject or in a sample taken from a subject provides a measured or derived biomarker value for determinating an indicator that can be used for assessing the presence, absence or degree of BaSIRS, VaSIRS, PaSIRS or InSIRS in a subject.

Additionally, unique combinations of host response specific biomarkers for identifying BaSIRS, VaSIRS, PaSIRS or InSIRS, and optionally pathogen specific biomarkers for identifying BIP, VIP or PIP, are disclosed that provide a means of more accurately identifying, compared to their use in isolation, BaSIRS, VaSIRS, PaSIRS or InSIRS and/or for distinguishing these systemic inflammatory conditions from each other. In certain embodiments, the host response specific and pathogen specific biomarker combinations are evaluated through analysis of their combined levels in a subject or in a sample taken from a subject, to thereby determine an indicator that is useful for assessing the presence, absence or degree of BaSIRS, VaSIRS, PaSIRS or InSIRS in a subject.

Accordingly, biomarker values can be measured biomarker raw data values, which are values of biomarkers measured for the subject, or alternatively could be derived biomarker values, which are values that have been derived from one or more measured biomarker values, for example by applying a function to the measured biomarker values. As used herein, biomarkers values to which a function has been applied are referred to as “derived biomarkers values” and the biomarkers to which the derived biomarker values correspond are referred to herein as “derived biomarkers”. As used herein, host response specific derived biomarker values and pathogen specific biomarker values to which a combining function has been applied are referred to as “compound biomarker values” and the biomarkers to which the compound biomarker values correspond are referred to herein as “compound biomarkers”.

The biomarker values may be determined in any one of a number of ways. An exemplary method of determining biomarker values is described by the present inventors in WO 2015/117204, which is incorporated herein by reference in its entirety. In one example, the process of determining biomarker values can include measuring the biomarker values, for example by performing tests on the subject or on sample(s) taken from the subject. More typically however, the step of determining the biomarker values includes having an electronic processing device receive or otherwise obtain biomarker values that have been previously measured or derived. This could include for example, retrieving the biomarker values from a data store such as a remote database, obtaining biomarker values that have been manually inputted using an input device, or the like. The biomarker values are combined by the electronic processing device, for example by adding, multiplying, subtracting, or dividing biomarker values, to provide one or more derived biomarker values. In its simplest form, a single derived biomarker value may represent an indicator value that is at least partially indicative of an indicator representing a presence, absence or degree of a condition. Alternatively, a plurality of derived biomarker values may be combined using a combining function to provide an indicator value. in other embodiments, at least one derived biomarker value is combined with one or more biomarker values to provide a compound biomarker value representing an indicator value. The combining step is performed so that multiple biomarker values that are measured or derived can be combined into a single indicator value, providing a more useful and straightforward mechanism for allowing the indicator to be interpreted and hence used in diagnosing the presence, absence or degree of BaSIRS, VaSIRS, PaSIRS or InSIRS in the subject.

Accordingly, an indicator is determined using a combination of the plurality of biomarker values, the indicator being at least partially indicative of the presence, absence or degree of BaSIRS, VaSIRS, PaSIRS or InSIRS. Assuming the method is performed using an electronic processing device, an indication of the indicator is optionally displayed or otherwise provided to the user. In this regard, the indication could be a graphical or alphanumeric representation of an indicator value. Alternatively however, the indication could be the result of a comparison of the indicator value to predefined thresholds or ranges, or alternatively could be an indication of the presence, absence, degree of BaSIRS, VaSIRS, PaSIRS or InSIRS, derived using the indicator.

In some embodiments in which a plurality of host response specific biomarkers and derived biomarker values are used, in order to ensure that an effective diagnosis can be determined, at least two of the biomarkers have a mutual correlation in respect of BaSIRS, VaSIRS, PaSIRS or InSIRS that lies within a mutual correlation range, the mutual correlation range being between ±0.9. This requirement means that the two biomarkers are not entirely correlated in respect of each other when considered in the context of the BaSIRS, VaSIRS, PaSIRS or InSIRS being diagnosed. In other words, at least two of the biomarkers in the combination respond differently as the condition changes, which adds significantly to their ability when combined to discriminate between at least two conditions, to diagnose the presence, absence or degree of BaSIRS, VaSIRS, PaSIRS or InSIRS in or of the subject. Representative biomarker combinations, which are also referred to herein as “derived biomarker combinations”, which meet these criteria, are listed in TABLES A to D.

Typically, the requirement that host response specific biomarkers have a low mutual correlation means that the biomarkers may relate to different biological attributes or domains such as, but not limited, to different molecular functions, different biological processes and different cellular components. Illustrative examples of molecular function include addition of, or removal of, one of more of the following moieties to, or from, a protein, polypeptide, peptide, nucleic acid (e.g., DNA, RNA): linear, branched, saturated or unsaturated alkyl (e.g., C₁-C₂₄ alkyl); phosphate; ubiquitin; acyl; fatty acid, lipid, phospholipid; nucleotide base; hydroxyl and the like. Molecular functions also include signaling pathways, including without limitation, receptor signaling pathways and nuclear signaling pathways. Non-limiting examples of molecular functions also include cleavage of a nucleic acid, peptide, polypeptide or protein at one or more sites; polymerization of a nucleic acid, peptide, polypeptide or protein; translocation through a cell membrane (e.g., outer cell membrane; nuclear membrane); translocation into or out of a cell organelle (e.g., Golgi apparatus, lysosome, endoplasmic reticulum, nucleus, mitochondria); receptor binding, receptor signaling, membrane channel binding, membrane channel influx or efflux; and the like.

Illustrative examples of biological processes include: stages of the cell cycle such as meiosis, mitosis, cell division, prophase, metaphase, anaphase, telophase and interphase, stages of cell differentiation; apoptosis; necrosis; chemotaxis; immune responses including adaptive and innate immune responses, pro-inflammatory immune responses, autoimmune responses, tolerogenic responses and the like. Other illustrative examples of biological processes include generating or breaking down adenosine triphosphate (ATP), saccharides, polysaccharides, fatty acids, lipids, phospholipids, sphingolipids, glycolipids, cholesterol, nucleotides, nucleic acids, membranes (e.g., cell plasma membrane, nuclear membrane), amino acids, peptides, polypeptides, proteins and the like. Representative examples of cellular components include organelles, membranes, as for example noted above, and others.

It will be understood that the use of host response specific biomarkers that have different biological attributes or domains provides further information than if the biomarkers were related to the same or common biological attributes or domains. In this regard, it will be appreciated if the at least two biomarkers are highly correlated to each other, the use of both biomarkers would add little diagnostic improvement compared to the use of a single one of the biomarkers. Accordingly, an indicator-determining method of the present invention in which a plurality of biomarkers and biomarker values are used preferably employ biomarkers that are not well correlated with each other, thereby ensuring that the inclusion of each biomarker in the method adds significantly to the discriminative ability of the indicator.

Further, it will be understood that the use of a combination of host response specific biomarkers that have a low mutual correlation with pathogen specific biomarkers adds significantly to the positive and negative discriminative ability of the biomarker indicator. Accordingly, an indicator-determining method of the present invention in which a plurality of biomarkers and biomarker values are used preferably employ host response biomarkers that are not well correlated with each other in combination with pathogen specific biomarkers, thereby ensuring that the inclusion of each biomarker in the method adds significantly to the discriminative ability of the indicator.

Despite this, in order to ensure that the indicator can accurately be used in performing the discrimination between at least two conditions (e.g., BaSIRS, VaSIRS, PaSIRS or InSIRS) or the diagnosis of the presence, absence or degree of BaSIRS, VaSIRS, PaSIRS or InSIRS, the indicator has a performance value that is greater than or equal to a performance threshold. The performance threshold may be of any suitable form but is to be typically indicative of an explained variance of at least 0.3, or an equivalent value of another performance measure.

Suitably, a combination of biomarkers is employed, which includes (1) host response specific biomarkers having a mutual correlation between ±0.9 and which combination provides an explained variance of at least 0.3, and; (2) pathogen specific biomarkers. In specific embodiments, host response specific biomarkers are used in combination with pathogen specific biomarkers when greater discriminatory power (positive or negative predictive value) is required. Also, this typically allows an indicator to be defined that is suitable for ensuring that an accurate discrimination and/or diagnosis can be obtained whilst minimizing the number of biomarkers that are required. Typically the mutual correlation range is one of ±0.8; ±0.7; ±0.6; ±0.5; ±0.4; ±0.3; ±0.2; and, ±0.1. Typically each BaSIRS, VaSIRS, PaSIRS or InSIRS biomarker has a condition correlation with the presence, absence or degree of BaSIRS, VaSIRS, PaSIRS or InSIRS that lies outside a condition correlation range, the condition correlation range being between ±0.3 and more typically ±0.9; ±0.8; ±0.7; ±0.6; ±0.5; and, ±0.4. Typically the performance threshold is indicative of an explained variance of at least one of 0.4; 0.5; 0.6; 0.7; 0.8; and 0.9.

It will be understood that in this context, the biomarkers used within the above-described method can define a biomarker profile for BaSIRS, VaSIRS, PaSIRS or InSIRS, which includes a minimal number of biomarkers, whilst maintaining sufficient performance to allow the biomarker profile to be used in making a clinically relevant diagnosis or differentiation. Minimizing the number of biomarkers used minimizes the costs associated with performing diagnostic tests and in the case of nucleic acid expression products, allows the test to be performed utilizing relatively straightforward techniques such as nucleic acid array, and polymerase chain reaction (PCR) processes, or the like, allowing the test to be performed rapidly in a clinical environment.

Furthermore, producing a single indicator value allows the results of the test to be easily interpreted by a clinician or other medical practitioner, so that test can be used for reliable diagnosis in a clinical environment.

Processes for generating suitable host response biomarker profiles are described for example in WO 2015/117204, which uses the term “biomarker signature” in place of “biomarker profile” as defined herein. It will be understood, therefore, that terms “biomarker profile” and “biomarker signature” are equivalent in scope. The biomarker profile-generating processes disclosed in WO 2015/117204 provide mechanisms for selecting a combination of biomarkers, and more typically derived biomarkers, that can be used to form a biomarker profile, which in turn can be used in diagnosing the presence, absence or degree of BaSIRS, VaSIRS, PaSIRS or InSIRS. In this regard, the biomarker profile defines the biomarkers that should be measured (i.e., the profile biomarkers), how derived biomarker values should be determined for measured biomarker values, and then how biomarker values should be subsequently combined to generate an indicator value. The biomarker profile can also specify defined indicator value ranges that indicate a particular presence, absence or degree of BaSIRS, VaSIRS, PaSIRS or InSIRS.

Processes for generating suitable pathogen specific biomarkers for bacteria are described for example in WO 2014/190394. The bacterial pathogen specific biomarkers disclosed in WO 2014/190394 provide mechanisms for selecting a combination of biomarkers that can be used to form a biomarker profile, which in turn can be used in diagnosing the presence, absence or degree of BIP, and for broadly categorizing the type of bacteria detected (if detected). Processes for generating suitable pathogen specific biomarkers for viruses are described herein and in the scientific literature. The virus pathogen specific biomarkers disclosed herein provide mechanisms for selecting a combination of biomarkers that can be used to form a biomarker profile, which in turn can be used in diagnosing the presence, absence or degree of VIP, and for broadly categorizing the type of viruses(s) detected (if detected) and for determining the presence, absence or degree of VIP that can be treated using currently available anti-viral therapies. Processes for generating suitable pathogen specific biomarkers for protozoans are described herein. The protozoan antigen specific biomarkers disclosed herein provide mechanisms for selecting a combination of biomarkers that can be used to form a biomarker profile, which in turn can be used in diagnosing the presence, absence or degree of PIP, and for broadly categorizing the type of protozoan detected (if detected).

Using the above-described methods a number of host response specific biomarkers have been identified that are particularly useful for assessing a likelihood that a subject has a presence, absence or degree of BaSIRS, VaSIRS, PaSIRS or InSIRS in a subject. Further, using the above-described methods a number of pathogen specific biomarkers have been identified that are particularly useful when combined with host response specific biomarkers for assessing a likelihood that a subject has a presence, absence or degree of bacterial, viral or protozoal infection in a subject. Combinations of host response specific biomarkers and pathogen-specific biomarkers are referred to herein as “compound biomarkers”. As used herein, the term “compound biomarkers” refers to a combination of host response specific biomarkers and at least one pathogen specific biomarker. Generally a host response specific biomarker is a biomarker of the host's immune system, which is altered, or whose level of expression is altered, as part of an inflammatory response to damage or insult resulting from a bacterial, viral or protozoal infection. A pathogen specific biomarker is a molecule or group of molecules of a pathogen, which is specific to a particular category, genus or type of bacteria, virus or protozoan. Compound biomarkers for BaSIRS, VaSIRS, PaSIRS or InSIRS are suitably a combination of both expression products of host genes (also referred to interchangeably herein as “BaSIRS, VaSIRS, PaSIRS or InSIRS biomarker genes”) and pathogen specific biomarkers, including polynucleotide, polypeptide, carbohydrate, lipid, lipopolysaccharide, metabolite. As used herein, polynucleotide expression products of BaSIRS, VaSIRS, PaSIRS or InSIRS biomarker genes are referred to herein as “BaSIRS, VaSIRS, PaSIRS or InSIRS biomarker polynucleotides.” Polypeptide expression products of the BaSIRS, VaSIRS, PaSIRS or InSIRS biomarker genes are referred to herein as “BaSIRS, VaSIRS, PaSIRS or InSIRS biomarker polypeptides.”

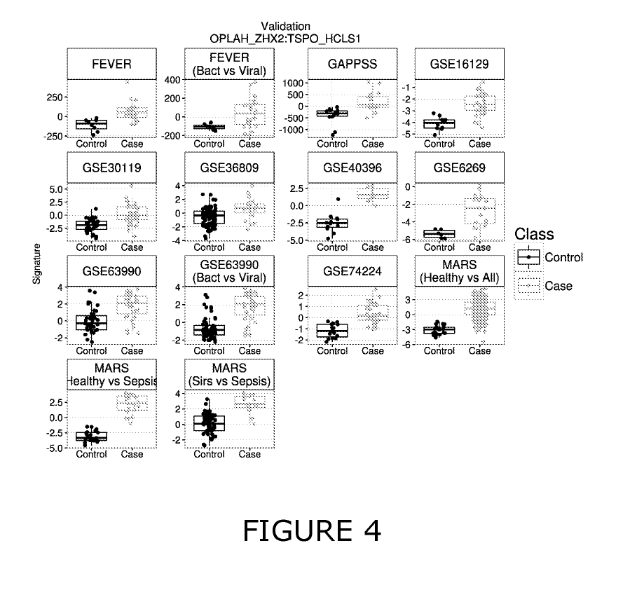

BaSIRS biomarkers are suitably selected from expression products of any one or more of the following BaSIRS genes: ADAM19, ADM, ALPL, CAMK1D, CASS4, CBLL1, CCNK, CD82, CLEC7A, CNNM3, COX15, CR1, DENND3, DOCK5, ENTPD7, EPHB4, EXTL3, FAM129A, FBXO28, FIG. 4, FOXJ3, GAB2, GALNT2, GAS7, GCC2, GRK5, HAL, HCLS1, HK3, ICK, IGFBP7, IK, IKZF5, IL2RB, IMPDH1, INPP5D, ITGA7, JARID2, KIAA0101, KIAA0355, KIAA0907, KLRD1, KLRF1, LAG3, LEPROTL1, LPIN2, MBIP, MCTP1, MGAM, MME, NCOA6, NFIC, NLRP1, NMUR1, NOV, NPAT, OPLAH, PARP8, PCOLCE2, PDGFC, PDS5B, PHF3, PIK3C2A, PLA2G7, POGZ, PRKD2, PRKDC, PRPF38B, PRSS23, PYHIN1, QRICH1, RAB32, RBM15, RBM23, RFC1, RNASE6, RUNX2, RYK, SAP130, SEMA4D, SIDT1, SMPDL3A, SPIN1, ST3GAL2, SYTL2, TGFBR3, TLE3, TLR5, TMEM165, TSPO, UTRN, YPEL1, ZFP36L2, ZHX2. Non-limiting examples of nucleotide sequences for these BaSIRS biomarkers are listed in SEQ ID NOs: 1-94. Non-limiting examples of amino acid sequences for these BaSIRS biomarkers are listed in SEQ ID NOs: 95-188.

VaSIRS biomarkers are suitably selected from expression products of any one or more of the following VaSIRS genes: ABAT, ABHD2, ABI1, ABLIM1, ACAA1, ACAP2, ACVR1B, AIF1, ALDH3A2, ANKRD49, AOAH, APBB1IP, APLP2, ARAP1, ARHGAP15, ARHGAP25, ARHGAP26, ARHGEF2, ARRB1, ARRB2, ASAP1, ATAD2B, ATF7IP2, ATM, ATP6V1B2, BACH1, BANP, BAZ2B, BCL2, BEX4, BMP2K, BRD1, BRD4, BTG1, C19orf66, C2orf68, CAMK1D, CAMK2G, CAP1, CASC3, CASP8, CBX7, CCND3, CCNG2, CCNT2, CCR7, CD37, CD93, ADGRE5, CDIPT, CEP170, CEP68, CHD3, CHMP1B, CHMP7, CHST11, CIAPIN1, CLEC4A, CLK4, CNPY3, CREB1, CREBBP, CRLF3, CRTC3, CSAD, CSF2RB, CSNK1D, CST3, CTBP2, CTDSP2, CUL1, CYLD, CYTH4, DCP2, DDX60, DGCR2, DGKA, DHX58, DIDO1, DOCK9, DOK3, DPEP2, DPF2, EIF2AK2, EIF3H, EMR2, ERBB2IP, ETS2, FAIM3, FAM134A, FAM65B, FBXO11, FBXO9, FCGRT, FES, FGR, FLOT2, FNBP1, FOXJ2, FOXO1, FOXO3, FRY, FYB, GABARAP, GCC2, GMIP, GNA12, GNAQ, GOLGA7, GPBP1L1, GPR97, GPS2, GPSM3, GRB2, GSK3B, GYPC, HAL, HCK, HERCS, HERC6, HGSNAT, HHEX, HIP1, HPCAL1, HPS1, ICAM3, IFI44, IFI6, IFIH1, IGSF6, IKBKB, IL10RB, IL13RA1, IL16, IL1RAP, IL27RA, IL4R, IL6R, IL6ST, INPP5D, IQSEC1, ISG15, ITGAX, ITGB2, ITPKB, ITSN2, JAK1, KBTBD2, KIAA0232, KIAA0247, KIAA0513, KLF3, KLF6, KLF7, KLHL2, LAP3, LAPTM5, LAT2, LCP2, LDLRAP1, LEF1, LILRA2, LILRB3, LIMK2, LPAR2, LPIN2, LRMP, LRP10, LST1, LTB, LYL1, LYN, LYST, MAML1, MANSC1, MAP1LC3B, MAP3K11, MAP3K3, MAP3K5, MAP4K4, MAPK1, MAPK14, MAPRE2, MARCH7, MARCH8, MARK3, MAST3, MAX, MBP, MCTP2, MED13, MEF2A, METTL3, MKLN1, MKRN1, MMP25, MORC3, MOSPD2, MPPE1, MSL1, MTMR3, MX1, MXI1, MYC, N4BP1, NAB1, NACA, NCBP2, NCOA1, NCOA4, NDE1, NDEL1, NDFIP1, NECAP2, NEK7, NFKB1, NFYA, NLRP1, NOD2, NOSIP, NPL, NR3C1, NRBF2, NSUN3, NUMB, OAS2, OASL, OGFRL1, OSBPL11, OSBPL2, PACSIN2, PAFAH1B1, PARP12, PBX3, PCBP2, PCF11, PCNX, PDCD6IP, PDE3B, PECAM1, PFDNS, PGS1, PHC2, PHF11, PHF2, PHF20, PHF20L1, PHF3, PIAS1, PIK3IP1, PINK1, PISD, PITPNA, PLEKHO1, PLEKHO2, PLXNC1, POLB, POLD4, POLR1D, PPARD, PPM1F, PPP1R11, PPP1R2, PPP2R5A, PPP3R1, PPP4R1, PRKAA1, PRKAG2, PRKCD, PRMT2, PRUNE, PSAP, PSEN1, PSTPIP1, PTAFR, PTEN, PTGER4, PTPN6, PTPRE, PUM2, R3HDM2, RAB11FIP1, RAB14, RAB31, RAB4B, RAB7A, RAF1, RALB, RARA, RASSF2, RBM23, RBMS1, RC3H2, RERE, RGS14, RGS19, RHOG, RIN3, RNASET2, RNF130, RNF141, RNF146, RNF19B, RPL10A, RPL22, RPS6KA1, RPS6KA3, RSAD2, RTN3, RTP4, RXRA, RYBP, SAFB2, SATB1, SEC62, SEMA4D, SERINC3, SERINCS, SERTAD2, SESN1, SETD2, SH2B3, SH2D3C, SIRPA, SIRPB1, SLCO3A1, SMAD4, SNN, SNRK, SNX27, SOAT1, SORL1, SOS2, SP3, SSBP2, SSFA2, ST13, ST3GAL1, STAM2, STAT1, STAT5A, STAT5B, STK38L, STX10, STX3, STX6, SYPL1, TAP1, TFE3, TFEB, TGFBI, TGFBR2, TGOLN2, TIAM1, TLE3, TLE4, TLR2, TM2D3, TMBIM1, TMEM127, TMEM204, TNFRSF1A, TNFSF13, TNIP1, TNK2, TNRC6B, TOPORS, TRAK1, TREM1, TRIB2, TRIMS, TRIOBP, TSC22D3, TYK2, TYROBP, UBE2D2, UBE2L6, UBN1, UBQLN2, UBXN2B, USP10, USP15, USP18, USP4, UTP14A, VAMP3, VAV3, VEZF1, VPS8, WASF2, WBP2, WDR37, WDR47, XAF1, XPC, XP06, YPEL5, YTHDF3, ZBP1, ZBTB18, ZC3HAV1, ZDHHC17, ZDHHC18, ZFAND5, ZFC3H1, ZFYVE16, ZMIZ1, ZNF143, ZNF148, ZNF274, ZNF292, ZXDC, ZYX. Non-limiting examples of nucleotide sequences for these VaSIRS biomarkers are listed in SEQ ID NOs: 189-601. Non-limiting examples of amino acid sequences for these VaSIRS biomarkers are listed in SEQ ID NOs: 602-1013.

PaSIRS biomarkers are suitably selected from expression products of any one or more of the following PaSIRS genes: ACSL4, ADK, ADSL, AHCTF1, APEX1, ARHGAP17, ARID1A, ARIH2, ASXL2, ATOX1, ATP2A2, ATP6V1B2, BCL11A, BCL3, BCL6, C3AR1, CAMK2G, CCND3, CCR7, CD52, CD55, CD63, CEBPB, CEP192, CHN2, CLIP4, CNOT7, CSNK1G2, CSTB, DNAJC10, EN01, ERLIN1, ETV6, EXOSC10, EXOSC2, EXOSC9, FBL, FBX011, FCER1G, FGR, FLII, FLOT1, FNTA, G6PD, GLG1, GNG5, GPI, GRINA, HCK, HERC6, HLA-DPA1, IL10RA, IMP3, IRF1, IRF8, JUNB, KIF1B, LAP3, LDHA, LY9, METAP1, MGEA5, MLLT10, MYD88, NFIL3, NFKBIA, NOSIP, NUMB, NUP160, PCBP1, PCID2, PCMT1, PGD, PLAUR, PLSCR1, POMP, PREPL, PRKCD, RAB27A, RAB7A, RALB, RBMS1, RITZ, RPL15, RPL22, RPL9, RPS14, RPS4X, RTN4, SEH1L, SERBP1, SERPINB1, SERTAD2, SETX, SH3GLB1, SLAMF7, SOCS3, SORT1, SPI1, SQRDL, STAT3, SUCLG2, TANK, TAP1, TCF4, TCIRG1, TIMP2, TMEM106B, TMEM50B, TNIP1, TOP2B, TPP1, TRAF3IP3, TRIB1, TRIT1, TROVE2, TRPC4AP, TSPO, TTC17, TUBA1B, UBE2L6, UFM1, UPP1, USP34, VAMP3, WARS, WAS, ZBED5, ZMYND11, ZNF266. Non-limiting examples of nucleotide sequences for these PaSIRS biomarkers are listed in SEQ ID NOs: 1014-1143. Non-limiting examples of amino acid sequences for these PaSIRS biomarkers are listed in SEQ ID NOs: 1144-1273.

InSIRS biomarkers are suitably selected from expression products of any one or more of the following InSIRS genes: ADAM19, ADRBK2, ADSL, AGA, AGPAT5, ANK3, ARHGAP5, ARHGEF6, ARL6IP5, ASCC3, ATP8A1, ATXN3, BCKDHB, BRCC3, BTN2A1, BZW2, C14orf1, CD28, CD40LG, CD84, CDA, CDK6, CDKN1B, CKAP2, CLEC4E, CLOCK, CLUAP1, CPA3, CREB1, CYP4F3, CYSLTR1, DIAPH2, EFHD2, EFTUD1, EIF5B, ENOSF1, ENTPD1, ERCC4, ESF1, EXOC7, EXTL3, FASTKD2, FCF1, FUT8, G3BP1, GAB2, GGPS1, GOLPH3L, HAL, HEATR1, HEBP2, HIBCH, HLTF, HRH4, IDE, IGF2R, IKBKAP, IP07, IQCB1, IQSEC1, KCMF1, KIAA0391, KLHL20, KLHL24, KRIT1, LANCL1, LARP1, LARP4, LRRC8D, MACF1, MANEA, MDH1, METTL5, MLLT10, MRPS10, MT01, MTRR, MXD1, MYH9, MY09A, NCBP1, NEK1, NFX1, NGDN, NIP7, NOL10, NOL8, NOTCH2, NR2C1, PELI1, PEX1, PHC3, PLCL2, POLR2A, PRKAB2, PRPF39, PRUNE, PSMDS, PTGS1, PWP1, RAB11FIP2, RABGAP1L, RAD50, RBM26, RCBTB2, RDX, REPS1, RFC1, RGS2, RIOK2, RMND1, RNF170, RNMT, RRAGC, S100PBP, SIDT2, SLC35A3, SLC35D1, SLCO3A1, SMC3, SMC6, STK17B, SUPT7L, SYNE2, SYT11, TBCE, TCF12, TCF7L2, TFIP11, TGS1, THOC2, TIA1, TLK1, TMEM87A, TNFSF8, TRAPPC2, TRIP11, TTC17, TTC27, VEZT, VNN3, VPS13A, VPS13B, VPS13C, WDR70, XP04, YEATS4, YTHDC2, ZMYND11, ZNF507, ZNF562. Non-limiting examples of nucleotide sequences for these InSIRS biomarkers are listed in SEQ ID NOs: 1274-1424. Non-limiting examples of amino acid sequences for these InSIRS biomarkers are listed in SEQ ID NOs: 1425-1575.

The present inventors have determined that certain BaSIRS biomarkers have strong diagnostic performance when combined with one or more other BaSIRS biomarkers. In particular, pairs of BaSIRS biomarkers have been identified, each of which forms a BaSIRS derived biomarker combination that is advantageously not a derived biomarker combination for VaSIRS, PaSIRS or InSIRS, and which is thus useful as a BaSIRS indicator of high specificity. Accordingly, in specific embodiments, an indicator is determined that correlates to a derived biomarker value corresponding to a ratio of BaSIRS biomarker values, which can be used in assessing a likelihood of a subject having a presence, absence or degree of BaSIRS. Exemplary BaSIRS derived biomarker combinations are listed in TABLE A.

It has also been determined that certain VaSIRS biomarkers have strong diagnostic performance when combined with one or more other VaSIRS biomarkers. In particular embodiments, pairs of VaSIRS biomarkers are employed, each of which forms a VaSIRS derived biomarker combination that is advantageously not a derived biomarker combination for BaSIRS, PaSIRS or InSIRS, and which is thus useful as a VaSIRS indicator of high specificity. In non-limiting examples of this type, an indicator is determined that correlates to a derived biomarker value corresponding to a ratio of VaSIRS biomarker values, which can be used in assessing a likelihood of a subject having a presence, absence or degree of VaSIRS. Representative VaSIRS derived biomarker combinations are listed in TABLE B.

Additionally, certain PaSIRS biomarkers have been identified with strong diagnostic performance when combined with one or more other PaSIRS biomarkers. In certain embodiments, pairs of PaSIRS biomarkers are utilized, each of which forms a VaSIRS derived biomarker combination that is advantageously not a derived biomarker combination for BaSIRS, VaSIRS or InSIRS, and which is useful, therefore, as a PaSIRS indicator of high specificity. Accordingly, in representative examples, an indicator is determined that correlates to a derived biomarker value corresponding to a ratio of PaSIRS biomarker values, which can be used in assessing a likelihood of a subject having a presence, absence or degree of PaSIRS. Non-limiting PaSIRS derived biomarker combinations are listed in TABLE C.

The present inventors have also determined that certain InSIRS biomarkers have strong diagnostic performance when combined with one or more other InSIRS biomarkers. In particular, pairs of InSIRS biomarkers have been identified, each of which forms an InSIRS derived biomarker combination that is advantageously not a derived biomarker combination for BaSIRS, VaSIRS or PaSIRS, and which is thus useful as a InSIRS indicator of high specificity. Accordingly, in specific embodiments, an indicator is determined that correlates to a derived biomarker value corresponding to a ratio of InSIRS biomarker values, which can be used in assessing a likelihood of a subject having a presence, absence or degree of InSIRS. Exemplary InSIRS derived biomarker combinations are listed in TABLE D.

In these embodiments, the indicator-determining methods suitably include: (1) determining a pair of SIRS biomarker values, wherein each biomarker value is a value measured for at least one corresponding SIRS biomarker (e.g., BaSIRS, VaSIRS, PaSIRS or InSIRS biomarker) of the subject and is at least partially indicative of a level of the SIRS biomarker in a sample taken from the subject; and (2) combining the biomarker values using a function. The function is suitably selected from multiplication, subtraction, addition or division. In particular embodiments, the function is a division and one member of the pair of host response specific biomarker values is divided by the other member of the pair to provide a ratio of levels of the pair of SIRS biomarkers. Thus, in these embodiments, if the host response SIRS biomarker values denote the levels of a pair of SIRS biomarkers (e.g., BaSIRS, VaSIRS, PaSIRS or InSIRS biomarkers), then the host response SIRS ‘derived biomarker’ values will be based on a ratio of the host response SIRS biomarker values. However, in other embodiments in which the host response SIRS biomarker values represent amplification amounts, or cycle times (e.g., PCR cycle times), which are a logarithmic representation of the level of the SIRS biomarkers within a sample, then the SIRS biomarker values may be combined in some other manner, such as by subtracting the cycle times to determine a host response derived biomarker value indicative of a ratio of the levels of the SIRS biomarkers.

In specific embodiments, the indicator-determining methods involve: (1) determining a first derived biomarker value using a first pair of host response specific biomarker values that are measured for a corresponding first and second SIRS biomarkers in a sample, wherein the first and second SIRS biomarkers are selected from biomarkers of a single SIRS etiological type (e.g., one of BaSIRS, VaSIRS, PaSIRS or inSIRS biomarkers), the first derived biomarker value being indicative of a ratio of levels of the first and second SIRS biomarkers in the sample, (2) determining a second derived biomarker value using a second pair of host response specific biomarker values that are measured for a corresponding third and fourth SIRS biomarkers in the sample, wherein the third and fourth SIRS biomarkers are selected from SIRS biomarkers of the same etiological type as the first and second SIRS biomarkers, the second derived biomarker value being indicative of a ratio of levels of the third and fourth SIRS biomarkers in the sample; and optionally (3) determining a third derived biomarker value using a third pair of host response specific biomarker values that are measured for a corresponding fifth and sixth SIRS biomarkers in the sample, wherein the fifth and sixth SIRS biomarkers are selected from SIRS biomarkers of a same etiological type as the first and second SIRS biomarkers, the third derived biomarker value being indicative of a ratio of levels of the fifth and sixth SIRS biomarkers in the sample.

In advantageous embodiments that provide higher levels of specificity for determining the indicator, the indicator-determining methods may further comprise: determining at least one pathogen specific biomarker value, wherein each pathogen specific biomarker value is a value measured for at least one corresponding pathogen specific biomarker (e.g., a BIP, VIP or PIP biomarker) of the subject and is at least partially indicative of a level of the pathogen specific biomarker in the sample. The pathogen to which the pathogen specific biomarker relates is typically one that associates with a SIRS of the same etiological type to which the host response specific biomarkers relate. Representative pathogen specific biomarker values are suitably selected from presence/absence, level, or PCR cycle time, and if positive, to include a descriptor of the pathogen category (e.g., Gram positive or Gram negative, virus type or protozoan species). Thus, the use of BaSIRS biomarkers in the indicator-determining methods of the present invention can be augmented through use of one or more BIP biomarkers to provide host response specific derived BaSIRS biomarker values and at least one BIP biomarker value to thereby determine a compound biomarker value that is at least partially indicative of the presence, absence or degree of BaSIRS. Likewise, the use of VaSIRS biomarkers in the indicator-determining methods of the present invention can be augmented through use of one or more VIP biomarkers to provide host response specific VaSIRS derived biomarker values and at least one VIP biomarker value to thereby determine a compound biomarker value that is at least partially indicative of the presence, absence or degree of VaSIRS. Similarly, the use of PaSIRS biomarkers in the indicator-determining methods of the present invention can be augmented through use of one or more PIP biomarkers to provide host response specific PaSIRS derived biomarker values and at least one PIP biomarker value to thereby determine a compound biomarker value that is at least partially indicative of the presence, absence or degree of PaSIRS.

Typically the pathogen specific biomarkers belong to pathogens associated with the development or progression of SIRS. A limited number of microorganisms (bacteria, viruses, protozoans) cause disease in humans, with only few causing the majority of infectious diseases, even fewer causing SIRS, and still even fewer number causing bacteremia, viremia or protozoan parasitemia. TABLE 1 lists common bacterial, viral and protozoal pathogens associated with human BaSIRS, VaSIRS and PaSIRS that can also be found in peripheral blood (in whole or part), respectively. Such pathogens have multiple methods of interacting with the host and its cells and if a host mounts a systemic inflammatory response to an infection it means that the immune system has been exposed to sufficient levels of novel pathogen molecules. Representative types of pathogen molecules that can elicit a systemic inflammatory response include proteins, nucleic acids (RNA and/or DNA), lipoproteins, lipoteichoic acid and lipopolysaccharides, many of which can be detected (and typed) circulating in blood at some stage during the disease pathogenesis.

Molecular nucleic acid-based tests have been developed to detect the major sepsis-causing bacterial pathogens in whole blood from patients with suspected sepsis (e.g., SeptiFast® from Roche, Iridica® from Abbott, Sepsis Panel from Biofire (Biomerieux), Prove-it® Sepsis from Mobidiag). Reference also can be made to U.S. Pat. Appl. Pub. No. 2016/0032364, which discloses methods of detecting and distinguishing a myriad of bacterial species through detection of 16S ribosomal ribonucleic acid (rRNA) using antisense probes. An alternative method is disclosed in U.S. Pat. Appl. Pub. No. 2014/0249037, which characterizes bacteria by amplifying bacterial 16S rRNA and characterizing the bacteria based on the 16S rRNA gene sequence.

In specific embodiments, bacterial pathogen Gram status (i.e., Gram-positive or Gram-negative) is detected using methods and kits disclosed in U.S. Pat. Appl. Pub. No. 2016/0145696, which is incorporated herein by reference, through interrogation of polymorphisms at nucleotide positions of bacterial 16S rRNA that correspond to positions 396 and 398 of the Escherichia coli 16S rRNA gene. Positions corresponding to positions 396 and 398 of SEQ ID NO:1576 in any prokaryotic 16S rRNA gene (or 16S rRNA molecule or DNA copy thereof) are readily identifiable by alignment with the E. coli 16S rRNA gene set forth in SEQ ID NO:1576. The general rules for differentiating Gram-positive and Gram-negative bacteria that can cause BaSIRS using these two pathogen biomarker SNP molecules are depicted in TABLE E.

TABLE E

| Gram Status | SNP 396 | SNP 398 |
|---|---|---|
| Negative | C | T/A/C |
| Positive | A/T/G | C |

Thus, the pathogen biomarker SNPs in TABLE E provide the means for determining the Gram status of a bacterium in a sample by analyzing nucleic acid from the sample for SNPs in the 16S rRNA gene (or 16S rRNA or DNA copy thereof) at positions corresponding to positions 396 and 398 of the 16S rRNA gene set forth in SEQ ID NO:1576, wherein a C at position 396 and a T, A or C at position 398 indicates that the bacterium in the sample is a Gram-negative bacterium; and an A, T or G at position 396 and a C at position 398 indicates that the bacterium is a Gram-positive bacterium. Bacteria that can be classified as Gram-positive or Gram-negative using SNPs at positions corresponding to 396 and 398 of the E. coli 16S rRNA gene set forth in SEQ ID NO:1576 include, for example, Acinetobacter spp., Actinobacillus spp., Actinomadura spp., Actinomyces spp., Actinoplanes spp., Aeromonas spp., Agrobacterium spp., Alistipes spp., Anaerococcus spp., Arthrobacter spp., Bacillus spp., Brucella spp., Bulleidia spp., Burkholderia spp., Cardiobacterium spp., Citrobacter spp., Clostridium spp., Corynebacterium spp., Dermatophilus spp., Dorea spp., Edwardsiella spp., Enterobacter spp., Enterococcus spp., Erysipelothrix spp., Escherichia spp., Eubacterium spp., Faecalibacterium spp., Filifactor spp., Finegoldia spp., Flavobacterium spp., Gallicola spp., Haemophilus spp., Helcococcus spp., Holdemania spp., Hyphomicrobium spp., Klebsiella spp., Lactobacillus spp., Legionella spp., Listeria spp., Methylobacterium spp., Micrococcus spp., Micromonospora spp., Mobiluncus spp., Moraxella spp., Morganella spp., Mycobacterium spp., Neisseria spp., Nocardia spp., Paenibacillus spp., Parabacteroides spp., Pasteurella spp., Entomophile's spp., Peptostreptococcus spp., Planococcus spp., Planomicrobium spp., Plesiomonas spp., Porphyromonas spp., Prevotella spp., Propionibacterium spp., Proteus spp., Providentia spp., Pseudomonas spp., Ralstonia spp., Rhodococcus spp., Roseburia spp., Ruminococcus spp., Salmonella spp., Sedimentibacter spp., Serratia spp., Shigella spp., Solobacterium spp., Sphingomonas spp., Sporanaerobacter spp., Staphylococcus spp., Stenotrophomonas spp., Streptococcus spp., Streptomyces spp., Tissierella spp., Vibrio spp., and Yersinia spp. Accordingly, in instances in which the pathogen specific biomarker is a bacterial biomarker, the biomarker is preferably a 16S rRNA gene, more preferably polymorphisms at nucleotide positions of bacterial 16S rRNA that correspond to positions 396 and 398 of the Escherichia coli 16S rRNA gene, which can be used to provide the Gram status of a bacterial pathogen.

For virus detection, numerous sensitive and specific assays are available in the art. For example, amplification of viral DNA and RNA (e.g., PCR) as well as viral antigen detection assays are known that are rapid and do not require lengthy incubation periods needed for viral isolation in cell cultures. To cover the possibility of a mixed infection, as well as to cover multiple possible viral causes or strains, there are commercially available assays capable of detecting more than one virus and/or strain at a time (e.g., BioMerieux, BioFire, FilmArray®, Respiratory Panel; Luminex, xTAG® Respiratory Viral Panel). Further, there are techniques that allow for amplification of viral DNA of unknown sequence which could be useful in situations where the clinical signs are generalized, for viruses with high mutation rates, for new and emerging viruses, or for detecting biological weapons of man-made nature (Clem et al., Virol J 4: 65, 2007; Liang et al., Science 257(5072:967-971), 1992; Nie X et al., J Virol Methods 91(1):37-49, 2001; Ralph et al., Proc Natl Acad Sci USA 90(22):10710-10714, 1993). Further, a microarray has been designed to detect every known virus for which there is DNA sequence information in GenBank (called “Virochip”) (Greninger et al., PLoS ONE, 5(10), e13381, 2010; Chiu et al., Proc Natl Acad Sci USA 105: 14124-14129, 2008).

In some instances, detection of host antibodies to an infecting virus remains the diagnostic gold standard, because either the virus cannot be grown, or the presence of virus in a biological fluid is transient (e.g., arboviral infections) and therefore cannot be detected at times when the patient is symptomatic. In some instances the ratio of IgM to IgG antibodies can be used to determine the recency of virus infection. IgM is usually produced early in the immune response and is non-specific, whereas IgG is produced later in the immune response and is specific. Examples of the use of this approach include the diagnosis of hepatitis E (Tripathy et al., PLoS ONE, 7(2), e31822, 2012), dengue (SA-Ngasang et al., Epidemiology and Infection, 134(04), 820, 2005), and Epstein-Barr Virus (Hess, R. D. Journal of Clinical Microbiology, 42(8), 3381-3387, 2004).

In specific embodiments, viruses that are capable of causing pathology in humans, as for example those listed in TABLE 1, which are capable of causing SIRS, and cause a viremia are detected and/or quantified using any suitable nucleic acid detection and/or amplification assay, with oligonucleotide primers and/or probes listed in TABLE F.

TABLE F

| Reagent | 5′-3′ Sequence | SEQ ID NO. | Virus Detected |
|---|---|---|---|
| Forward (F) | CATC/TCTGTTGTATATGAGGCCCAT | 1577 | Influenza A |
| Reverse (R) | GGACTGCAGCGTAGACGCTT | 1578 | Influenza A |
| Probe (P) | CTCAGTTATTCTGCTGGTGCACTTGCCA | 1579 | Influenza A |
| F | AAATACGGTGGATTAAATAAAAGCAA | 1580 | Influenza B |
| R | CCAGCAATAGCTCCGAAGAAA | 1581 | Influenza B |
| P | CACCCATATTGGGCAATTTCCTATGGC | 1582 | Influenza B |
| F | ATCCCTACAATCCCCAAAGTCAAGGAGT | 1583 | HIV-1 |
| R | CCTGCACTGTACCCCCCAATCC | 1584 | HIV-1 |
| P | ACAGCAGTACAAATGGCA | 1585 | HIV-1 |
| F | ACTGATGGCAGTTCATTGCATGAATTTTAAAAG | 1586 | HIV-2 |
| R | GGCCATTGTTTAACTTTTGGGCCATCCA | 1587 | HIV-2 |
| P | ATAAGCCCCATAGCC | 1588 | HIV-2 |
| F | GGACCCCTGCTCGTGTTACA | 1589 | HBV |
| R | GAGAGAAGTCCACCMCGAGTCTAG | 1590 | HBV |
| P | TGTTGACAARAATCCTCACCATACCRCAGA | 1591 | HBV |
| F | GTGGTCTGCGGAACCGGTGA | 1592 | HCV |
| R | CGCAAGCACCCTATCAGGCAGT | 1593 | HCV |
| P | CCGAGTAGTGTTGGGTCGCGAAAGG | 1594 | HCV |
| F-HSV-1 | GCAGTTTACGTACAACCACATACAGC | 1595 | HSV-1 |
| F-HSV-2 | TGCAGTTTACGTATAACCACATACAGC | 1596 | HSV-2 |
| R | AGCTTGCGGGCCTCGTT | 1597 | HSV-1/2 |
| P-HSV-1 | CGGCCCAACATATCGTTGACATGGC | 1598 | HSV-1 |
| P-HSV-2 | CGCCCCAGCATGTCGTTCACGT | 1599 | HSV-2 |
| F | AACAGATGTAAGCAGCTCCGTTATC | 1600 | RSV |
| R | CGATTTTTATTGGATGCTGTACATTT | 1601 | RSV |
| P | TGCCATAGCATGACACAATGGCTCCT | 1602 | RSV |
| F | TCCTCCGGCCCCTGAAT | 1603 | Rhinovirus |
| R | GAAACACGGACACCCAAAGTAGT | 1604 | Rhinovirus |
| P | YGGCTAACCTWAACCC | 1605 | Rhinovirus |
| F | CCGCTCCTACCTGCAATATCA | 1606 | EBV |
| R | GGAAACCAGGGAGGCAAATG | 1607 | EBV |
| P | TGCAGCTTTGACGATGG | 1608 | EBV |
| F | GCTGACGCGTTTGGTCATC | 1609 | CMV |
| R | ACGATTCACGGAGCACCAG | 1610 | CMV |
| P | TCGGCGGATCACCACGTTCG | 1611 | CMV |
| F | TCGAAATAAGCATTAATAGGCACACT | 1612 | HHV6 |
| R | CGGAGTTAAGGCATTGGTTGA | 1613 | HHV6 |
| P | CCAAGCAGTTCCGTTTCTCTGAGCCA | 1614 | HHV6 |
| F | CASRGTGATCAAARTGRRARYGAGCT | 1615 | Measles |
| R | CCTGCCATGGYYTGCA | 1616 | Measles |
| P | TCYGATRCAGTRTCAAT | 1617 | Measles |
| F | TCAGCGATCTCTCCACCAAAG | 1618 | WNV |
| R | GGGTCAGCACGTTTGTCATTG | 1619 | WNV |
| P | TGCCCGACCATGGGAGAAGCTC | 1620 | WNV |
| F | ACWCARHTVAAYYTNAARTAYGC | 1621 | Coronavirus |
| R | TCRCAYTTDGGRTARTCCA | 1622 | Coronavirus |
| F | GCACAGCCACGTGACGAA | 1623 | Bocavirus |
| R | TGGACTCCCTTTTCTTTTGTAGGA | 1624 | Bocavirus |
| P | TGAGCTCAGGGAATATGAAAGACAAGCATC | 1625 | Bocavirus |
| F | CCCTGAATGCGGCTAATCC | 1626 | Enterovirus |
| R | ATTGTCACCATAAGCAGCCA | 1627 | Enterovirus |
| P | AACCGACTACTTTGGGTGTCCGTGTTTC | 1628 | Enterovirus |
| F | TTCCAGCATAATAACTCWGGCTTTG | 1629 | Adenovirus |
| R | AATTTTTTCTGWGTCAGGCTTGG | 1630 | Adenovirus |
| P | CCATACCCCCTTATTGG | 1631 | Adenovirus |
| F | CAGTGGTTGATGCTCAAGATGGA | 1632 | Rotavirus |
| R | TCATTGTAATCATATTGAATCCCCA | 1633 | Rotavirus |
| P | ACAACTGCAGCTTCAAAAGAAGWGT | 1634 | Rotavirus |
| F | TCAATATGCTGAAACGCGCGAGAAACCG | 1635 | Dengue |
| R | TTGCACCAACAGTCAATGTCTTCAGGTTC | 1636 | Deng ue |
| P | GAAGAATGGAGCGATCAAAGTG | 1637 | Dengue |
| F | GTAACASWWGCCTCTGGGSCCAAAAG | 1638 | Parechovirus |
| R | GGCCCCWGRTCAGATCCAYAGT | 1639 | Parechovirus |
| P | CCTRYGGGTACCTYCWGGGCATCCTTC | 1640 | Parechovirus |
| F | AGTCTTTAGGGTCTTCTACCTT | 1641 | BK virus |
| R | GGTGCCAACCTATGGAACAG | 1642 | BK virus |
| P | TCATCACTGGCAAACAT | 1643 | BK virus |
| F | ACAGGAATTGGCTCAGATATGYG | 1644 | Parainfluenza |
| R | GACTTCCCTATATCTGCACATCCTTGAGTG | 1645 | Parainfluenza |
| P | ACCATGCAGACGGC | 1646 | Parainfluenza |
| F | CACTTCCGAATGGCTGA | 1647 | TTV |
| R | GCCTTGCCCATAGCCCGC | 1648 | TTV |
| P | TCCCGAGCCCGAATTGCCCCT | 1649 | TTV |
| F | GAACCATCACTCCACAGAGGAG | 1650 | Coxsackie |
| R | GTACCTGTGGTGGGCATTG | 1651 | Coxsackie |
| P | CAGCCATTGGGAATTTCTTTAGCCGTG | 1652 | Coxsackie |
| F | TGGCCCATTTTCAAGGAAGT | 1653 | Parvo B19 |
| R | CTGAAGTCATGCTTGGGTATTTTTC | 1654 | Parvo B19 |
| P | CCGGAAGTTCCCGCTTACAAC | 1655 | Parvo B19 |

Current diagnosis of protozoal infections is achieved by pathogen detection using a variety of methods including light microscopy, or antigen or nucleic acid detection using different techniques such as tissue biopsy and histology, fecal or blood smears and staining, ELISA, lateral flow immunochromatography, and nucleic acid amplification. Common protozoan human pathogens, which can be detected using these techniques, include Plasmodium (malaria), Leishmania (leishmaniasis), Trypanosoma (sleeping sickness and Chagas disease), Cryptosporidium, Giardia, Toxoplasma, Babesia, Balantidium and Entamoeba. Common and well-known protozoan human pathogens that can be found in peripheral blood (causing a parasitemia—see TABLE 1 for a list) include Plasmodium falciparum, Plasmodium ovale, Plasmodium malariae, Plasmodium vivax, Leishmania donovani, Trypanosoma brucei, Trypanosoma cruzi, Toxoplasma gondii and Babesia microti.

In specific embodiments, protozoans that are capable of causing pathology in humans, as for example those listed in TABLE 1, which are capable of causing SIRS and cause a parasitemia are detected and/or quantified using any suitable nucleic acid detection and/or amplification assay, with oligonucleotide primers and/or probes in TABLE G.

TABLE G

| Reagent | 5′-3′ Sequence | SEQ ID NO. | Organisms Detected |
|---|---|---|---|
| Forward (F) | TTTCATTAATCAAGAACGAAAGTTAGGGG | 1656 | Toxoplasma gondii and |
| | | | Babesia microti |
| F2 | TTCCATTAATCAAGAACGAAAGTTAAGGG | 1657 | Plasmodium ovale, |
| | | | falciparum, malariae, vivax |
| F3 | AAACGATGACACCCATGAATTGGGGA | 1658 | Trypanosoma cruzi, brucei |
| | | | and Leishmania donovani |
| Probe (Pr) | CGTAGTCCTAACCATAAAC | 1659 | Babesia microti |
| Pr2 | AAACTATGCCGACTAGG | 1660 | Plasmodium ovale, |
| | | | falciparum, malariae, vivax |
| Pr3 | GACTTCTCCTGCACCTTAT | 1661 | Toxoplasma gondii |
| Pr4 | ACGGGAATATCCTCAGCACGTT | 1662 | Trypanosoma cruzi, brucei |
| | | | and Leishmania donovani |
| Reverse (R) | TCAAAGTCTTTGGGTTCTGGGGGG | 1663 | Toxoplasma gondii and |
| | | | Babesia microti |
| R2 | TCAAAGTCTTTGGGTTCTGGGGCG | 1664 | Plasmodium ovale, |
| | | | falciparum, malariae, vivax |
| R3 | CGTTCGCAAGAGTGAAACTTAAAG | 1665 | Trypanosoma cruzi, brucei |
| | | | and Leishmania donovani |

The indicator-determining methods of the present invention typically include obtaining a sample from a subject that typically has at least one clinical sign of SIRS. The sample typically comprises a biological fluid and in preferred embodiments comprises blood, suitably peripheral blood. The sample will typically include one or more BaSIRS, VaSIRS, PaSIRS or InSIRS biomarkers (e.g., polynucleotide or polypeptide expression products of BaSIRS, VaSIRS, PaSIRS or InSIRS genes) and none, one or more BIP, VIP or PIP biomarkers, quantifying at least two (e.g., 2, 3, 4, 5, 6, 7, 8, 9, 10 or more) of the BaSIRS, VaSIRS, PaSIRS or InSIRS host response specific biomarkers and optionally quantifying at least one (e.g., 1, 2, 3, 4, 5, 6, 7, 8, 9, 10 or more) of the BIP, VIP or PIP pathogen specific biomarkers) within the sample to determine biomarker values. This can be achieved using any suitable technique, and will depend on the nature of the BaSIRS, VaSIRS, PaSIRS, InSIRS, BIP, VIP or PIP biomarkers. Suitably, a BaSIRS, VaSIRS, PaSIRS or InSIRS host response specific biomarker value corresponds to the level of a respective BaSIRS, VaSIRS, PaSIRS or InSIRS biomarkers or to a function that is applied to that level. Suitably, an individual measured BIP, VIP or PIP pathogen specific biomarker value corresponds to the level of a respective BIP, VIP or PIP biomarker or to a function that is applied to that level or amount.

The host response specific derived biomarker values can be used alone or in combination with the at least one pathogen specific biomarker value to at least partially determine the indicator. For example, the indicator may be determined directly simply by combining the host response specific derived biomarker values using a combining function. Alternatively, the host response specific derived biomarker values and the at least one pathogen specific biomarker value are combined using a combining function to provide a compound biomarker value that is used to directly determine the indicator. In other embodiments, the host response specific derived biomarker values and optionally the at least one pathogen specific biomarker value are subjected to further processing, such as comparing the derived biomarker value to a reference, or using a cut-off value for pathogen specific biomarker, or the like, as will be described in more detail below, for determining the indicator. In certain of these embodiments, the indicator-determining methods additionally involve: combining the at least one pathogen specific biomarker value and the first, second and optionally third host response specific derived biomarker values using a combining function to provide a compound biomarker value and determining the indicator based at least in part on the compound biomarker value. Thus, in these embodiments, two or more pairs of host response specific derived biomarker values can be used in combination with one or more pathogen specific biomarker values, to provide a compound biomarker value that can assist in increasing the ability of the indicator to reliably determine the likelihood of a subject having, or not having, BaSIRS, VaSIRS, PaSIRS or InSIRS.

As disclosed herein, a combination of host response specific derived biomarker values and optionally at least one pathogen specific biomarker value can be combined using a combining function such as an additive model; a linear model; a support vector machine; a neural network model; a random forest model; a regression model; a genetic algorithm; an annealing algorithm; a weighted sum; a nearest neighbor model; and a probabilistic model. Various combinations of host response derived biomarkers and pathogen specific biomarkers are envisaged.

In some embodiments, the indicator is compared to an indicator reference, with a likelihood being determined in accordance with results of the comparison. The indicator reference may be derived from indicators determined for a number of individuals in a reference population. The reference population typically includes individuals having different characteristics, such as a plurality of individuals of different sexes; and/or ethnicities, with different groups being defined based on different characteristics, with the subject's indicator being compared to indicator references derived from individuals with similar characteristics. The reference population can also include a plurality of healthy individuals, a plurality of individuals suffering from BaSIRS, VaSIRS, PaSIRS or InSIRS, a plurality of individuals showing clinical signs of BaSIRS, VaSIRS, PaSIRS or InSIRS, and/or first and second groups of individuals, each group of individuals suffering from a respective diagnosed SIRS.

The indicator can also be used for determining a likelihood of the subject having a first or second condition, wherein the first condition is BaSIRS, VaSIRS, PaSIRS or InSIRS and the second condition is a healthy condition; in other words to distinguish between these conditions. In this case, this would typically be achieved by comparing the indicator to first and second indicator references, the first and second indicator references being indicative of first and second conditions and determining the likelihood in accordance with the results of the comparison. In particular, this can include determining first and second indicator probabilities using the results of the comparisons and combining the first and second indicator probabilities, for example using a Bayes method, to determine a condition probability corresponding to the likelihood of the subject having one of the conditions. In this situation the first and second conditions could include BaSIRS, VaSIRS, PaSIRS or InSIRS, or BaSIRS, VaSIRS, PaSIRS or InSIRS and a healthy condition. In this case, the first and second indicator references are distributions of indicators determined for first and second groups of a reference population, the first and second group consisting of individuals diagnosed with the first or second condition respectively.

In specific embodiments, the indicator-determining methods of the present invention are performed using at least one electronic processing device, such as a suitably programmed computer system or the like. In this case, the electronic processing device typically obtains at least one pair of measured host response specific biomarker values, and at least one pathogen specific biomarker value, either by receiving these from a measuring or other quantifying device, or by retrieving these from a database or the like. The processing device then determines a first derived biomarker value indicative of a ratio of levels of first and second host response specific biomarkers in a sample under test. In some embodiments, the processing device determines a second derived biomarker value indicative of a ratio of levels of third and fourth host response specific biomarkers, and optionally a third derived biomarker value indicative of a ratio of levels of fifth and sixth host response specific biomarkers in the sample. In its simplest form, the processing device may at least partially determine the indicator using only the first host response specific derived biomarker value. In other embodiments, the processing device combines the first host response specific derived biomarker value and the at least one pathogen specific biomarker value to provide a compound biomarker value that is used to at least partially determine the indicator. In still other embodiments, the processing device combines the first host response specific derived biomarker value, the second host response specific derived biomarker value, and optionally the third host response specific derived biomarker value to provide a combined derived biomarker value that is used to at least partially determine the indicator. In further embodiments, the processing device combines the first host response specific derived biomarker value, the second host response specific derived biomarker value, and optionally the third host response specific derived biomarker value and the at least one pathogen specific biomarker value to provide a compound derived biomarker value that is used to at least partially determine the indicator.

The processing device can then generate a representation of the indicator, for example by generating an alphanumeric indication of the indicator, a graphical indication of a comparison of the indicator to one or more indicator references or an alphanumeric indication of a likelihood of the subject having at least one medical condition.

The indicator-determining methods of the present invention are based on determining the level of individual host response specific biomarkers and optionally pathogen specific biomarkers to thereby determine their biomarker values. It should be understood, however, that a biomarker level does not need to be an absolute amount of biomarker. Instead, biomarker levels may correspond for example to a relative amount or concentration of a biomarker as well as any value or parameter which correlates thereto or can be derived therefrom. For example, in some embodiments of the indicator-determining methods, which employ a pair of host response specific biomarker polynucleotides and at least one pathogen specific biomarker polynucleotide, the methods may involve quantifying the host response specific biomarker polynucleotides and the at least one pathogen specific biomarker polynucleotide for example by nucleic acid amplification (e.g., by PCR) of the host response specific biomarker polynucleotides and the at least one pathogen specific polynucleotide in the sample, determining an amplification amount representing a degree of amplification required to obtain a defined level of each of the pair of host response specific biomarker polynucleotides and of the at least one pathogen specific polynucleotide and determining the indicator by first determining a difference between the amplification amounts of the pair of host response specific biomarker polynucleotides to provide a difference amplification amount and then combining the difference amplification amount and the amplification amount of the pathogen specific polynucleotide to thereby determine an indicator value that is at least partially indicative of the presence, absence or degree of the corresponding SIRS condition under test. In this regard, the amplification amount is generally a cycle time, a number of cycles, a cycle threshold and an amplification time.

Accordingly, in some embodiments, the methods may broadly comprise: determining a host response specific derived biomarker value by determining a difference between the amplification amounts of a first pair of host response specific biomarker polynucleotides; determining at least one pathogen specific biomarker value; and determining the indicator by combining the host response specific derived biomarker value and then the at least one pathogen specific biomarker value. In further illustrations of these embodiments, the methods may include: determining a first host response specific derived biomarker value by determining a difference between the amplification amounts of a first pair of host response specific biomarker polynucleotides; determining a second host response specific derived biomarker value by determining a difference between the amplification amounts of a second pair of host response specific biomarker polynucleotides; optionally determining a third host response specific derived biomarker value by determining a difference between the amplification amounts of a third pair of host response specific biomarker polynucleotides; determining at least one pathogen specific biomarker value; and determining the indicator by adding the first, second and/or third derived biomarker values to provide a combined derived biomarker value and combining the combined derived biomarker value and the pathogen specific biomarker value(s) to thereby determine an indicator value that is at least partially indicative of the presence, absence or degree of the corresponding SIRS condition under test.

In some embodiments, the presence, absence or degree of BaSIRS, VaSIRS, PaSIRS or InSIRS in a subject is established by determining one or more of BaSIRS, VaSIRS, PaSIRS or InSIRS host response specific biomarker values, wherein individual BaSIRS, VaSIRS, PaSIRS or InSIRS biomarker values are indicative of a value measured or derived for a BaSIRS, VaSIRS, PaSIRS or InSIRS biomarker in a subject or in a sample taken from the subject. These biomarkers are referred to herein as “sample BaSIRS, VaSIRS, PaSIRS or InSIRS biomarkers”. In accordance with the present invention, a sample BaSIRS, VaSIRS, PaSIRS or InSIRS biomarker corresponds to a reference BaSIRS, VaSIRS, PaSIRS or InSIRS biomarker (also referred to herein as a “corresponding BaSIRS, VaSIRS, PaSIRS or InSIRS biomarker”). By “corresponding BaSIRS, VaSIRS, PaSIRS or InSIRS biomarker” is meant a BaSIRS, VaSIRS, PaSIRS or InSIRS biomarker that is structurally and/or functionally similar to a reference BaSIRS, VaSIRS, PaSIRS or InSIRS biomarker as set forth for example in SEQ ID NOs: 1-1575. Representative corresponding BaSIRS, VaSIRS, PaSIRS or InSIRS biomarkers include expression products of allelic variants (same locus), homologues (different locus), and orthologues (different organism) of reference BaSIRS, VaSIRS, PaSIRS or InSIRS biomarker genes. Nucleic acid variants of reference BaSIRS, VaSIRS, PaSIRS or InSIRS biomarker genes and encoded BaSIRS, VaSIRS, PaSIRS or InSIRS biomarker polynucleotide expression products can contain nucleotide substitutions, deletions, inversions and/or insertions. Variation can occur in either or both the coding and non-coding regions. The variations can produce both conservative and non-conservative amino acid substitutions (as compared in the encoded product). For nucleotide sequences, conservative variants include those sequences that, because of the degeneracy of the genetic code, encode the amino acid sequence of a reference BaSIRS, VaSIRS, PaSIRS or InSIRS polypeptide.

Generally, variants of a particular BaSIRS, VaSIRS, PaSIRS or InSIRS biomarker gene or polynucleotide will have at least about 40%, 45%, 50%, 51%, 52%, 53%, 54%, 55%, 56%, 57%, 58%, 59% 60%, 61%, 62%, 63%, 64%, 65%, 66%, 67%, 68%, 69% 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more sequence identity to that particular nucleotide sequence as determined by sequence alignment programs known in the art using default parameters. In some embodiments, the BaSIRS, VaSIRS, PaSIRS or InSIRS biomarker gene or polynucleotide displays at least about 40%, 45%, 50%, 51%, 52%, 53%, 54%, 55%, 56%, 57%, 58%, 59% 60%, 61%, 62%, 63%, 64%, 65%, 66%, 67%, 68%, 69% 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a nucleotide sequence selected from any one of SEQ ID NO: 1-94, 189-601, 1014-1143 and 1274-1424, as summarized in TABLES 3, 5, 7 and 9.

Corresponding BaSIRS, VaSIRS, PaSIRS or InSIRS biomarkers also include amino acid sequences that display substantial sequence similarity or identity to the amino acid sequence of a reference BaSIRS, VaSIRS, PaSIRS or InSIRS biomarker polypeptide. In general, an amino acid sequence that corresponds to a reference amino acid sequence will display at least about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 97, 88, 89, 90, 91, 92, 93, 94, 95, 96, 97, 98, 99% or even up to 100% sequence similarity or identity to a reference amino acid sequence selected from any one of SEQ ID NO: 95-188, 602-103, 1144-1273 and 1425-1575, as summarized in TABLES 4, 6, 8 and 10.

In some embodiments, calculations of sequence similarity or sequence identity between sequences are performed as follows:

To determine the percentage identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In some embodiments, the length of a reference sequence aligned for comparison purposes is at least 30%, usually at least 40%, more usually at least 50%, 60%, and even more usually at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide at the corresponding position in the second sequence, then the molecules are identical at that position. For amino acid sequence comparison, when a position in the first sequence is occupied by the same or similar amino acid residue (i.e., conservative substitution) at the corresponding position in the second sequence, then the molecules are similar at that position.

The percentage identity between the two sequences is a function of the number of identical amino acid residues shared by the sequences at individual positions, taking into account the number of gaps, and the length of each gap, which need to be introduced for optimal alignment of the two sequences. By contrast, the percentage similarity between the two sequences is a function of the number of identical and similar amino acid residues shared by the sequences at individual positions, taking into account the number of gaps, and the length of each gap, which need to be introduced for optimal alignment of the two sequences.

The comparison of sequences and determination of percentage identity or percentage similarity between sequences can be accomplished using a mathematical algorithm. In certain embodiments, the percentage identity or similarity between amino acid sequences is determined using the Needleman and Wunsch, (1970, J. Mol. Biol. 48: 444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In specific embodiments, the percent identity between nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. An non-limiting set of parameters (and the one that should be used unless otherwise specified) includes a Blossum 62 scoring matrix with a gap penalty of 12, a gap extend penalty of 4, and a frameshift gap penalty of 5.

In some embodiments, the percentage identity or similarity between amino acid or nucleotide sequences can be determined using the algorithm of E. Meyers and W. Miller (1989, Cabios, 4: 11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al., (1990, J Mol Biol., 215: 403-10). BLAST nucleotide searches can be performed with the NBLAST program, score=100, wordlength=12 to obtain nucleotide sequences homologous to 53010 nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997, Nucleic Acids Res, 25: 3389-3402). When utilizing BLAST and Gapped BLAST programs, the default parameters of the respective programs (e.g., XBLAST and NBLAST) can be used.

Corresponding BaSIRS, VaSIRS, PaSIRS or InSIRS biomarker polynucleotides also include nucleic acid sequences that hybridize to reference BaSIRS, VaSIRS, PaSIRS or InSIRS biomarker polynucleotides, or to their complements, under stringency conditions described below. As used herein, the term “hybridizes under low stringency, medium stringency, high stringency, or very high stringency conditions” describes conditions for hybridization and washing. “Hybridization” is used herein to denote the pairing of complementary nucleotide sequences to produce a DNA-DNA hybrid or a DNA-RNA hybrid. Complementary base sequences are those sequences that are related by the base-pairing rules. In DNA, A pairs with T and C pairs with G. In RNA, U pairs with A and C pairs with G. In this regard, the terms “match” and “mismatch” as used herein refer to the hybridization potential of paired nucleotides in complementary nucleic acid strands. Matched nucleotides hybridize efficiently, such as the classical A-T and G-C base pair mentioned above. Mismatches are other combinations of nucleotides that do not hybridize efficiently.

Guidance for performing hybridization reactions can be found in Ausubel et al., (1998, supra), Sections 6.3.1-6.3.6. Aqueous and non-aqueous methods are described in that reference and either can be used. Reference herein to low stringency conditions include and encompass from at least about 1% v/v to at least about 15% v/v formamide and from at least about 1 M to at least about 2 M salt for hybridization at 42° C., and at least about 1 M to at least about 2 M salt for washing at 42° C. Low stringency conditions also may include 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at room temperature. One embodiment of low stringency conditions includes hybridization in 6×sodium chloride/sodium citrate (SSC) at about 45° C., followed by two washes in 0.2×SSC, 0.1% SDS at least at 50° C. (the temperature of the washes can be increased to 55° C. for low stringency conditions). Medium stringency conditions include and encompass from at least about 16% v/v to at least about 30% v/v formamide and from at least about 0.5 M to at least about 0.9 M salt for hybridization at 42° C., and at least about 0.1 M to at least about 0.2 M salt for washing at 55° C. Medium stringency conditions also may include 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at 60-65° C. One embodiment of medium stringency conditions includes hybridizing in 6×SSC at about 45° C., followed by one or more washes in 0.2×SSC, 0.1% SDS at 60° C. High stringency conditions include and encompass from at least about 31% v/v to at least about 50% v/v formamide and from about 0.01 M to about 0.15 M salt for hybridization at 42° C., and about 0.01 M to about 0.02 M salt for washing at 55° C. High stringency conditions also may include 1% BSA, 1 mM EDTA, 0.5 M NaHPO₄ (pH 7.2), 7% SDS for hybridization at 65° C., and (i) 0.2×SSC, 0.1% SDS; or (ii) 0.5% BSA, 1 mM EDTA, 40 mM NaHPO₄ (pH 7.2), 1% SDS for washing at a temperature in excess of 65° C. One embodiment of high stringency conditions includes hybridizing in 6×SSC at about 45° C., followed by one or more washes in 0.2×SSC, 0.1% SDS at 65° C.

In certain embodiments, a corresponding BaSIRS, VaSIRS, PaSIRS or InSIRS biomarker polynucleotide is one that hybridizes to a disclosed nucleotide sequence under very high stringency conditions. One embodiment of very high stringency conditions includes hybridizing 0.5 M sodium phosphate, 7% SDS at 65° C., followed by one or more washes at 0.2×SSC, 1% SDS at 65° C.

Other stringency conditions are well known in the art and a skilled addressee will recognize that various factors can be manipulated to optimize the specificity of the hybridization. Optimization of the stringency of the final washes can serve to ensure a high degree of hybridization. For detailed examples, see Ausubel et al., supra at pages 2.10.1 to 2.10.16 and Sambrook et al. (1989, supra) at sections 1.101 to 1.104.

Generally, a sample is processed prior to BaSIRS, VaSIRS, PaSIRS, InSIRS, BIP, VIP or PIP biomarker detection or quantification. For example, nucleic acid and/or proteins may be extracted, isolated, and/or purified from a sample prior to analysis. Various DNA, mRNA, and/or protein extraction techniques are well known to those skilled in the art. Processing may include centrifugation, ultracentrifugation, ethanol precipitation, filtration, fractionation, resuspension, dilution, concentration, etc. In some embodiments, methods and systems provide analysis (e.g., quantification of RNA or protein biomarkers) from raw sample (e.g., biological fluid such as blood, serum, etc.) without or with limited processing.

Methods may comprise steps of homogenizing a sample in a suitable buffer, removal of contaminants and/or assay inhibitors, adding a BaSIRS, VaSIRS, PaSIRS, InSIRS, BIP, VIP or PIP biomarker capture reagent (e.g., a magnetic bead to which is linked an oligonucleotide complementary to a target BaSIRS, VaSIRS, PaSIRS, InSIRS, BIP, VIP or PIP nucleic acid biomarker), incubated under conditions that promote the association (e.g., by hybridization) of the target biomarker with the capture reagent to produce a target biomarker:capture reagent complex, incubating the target biomarker:capture complex under target biomarker-release conditions. In some embodiments, multiple BaSIRS, VaSIRS, PaSIRS, InSIRS, BIP, VIP or PIP biomarkers are isolated in each round of isolation by adding multiple BaSIRS, VaSIRS, PaSIRS, InSIRS, BIP, VIP or PIP biomarkers capture reagents (e.g., specific to the desired biomarkers) to the solution. For example, multiple BaSIRS, VaSIRS, PaSIRS, InSIRS, BIP, VIP or PIP biomarker capture reagents, each comprising an oligonucleotide specific for a different target BaSIRS, VaSIRS, PaSIRS, InSIRS, BIP, VIP or PIP biomarker can be added to the sample for isolation of multiple BaSIRS, VaSIRS, PaSIRS, InSIRS, BIP, VIP or PIP biomarker. It is contemplated that the methods encompass multiple experimental designs that vary both in the number of capture steps and in the number of target BaSIRS, VaSIRS, PaSIRS, InSIRS, BIP, VIP or PIP biomarker captured in each capture step. In some embodiments, capture reagents are molecules, moieties, substances, or compositions that preferentially (e.g., specifically and selectively) interact with a particular biomarker sought to be isolated, purified, detected, and/or quantified. Any capture reagent having desired binding affinity and/or specificity to the particular BaSIRS, VaSIRS, PaSIRS, InSIRS, BIP, VIP or PIP biomarker can be used in the present technology. For example, the capture reagent can be a macromolecule such as a peptide, a protein (e.g., an antibody or receptor), an oligonucleotide, a nucleic acid, (e.g., nucleic acids capable of hybridizing with the VaSIRS biomarkers), vitamins, oligosaccharides, carbohydrates, lipids, or small molecules, or a complex thereof. As illustrative and non-limiting examples, an avidin target capture reagent may be used to isolate and purify targets comprising a biotin moiety, an antibody may be used to isolate and purify targets comprising the appropriate antigen or epitope, and an oligonucleotide may be used to isolate and purify a complementary oligonucleotide.

Any nucleic acids, including single-stranded and double-stranded nucleic acids, that are capable of binding, or specifically binding, to a target BaSIRS, VaSIRS, PaSIRS, InSIRS, BIP, VIP or PIP biomarker can be used as the capture reagent. Examples of such nucleic acids include DNA, RNA, aptamers, peptide nucleic acids, and other modifications to the sugar, phosphate, or nucleoside base. Thus, there are many strategies for capturing a target and accordingly many types of capture reagents are known to those in the art.

In addition, BaSIRS, VaSIRS, PaSIRS, InSIRS, BIP, VIP or PIP biomarker capture reagents may comprise a functionality to localize, concentrate, aggregate, etc. the capture reagent and thus provide a way to isolate and purify the target BaSIRS, VaSIRS, PaSIRS, InSIRS, BIP, VIP or PIP biomarker when captured (e.g., bound, hybridized, etc.) to the capture reagent (e.g., when a target:capture reagent complex is formed). For example, in some embodiments the portion of the capture reagent that interacts with the BaSIRS, VaSIRS, PaSIRS, InSIRS, BIP, VIP or PIP biomarker (e.g., an oligonucleotide) is linked to a solid support (e.g., a bead, surface, resin, column, and the like) that allows manipulation by the user on a macroscopic scale. Often, the solid support allows the use of a mechanical means to isolate and purify the target:capture reagent complex from a heterogeneous solution. For example, when linked to a bead, separation is achieved by removing the bead from the heterogeneous solution, e.g., by physical movement. In embodiments in which the bead is magnetic or paramagnetic, a magnetic field is used to achieve physical separation of the capture reagent (and thus the target BaSIRS, VaSIRS, PaSIRS, InSIRS, BIP, VIP or PIP biomarker) from the heterogeneous solution.

The BaSIRS, VaSIRS, PaSIRS, InSIRS, BIP, VIP or PIP biomarkers may be quantified or detected using any suitable means. In specific embodiments, the BaSIRS, VaSIRS, PaSIRS, InSIRS, BIP, VIP or PIP biomarkers are quantified using reagents that determine the level, abundance or amount of individual BaSIRS, VaSIRS, PaSIRS, InSIRS, BIP, VIP or PIP biomarkers. Non-limiting reagents of this type include reagents for use in nucleic acid- and protein-based assays.

In illustrative nucleic acid-based assays, nucleic acid is isolated from cells contained in the biological sample according to standard methodologies (Sambrook, et al., 1989, supra; and Ausubel et al., 1994, supra). The nucleic acid is typically fractionated (e.g., poly A⁺ RNA) or whole cell RNA. Where RNA is used as the subject of detection, it may be desired to convert the RNA to a complementary DNA (cDNA). In some embodiments, the nucleic acid is amplified by a template-dependent nucleic acid amplification reaction. A number of template dependent processes are available to amplify the BaSIRS, VaSIRS, PaSIRS, InSIRS, BIP, VIP or PIP biomarker sequences present in a given template sample. An exemplary nucleic acid amplification technique is the polymerase chain reaction (referred to as PCR), which is described in detail in U.S. Pat. Nos. 4,683,195, 4,683,202 and 4,800,159, Ausubel et al. (supra), and in Innis et al., (“PCR Protocols”, Academic Press, Inc., San Diego Calif., 1990). Briefly, in PCR, two primer sequences are prepared that are complementary to regions on opposite complementary strands of the biomarker sequence. An excess of deoxynucleotide triphosphates are added to a reaction mixture along with a DNA polymerase, e.g., Taq polymerase. If a cognate BaSIRS, VaSIRS, PaSIRS, InSIRS, BIP, VIP or PIP biomarker sequence is present in a sample, the primers will bind to the biomarker and the polymerase will cause the primers to be extended along the biomarker sequence by adding on nucleotides. By raising and lowering the temperature of the reaction mixture, the extended primers will dissociate from the biomarker to form reaction products, excess primers will bind to the biomarker and to the reaction products and the process is repeated. A reverse transcriptase PCR amplification procedure may be performed in order to quantify the amount of mRNA amplified. Methods of reverse transcribing RNA into cDNA are well known and described in Sambrook et al., 1989, supra. Alternative methods for reverse transcription utilize thermostable, RNA-dependent DNA polymerases. These methods are described in WO 90/07641. Polymerase chain reaction methodologies are well known in the art. In specific embodiments in which whole cell RNA is used, cDNA synthesis using whole cell RNA as a sample produces whole cell cDNA.

Detection and/or quantification of the amplified target polynucleotides may be facilitated by attachment of a heterologous detectable label to an oligonucleotide primer or probe that is used in the amplification reaction, illustrative examples of which include radioisotopes, fluorophores, chemiluminophores, bioluminescent molecules, lanthanide ions (e.g., Eu³⁴), enzymes, colloidal particles, dye particles and fluorescent microparticles or nanoparticles, as well as antigens, antibodies, haptens, avidin/streptavidin, biotin, enzyme cofactors/substrates, enzymes, and the like. A label can optionally be attached to or incorporated into an oligonucleotide probe or primer to allow detection and/or quantitation of a target polynucleotide representing the target sequence of interest. The target polynucleotide may be the expressed target sequence RNA itself, a cDNA copy thereof, or an amplification product derived therefrom, and may be the positive or negative strand, so long as it can be specifically detected in the assay being used. In certain multiplex formats, labels used for detecting different targets may be distinguishable. The label can be attached directly (e.g., via covalent linkage) or indirectly, e.g., via a bridging molecule or series of molecules (e.g., a molecule or complex that can bind to an assay component, or via members of a binding pair that can be incorporated into assay components, e.g., biotin-avidin or streptavidin). Many labels are commercially available in activated forms which can readily be used for such conjugation (for example through amine acylation), or labels may be attached through known or determinable conjugation schemes, many of which are known in the art.

Labels useful in the invention described herein include any substance which can be detected when bound to or incorporated into the biomolecule of interest. Any effective detection method can be used, including optical, spectroscopic, electrical, piezoelectrical, magnetic, Raman scattering, surface plasmon resonance, colorimetric, calorimetric, etc. A label is typically selected from a chromophore, a lumiphore, a fluorophore, one member of a quenching system, a chromogen, a hapten, an antigen, a magnetic particle, a material exhibiting nonlinear optics, a semiconductor nanocrystal, a metal nanoparticle, an enzyme, an antibody or binding portion or equivalent thereof, an aptamer, and one member of a binding pair, and combinations thereof. Quenching schemes may be used, wherein a quencher and a fluorophore as members of a quenching pair may be used on a probe, such that a change in optical parameters occurs upon binding to the target introduce or quench the signal from the fluorophore. One example of such a system is a molecular beacon. Suitable quencher/fluorophore systems are known in the art. The label may be bound through a variety of intermediate linkages. For example, a polynucleotide may comprise a biotin-binding species, and an optically detectable label may be conjugated to biotin and then bound to the labeled polynucleotide. Similarly, a polynucleotide sensor may comprise an immunological species such as an antibody or fragment, and a secondary antibody containing an optically detectable label may be added.

Chromophores useful in the methods described herein include any substance which can absorb energy and emit light. For multiplexed assays, a plurality of different signaling chromophores can be used with detectably different emission spectra. The chromophore can be a lumiphore or a fluorophore. Typical fluorophores include fluorescent dyes, semiconductor nanocrystals, lanthanide chelates, polynucleotide-specific dyes and green fluorescent protein.

In certain advantageous embodiments, the template-dependent amplification reaction involves quantification of transcripts. For example, RNA or DNA may be quantified using a quantitative real-time PCR technique (Higuchi, 1992, et al., Biotechnology 10: 413-417). By determining the concentration of the amplified products of the target DNA in PCR reactions that have completed the same number of cycles and are in their linear ranges, it is possible to determine the relative levels of the specific target sequence in the original DNA mixture. If the DNA mixtures are cDNAs synthesized from RNAs isolated from different tissues or cells, the relative abundance of the specific mRNA from which the target sequence was derived can be determined for the respective tissues or cells. This direct proportionality between the concentration of the PCR products and the relative mRNA abundance is only true in the linear range of the PCR reaction. The final concentration of the target DNA in the plateau portion of the curve is determined by the availability of reagents in the reaction mix and is independent of the original concentration of target DNA. In specific embodiments, quantitative PCR (qPCR) is combined with fluorescence chemistry to enable real-time monitoring of the amplification reaction using detection of a fluorescent light signal. In illustrative examples, the qPCR methods use a sequence nonspecific fluorescent reporter dye such as SYBR green (see, Wittwer et al., Biotechniques 22(1):176-181, 1997). In other examples, the qPCR methods use a sequence specific fluorescent reporter such as a TAQMAN probe (see, Heid, et al., Genome Res. 6(10):986-994, 1996). During execution of the PCR cycling program, the samples are excited using a light source. A fluorescent signal, indicating the amount of PCR amplification product produced, is monitored in each reaction well using a photodetector or CCD/CMOS camera. By monitoring the fluorescence in the sample during the reaction precise quantitative measurements can be made. The probe based PCR method is considered to more accurate than the SYBR green method. PCR or qPCR is typically performed in plastic 96 or 384 well microtiter plates, each reaction having a volume in the order of 5-50 μL. PCR can however be carried out in very small (nanoliter) volumes. Other quantification strategies may be employed such as Molecular Beacon Probes (see, Tyagi et al., Nature Biotechnology 14: 303-308, 1996; or Situma et al., Analytical Biochemistry 363: 35-45, 2007).

Real-time PCR can be performed to detect a single gene or RNA molecule, however, multiple genes or RNA molecules may be detected in one reaction, i.e., by multiplexing. Detection of nucleic acids by multiplexing is described by Kosman, et al. (Science, 305: 846, 2004); Sakai et al. (BioScience Trends 2(4):164-168, 2008); or Gu et al. (Journal of Clinical Microbiology, 41(10): 4636-4641, 2003). For example, one or more biomarker mRNAs may be detected simultaneously, optionally with one or more housekeeping mRNAs in a single reaction. In certain embodiments, multiple biomarkers (e.g., target polynucleotides) are analyzed using real-time quantitative multiplex RT-PCR platforms and other multiplexing technologies such as GenomeLab GeXP Genetic Analysis System (Beckman Coulter, Foster City, Calif.), SmartCycler® 9600 or GeneXpert® Systems (Cepheid, Sunnyvale, Calif.), ABI 7900 HT Fast Real Time PCR system (Applied Biosystems, Foster City, Calif.), LightCycler® 480 System (Roche Molecular Systems, Pleasanton, Calif.), xMAP 100 System (Luminex, Austin, Tex.) Solexa Genome Analysis System (Illumina, Hayward, Calif.), OpenArray Real Time qPCR (BioTrove, Woburn, Mass.) and BeadXpress System (Illumina, Hayward, Calif.). In illustrative examples, multiplexed, tandem PCR (MT-PCR) is employed, which uses a two-step process for gene expression profiling from small quantities of RNA or DNA, as described for example in U.S. Pat. Appl. Pub. No. 20070190540. In the first step, RNA is converted into cDNA and amplified using multiplexed gene specific primers. In the second step each individual gene is quantitated by real-time PCR.

In certain embodiments, target nucleic acids are quantified using blotting techniques, which are well known to those of skill in the art. Southern blotting involves the use of DNA as a target, whereas Northern blotting involves the use of RNA as a target. Each provides different types of information, although cDNA blotting is analogous, in many aspects, to blotting or RNA species. Briefly, a probe is used to target a DNA or RNA species that has been immobilized on a suitable matrix, often a filter of nitrocellulose. The different species should be spatially separated to facilitate analysis. This often is accomplished by gel electrophoresis of nucleic acid species followed by “blotting” on to the filter. Subsequently, the blotted target is incubated with a probe (usually labeled) under conditions that promote denaturation and rehybridization. Because the probe is designed to base pair with the target, the probe will bind a portion of the target sequence under renaturing conditions. Unbound probe is then removed, and detection is accomplished as described above. Following detection/quantification, one may compare the results seen in a given subject with a control reaction or a statistically significant reference group or population of control subjects as defined herein. In this way, it is possible to correlate the amount of BaSIRS, VaSIRS, PaSIRS, InSIRS, BIP, VIP or PIP biomarker nucleic acid detected with the progression or severity of the disease.

Also contemplated are biochip-based technologies such as those described by Hacia et al. (1996, Nature Genetics 14: 441-447) and Shoemaker et al. (1996, Nature Genetics 14: 450-456). Briefly, these techniques involve quantitative methods for analyzing large numbers of genes rapidly and accurately. By tagging genes with oligonucleotides or using fixed nucleic acid probe arrays, one can employ biochip technology to segregate target molecules as high-density arrays and screen these molecules on the basis of hybridization. See also Pease et al. (1994, Proc. Natl. Acad. Sci. U.S.A. 91: 5022-5026); Fodor et al. (1991, Science 251: 767-773). Briefly, nucleic acid probes to BaSIRS, VaSIRS, PaSIRS, InSIRS, BIP, VIP or PIP biomarker polynucleotides are made and attached to biochips to be used in screening and diagnostic methods, as outlined herein. The nucleic acid probes attached to the biochip are designed to be substantially complementary to specific expressed BaSIRS, VaSIRS, PaSIRS, InSIRS, BIP, VIP or PIP biomarker nucleic acids, i.e., the target sequence (either the target sequence of the sample or to other probe sequences, for example in sandwich assays), such that hybridization of the target sequence and the probes of the present invention occur. This complementarity need not be perfect; there may be any number of base pair mismatches, which will interfere with hybridization between the target sequence and the nucleic acid probes of the present invention. However, if the number of mismatches is so great that no hybridization can occur under even the least stringent of hybridization conditions, the sequence is not a complementary target sequence. In certain embodiments, more than one probe per sequence is used, with either overlapping probes or probes to different sections of the target being used. That is, two, three, four or more probes, with three being desirable, are used to build in a redundancy for a particular target. The probes can be overlapping (i.e. have some sequence in common), or separate.

In an illustrative biochip analysis, oligonucleotide probes on the biochip are exposed to or contacted with a nucleic acid sample suspected of containing one or more BaSIRS, VaSIRS, PaSIRS, InSIRS, BIP, VIP or PIP biomarker polynucleotides under conditions favoring specific hybridization. Sample extracts of DNA or RNA, either single or double-stranded, may be prepared from fluid suspensions of biological materials, or by grinding biological materials, or following a cell lysis step which includes, but is not limited to, lysis effected by treatment with SDS (or other detergents), osmotic shock, guanidinium isothiocyanate and lysozyme. Suitable DNA, which may be used in the method of the invention, includes cDNA. Such DNA may be prepared by any one of a number of commonly used protocols as for example described in Ausubel, et al., 1994, supra, and Sambrook, et al., 1989, supra.

Suitable RNA, which may be used in the method of the invention, includes messenger RNA, complementary RNA transcribed from DNA (cRNA) or genomic or subgenomic RNA. Such RNA may be prepared using standard protocols as for example described in the relevant sections of Ausubel, et al. 1994, supra and Sambrook, et al. 1989, supra).

cDNA may be fragmented, for example, by sonication or by treatment with restriction endonucleases. Suitably, cDNA is fragmented such that resultant DNA fragments are of a length greater than the length of the immobilized oligonucleotide probe(s) but small enough to allow rapid access thereto under suitable hybridization conditions. Alternatively, fragments of cDNA may be selected and amplified using a suitable nucleotide amplification technique, as described for example above, involving appropriate random or specific primers.

Usually the target BaSIRS, VaSIRS, PaSIRS, InSIRS, BIP, VIP or PIP biomarker polynucleotides are detectably labeled so that their hybridization to individual probes can be determined. The target polynucleotides are typically detectably labeled with a heterologous label or reporter molecule illustrative examples of which include those mentioned above in respect for the primers or probes used in.

The hybrid-forming step can be performed under suitable conditions for hybridizing oligonucleotide probes to test nucleic acid including DNA or RNA. In this regard, reference may be made, for example, to NUCLEIC ACID HYBRIDIZATION, A PRACTICAL APPROACH (Homes and Higgins, eds.) (IRL press, Washington D.C., 1985). In general, whether hybridization takes place is influenced by the length of the oligonucleotide probe and the polynucleotide sequence under test, the pH, the temperature, the concentration of mono- and divalent cations, the proportion of G and C nucleotides in the hybrid-forming region, the viscosity of the medium and the possible presence of denaturants. Such variables also influence the time required for hybridization. The preferred conditions will therefore depend upon the particular application. Such empirical conditions, however, can be routinely determined without undue experimentation.

After the hybrid-forming step, the probes are washed to remove any unbound nucleic acid with a hybridization buffer. This washing step leaves only bound target polynucleotides. The probes are then examined to identify which probes have hybridized to a target polynucleotide.

The hybridization reactions are then detected to determine which of the probes has hybridized to a corresponding target sequence. Depending on the nature of the reporter molecule associated with a target polynucleotide, a signal may be instrumentally detected by irradiating a fluorescent label with light and detecting fluorescence in a fluorimeter; by providing for an enzyme system to produce a dye which could be detected using a spectrophotometer; or detection of a dye particle or a colored colloidal metallic or non-metallic particle using a reflectometer; in the case of using a radioactive label or chemiluminescent molecule employing a radiation counter or autoradiography. Accordingly, a detection means may be adapted to detect or scan light associated with the label which light may include fluorescent, luminescent, focused beam or laser light. In such a case, a charge couple device (CCD) or a photocell can be used to scan for emission of light from a probe:target polynucleotide hybrid from each location in the micro-array and record the data directly in a digital computer. In some cases, electronic detection of the signal may not be necessary. For example, with enzymatically generated color spots associated with nucleic acid array format, visual examination of the array will allow interpretation of the pattern on the array. In the case of a nucleic acid array, the detection means is suitably interfaced with pattern recognition software to convert the pattern of signals from the array into a plain language genetic profile. In certain embodiments, oligonucleotide probes specific for different VaSIRS biomarker polynucleotides are in the form of a nucleic acid array and detection of a signal generated from a reporter molecule on the array is performed using a ‘chip reader’. A detection system that can be used by a ‘chip reader’ is described for example by Pirrung et al. (U.S. Pat. No. 5,143,854). The chip reader will typically also incorporate some signal processing to determine whether the signal at a particular array position or feature is a true positive or maybe a spurious signal. Exemplary chip readers are described for example by Fodor et al. (U.S. Pat. No. 5,925,525). Alternatively, when the array is made using a mixture of individually addressable kinds of labeled microbeads, the reaction may be detected using flow cytometry.

In certain embodiments, the BaSIRS, VaSIRS, PaSIRS, InSIRS, BIP, VIP or PIP biomarker is a target RNA (e.g., mRNA) or a DNA copy of the target RNA whose level or abundance is measured using at least one nucleic acid probe that hybridizes under at least low, medium, or high stringency conditions to the target RNA or to the DNA copy, wherein the nucleic acid probe comprises at least 15 (e.g., 15, 16, 17, 18, 19, 20, 21, 22, 23, 24, 25, 26, 27, 28, 29, 30, or more) contiguous nucleotides of BaSIRS, VaSIRS, PaSIRS, BIP, VIP or PIP biomarker polynucleotide. In some embodiments, the measured level or abundance of the target RNA or its DNA copy is normalized to the level or abundance of a reference RNA or a DNA copy of the reference RNA. Suitably, the nucleic acid probe is immobilized on a solid or semi-solid support. In illustrative examples of this type, the nucleic acid probe forms part of a spatial array of nucleic acid probes. In some embodiments, the level of nucleic acid probe that is bound to the target RNA or to the DNA copy is measured by hybridization (e.g., using a nucleic acid array). In other embodiments, the level of nucleic acid probe that is bound to the target RNA or to the DNA copy is measured by nucleic acid amplification (e.g., using a polymerase chain reaction (PCR)). In still other embodiments, the level of nucleic acid probe that is bound to the target RNA or to the DNA copy is measured by nuclease protection assay.

Sequencing technologies such as Sanger sequencing, pyrosequencing, sequencing by ligation, massively parallel sequencing, also called “Next-generation sequencing” (NGS), and other high-throughput sequencing approaches with or without sequence amplification of the target can also be used to detect or quantify the presence of BaSIRS, VaSIRS, PaSIRS, InSIRS, BIP, VIP or PIP nucleic acid biomarker in a sample. Sequence-based methods can provide further information regarding alternative splicing and sequence variation in previously identified genes. Sequencing technologies include a number of steps that are grouped broadly as template preparation, sequencing, detection and data analysis. Current methods for template preparation involve randomly breaking genomic DNA into smaller sizes from which each fragment is immobilized to a support. The immobilization of spatially separated fragment allows thousands to billions of sequencing reaction to be performed simultaneously. A sequencing step may use any of a variety of methods that are commonly known in the art. One specific example of a sequencing step uses the addition of nucleotides to the complementary strand to provide the DNA sequence. The detection steps range from measuring bioluminescent signal of a synthesized fragment to four-color imaging of single molecule. In some embodiments in which NGS is used to detect or quantify the presence of BaSIRS, VaSIRS, PaSIRS, InSIRS, BIP, VIP or PIP nucleic acid biomarker in a sample, the methods are suitably selected from semiconductor sequencing (Ion Torrent; Personal Genome Machine); Helicos True Single Molecule Sequencing (tSMS) (Harris et al. 2008, Science 320:106-109); 454 sequencing (Roche) (Margulies et al. 2005, Nature, 437, 376-380); SOLiD technology (Applied Biosystems); SOLEXA sequencing (Illumina); single molecule, real-time (SMRT™) technology of Pacific Biosciences; nanopore sequencing (Soni and Meller, 2007. Clin Chem 53: 1996-2001); DNA nanoball sequencing; sequencing using technology from Dover Systems (Polonator), and technologies that do not require amplification or otherwise transform native DNA prior to sequencing (e.g., Pacific Biosciences and Helicos), such as nanopore-based strategies (e.g., Oxford Nanopore, Genia Technologies, and Nabsys).

In other embodiments, BaSIRS, VaSIRS, PaSIRS or InSIRS biomarker protein levels are assayed using protein-based assays known in the art. For example, when BaSIRS, VaSIRS, PaSIRS or InSIRS biomarker protein is an enzyme, the protein can be quantified based upon its catalytic activity or based upon the number of molecules of the protein contained in a sample. Antibody-based techniques may be employed including, for example, immunoassays, such as the enzyme-linked immunosorbent assay (ELISA) and the radioimmunoassay (RIA).

In other embodiments, BIP, VIP or PIP biomarker proteins, carbohydrates, lipids, metabolites or combinations of such pathogenic molecules are assayed using assays known in the art. Such assays could include, by example; enzyme immunoassay, mass spectrometry, liquid chromatography, lateral immunochromatography, or other methods capable of quantifying such molecules.

In specific embodiments, protein-capture arrays that permit simultaneous detection and/or quantification of a large number of proteins are employed. For example, low-density protein arrays on filter membranes, such as the universal protein array system (Ge, 2000 Nucleic Acids Res. 28(2):e3) allow imaging of arrayed antigens using standard ELISA techniques and a scanning charge-coupled device (CCD) detector. Immuno-sensor arrays have also been developed that enable the simultaneous detection of clinical analytes. It is now possible using protein arrays, to profile protein expression in bodily fluids, such as in sera of healthy or diseased subjects, as well as in subjects pre- and post-drug treatment.

Exemplary protein capture arrays include arrays comprising spatially addressed antigen-binding molecules, commonly referred to as antibody arrays, which can facilitate extensive parallel analysis of numerous proteins defining a proteome or subproteome. Antibody arrays have been shown to have the required properties of specificity and acceptable background, and some are available commercially (e.g., BD Biosciences, Clontech, Bio-Rad and Sigma). Various methods for the preparation of antibody arrays have been reported (see, e.g., Lopez et al., 2003 J. Chromatogram. B 787:19-27; Cahill, 2000 Trends in Biotechnology 7:47-51; U.S. Pat. App. Pub. 2002/0055186; U.S. Pat. App. Pub. 2003/0003599; PCT publication WO 03/062444; PCT publication WO 03/077851; PCT publication WO 02/59601; PCT publication WO 02/39120; PCT publication WO 01/79849; PCT publication WO 99/39210). The antigen-binding molecules of such arrays may recognize at least a subset of proteins expressed by a cell or population of cells, illustrative examples of which include growth factor receptors, hormone receptors, neurotransmitter receptors, catecholamine receptors, amino acid derivative receptors, cytokine receptors, extracellular matrix receptors, antibodies, lectins, cytokines, serpins, proteases, kinases, phosphatases, ras-like GTPases, hydrolases, steroid hormone receptors, transcription factors, heat-shock transcription factors, DNA-binding proteins, zinc-finger proteins, leucine-zipper proteins, homeodomain proteins, intracellular signal transduction modulators and effectors, apoptosis-related factors, DNA synthesis factors, DNA repair factors, DNA recombination factors and cell-surface antigens.

Individual spatially distinct protein-capture agents are typically attached to a support surface, which is generally planar or contoured. Common physical supports include glass slides, silicon, microwells, nitrocellulose or PVDF membranes, and magnetic and other microbeads.

Particles in suspension can also be used as the basis of arrays, providing they are coded for identification; systems include color coding for microbeads (e.g., available from Luminex, Bio-Rad and Nanomics Biosystems) and semiconductor nanocrystals (e.g., QDots™, available from Quantum Dots), and barcoding for beads (UltraPlex™, available from Smartbeads) and multimetal microrods (Nanobarcodes™ particles, available from Surromed). Beads can also be assembled into planar arrays on semiconductor chips (e.g., available from LEAPS technology and BioArray Solutions). Where particles are used, individual protein-capture agents are typically attached to an individual particle to provide the spatial definition or separation of the array. The particles may then be assayed separately, but in parallel, in a compartmentalized way, for example in the wells of a microtiter plate or in separate test tubes.

In operation, a protein sample, which is optionally fragmented to form peptide fragments (see, e.g., U.S. Pat. App. Pub. 2002/0055186), is delivered to a protein-capture array under conditions suitable for protein or peptide binding, and the array is washed to remove unbound or non-specifically bound components of the sample from the array. Next, the presence or amount of protein or peptide bound to each feature of the array is detected using a suitable detection system. The amount of protein bound to a feature of the array may be determined relative to the amount of a second protein bound to a second feature of the array. In certain embodiments, the amount of the second protein in the sample is already known or known to be invariant.

In specific embodiments, the BaSIRS, VaSIRS, PaSIRS, InSIRS, BIP, VIP or PIP biomarker is a target polypeptide whose level is measured using at least one antigen-binding molecule that is immuno-interactive with the target polypeptide. In these embodiments, the measured level of the target polypeptide is normalized to the level of a reference polypeptide. Suitably, the antigen-binding molecule is immobilized on a solid or semi-solid support. In illustrative examples of this type, the antigen-binding molecule forms part of a spatial array of antigen-binding molecule. In some embodiments, the level of antigen-binding molecule that is bound to the target polypeptide is measured by immunoassay (e.g., using an ELISA).

All the essential reagents required for detecting and quantifying the BaSIRS, VaSIRS, PaSIRS, InSIRS, BIP, VIP or PIP biomarkers of the invention may be assembled together in a kit. In some embodiments, the kit comprises a reagent that permits quantification of at least one BaSIRS, VaSIRS, PaSIRS, InSIRS biomarker in combination with at least one BIP, VIP or PIP biomarker. In some embodiments the kit comprises: (i) a reagent that allows quantification (e.g., determining the level) of a first BaSIRS, VaSIRS, PaSIRS or InSIRS biomarker; and (ii) a reagent that allows quantification (e.g., determining the level) of a second BaSIRS, VaSIRS, PaSIRS or InSIRS biomarker, wherein the first and second biomarkers form a pair of derived biomarkers, as defined herein; and (iii) a reagent that allows quantification (e.g., determining the level or abundance) of a BIP, VIP or PIP biomarker. In some embodiments, the kit further comprises (iv) a reagent that allows quantification (e.g., determining the level or abundance) of a third BaSIRS, VaSIRS, PaSIRS or InSIRS biomarker; and (v) a reagent that allows quantification (e.g., determining the level or abundance) of a fourth BaSIRS, VaSIRS, PaSIRS or InSIRS biomarker, wherein the third and fourth biomarkers form a pair of derived biomarkers, as defined herein; and, (vi) a reagent that allows quantification (e.g., determining the level or abundance) of a second BIP, VIP or PIP biomarker. In some embodiments, the kit further comprises (vii) a reagent that allows quantification (e.g., determining the level or abundance) of a fifth BaSIRS, VaSIRS, PaSIRS or InSIRS biomarker; and (viii) a reagent that allows quantification (e.g., determining the level or abundance) of a sixth BaSIRS, VaSIRS, PaSIRS or InSIRS biomarker, wherein the fifth and sixth biomarkers form a pair of derived biomarkers, as defined herein; and, (ix) a reagent that allows quantification (e.g., determining the level or abundance) of a third BIP, VIP or PIP biomarker.

In the context of the present invention, “kit” is understood to mean a product containing the different reagents necessary for carrying out the methods of the invention packed so as to allow their transport and storage. Materials suitable for packing the components of the kit include crystal, plastic (polyethylene, polypropylene, polycarbonate and the like), bottles, vials, paper, envelopes and the like. Additionally, the kits of the invention can contain instructions for the simultaneous, sequential or separate use of the different components contained in the kit. The instructions can be in the form of printed material or in the form of an electronic support capable of storing instructions such that they can be read by a subject, such as electronic storage media (magnetic disks, tapes and the like), optical media (CD-ROM, DVD) and the like. Alternatively or in addition, the media can contain Internet addresses that provide the instructions.

Reagents that allow quantification of a BaSIRS, VaSIRS, PaSIRS, InSIRS, BIP, VIP or PIP biomarker include compounds or materials, or sets of compounds or materials, which allow quantification of the BaSIRS, VaSIRS, PaSIRS, InSIRS, BIP, VIP or PIP biomarkers. In specific embodiments, the compounds, materials or sets of compounds or materials permit (i) determining the expression level of a gene (e.g., BaSIRS, VaSIRS, PaSIRS or InSIRS biomarker gene), and (ii) determining the presence, absence, type, sequence of nucleic acid (e.g., BIP, VIP or PIP biomarker gene), including without limitation the extraction of RNA or DNA material, the determination of the level of a corresponding RNA, DNA etc., the determination of a particular nucleic acid sequence, primers for the synthesis of a corresponding cDNA and DNA, a thermostable DNA polymerase, primers for amplification of DNA, and/or probes capable of specifically hybridizing with the RNAs, corresponding cDNAs encoded by the genes, DNAs, TaqMan probes, etc.

The kits may also optionally include appropriate reagents for detection of labels, positive and negative controls, washing solutions, blotting membranes, microtiter plates, dilution buffers and the like. For example, a nucleic acid-based detection kit may include (i) a BaSIRS, VaSIRS, PaSIRS, InSIRS, BIP, VIP or PIP biomarker polynucleotide (which may be used as a positive control), (ii) a primer or probe that specifically hybridizes to a BaSIRS, VaSIRS, PaSIRS, InSIRS, BIP, VIP or PIP biomarker polynucleotide. Also included may be enzymes suitable for amplifying nucleic acids including various polymerases (reverse transcriptase, Taq, Sequenase™ DNA ligase etc. depending on the nucleic acid amplification technique employed), deoxynucleotides and buffers to provide the necessary reaction mixture for amplification. Such kits also generally will comprise, in suitable means, distinct containers for each individual reagent and enzyme as well as for each primer or probe. Alternatively, a protein-based detection kit may include (i) a BaSIRS, VaSIRS, PaSIRS, InSIRS, BIP, VIP or PIP biomarker polypeptide (which may be used as a positive control), (ii) an antibody that binds specifically to a BaSIRS, VaSIRS, PaSIRS, InSIRS, BIP, VIP or PIP biomarker polypeptide. The kit can also feature various devices (e.g., one or more) and reagents (e.g., one or more) for performing one of the assays described herein; and/or printed instructions for using the kit to quantify the expression of a BaSIRS, VaSIRS, PaSIRS, InSIRS biomarker gene in combination with the determination of the presence, absence, type, sequence of nucleic acid of a BIP, VIP or PIP biomarker gene.

The reagents described herein, which may be optionally associated with detectable labels, can be presented in the format of a microfluidics card, a chip or chamber, a Point-of-Care cartridge, a microarray or a kit adapted for use with the assays described in the examples or below, e.g., RT-PCR or Q PCR techniques described herein.

The reagents also have utility in compositions for detecting and quantifying the biomarkers of the invention. For example, a reverse transcriptase may be used to reverse transcribe RNA transcripts, including mRNA, in a nucleic acid sample, to produce reverse transcribed transcripts, including reverse transcribed mRNA (also referred to as “cDNA”). In specific embodiments, the reverse transcribed mRNA is whole cell reverse transcribed mRNA (also referred to herein as “whole cell cDNA”). The nucleic acid sample is suitably derived from components of the immune system, representative examples of which include components of the innate and adaptive immune systems as broadly discussed for example above. In specific embodiments, the reverse transcribed RNA is derived blood cells (e.g., peripheral blood cells). Suitably, the reverse transcribed RNA is derived leukocytes.

The reagents are suitably used to quantify the reverse transcribed transcripts. For example, oligonucleotide primers that hybridize to the reverse transcribed transcript can be used to amplify at least a portion of the reverse transcribed transcript via a suitable nucleic acid amplification technique, e.g., RT-PCR or qPCR techniques described herein. Alternatively, oligonucleotide probes may be used to hybridize to the reverse transcribed transcript for the quantification, using a nucleic acid hybridization analysis technique (e.g., microarray analysis), as described for example above. Thus, in some embodiments, a respective oligonucleotide primer or probe is hybridized to a complementary nucleic acid sequence of a reverse transcribed transcript in the compositions of the invention. The compositions typically comprise labeled reagents for detecting and/or quantifying the reverse transcribed transcripts. Representative reagents of this type include labeled oligonucleotide primers or probes that hybridize to RNA transcripts or reverse transcribed RNA, labeled RNA, labeled reverse transcribed RNA as well as labeled oligonucleotide linkers or tags (e.g., a labeled RNA or DNA linker or tag) for labeling (e.g., end labeling such as 3′ end labeling) RNA or reverse transcribed RNA. The primers, probes, RNA or reverse transcribed RNA (i.e., cDNA) (whether labeled or non-labeled) may be immobilized or free in solution. Representative reagents of this type include labeled oligonucleotide primers or probes that hybridize to reverse transcribed and transcripts as well as labeled reverse transcribed transcripts. The label can be any reporter molecule as known in the art, illustrative examples of which are described above and elsewhere herein.

The present invention also encompasses non-reverse transcribed RNA embodiments in which cDNA is not made and the RNA transcripts are directly the subject of the analysis. Thus, in other embodiments, reagents are suitably used to quantify RNA transcripts directly. For example, oligonucleotide probes can be used to hybridize to transcripts for quantification of immune system biomarkers of the invention, using a nucleic acid hybridization analysis technique (e.g., microarray analysis), as described for example above. Thus, in some embodiments, a respective oligonucleotide probe is hybridized to a complementary nucleic acid sequence of an immune system biomarker transcript in the compositions of the invention. In illustrative examples of this type, the compositions may comprise labeled reagents that hybridize to transcripts for detecting and/or quantifying the transcripts. Representative reagents of this type include labeled oligonucleotide probes that hybridize to transcripts as well as labeled transcripts. The primers or probes may be immobilized or free in solution.

### 3. Management, Treatment and Predictive Medicine Embodiments

The present invention also extends to the management of BaSIRS, VaSIRS, PaSIRS or InSIRS, or prevention of further progression of BaSIRS, VaSIRS, PaSIRS or InSIRS, or assessment of the efficacy of therapies in subjects following positive diagnosis for the presence of BaSIRS, VaSIRS, PaSIRS or InSIRS, in a subject. Once a subject is positively identified as having BaSIRS, VaSIRS, PaSIRS or InSIRS, the subject may be administered a therapeutic agent for treating the BaSIRS, VaSIRS, PaSIRS or InSIRS such as an anti-bacterial, anti-viral or anti-protozoal agent, illustrative examples of which include:

Anti-bacterial agents: Amikacin, Gentamicin, Kanamycin, Neomycin, Netilmicin, Tobramycin, Paromomycin, Streptomycin, Spectinomycin, Geldanamycin, Herbimycin, Rifaximin, Loracarbef, Ertapenem, Doripenem, Imipenem/Cilastatin, Meropenem, Cefadroxil, Cefazolin, Cefalotin or Cefalothin, Cefalexin, Cefaclor, Cefamandole, Cefoxitin, Cefprozil, Cefuroxime, Cefixime, Cefdinir, Cefditoren, Cefoperazone, Cefotaxime, Cefpodoxime, Ceftazidime, Ceftibuten, Ceftizoxime, Ceftriaxone, Cefepime, Ceftaroline fosamil, Ceftobiprole, Teicoplanin, Vancomycin, Telavancin, Dalbavancin, Oritavancin, Clindamycin, Lincomycin, Daptomycin, Azithromycin, Clarithromycin, Dirithromycin, Erythromycin, Roxithromycin, Troleandomycin, Telithromycin, Spiramycin, Aztreonam, Furazolidone, Nitrofurantoin, Linezolid, Posizolid, Radezolid, Torezolid, Amoxicillin, Ampicillin, Azlocillin, Carbenicillin, Cloxacillin, Dicloxacillin, Flucloxacillin, Mezlocillin, Methicillin, Nafcillin, Oxacillin, Penicillin G, Penicillin V, Piperacillin, Penicillin G, Temocillin, Ticarcillin, Amoxicillin/clavulanate, Ampicillin/sulbactam, Piperacillin/tazobactam, Ticarcillin/clavulanate, Bacitracin, Colistin, Polymyxin B, Ciprofloxacin, Enoxacin, Gatifloxacin, Gemifloxacin, Levofloxacin, Lomefloxacin, Moxifloxacin, Nalidixic acid, Norfloxacin, Ofloxacin, Trovafloxacin, Grepafloxacin, Sparfloxacin, Temafloxacin, Mafenide, Sulfacetamide, Sulfadiazine, Silver sulfadiazine, Sulfadimethoxine, Sulfamethizole, Sulfamethoxazole, Sulfanilimide, Sulfasalazine, Sulfisoxazole, Trimethoprim-Sulfamethoxazole, Sulfonamidochrysoidine, Demeclocycline, Doxycycline, Minocycline, Oxytetracycline, Tetracycline, Clofazimine, Dapsone, Capreomycin, Cycloserine, Ethambutol, Ethionamide, Isoniazid, Pyrazinamide, Rifampicin, Rifabutin, Rifapentine, Streptomycin, Arsphenamine, Chloramphenicol, Fosfomycin, Fusidic acid, Metronidazole, Mupirocin, Platensimycin, Quinupristin/Dalfopristin, Thiamphenicol, Tigecycline, Tinidazole, and Trimethoprim;

Anti-viral agents: asunaprevir, acyclovir, acyclovir, adefovir, amantadine, amprenavir, ampligen, arbidol, atazanavir, atripla, bacavir, boceprevir, cidofovir, combivir, complera, daclatasvir, darunavir, delavirdine, didanosine, docosanol, dolutegravir, edoxudine, efavirenz, emtricitabine, enfuvirtide, entecavir, famciclovir, fomivirsen, fosamprenavir, foscarnet, fosfonet, ganciclovir, ibacitabine, imunovir, idoxuridine, imiquimod, indinavir, inosine, interferon type III, interferon type II, interferon type I, lamivudine, lopinavir, loviride, maraviroc, moroxydine, methisazone, nelfinavir, nevirapine, nexavir, neuraminidase blocking agents, oseltamivir, peginterferon alfa-2a, penciclovir, peramivir, pleconaril, podofilox, podophyllin, podophyllotoxin, raltegravir, monoclonal antibody respigams, ribavirin, inhaled rhibovirons, rimantadine, ritonavir, pyrimidine, saquinavir, stavudine, stribild, tenofovir, tenofovir disoproxil, tenofovir alafenamide fumarate (TAF), tipranavir, trifluridine, trizivir, tromantadine, truvada, valaciclovir, valganciclovir, vicriviroc, vidarabine, viperin, viramidine, zalcitabine, zanamivir, zidovudine, or salts and combinations thereof; and

Anti-protozoal agents: Eflornithine, Furazolidone, Melarsoprol, Metronidazole, Ornidazole, Paromomycin sulfate, Pentamidine, Pyrimethamine, Tinidazole.

In a related aspect, the present invention contemplates the use of the indicator-determining methods, apparatus, compositions and kits disclosed herein in methods of treating, preventing or inhibiting the development or progression of BaSIRS, VaSIRS, PaSIRS or InSIRS in a subject. These methods (also referred to herein as “treatment methods”) generally comprise: exposing the subject to a treatment regimen for treating BaSIRS, VaSIRS, PaSIRS or InSIRS, or avoiding exposing the subject to a treatment regimen for treating a SIRS other than BaSIRS, VaSIRS, PaSIRS or InSIRS based on an indicator obtained from an indicator-determining method as disclosed herein.

Typically, the treatment regimen involves the administration of therapeutic agents effective amounts to achieve their intended purpose. The therapeutic agents are typically administered in the form a pharmaceutical composition that suitably includes a pharmaceutically acceptable carrier. The dose of active compounds administered to a subject should be sufficient to achieve a beneficial response in the subject over time such as a reduction in, or relief from, the symptoms of BaSIRS, VaSIRS, PaSIRS or InSIRS. The quantity of the of therapeutic agents to be administered may depend on the subject to be treated inclusive of the age, sex, weight and general health condition thereof. In this regard, precise amounts of the active agents(s) for administration will depend on the judgment of the practitioner. In determining the effective amount of the active agent(s) to be administered in the treatment or prevention of BaSIRS, VaSIRS, PaSIRS or InSIRS, the medical practitioner or veterinarian may evaluate severity of any symptom or clinical sign associated with the presence of BaSIRS, VaSIRS, PaSIRS or InSIRS or degree of BaSIRS, VaSIRS, PaSIRS or InSIRS including, inflammation, blood pressure anomaly, tachycardia, tachypnea fever, chills, vomiting, diarrhea, skin rash, headaches, confusion, muscle aches, seizures. In any event, those of skill in the art may readily determine suitable dosages of the therapeutic agents and suitable treatment regimens without undue experimentation.

The therapeutic agents may be administered in concert with adjunctive (palliative) therapies to increase oxygen supply to major organs, increase blood flow to major organs and/or to reduce the inflammatory response. Illustrative examples of such adjunctive therapies include non-steroidal-anti-inflammatory drugs (NSAIDs), intravenous saline and oxygen.

The present invention can be practiced in the field of predictive medicine for the purpose of diagnosis or monitoring the presence or development of BaSIRS, VaSIRS, PaSIRS or InSIRS in a subject, and/or monitoring response to therapy efficacy. The biomarker profiles and corresponding indicators of the present invention further enable determination of endpoints in pharmacotranslational studies. For example, clinical trials can take many months or even years to establish the pharmacological parameters for a medicament to be used in treating or preventing BaSIRS, VaSIRS, PaSIRS or InSIRS. However, these parameters may be associated with a biomarker profile and corresponding indicator of a health state (e.g., a healthy condition). Hence, the clinical trial can be expedited by selecting a treatment regimen (e.g., medicament and pharmaceutical parameters), which results in a biomarker profile associated with a desired health state (e.g., healthy condition). In these embodiments, the methods may comprise: (1) obtaining a biomarker profile of a sample taken from the subject after treatment of the subject with the treatment regimen, wherein the sample biomarker profile comprises (a) for each of a plurality of derived biomarkers as broadly defined above and elsewhere herein a plurality of host response specific derived biomarker values, and optionally (b) if the SIRS condition is an IpSIRS, a pathogen specific biomarker value as broadly defined above and elsewhere herein for a pathogen biomarker associated with the SIRS condition; and (2) comparing the sample biomarker profile to a reference biomarker profile that is correlated with a presence, absence or degree of the SIRS condition to thereby determine whether the treatment regimen is effective for changing the health status of the subject to the desired health state. Accordingly, this aspect of the present invention advantageously provides methods of monitoring the efficacy of a particular treatment regimen in a subject (for example, in the context of a clinical trial) already diagnosed with BaSIRS, VaSIRS, PaSIRS or InSIRS. These methods take advantage of derived biomarker values that correlate with treatment efficacy to determine, for example, whether derived biomarker values of a subject undergoing treatment partially or completely normalize during the course of or following therapy or otherwise shows changes associated with responsiveness to the therapy.

Accordingly, the invention also contemplates methods of correlating a biomarker profile with an effective treatment regimen for a SIRS condition selected from BaSIRS, VaSIRS, PaSIRS and InSIRS. In these embodiments, the methods may comprise: (1) determining a biomarker profile of a sample taken from a subject with the SIRS condition and for whom an effective treatment has been identified, wherein the biomarker profile comprises: (a) for each of a plurality of derived biomarkers as broadly defined above and elsewhere herein a plurality of host response specific derived biomarker values, and optionally (b) if the SIRS condition is an IpSIRS, a pathogen specific biomarker value as broadly defined above and elsewhere herein for a pathogen biomarker associated with the SIRS condition; and (2) correlating the biomarker profile so determined with an effective treatment regimen for the SIRS condition. In specific embodiments, an indicator or biomarker profile is correlated to a global probability or a particular outcome, using receiver operating characteristic (ROC) curves.

The invention further provides methods of determining whether a treatment regimen is effective for treating a subject with a SIRS condition selected from BaSIRS, VaSIRS, PaSIRS and InSIRS. In some embodiments, these methods comprise: (1) determining a post-treatment biomarker profile of a sample taken from the subject after treatment with a treatment regimen, wherein the biomarker profile comprises: (a) for each of a plurality of derived biomarkers as broadly defined above and elsewhere herein a plurality of host response specific derived biomarker values, and optionally (b) if the SIRS condition is an IpSIRS, a pathogen specific biomarker value as broadly defined above and elsewhere herein for a pathogen biomarker associated with the SIRS condition; and (2) determining a post-treatment indicator using the post-treatment biomarker profile, wherein the post-treatment indicator is at least partially indicative of the presence, absence or degree of the SIRS condition, wherein the post-treatment indicator indicates whether the treatment regimen is effective for treating the SIRS condition in the subject on the basis that post-treatment indicator indicates the presence of a healthy condition or the presence of the SIRS condition of a lower degree relative to the degree of the SIRS condition in the subject before treatment with the treatment regimen.

The invention can also be practiced to evaluate whether a subject is responding (i.e., a positive response) or not responding (i.e., a negative response) to a treatment regimen. This aspect of the invention provides methods of correlating a biomarker profile with a positive or negative response to a treatment regimen for treating a SIRS condition selected from BaSIRS, VaSIRS, PaSIRS and InSIRS. In some embodiments, these methods comprise: (1) determining a biomarker profile of a sample taken from a subject with the SIRS condition following commencement of the treatment regimen, wherein the reference biomarker profile comprises: (a) for each of a plurality of derived biomarkers as broadly defined above and elsewhere herein a plurality of host response specific derived biomarker values, and optionally (b) if the SIRS condition is an IpSIRS, a pathogen specific biomarker value as broadly defined above and elsewhere herein for a pathogen biomarker associated with the SIRS condition; and (2) correlating the sample biomarker profile with a positive or negative response to the treatment regimen

The invention also encompasses methods of determining a positive or negative response to a treatment regimen by a subject with a SIRS condition selected from BaSIRS, VaSIRS, PaSIRS and InSIRS. In some embodiments, these methods comprise: (1) correlating a reference biomarker profile with a positive or negative response to the treatment regimen, wherein the biomarker profile comprises: (a) for each of a plurality of derived biomarkers as broadly defined above and elsewhere herein a plurality of host response specific derived biomarker values, and optionally (b) if the SIRS condition is an IpSIRS, a pathogen specific biomarker value as broadly defined above and elsewhere herein for a pathogen biomarker associated with the SIRS condition; (2) detecting a biomarker profile of a sample taken from the subject, wherein the sample biomarker profile comprises (i) a plurality of host response specific derived biomarker values for each of the plurality of derived biomarkers in the reference biomarker profile, and optionally (ii) a pathogen specific biomarker value for the pathogen biomarker in the reference biomarker profile, wherein the sample biomarker profile indicates whether the subject is responding positively or negatively to the treatment regimen.

In related embodiments, the present invention further contemplates methods of determining a positive or negative response to a treatment regimen by a subject with a SIRS condition selected from BaSIRS, VaSIRS, PaSIRS and InSIRS. In some embodiments, these methods comprise: (1) correlating a reference biomarker profile with a positive or negative response to the treatment regimen, wherein the biomarker profile comprises: (a) for each of a plurality of derived biomarkers as broadly defined above and elsewhere herein a plurality of host response specific derived biomarker values, and optionally (b) a pathogen specific biomarker value as broadly defined above and elsewhere herein for a pathogen biomarker associated with the SIRS condition; (2) detecting a biomarker profile of a sample taken from the subject, wherein the sample biomarker profile comprises (i) a plurality of host response specific derived biomarker values for each of the plurality of derived biomarkers in the reference biomarker profile, and optionally (ii) if the SIRS condition is an IpSIRS, a pathogen specific biomarker value for the pathogen biomarker in the reference biomarker profile, wherein the sample biomarker profile indicates whether the subject is responding positively or negatively to the treatment regimen.

The invention also contemplates methods of determining a positive or negative response to a treatment regimen by a subject with a SIRS condition selected from BaSIRS, VaSIRS, PaSIRS and InSIRS. In certain embodiments, these methods comprise: (1) obtaining a biomarker profile of a sample taken from the subject following commencement of the treatment regimen, wherein the biomarker profile comprises: (a) for each of a plurality of derived biomarkers as broadly defined above and elsewhere herein a plurality of host response specific derived biomarker values, and optionally (b) if the SIRS condition is an IpSIRS, a pathogen specific biomarker value as broadly defined above and elsewhere herein for a pathogen biomarker associated with the SIRS condition, wherein the sample biomarker profile is correlated with a positive or negative response to the treatment regimen; and (2) and determining whether the subject is responding positively or negatively to the treatment regimen.

The above methods can be practiced to identify responders or non-responders relatively early in the treatment process, i.e., before clinical manifestations of efficacy. In this way, the treatment regimen can optionally be discontinued, a different treatment protocol can be implemented and/or supplemental therapy can be administered. Thus, in some embodiments, a sample BaSIRS, VaSIRS, PaSIRS, InSIRS in combination with BIP, VIP or PIP biomarker profile is obtained within about 2 hours, 4 hours, 6 hours, 12 hours, 1 day, 2 days, 3 days, 4 days, 5 days, 1 week, 2 weeks, 3 weeks, 4 weeks, 6 weeks, 8 weeks, 10 weeks, 12 weeks, 4 months, six months or longer of commencing therapy.

### 4. Device Embodiments

The present invention also contemplates embodiments in which the indicator-determining method of the invention is implemented using one or more processing devices. In representative embodiments of this type, the method that is implemented by the processing device(s) determines an indicator used in assessing a likelihood of a subject having a presence, absence or degree of BaSIRS or VaSIRS, wherein the method comprises: (1) determining a plurality of host response specific biomarker values including a plurality of BaSIRS biomarker values and a plurality of VaSIRS biomarker values, the plurality of BaSIRS biomarker values being indicative of values measured for a corresponding plurality of BaSIRS biomarkers in a sample taken from the subject, the plurality of VaSIRS biomarker values being indicative of values measured for a corresponding plurality of VaSIRS biomarkers in the sample; (2) determining a plurality of host response specific derived biomarker values including at least one BaSIRS derived biomarker value and at least one VaSIRS derived biomarker value, each derived BaSIRS biomarker value being determined using at least a subset of the plurality of BaSIRS biomarker values, and being indicative of a ratio of levels of a corresponding at least a subset of the plurality of BaSIRS biomarkers, and each derived VaSIRS biomarker value being determined using at least a subset of the plurality of VaSIRS biomarker values, and being indicative of a ratio of levels of a corresponding at least a subset of the plurality of VaSIRS biomarkers; (3) determining the indicator using the plurality of host response specific derived biomarker values, wherein the at least a subset of BaSIRS biomarkers forms a BaSIRS derived biomarker combination which is not a derived biomarker combination for VaSIRS, PaSIRS or InSIRS, and wherein the at least a subset of VaSIRS biomarkers forms a VaSIRS derived biomarker combination which is not a derived biomarker combination for BaSIRS, PaSIRS or InSIRS, wherein the BaSIRS derived biomarker combination is suitably selected from TABLE A and wherein the VaSIRS derived biomarker combination is suitably selected from TABLE B; (4) retrieving previously determined indicator references from a database, the indicator references being determined based on indicators determined from a reference population consisting of individuals diagnosed with BaSIRS or VaSIRS; (5) comparing the indicator to the indicator references to thereby determine a probability indicative of the subject having or not having BaSIRS or VaSIRS; and (6) generating a representation of the probability, the representation being displayed to a user to allow the user to assess the likelihood of a biological subject having BaSIRS or VaSIRS.

In some embodiments, the indicator-determining method that is implemented by the processing device(s) determines an indicator used in assessing a likelihood of a subject having a presence, absence or degree of BaSIRS, VaSIRS or PaSIRS, wherein the method comprises: (1) determining a plurality of host response specific biomarker values including a plurality of BaSIRS biomarker values, a plurality of VaSIRS biomarker values and a plurality of PaSIRS biomarker values, the plurality of BaSIRS biomarker values being indicative of values measured for a corresponding plurality of BaSIRS biomarkers in a sample taken from the subject, the plurality of VaSIRS biomarker values being indicative of values measured for a corresponding plurality of VaSIRS biomarkers in the sample, and the plurality of PaSIRS biomarker values being indicative of values measured for a corresponding plurality of PaSIRS biomarkers in the sample; (2) determining a plurality of host response specific derived biomarker values including at least one BaSIRS derived biomarker value, at least one VaSIRS derived biomarker value and at least one PaSIRS derived biomarker value, each derived BaSIRS biomarker value being determined using at least a subset of the plurality of BaSIRS biomarker values, and being indicative of a ratio of levels of a corresponding at least a subset of the plurality of BaSIRS biomarkers, each derived VaSIRS biomarker value being determined using at least a subset of the plurality of VaSIRS biomarker values, and being indicative of a ratio of levels of a corresponding at least a subset of the plurality of VaSIRS biomarkers, and each derived PaSIRS biomarker value being determined using at least a subset of the plurality of PaSIRS biomarker values, and being indicative of a ratio of levels of a corresponding at least a subset of the plurality of PaSIRS biomarkers; (3) determining the indicator using the plurality of host response specific derived biomarker values, wherein the at least a subset of BaSIRS biomarkers forms a BaSIRS derived biomarker combination which is not a derived biomarker combination for VaSIRS, PaSIRS or InSIRS, wherein the at least a subset of VaSIRS biomarkers forms a VaSIRS derived biomarker combination which is not a derived biomarker combination for BaSIRS, PaSIRS or InSIRS, and wherein the at least a subset of PaSIRS biomarkers forms a PaSIRS derived biomarker combination which is not a derived biomarker combination for BaSIRS, VaSIRS or InSIRS, wherein the BaSIRS derived biomarker combination is suitably selected from TABLE A, wherein the VaSIRS derived biomarker combination is suitably selected from TABLE B, and wherein the PaSIRS derived biomarker combination is suitably selected from TABLE C; (4) retrieving previously determined indicator references from a database, the indicator references being determined based on indicators determined from a reference population consisting of individuals diagnosed with BaSIRS, VaSIRS or PaSIRS; (5) comparing the indicator to the indicator references to thereby determine a probability indicative of the subject having or not having BaSIRS, VaSIRS or PaSIRS; and (6) generating a representation of the probability, the representation being displayed to a user to allow the user to assess the likelihood of a biological subject having BaSIRS, VaSIRS or PaSIRS.

In other embodiments, the method that is implemented by the processing device(s) determines an indicator used in assessing a likelihood of a subject having a presence, absence or degree of BaSIRS, VaSIRS or InSIRS, wherein the method comprises: (1) determining a plurality of host response specific biomarker values including a plurality of BaSIRS biomarker values, a plurality of VaSIRS biomarker values and a plurality of InSIRS biomarker values, the plurality of BaSIRS biomarker values being indicative of values measured for a corresponding plurality of BaSIRS biomarkers in a sample taken from the subject, the plurality of VaSIRS biomarker values being indicative of values measured for a corresponding plurality of VaSIRS biomarkers in the sample, and the plurality of InSIRS biomarker values being indicative of values measured for a corresponding plurality of InSIRS biomarkers in the sample; (2) determining a plurality of host response specific derived biomarker values including at least one BaSIRS derived biomarker value, at least one VaSIRS derived biomarker value and at least one InSIRS derived biomarker value, each derived BaSIRS biomarker value being determined using at least a subset of the plurality of BaSIRS biomarker values, and being indicative of a ratio of levels of a corresponding at least a subset of the plurality of BaSIRS biomarkers, each derived VaSIRS biomarker value being determined using at least a subset of the plurality of VaSIRS biomarker values, and being indicative of a ratio of levels of a corresponding at least a subset of the plurality of VaSIRS biomarkers, and each derived InSIRS biomarker value being determined using at least a subset of the plurality of InSIRS biomarker values, and being indicative of a ratio of levels of a corresponding at least a subset of the plurality of InSIRS biomarkers; (3) determining the indicator using the plurality of host response specific derived biomarker values, wherein the at least a subset of BaSIRS biomarkers forms a BaSIRS derived biomarker combination which is not a derived biomarker combination for VaSIRS, PaSIRS or InSIRS, wherein the at least a subset of VaSIRS biomarkers forms a VaSIRS derived biomarker combination which is not a derived biomarker combination for BaSIRS, PaSIRS or InSIRS, and wherein the at least a subset of InSIRS biomarkers forms a InSIRS derived biomarker combination which is not a derived biomarker combination for BaSIRS, VaSIRS or PaSIRS, wherein the BaSIRS derived biomarker combination is suitably selected from TABLE A, wherein the VaSIRS derived biomarker combination is suitably selected from TABLE B, and wherein the InSIRS derived biomarker combination is suitably selected from TABLE D; (4) retrieving previously determined indicator references from a database, the indicator references being determined based on indicators determined from a reference population consisting of individuals diagnosed with BaSIRS, VaSIRS or InSIRS; (5) comparing the indicator to the indicator references to thereby determine a probability indicative of the subject having or not having BaSIRS, VaSIRS or InSIRS; and (6) generating a representation of the probability, the representation being displayed to a user to allow the user to assess the likelihood of a biological subject having BaSIRS, VaSIRS or InSIRS.

In still other embodiments, the method that is implemented by the processing device(s) determines an indicator used in assessing a likelihood of a subject having a presence, absence or degree of BaSIRS, VaSIRS, PaSIRS or InSIRS, wherein the method comprises: (1) determining a plurality of host response specific biomarker values including a plurality of BaSIRS biomarker values, a plurality of VaSIRS biomarker values, a plurality of PaSIRS biomarker values and a plurality of InSIRS biomarker values, the plurality of BaSIRS biomarker values being indicative of values measured for a corresponding plurality of BaSIRS biomarkers in a sample taken from the subject, the plurality of VaSIRS biomarker values being indicative of values measured for a corresponding plurality of VaSIRS biomarkers in the sample, the plurality of PaSIRS biomarker values being indicative of values measured for a corresponding plurality of PaSIRS biomarkers in the sample, and the plurality of InSIRS biomarker values being indicative of values measured for a corresponding plurality of InSIRS biomarkers in the sample; (2) determining a plurality of host response specific derived biomarker values including at least one BaSIRS derived biomarker value, at least one VaSIRS derived biomarker value, at least one PaSIRS derived biomarker value and at least one InSIRS derived biomarker value, each derived BaSIRS biomarker value being determined using at least a subset of the plurality of BaSIRS biomarker values, and being indicative of a ratio of levels of a corresponding at least a subset of the plurality of BaSIRS biomarkers, each derived VaSIRS biomarker value being determined using at least a subset of the plurality of VaSIRS biomarker values, and being indicative of a ratio of levels of a corresponding at least a subset of the plurality of VaSIRS biomarkers, each derived PaSIRS biomarker value being determined using at least a subset of the plurality of PaSIRS biomarker values, and being indicative of a ratio of levels of a corresponding at least a subset of the plurality of PaSIRS biomarkers, and each derived InSIRS biomarker value being determined using at least a subset of the plurality of InSIRS biomarker values, and being indicative of a ratio of levels of a corresponding at least a subset of the plurality of InSIRS biomarkers; (3) determining the indicator using the plurality of host response specific derived biomarker values, wherein the at least a subset of BaSIRS biomarkers forms a BaSIRS derived biomarker combination which is not a derived biomarker combination for VaSIRS, PaSIRS or InSIRS, wherein the at least a subset of VaSIRS biomarkers forms a VaSIRS derived biomarker combination which is not a derived biomarker combination for BaSIRS, PaSIRS or InSIRS, wherein the at least a subset of PaSIRS biomarkers forms a PaSIRS derived biomarker combination which is not a derived biomarker combination for BaSIRS, VaSIRS or InSIRS, and wherein the at least a subset of InSIRS biomarkers forms a InSIRS derived biomarker combination which is not a derived biomarker combination for BaSIRS, VaSIRS or PaSIRS, wherein the BaSIRS derived biomarker combination is suitably selected from TABLE A, wherein the VaSIRS derived biomarker combination is suitably selected from TABLE B, wherein the PaSIRS derived biomarker combination is suitably selected from TABLE C, and wherein the InSIRS derived biomarker combination is suitably selected from TABLE D; (4) retrieving previously determined indicator references from a database, the indicator references being determined based on indicators determined from a reference population consisting of individuals diagnosed with BaSIRS, VaSIRS, PaSIRS or InSIRS; (5) comparing the indicator to the indicator references to thereby determine a probability indicative of the subject having or not having BaSIRS, VaSIRS, PaSIRS or InSIRS; and (6) generating a representation of the probability, the representation being displayed to a user to allow the user to assess the likelihood of a biological subject having BaSIRS, VaSIRS, PaSIRS or InSIRS.

In any of the above embodiments, the method that is implemented by the processing device(s) determines an indicator used in assessing a likelihood of a subject having a presence, absence or degree of BaSIRS or VaSIRS, or optionally one of PaSIRS or InSIRS, wherein the methods further comprise: (a) determining a plurality of pathogen specific biomarker values including at least one bacterial biomarker value and at least one viral biomarker value, and optionally at least one protozoal biomarker value, the least one bacterial biomarker value being indicative of a value measured for a corresponding bacterial biomarker in the sample, the least one viral biomarker value being indicative of a value measured for a corresponding viral biomarker in the sample, and the least one protozoal biomarker value being indicative of a value measured for a corresponding protozoal biomarker in the sample; (b) determining the indicator using the host response specific derived biomarker values in combination with the pathogen specific biomarker values; (c) retrieving previously determined indicator references from a database, the indicator references being determined based on indicators determined from a reference population consisting of individuals diagnosed with BaSIRS, VaSIRS or optionally one of PaSIRS or InSIRS; (d) comparing the indicator to the indicator references to thereby determine a probability indicative of the subject having or not having BaSIRS, VaSIRS, PaSIRS or InSIRS; and (6) generating a representation of the probability, the representation being displayed to a user to allow the user to assess the likelihood of the subject having BaSIRS or VaSIRS, or optionally one of PaSIRS or InSIRS.

Similarly apparatus can be provided for determining the likelihood of a subject having BaSIRS or VaSIRS, or optionally one of PaSIRS or InSIRS, the apparatus including: (A) a sampling device that obtains a sample taken from a subject, the sample including a plurality of host response specific biomarkers, and optionally at least one pathogen specific biomarker selected from BIP and VIP biomarkers, and optionally PIP biomarkers, wherein the host response specific biomarkers include a plurality of BaSIRS biomarkers, a plurality of VaSIRS biomarkers, and optionally one or both of a plurality of PaSIRS biomarkers and a plurality of InSIRS biomarkers; (B) a measuring device that quantifies for each of the host response specific biomarkers within the sample a corresponding host response specific biomarker value, and optionally that quantifies for each of the pathogen specific biomarkers within the sample a corresponding pathogen specific biomarker value; (C) at least one processing device that: (i) receives the host response specific biomarker values, and optionally receives the pathogen specific biomarker values from the measuring device; (ii) determines for at least a subset of the plurality of biomarker values of a specific SIRS type, a host response specific derived biomarker value indicative of a ratio of levels of a corresponding at least a subset of the plurality of host response specific biomarkers; (iii) determines an indicator that is at least partially indicative of the presence, absence or degree of BaSIRS or VaSIRS, or optionally one of PaSIRS or InSIRS using the host response specific derived biomarker values in combination with the pathogen specific biomarker values; (iv) compares the indicator to at least one indicator reference; (v) determines a likelihood of the subject having or not having a BaSIRS, VaSIRS, or optionally one of PaSIRS or InSIRS using the results of the comparison; and (v) generates a representation of the indicator and the likelihood for display to a user.

In order that the invention may be readily understood and put into practical effect, particular preferred embodiments will now be described by way of the following non-limiting examples.

### EXAMPLES

### Example 1

### General Approach—BaSIRS, VaSIRS, PaSIRS and InSIRS Host Response Specific Biomarker Derivation (Derived Biomarkers)

An illustrative process for the identification of BaSIRS, VaSIRS, PaSIRS and InSIRS host response biomarkers for use in diagnostic algorithms will now be described.

Gene expression data (derived from clinical trials performed by the inventors and/or from Gene Expression Omnibus) were analyzed using a variety of statistical approaches to identify derived biomarkers (ratios) and largely follows the method described in WO 2015/117204. Individual and derived markers were graded based on performance (Area Under Curve). Datasets derived from GEO (which are all MIAME-compliant) were used with the following restrictions; peripheral blood samples were used, appropriate controls were used, an appropriate number of samples were used to provide significance following False-Discovery Rate (FDR) adjustment, all data passed standard quality control metrics, principle component analysis did not reveal any artifacts or potential biases. The datasets were allocated into two groups (or combined samples from all datasets split evenly into two groups)—“discovery” and “validation”. The datasets in the “discovery” groups were deliberately chosen to enable the identification of specific BaSIRS, VaSIRS, PaSIRS and InSIRS biomarker profiles that could be used generically for a variety of known bacterial pathogens that cause BaSIRS, all Baltimore virus classification groups and across different mammalian species, a variety of protozoans with high morbidity that cause systemic inflammation and a variety of different non-infectious SIRS conditions. The studies therefore included; (a) for BaSIRS; Gram positive and Gram negative bacteria, a variety of different affected body systems, across a range of severity (b) for VaSIRS; DNA and RNA viruses, multiple mammalian species (human, macaque, chimpanzee, pig, mice, rat), high likelihood of generating a systemic inflammatory response (c) for PaSIRS; a variety of malarial (Plasmodium) species, a variety of protozoal species including Plasmodium, Leishmania and Toxoplasma (d) for InSIRS; a variety of non-infectious causes of systemic inflammation (e.g., trauma, asthma, allergy, cancer). For all studies the following parameters were also considered to be important: experimentally-infected subjects where a control sample was taken prior to inoculation, samples taken over time, in particular early-stage samples with a low likelihood of secondary complications from other infections (e.g., viral etiology with a secondary bacterial infection or a protozoan infection with a secondary bacterial infection).

Prior to analysis each dataset was filtered to include only the top genes (usually between 3000 and 6000 (of 35,000) depending upon data quality, level of expression and commonality across the datasets) as measured by the mean gene expression level across all samples in the dataset. This ensured that only those genes with relatively strong expression were analyzed and that a limited number of candidates were taken forward to the next compute-time intensive step. Receiver Operating Characteristic (ROC) curves and the area under theses curves (also referred to herein as Area Under Curve (AUC)) were then calculated across all derived biomarkers using the difference in the log 2 of the expression values for each derived biomarker. This resulted in approximately 36,000,000 (6000×5999) derived biomarkers per dataset. An AUC>0.5 was defined as a derived biomarker value being higher in cases than controls, i.e. where the numerator is potentially up-regulated in cases and/or the denominator is potentially down-regulated in cases. Generally, a ‘numerator’ biomarker of an individual biomarker pair disclosed herein is up-regulated or expressed at a higher level relative to a control (e.g., a healthy control) and a ‘denominator’ biomarker of the biomarker pair is unchanged or expressed at about the same level, or is down-regulated or expressed at a lower level, relative to a control (e.g., a healthy control). “Discovery” datasets were then combined by taking the mean AUC for each derived biomarker. Resulting derived biomarkers were then filtered by keeping only those with a mean AUC greater than a pre-determined threshold across all relevant datasets relevant to each of BaSIRS, VaSIRS, PaSIRS and InSIRS. The pool of remaining derived biomarkers after this step was a small percentage of the original number but still contained a large number of derived biomarkers with many that were common to each of the conditions of BaSIRS, VaSIRS, PaSIRS and InSIRS.

To ensure that the derived biomarkers were specific to either bacterial, viral, protozoan or non-infectious systemic inflammation a number of additional datasets (listed in TABLES 13, 18, 22 and 23) were used to identify derived biomarkers of generalized, non-infectious and infectious inflammation. Appropriate datasets from this list were used to provide specificity—by example, for identification of specific VaSIRS derived biomarkers datasets for systemic inflammation other than VaSIRS were used, and for identification of specific BaSIRS derived biomarkers datasets for systemic inflammation other than BaSIRS were used. These datasets were subjected to the same restrictions as the “discovery” and “validation” datasets including; peripheral blood samples were used, appropriate controls were used, an appropriate number of samples were used to provide significance following False-Discovery Rate (FDR) adjustment, all data passed standard quality control metrics, principle component analysis did not reveal any artifacts or potential biases. Derived biomarkers that had strong performance, based on an AUC threshold in more than a set number of these individual datasets, were removed (“subtracted”) from the list of identified BaSIRS, VaSIRS, PaSIRS or InSIRS derived biomarkers to ensure specificity Each unique pool of biomarkers, one for each of BaSIRS, VaSIRS, PaSIRS and InSIRS, was then taken forward to the next steps (validation and greedy search). Without this “subtraction” step derived biomarkers common to the SIRS conditions would be taken forward, which would result in different outcomes with respect to AUC performance of derived biomarkers and the final selection of the best combination of derived biomarkers (see Example 2).

A further filtering step was then applied. Only derived biomarkers with an AUC greater than a set threshold in a set number of the discovery and validation datasets for each condition (BaSIRS, VaSIRS, PaSIRS, InSIRS) were retained. Generally, a cut-off of around AUC of 0.75 or higher was chosen for the following reasons: 1). simple diagnostic heuristics for the diagnosis of influenza have an AUC between 0.7 and 0.79 (Ebell, M. H., & Afonso, A. (2011). A Systematic Review of Clinical Decision Rules for the Diagnosis of Influenza. The Annals of Family Medicine, 9(1), 69-77); 2). clinicians can predict patients that are ultimately blood culture positive from those with suspected infection with an AUC of 0.77 (Fischer, J. E., Harbarth, S., Agthe, A. G., Benn, A., Ringer, S. A., Goldmann, D. A., & Fanconi, S. (2004). Quantifying uncertainty: physicians' estimates of infection in critically ill neonates and children. Clinical Infectious Diseases: an Official Publication of the Infectious Diseases Society of America, 38(10), 1383-1390); 3). The use of polymerase chain reaction-based tests, compared to conventional tests, for respiratory pathogens in patients with suspected lower respiratory tract infections (LRTI) increased the diagnostic yield from 21% to 43% of cases (that is, molecular-based pathogen tests in this study only detected a pathogen in 43% of suspected LRTI) (Oosterheert, J. J., van Loon, A. M., Schuurman, R., Hoepelman, A. I. M., Hak, E., Thijsen, S., et al. (2005). Impact of rapid detection of viral and atypical bacterial pathogens by real-time polymerase chain reaction for patients with lower respiratory tract infection. Clinical Infectious Diseases, 41(10), 1438-1444); 4). the sensitivity of point-of-care tests for influenza is about 70% (Foo, H., & Dwyer, D. E. (2009). Rapid tests for the diagnosis of influenza. Australian Prescriber 32:64-67); 5). The performance of clinical algorithms and lack of trust in diagnostic tests for diagnosing malaria in febrile children in high incidence areas does not result or warrant the withholding anti-malarial drugs (Chandramohan, D., Jaffar, S., & Greenwood, B. (2002). Use of clinical algorithms for diagnosing malaria. Tropical Medicine & International Health: TM & IH, 7(1), 45-52; Bisoffi, Z., Sirima, B. S., Angheben, A., Lodesani, C., Gobbi, F., Tinto, H., & Van den Ende, J. (2009). Rapid malaria diagnostic tests vs. clinical management of malaria in rural Burkina Faso: safety and effect on clinical decisions. A randomized trial. Tropical Medicine & International Health: TM & IH, 14(5), 491-498; Amexo, M., Tolhurst, R., Barnish, G., & Bates, I. (2004). Malaria misdiagnosis: effects on the poor and vulnerable. The Lancet, 364(9448), 1896-1898). Thus, current existing diagnostic procedures and tests for bacterial, viral or protozoan infections do not have either good diagnostic performance or clinician trust, and in many instances no pathogen or antibody response is detected in samples taken at the time a patient presents with clinical signs. BaSIRS, VaSIRS, PaSIRS or InSIRS signatures with an AUC of at least 0.75 will therefore likely have greater clinical utility than most existing bacterial, viral or protozoal diagnostic assays, and at the critical time when the patient presents with clinical signs. Following this filtering step, usually a limited number of derived biomarkers remained, which were considered to be specific to the condition under investigation.

### Example 2

### BaSIRS, VaSIRS, PaSIRS and InSIRS Host Response Biomarker Derivation (General Approach—Combination of Derived Biomarkers)

Next, a search for the best combination and number of derived biomarkers for each of BaSIRS, VaSIRS, PaSIRS and InSIRS in each of the derived biomarker pools was performed with the aim of finding a minimal set of derived biomarkers with optimal commercial utility. Optimal commercial utility in this instance means consideration of the following non-limiting factors; diagnostic performance, clinical utility, diagnostic noise (introduced by using too many derived biomarkers), transferability to available molecular chemistries (e.g., PCR, microarray, DNA sequencing), transferability to available point-of-care platforms (e.g., Biocartis Idylla, Cepheid GeneXpert, Becton Dickinson BD Max, Curetis Unyvero, Oxford Nanopore Technologies MinION), cost of assay manufacture (the more reagents and biomarkers the larger the cost), ability to multiplex biomarkers, availability of suitable reporter dyes, complexity of results interpretation.

To be able to determine the best combination of derived markers all study datasets for each of BaSIRS, VaSIRS, PaSIRS or InSIRS needed to be combined. As such, each dataset was normalized individually using mean centering to zero and variance set to one. The mean of a biomarker in a dataset was calculated in three steps: (a) calculation of the mean of the cases, (b) calculation of the mean of the controls, and (c) calculation of the mean of the preceding two values. Once the mean for each biomarker had been calculated, the expression value for that biomarker in each sample was adjusted by subtracting the mean value. The values were further adjusted by dividing by the variance. This was performed for all biomarker expression values for every sample in every dataset. All of the datasets for each condition category were then combined into four separate (bacterial, viral, protozoal and InSIRS) expression matrices.

Following normalization, a search (greedy) for the best performing pair of derived biomarkers was performed (by AUC in the normalized dataset) using the corresponding specific derived biomarker pool for each of the bacterial, viral, protozoal and InSIRS expression matrices. This was accomplished by first identifying the best performing derived biomarker. Each of the other remaining derived biomarkers was then added and, as long as neither biomarker in the newly added derived biomarker was already part of the first derived biomarker, the AUC was calculated. This process continued and an AUC plot was generated based on sequential adding of derived biomarkers.

### Example 3

### Host Response Specific Biomarkers are Grouped Based on their Correlation to BaSIRS (OPLAH, ZHX2, TSPO, HCLS1), VaSIRS (ISG15, IL16, OASL and ADGRE5), PaSIRS (TTC17, G6PD, HERC6, LAP3, NUP160 and TPP1) and InSIRS (ARL6IP5, ENTPD1, HEATR1 and TNFSF8) Biomarkers, and Based on Greedy Search Results

The individual host response specific biomarkers in the signature for BaSIRS are: TSPO, HCLS1, OPLAH and ZHX2. The individual host response specific biomarkers in the signature for VaSIRS are: ISG15, IL16, OASL and ADGRE5. The individual host response specific biomarkers in the signature for PaSIRS are: TTC17, G6PD, HERC6, LAP3, NUP160 and TPP1. The individual host response specific biomarkers in the signature for InSIRS are: ARL6IP5, ENTPD1, HEATR1 and TNFSF8. There were 94, 413, 130 and 151 unique biomarkers in the lists of 102, 473, 523 and 164 host response specific derived biomarkers with an AUC over a set threshold for BaSIRS, VaSIRS, PaSIRS and InSIRS, respectively. For each unique biomarker, a correlation coefficient was calculated.

Two pairs of derived biomarkers (OPLAH/ZHX2; TSPO/HCLS1) were discovered that provided the highest AUC across all of the bacterial datasets studied after non-bacterial derived biomarkers had been subtracted. Biomarkers as ratios that provided an AUC above a set threshold were then allocated to one of four Groups, as individual biomarkers, based on their correlation to either OPLAH (Group A BaSIRS biomarkers), ZHX2 (Group B BaSIRS biomarkers), TSPO (Group C BaSIRS biomarkers) or HCSL1 (Group D BaSIRS biomarkers), as presented in TABLE 24.

Two pairs of derived biomarkers (IL16/ISG15; ADGRE5/OASL) were discovered that provided the highest AUC across all of the viral datasets studied after non-viral derived biomarkers had been subtracted. Biomarkers as ratios that provided an AUC above a set threshold were then allocated to one of four Groups, as individual biomarkers, based on their correlation to either ISG15 (Group A VaSIRS biomarkers), IL16 (Group B VaSIRS biomarkers), OASL (Group C VaSIRS biomarkers) or ADGRE5 (Group D VaSIRS biomarkers), as presented in TABLE 26.

Three pairs of derived biomarkers (TTC17/G6PD; HERC6/LAP3; NUP160/TPP1) were discovered that provided the highest AUC across all of the protozoan datasets studied after non-protozoan derived biomarkers had been subtracted. Biomarkers as ratios that provided an AUC above a set threshold were then allocated to one of six Groups, as individual biomarkers, based on their correlation to either TTC17 (Group A PaSIRS biomarkers), G6PD (Group B PaSIRS biomarkers), HERC6 (Group C PaSIRS biomarkers), LAP3 (Group D PaSIRS biomarkers), NUP160 (Group E PaSIRS biomarkers) or TPP1 (Group F PaSIRS biomarkers), as presented in TABLE 27.

Two pairs of derived biomarkers (ARL6IP5/ENTPD1; HEATR1/TNFSF8) were discovered that provided the highest AUC across all of the InSIRS datasets studied after infectious SIRS (bacterial, viral, protozoal) derived biomarkers had been subtracted. Biomarkers as ratios that provided an AUC above a set threshold were then allocated to one of four Groups, as individual biomarkers, based on their correlation to either ARL6IP5 (Group A InSIRS biomarkers), ENTPD1 (Group B InSIRS biomarkers), HEATR1 (Group C InSIRS biomarkers) or TNFSF8 (Group D InSIRS biomarkers), as presented in TABLE 28.

Following greedy searches, the best host response derived biomarkers, including any combination of such biomarkers, for BaSIRS, VaSIRS, PaSIRS and InSIRS are:
BaSIRS—TSPO:HCLS1, OPLAH:ZHX2, TSPO:RNASE6, GAS7:CAMK1D, STGAL2:PRKD2, PCOLE2:NMUR1, CR1:HAL
VaSIRS—ISG15:1L16, OASL:ADGRE5, TAP1:TGFBR2, IFIH1:CRLF3, IFI44:IL4R, EIFAK2:SYPL1, OAS2:LEF1, STAT1/PCBP2
PaSIRS—TTC17:G6PD, HERC6:LAP3, NUP160:TPP1, RPL15:GP1, ARID1A:CSTB,
AHCTF1:WARS, FBXO11:TANK, ADSL:ENO1, RPL9:TNIP1, ASXL2:IRF1.
InSIRS—ENTPD1:ARL6IP5, TNFSF8:HEATR1, ADAM19:POLR2A, SYNE2:VPS13C,
TNFSF8: NIP7, CDA: EFHD2, ADAM19:MLLT10, CDA: PTGS1, ADAM19:EXOC7, TNFSF8:TRIP11.

### Example 4

### BaSIRS Host Response Biomarker Derivation

A step-wise procedure was undertaken to identify biomarkers useful in determining a host systemic immune response to bacterial infection, which largely employs the same steps that were used to identify host systemic immune response biomarkers of viral infection, as described in Australian provisional patent application 2015903986.

In brief, bacterial derived biomarkers were discovered that are capable of determining a specific mammalian systemic host response to bacteria. This was achieved using a step-wise approach of derived biomarker discovery, subtraction and validation. Data pre-processing included; log 2 transformation (if gene expression data was from arrays), choice of the most intense probe to represent a gene, and choice of those ˜40% of genes with the largest variance within our own in-house datasets, which equalled approximately 3700 genes (which were then applied to publicly available datasets).

Discovery of a large pool of derived biomarkers was performed using carefully selected samples from in-house datasets (“Fever”, “MARS” and “GAPPSS”, n=6) and Gene Expression Omnibus (GSE) datasets (n=7)). Samples were pre-selected and categorized into InSIRS or BaSIRS using other known host response signatures and then split into two groups used for either “discovery” (n=984) or “validation” (n=1045) (see TABLES 11 and 12 for details on the datasets and samples in each group).

Derived biomarkers were computed for every combination in both the Discovery and Validation datasets, resulting in a total of 13,671,506 binary combinations. A total of 255 derived biomarkers had an AUC>0.8 across all discovery datasets and 102 that had an AUC>0.85 across the validation datasets (see TABLE 15). These same 102 derived biomarkers were then tested on other datasets containing samples derived from subjects with systemic inflammation not related to BaSIRS (see TABLE 13 for a list of these datasets). Other non-BaSIRS systemic conditions in these datasets included; viral infection, asthma, coronary artery disease, stress, sarcoidosis and cancer. The mean AUC range for the 102 derived biomarkers across these datasets was between 0.28 and 0.53 indicating specificity of the derived biomarkers for BaSIRS.

Datasets were then merged so that a greedy search could be performed with the aim of finding the best combination of derived biomarkers for separating InSIRS and BaSIRS subjects. Merging of datasets was achieved in the following manner. Each dataset was normalized by mean centering to zero and forcing gene variance to one as follows: The mean of a gene in a dataset was calculated in three steps: (a) calculation of the mean of the cases, (b) calculation of the mean of the controls, and (c) calculation of the mean of those two values. Once the mean was calculated, the expression values for that gene in each sample were adjusted by subtracting the mean value. An expression matrix was then standardized to unit variance by dividing by the genes variance. All datasets were then combined into a single “expression” matrix after normalizing each dataset individually. The matrix had dimensions of 102 biomarkers and 984 samples.

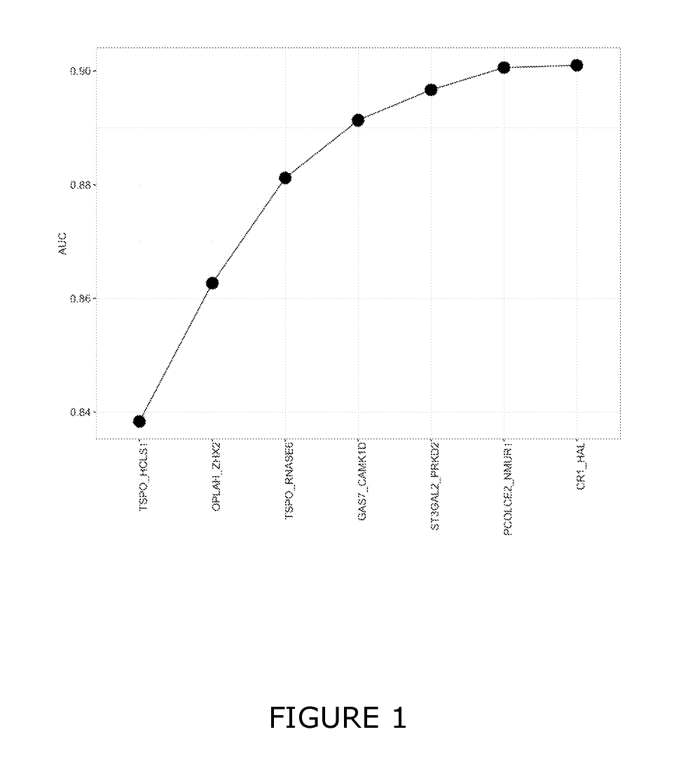

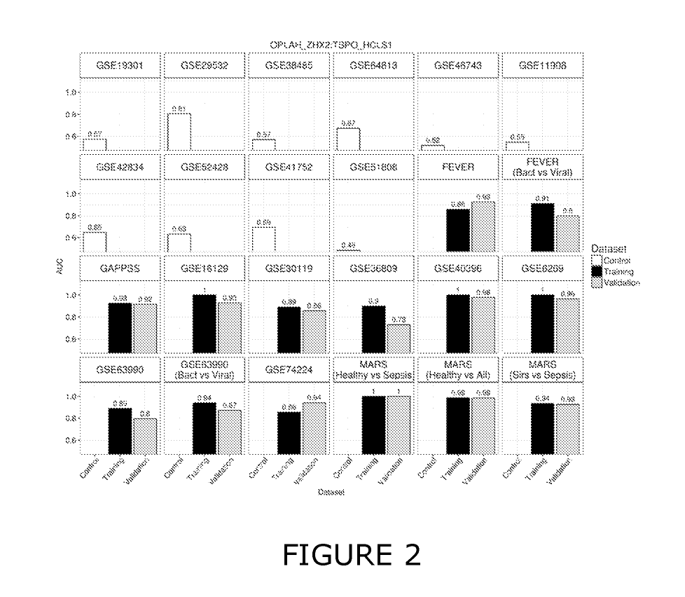

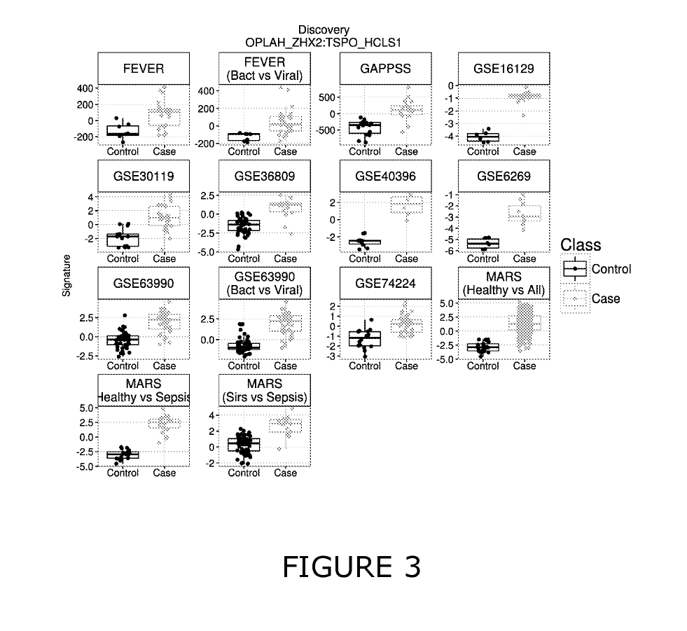

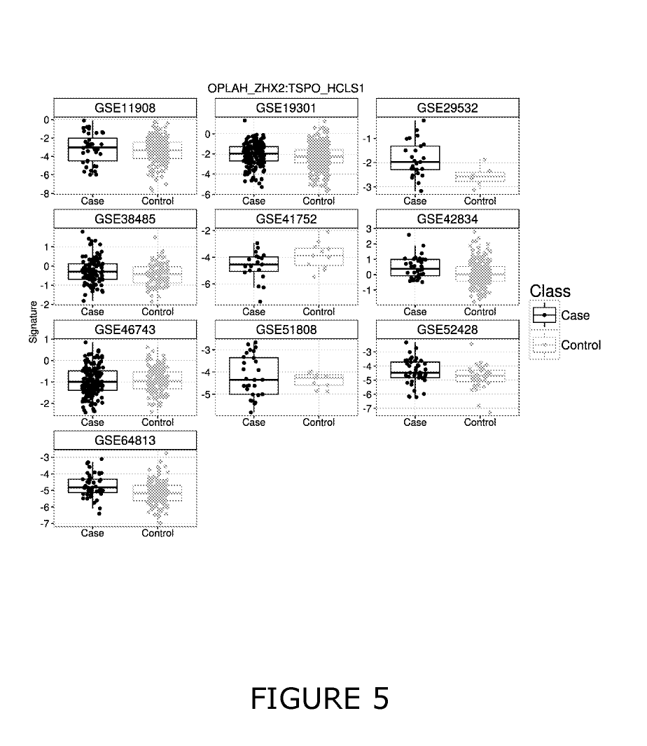

The best combinations of derived ratios were then determined using a greedy search. A number of factors, including the use of a limited number of derived biomarkers, ease of porting onto a Point-of-Care platform, and performance based on AUC, were used to select the final combination of derived biomarkers. FIG. 1 and TABLE 29 show the AUC performance of the successive addition of individual derived biomarkers in the balanced-scale discovery datasets. The final BaSIRS signature chosen was OPLAH/ZHX2:TSPO/HCLS1 which had an AUC in the balance-scaled data of 0.863. Performance of this signature in each of the individual un-scaled (i.e. raw data) Validation, Discovery and non-BaSIRS datasets is shown in TABLE 14. The mean AUC for this signature in the Discovery, Validation and Non-BaSIRS datasets was 0.923, 0.880 and 0.614 respectively. The performance of this signature is also demonstrated in graphical form in FIGS. 2-5.

Some numerators and denominators occurred more often in the 102 derived biomarkers, perhaps indicating that specific pathways are involved in the immune response to bacteria, or that some biomarkers are expressed in such a manner that makes them more suitable as a numerator or denominator. TABLE 30 lists those individual BaSIRS biomarkers that appear more than once as either a numerator or denominator that are a component of the 102 derived biomarkers with a mean AUC>0.85.

### Example 5

### VaSIRS Host Response Biomarker Derivation

A step-wise procedure was undertaken to identify biomarkers useful in determining a host systemic immune response to viral infection which largely the same as described in Australian provisional patent application 2015903986.

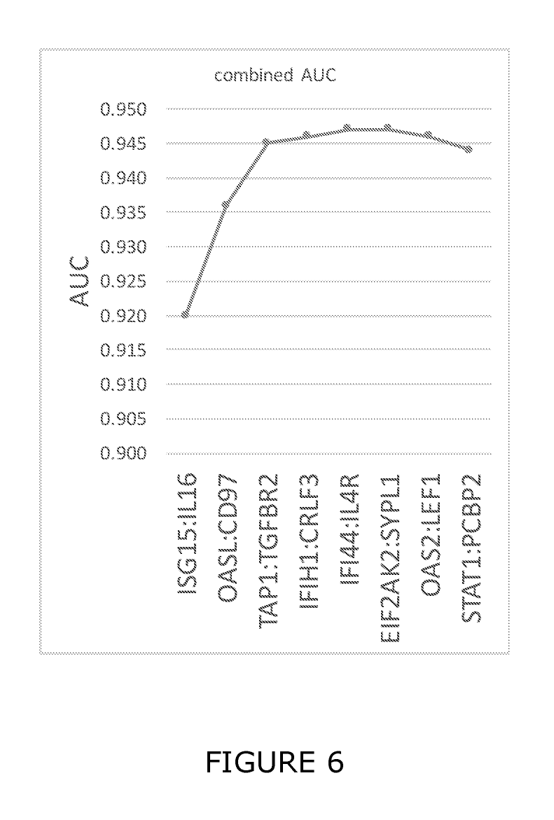

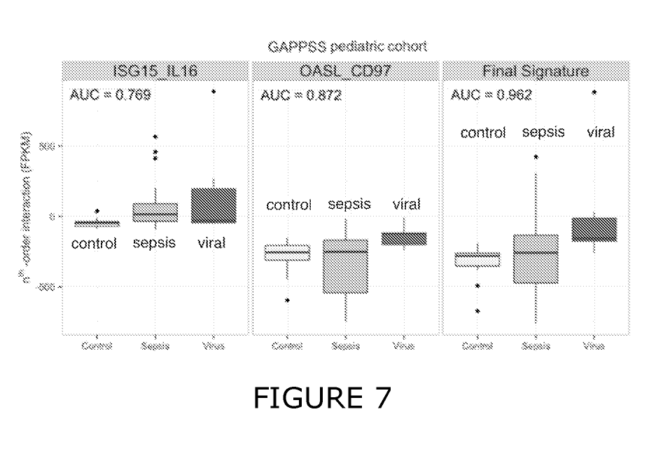

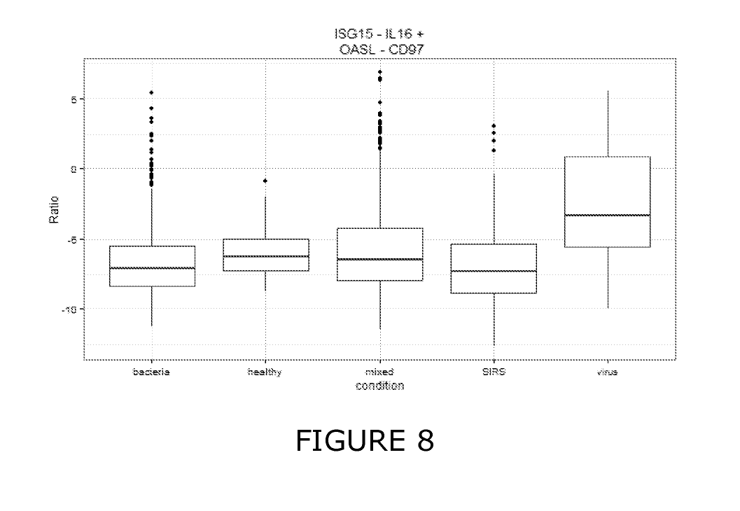

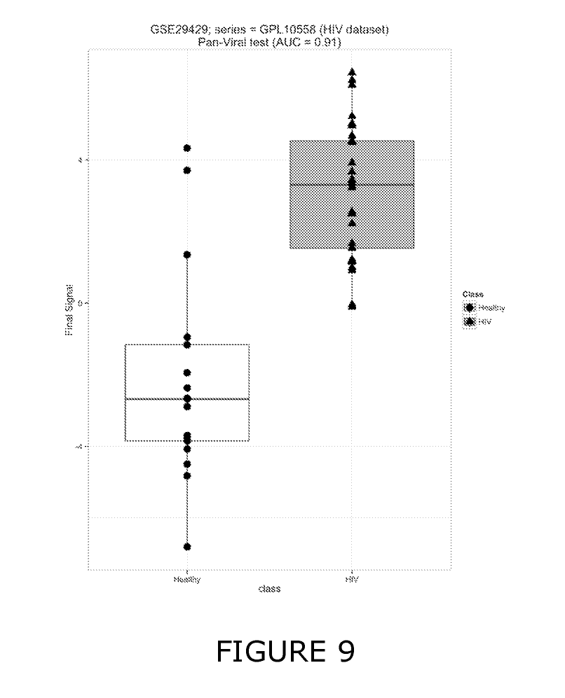

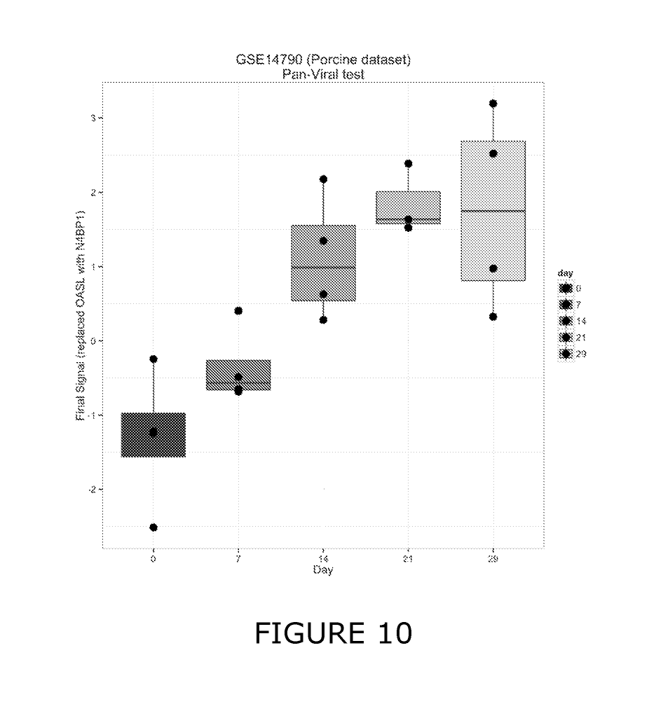

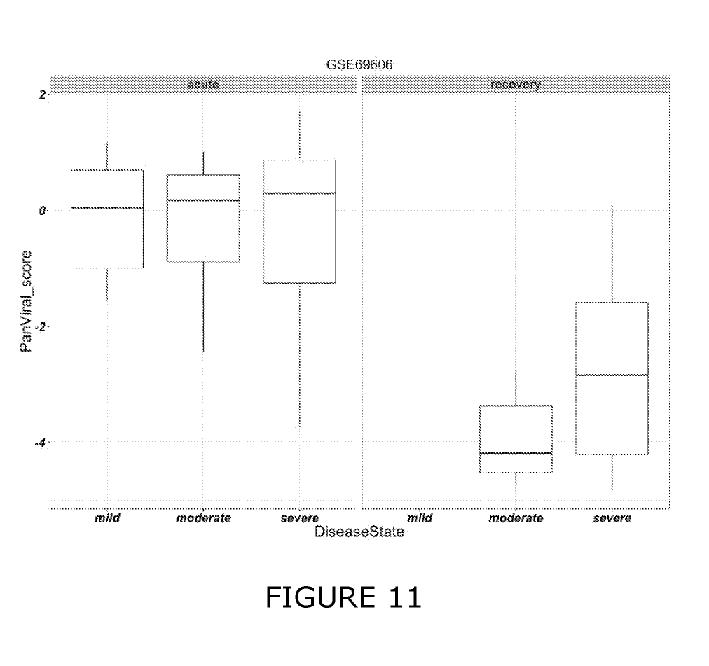

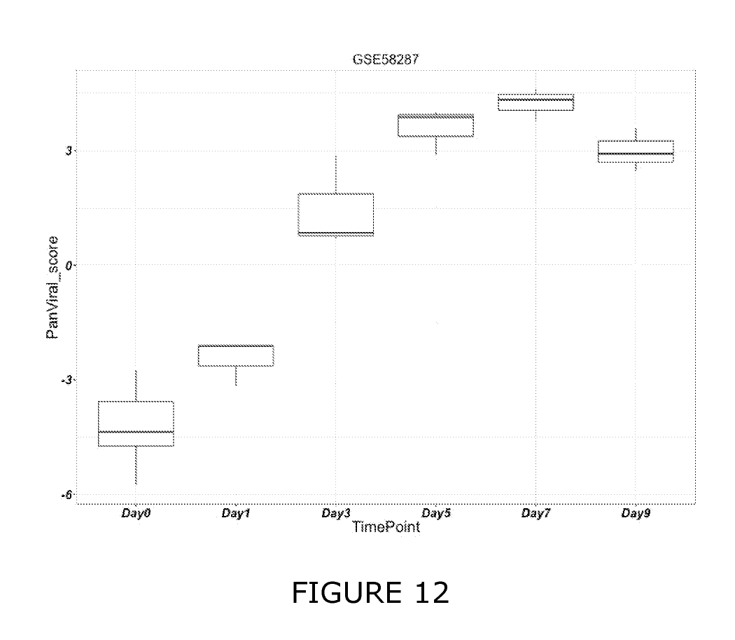

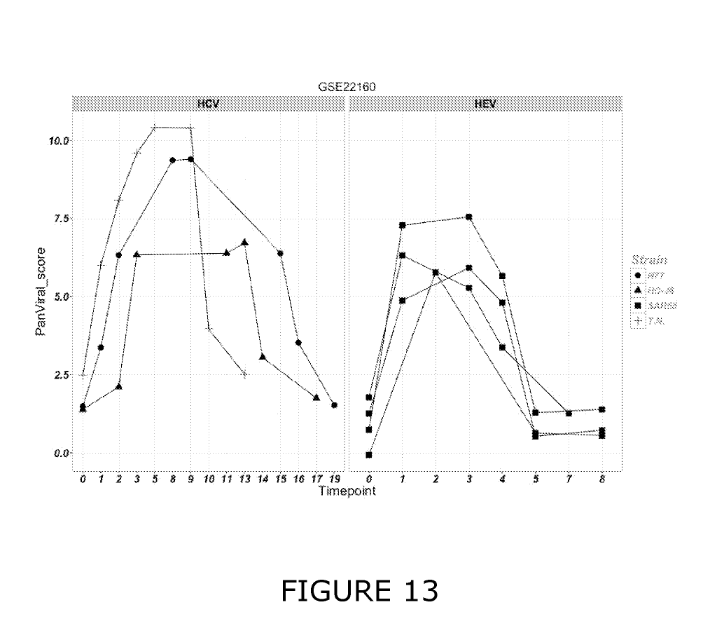

In brief, “pan-viral” derived biomarkers were discovered that are capable of determining a specific mammalian systemic host response to viruses belonging to any of the seven Baltimore virus classification groups. This was achieved using a step-wise approach of derived biomarker discovery, subtraction and validation. Discovery of a large pool of derived biomarkers was performed using a set of four “core” datasets containing samples from subjects with no known infectious co-morbidities and a confirmed viral infection. Derived biomarkers in this large pool were then removed, or subtracted, if they had diagnostic performance, above a set threshold, in other datasets containing samples derived from subjects with other systemic inflammatory conditions, such as bacterial sepsis, allergy, autoimmune disease and sarcoidosis. Derived biomarkers for age, gender, body mass index and race were also subtracted from the pool. Following these steps there remained a total of 473 derived biomarkers with an AUC>0.8 in at least 11 of 14 individual viral datasets (see TABLE 20 for a list of these derived biomarkers and their performance). Using a greedy search on combined datasets, derived biomarkers and combinations of derived biomarkers were then identified that provided good diagnostic performance (AUC=0.936) in the viral datasets (n=14) (See FIG. 6 and TABLE 31). Validation of the diagnostic performance of a “pan-viral” signature, composed of the two derived biomarkers of ISG15/IL16 and OASL/ADGRE5, in a number of other validation datasets was then determined and some results are shown in FIGS. 7-13. Thus, the combination of four biomarkers consisting of ISG15/IL16 and OASL/ADGRE5, and other biomarkers correlated to each of these individual biomarkers, is considered to be a “pan-viral” diagnostic signature that provides strong diagnostic performance across various mammals, including humans, and across different virus types based on Baltimore classification groups I-VII.

Some numerators and denominators occurred more often in the 473 derived biomarkers, perhaps indicating that specific pathways are involved in the immune response to viruses, or that some biomarkers are expressed in such a manner that makes them more suitable as a numerator or denominator. TABLE 31 lists those individual VaSIRS biomarkers that appear more than once as either a numerator or denominator that are a component of the 473 derived biomarkers with a mean AUC>0.8.

### Example 6

### PaSIRS Host Response Biomarker Derivation

A step-wise procedure was undertaken to identify biomarkers useful in determining a host systemic immune response to protozoal infection.

Four suitable datasets were identified in Gene Expression Omnibus covering studies on malaria and leishmania protozoal organisms—see TABLE 21 for details of the number and type of samples in each patient cohort for biomarker discovery. The data was preprocessed by cleaning duplicate genes and performing balanced univariate scaling on all the datasets. All the datasets were then merged by gene name which resulted in 4421 potential target genes.

AUCs were then calculated for all possible combinations of two biomarkers (19,540,820 derived biomarkers). A cut-off of 0.9 was applied and, as such, 9329 derived biomarkers were taken through to the next step of derived biomarker identification.

Sixteen gene expression omnibus datasets were then identified that contained patients or subjects with other conditions, or systemic inflammation due to causes other than protozoal infection (see TABLE 23). These datasets were then merged, as described for the protozoal datasets above, and an AUC calculated for each of the 9329 derived biomarkers. Only derived biomarkers that had an AUC <0.7 in this non-specific merged dataset were taken forward to the next step. As a result 523 derived biomarkers that were considered to be specific to protozoal systemic inflammation were taken forward to the next step.

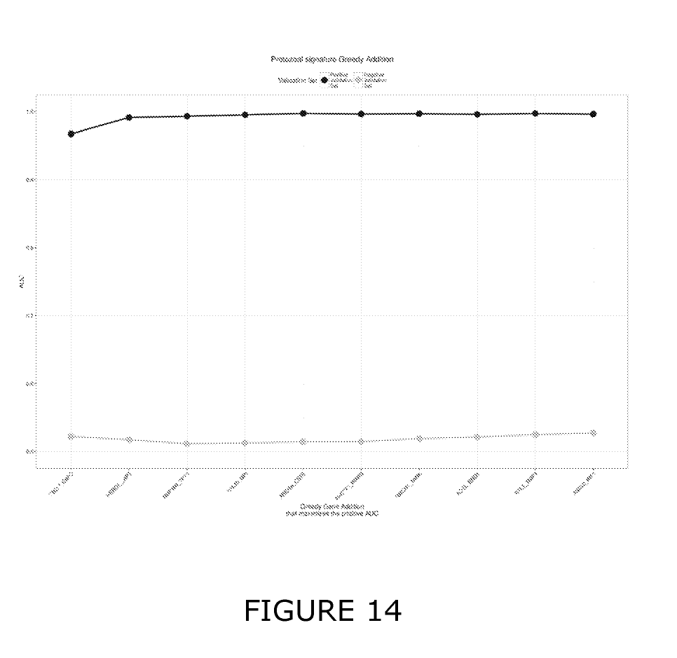

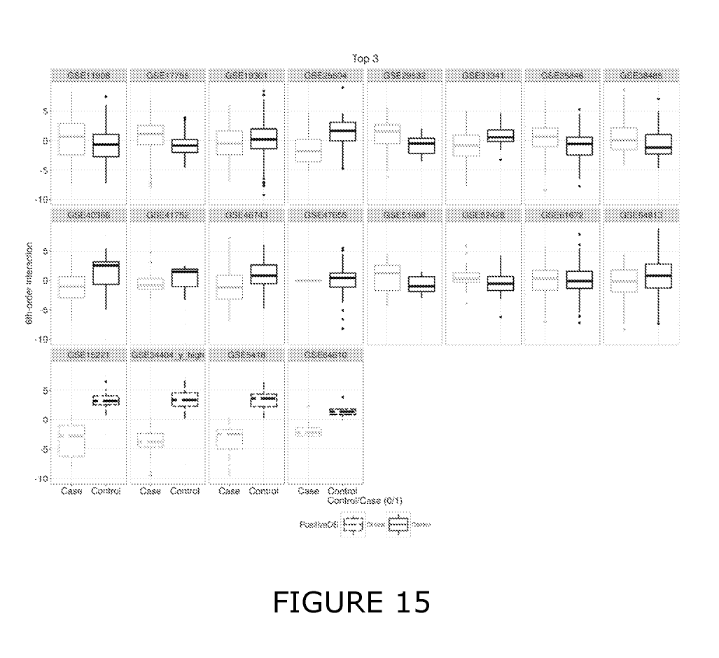

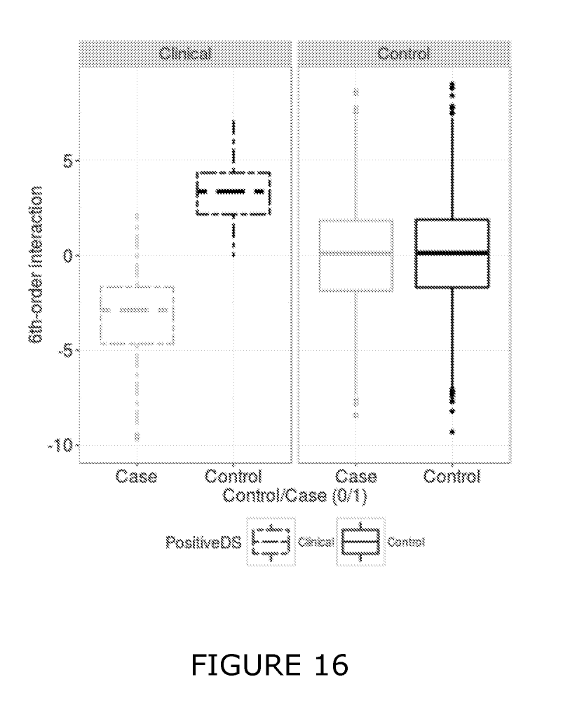

A greedy search was then applied to the protozoal (including the four “discovery” datasets and five “validation” datasets—see TABLE 22) and non-protozoal datasets using all 523 derived biomarkers. The search parameters were set to maximize the difference in AUC between the protozoal and non-protozoal datasets. FIG. 14 shows the results of this greedy search in the form of a plot of AUC versus identified derived biomarkers when added sequentially. TABLE 32 shows the AUC obtained using a single derived biomarker and when using a combination of two and three derived biomarkers. A combination of three derived biomarkers resulted in an AUC of 0.99 and such a combination is considered to be the best through a balance of diagnostic performance, fewest biomarkers and least likelihood of introduction of noise. TABLE 32 identifies the three derived biomarkers and the AUC obtained in the merged datasets used in this study. Performance of these derived biomarkers across all of the datasets used is shown in the box and whisker plots of FIGS. 15 and 16. From these figures it can be clearly seen that the derived biomarkers provide good separation of patients with systemic inflammation due to a protozoal infection compared to control subjects and that these same derived biomarkers have little or no diagnostic utility in patients with systemic inflammation due to causes other than protozoal infection. Performance (AUC) of each of the derived biomarkers alone across each of the protozoal datasets is shown in TABLE 34.

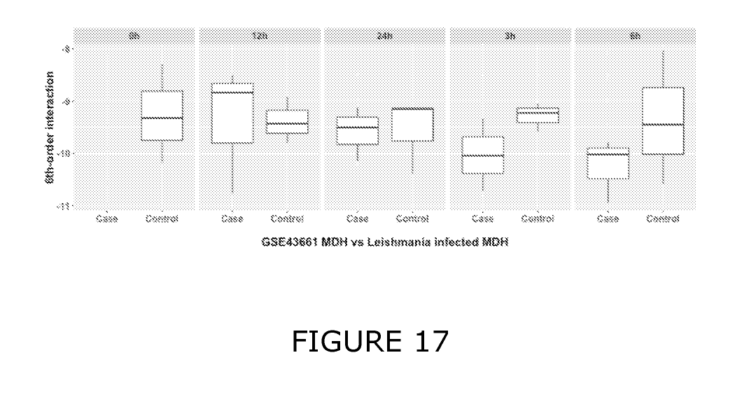

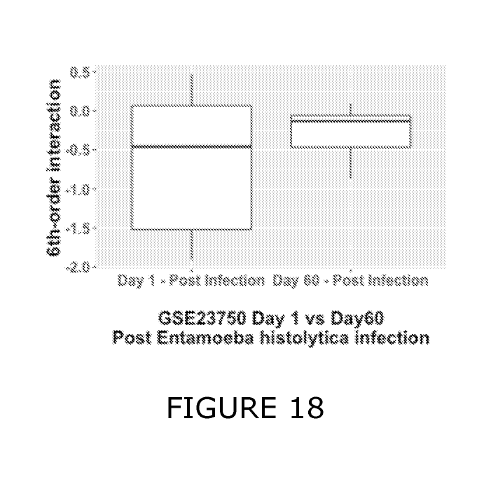

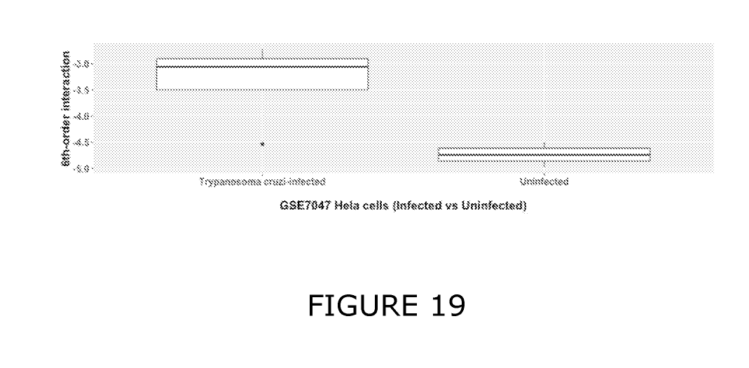

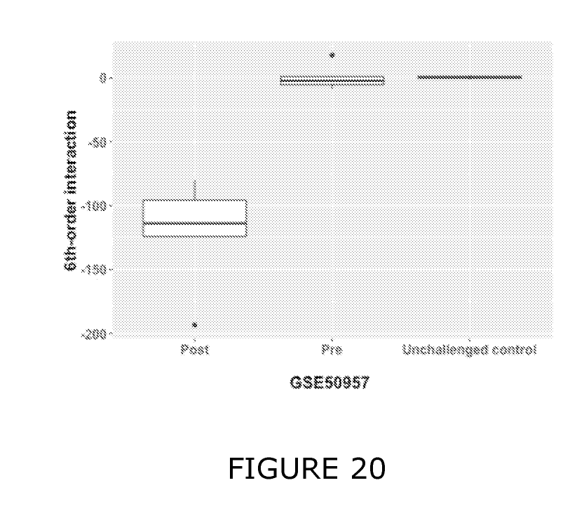

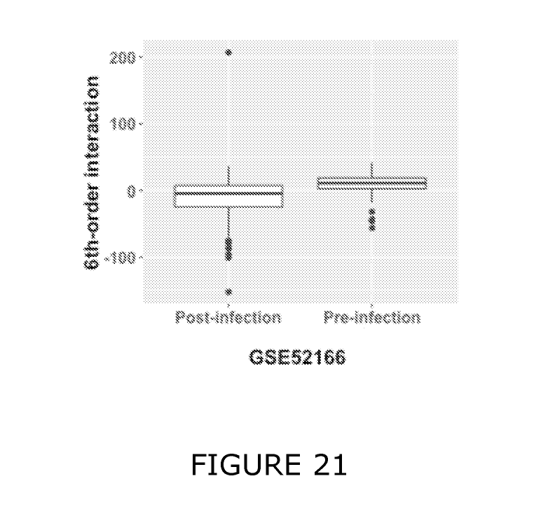

Validation of these derived biomarkers was then performed on five independent datasets obtained from gene expression omnibus (GEO). These datasets represented studies in four types of protozoans, in blood and tissues other than blood, and in vitro and in vivo (see TABLE 21). Because some of these datasets used tissues other than whole blood, and the signature is designed to detect systemic inflammation using circulating leukocytes, diagnostic performance was not expected to be as strong. FIGS. 15-21 shows the performance of the final PaSIRS signature in these datasets, and other datasets, as box and whisker plots.

Some numerators and denominators occurred more often in the 523 derived biomarkers, perhaps indicating that specific pathways are involved in the immune response to protozoans, or that some biomarkers are expressed in such a manner that makes them more suitable as a numerator or denominator. TABLE 33 lists those biomarkers that appear more than once in the 523 derived biomarkers.

### Example 7

### InSIRS Host Response Biomarker Derivation

A step-wise procedure was undertaken to identify biomarkers useful in determining a host systemic immune response to non-infectious causes, which largely employs the same steps that were used to identify host systemic immune response biomarkers of viral infection, as described in Australian provisional patent application 2015903986.

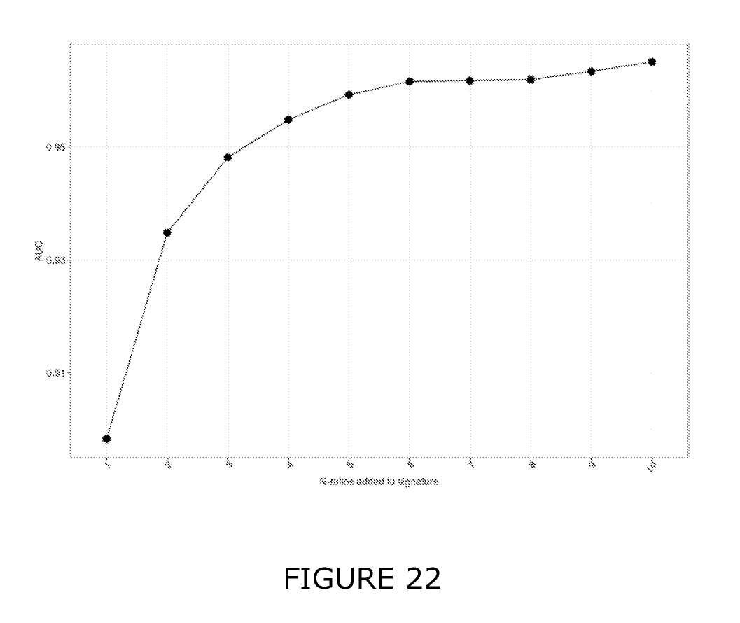

In brief, InSIRS derived biomarkers were discovered that are capable of determining a specific mammalian systemic host response to non-infectious causes. This was achieved using a step-wise approach of derived biomarker discovery, subtraction and validation. Discovery of a large pool of derived biomarkers was performed using a set of datasets containing samples from subjects with no known infectious co-morbidities. Derived biomarkers in this large pool were then removed, or subtracted, if they had diagnostic performance, above a set threshold, in other datasets containing samples derived from subjects with infectious systemic inflammatory conditions, such as bacterial sepsis, viral systemic inflammation and protozoal systemic inflammation. Derived biomarkers for age, gender and race were also subtracted from the pool. Following these steps there remained a total of 164 derived biomarkers with an AUC>0.82 (see TABLE 37 for a list of these derived biomarkers and their performance). Using a greedy search on combined datasets, derived biomarkers and combinations of derived biomarkers were then identified that provided good diagnostic performance (AUC=0.935) in the non-infectious SIRS datasets (See FIG. 22 and TABLE 35). Validation of the diagnostic performance of a InSIRS signature, composed of the two derived biomarkers of ARL6IP5/ENTPD1 and HEATR1/TNFSF8, in a number of other validation datasets was then determined. Thus, the combination of four biomarkers consisting of ARL6IP5/ENTPD1 and HEATR1/TNFSF8, and other biomarkers correlated to each of these individual biomarkers, is considered to be a InSIRS diagnostic signature that provides strong diagnostic performance.

Some numerators and denominators occurred more often in the 164 derived biomarkers, perhaps indicating that specific pathways are involved in the immune response to non-infectious insult, or that some biomarkers are expressed in such a manner that makes them more suitable as a numerator or denominator. TABLE 37 lists those individual InSIRS biomarkers that appear more than once as either a numerator or denominator that are a component of the 164 derived biomarkers with a mean AUC>0.82.

### Example 8

### BaSIRS, VaSIRS, PaSIRS and InSIRS Host Response Biomarker Performance (Derived Biomarkers and Combined Derived Biomarkers)

Following normalization of each of the BaSIRS, VaSIRS, PaSIRS and InSIRS datasets and a greedy search the best performing individual host response specific derived BaSIRS, VaSIRS, PaSIRS and InSIRS biomarkers were: TSPO:HCLS1; ISG15:IL16; TTC17:G6PD; and ARL6IP5:ENTPD1, with AUCs of 0.84, 0.92, 0.96 and 0.89, respectively. The best second unique host response derived biomarkers to add to the first BaSIRS, VaSIRS, PaSIRS and InSIRS derived biomarkers were: OPLAH:ZHX2; OASL:ADGRE5; HERC6:LAP3; and HEATR1:TNFSF8, respectively. The AUCs obtained across the normalized datasets using the two host response specific derived biomarkers for BaSIRS, VaSIRS, PaSIRS and InSIRS was 0.86, 0.936, 0.99 and 0.93, a 0.2, 0.016, 0.3 and 0.36 improvement over the use of single host response specific derived biomarkers (see FIGS. 1, 6, 14 and 22). The addition of third host response specific derived biomarkers (TSPO:RNASE6, TAP1:TGFBR2, NUP160:TPP1 and ADAM19:POLR2A) only improved the AUC by 0.2, 0.009, 0.0 and 0.006 and it is possible that a third derived biomarker created overfitting and noise. However, it was considered that embodiments of optimal signatures consist essentially of the following derived biomarkers: OPLAH:ZHX2/TSPO:HCLS1 (BaSIRS); ISG15:IL16/OASL:ADGRE5 (VaSIRS); TTC17:G6PD/HERC6:LAP3/NUP160:TPP1 (PaSIRS); and ARL6IP5:ENTPD1/HEATR1:TNFSF8 (InSIRS). FIGS. 1, 6, 14 and 22 show the effect on the overall AUC of sequentially adding derived biomarkers to TSPO:HCLS1, ISG15:IL16, TTC17:G6PD and ARL6IP5:ENTPD1.

TABLES 28, 30, 32 and 35 show the performance (AUC) of some of the top host response specific derived biomarkers individually and when added sequentially to the top performing derived biomarkers for the combined datasets.

### Example 8

### BaSIRS, VaSIRS, PaSIRS and InSIRS Host Response Specific Biomarker Frequent Denominators and Numerators

The BaSIRS, VaSIRS, PaSIRS and InSIRS individual biomarkers can be grouped based on the number of times they appear as numerators or denominators in the top performing derived biomarkers.

TABLES 29, 31, 33 and 36 show the frequency of individual biomarkers that appear often in the numerator and denominator positions of the derived biomarkers for BaSIRS, VaSIRS, PaSIRS and InSIRS, respectively. For BaSIRS, PDGFC and TSPO are the most frequent numerators appearing 28 and 11 times, respectively, and INPP5D and KLRD1 are the most frequent denominators appearing 6 times each. For VaSIRS, OASL and USP18 are the most frequent numerators appearing 344 and 50 times, respectively, and ABLIM and IL16 are the most frequent denominators appearing 12 and 9 times, respectively. For PaSIRS, ARID1A and CEP192 are the most frequent numerators appearing 62 and 35 times, respectively, and SQRDL and CEBPB are the most frequent denominators appearing 45 and 40 times, respectively. For InSIRS, TNFSF8 and ADAM19 are the most frequent numerators appearing 90 and 17 times, respectively, and MACF1 and ARL6IP5 are the most frequent denominators appearing 8 and 6 times respectively.

### Example 9

### Example Applications of a Combination of BaSIRS, VaSIRS, PaSIRS and InSIRS Host Response Biomarker Profiles

Use of the BaSIRS, VaSIRS, PaSIRS and InSIRS biomarker profiles in combination in patient populations and the benefits with respect to differentiating various conditions, will now be described.

An assay capable of differentiating patients presenting with clinical signs of systemic inflammation can be used in multiple settings in both advanced and developing countries including: Intensive Care Units (medical and surgical ICU), medical wards, Emergency Departments (ED) and medical clinics. An assay capable of differentiating such patients can be used to identify those patients that (1) need to be isolated from others as part of managing spread of disease; (2) need specific treatments or management procedures; (3) do not need treatment. Such an assay can also be used as part of efforts to ensure judicious use of medical facilities and therapies including antibiotic, anti-viral and anti-protozoal medicines, detection of re-activation of latent or dormant viruses, determination of the severity of a BaSIRS, VaSIRS, PaSIRS or InSIRS, and determination of the etiology of an infection causing the presenting systemic inflammation. Such an assay can also be used to determine whether isolated microorganisms (bacterium, virus, protozoa) are more likely to be true pathogens or a contaminant/commensal/pathobiont/resident/residual microorganism.

### Detecting an Immune Response to Key Pathogens when Patients Present

There are a limited number of human pathogens that cause a bacteremia, viremia or parasitemia and of those that do, their presence in blood is often only for a short period as part of the pathogenesis, making direct detection of the pathogen difficult when using blood as a sample. Further, it takes 10-14 days following an initial infection for specific immunoglobulin G antibodies to appear in blood which can persist for some time making the determination of when a patient became infected difficult. Systemic infection with a pathogen causes a detectable systemic immune response (BaSIRS, VaSIRS, PaSIRS) prior to, and during, the development of peak clinical signs. As such, host response biomarkers are useful for early diagnosis, diagnosis and monitoring in the key periods of pathogen incubation, and when patients present with clinical signs. TABLE 1 lists common human pathogens that are known to cause SIRS and a bacteremia, viremia or parasitemia.

### Detecting a Specific Immune Response to Key Pathogens for which there are Tailored Therapies

It is important to be able to distinguish bacterial, viral and protozoan systemic infections so that appropriate therapies can be administered. Most systemic bacterial infections require immediate treatment with antibiotics and the risk to the patient of missing such a diagnosis is high. For most viruses there are no available anti-viral compounds; however, it is important that viruses, as for example shown in TABLE 2 be detected and identified because 1) they can be treated with anti-viral medication 2) most other viral infections cause transient clinical signs and are not life-threatening. Systemic protozoal infections also require immediate treatment with anti-protozoal therapies; however, in many instances such therapies are administered without a proper diagnostic work-up or even in the face of negative diagnostic test results. In many viral and protozoal infections it is also important to know if there is a co-infection with bacteria so that antibiotics can be prescribed since, in many instances, a systemic bacterial infection can be more life-threatening. The host response biomarkers described herein can determine the extent of systemic inflammation due to a bacterial, viral or protozoal infection and, as such, judgment can be made as to whether antibiotic prescription is appropriate. Further, once it has been determined that systemic inflammation is due to a bacterium, virus or protozoan, other more specific diagnostic tests can be used downstream to identify the pathogen.

### Detecting an Immune Response to Key Pathogens that Cause Respiratory Disease

It is known that the respiratory tract has its own microbiome and virome and that interactions between different bacteria (whether known pathogens, commensals or pathobionts), different viruses and host immune defenses (including innate, cellular, adaptive, physical barriers) determine whether respiratory disease is induced or not (Bosch, A. A. T. M., Biesbroek, G., Trzcinski, K., Sanders, E. A. M., & Bogaert, D. (2013). Viral and Bacterial Interactions in the Upper Respiratory Tract. PLoS Pathogens, 9(1), e1003057-12). Further, it is known that respiratory clinical signs are common in patients with malaria (Taylor, W. R. J., Hanson, J., Turner, G. D. H., White, N. J., & Dondorp, A. M. (2012). Respiratory manifestations of malaria. Chest, 142(2), 492-505). It is also known that both bacteria and viruses are commonly isolated in respiratory tract samples (e.g., Bronchial Alveolar Lavage) from both healthy and diseased subjects, and that different bacteria and viruses can potentiate the pathogenic effects of each other (McCullers, J. A. (2006). Insights into the Interaction between Influenza Virus and Pneumococcus. Clinical Microbiology Reviews, 19(3), 571-582). Therefore, isolating a known pathogen or commensal from a respiratory sample does not necessarily mean it is a causative organism and/or whether it is contributing to respiratory pathology and a host systemic inflammatory response. As such, in patients presenting to medical facilities with respiratory clinical signs in combination with systemic inflammation, it is important to determine an etiology and the extent of systemic inflammation and whether it is due to an infectious organism. The host response biomarkers described herein can determine the extent of systemic inflammation in patients with respiratory clinical signs and whether it is due to a bacterial, viral or protozoal infection. As such, judgment can be made regarding appropriate management procedures, specific anti-viral or anti-protozoal treatments and/or antibiotic treatments.

### Differentiating Patients with Bacterial and Viral Conditions in ICU

It has been shown that greater than 50% and 80% of patients in medical and surgical ICUs respectively have SIRS (Brun-Buisson C (2000) The epidemiology of the systemic inflammatory response. Intensive Care Med 26 Suppl 1: S64-S74). From a clinician's perspective these patients present with non-specific clinical signs and the source and type of infection, if there is one, must be determined quickly so that appropriate therapies can be administered. Patients with InSIRS have a higher likelihood of being infected with bacteria (compared to patients without SIRS), and have a much higher 28-day mortality (Comstedt P, Storgaard M, Lassen A T (2009) The Systemic Inflammatory Response Syndrome (SIRS) in acutely hospitalised medical patients: a cohort study. Scand J Trauma Resusc Emerg Med 17: 67. doi:10.1186/1757-7241-17-67). Further, patients with prolonged sepsis (BaSIRS) have a higher frequency of viral infections, possibly due to reactivation of latent viruses as a result of immunosuppression (Walton, A. H., Muenzer, J. T., Rasche, D., Boomer, J. S., & Sato, B. (2014). Reactivation of multiple viruses in patients with sepsis. PLoS ONE). The higher the prevalence of SIRS in ICU, the higher the risk of infection and death will be in SIRS-affected patients. The re-activation of viruses in ICU patients with BaSIRS, and the benefits of early intervention in patients with BaSIRS (Rivers E P (2010) Point: Adherence to Early Goal-Directed Therapy: Does It Really Matter? Yes. After a Decade, the Scientific Proof Speaks for Itself. Chest 138: 476-480) creates a need for triaging patients with clinical signs of SIRS to determine whether they have a viral or bacterial infection, or both. Monitoring intensive care patients on a regular basis with biomarkers of the present invention will allow medical practitioners to determine the presence, or absence, of a bacterial or viral infection. If positive, further diagnostic tests could then be performed on appropriate clinical samples to determine the type of infection so that appropriate therapy can be administered. For example, if a patient tested positive for a viral infection, and further testing demonstrated the presence of a herpes virus, then appropriate anti-herpes viral therapies could be administered.

In pediatric ICUs the incidence of viral infections is reportedly low (1%), consisting mostly of enterovirus, parechovirus and respiratory syncytial virus infections (Verboon-Maciolek, M. A., Krediet, T. G., Gerards, L. J., Fleer, A., & van Loon, T. M. (2005). Clinical and epidemiologic characteristics of viral infections in a neonatal intensive care unit during a 12-year period. The Pediatric Infectious Disease Journal, 24(10), 901-904). However, because viral infections often predispose infants to bacterial infections, and the mortality rate of virus-infected patients is high, and such patients present with similar clinical signs, it is important to either rule in or rule out the possibility of a bacterial or viral infection so that other appropriate therapies can be administered, and appropriate downstream diagnostic tests and management procedures can be performed.

Determining which patients have which type of infection in the ICU will allow for early intervention, appropriate choice of therapies, when to start and stop therapies, whether a patient needs to be isolated, when to start and stop appropriate patient management procedures, and in determining how a patient is responding to therapy. Information provided by the BaSIRS, VaSIRS, PaSIRS and InSIRS biomarkers of the present invention will therefore allow medical intensivists to tailor and modify therapies and management procedures to ensure infected patients survive and spend less time in intensive care. Less time in intensive care leads to considerable savings in medical expenses including through less occupancy time and through appropriate use and timing of medications.

### Differentiating Patients with Systemic Inflammation Due to an Infection in Hospital Wards

In a study in a U.S. hospital of over 4000 inpatients over an 11-week period at least one episode of fever occurred in 1,194 patients (29%) (McGowan J E J, Rose R C, Jacobs N F, Schaberg D R, Haley R W (1987) Fever in hospitalized patients. With special reference to the medical service. Am J Med 82: 580-586). The rate of fever was highest on medical and surgical services and the authors found that both infectious and non-infectious processes played important roles in the cause. However, determining the cause of fever was complicated by the fact that over 390 different factors were identified. In this study, a review of 341 episodes of fever in 302 patients on the medical service identified a single potential cause in 56%, multiple factors were present in 26%, and no potential causes were found in 18%. Of all factors identified, 44% were community-acquired infections, 9% were nosocomial infections, 20% possibly involved infection, and 26% were non-infectious processes. Thus, fever is common in hospital surgical and medical wards, there are many causes including infectious and non-infectious, diagnosis is difficult and in many instances a cause is not found. The biomarkers outlined herein can differentiate bacterial, viral and protozoal infections from other causes of SIRS which will assist medical practitioners in determining the cause of fever, ensuring that resources are not wasted on unnecessary diagnostic procedures and that patients are managed and treated appropriately.

The estimated number of hospital acquired infections (HAI) in the USA in 2002 was 1.7 million of which approximately 100,000 caused patient death (Klevens et al., Estimating Health Care-Associated Infections and Deaths in U.S. Hospitals. Public Health Reports, March-April 2007 Vol 122, p 160-166, 2002). Common sites and microorganism for HAIs include the respiratory and urinary tracts, and canulas with Staphylococcus and E. coli (Spelman, D. W. (2002). 2: Hospital-acquired infections. The Medical Journal of Australia, 176(6), 286-291). Viruses are also an important cause of HAI where it has been reported that between 5 and 32% of all nosocomial infections are due to viruses, depending upon the hospital location and patient type (Aitken, C., & Jeffries, D. J. (2001). Nosocomial spread of viral disease. Clinical Microbiology Reviews, 14(3), 528-546); Valenti, W. M., Menegus, M. A., Hall, C. B., Pincus, P. H., & Douglas, R. G. J. (1980). Nosocomial viral infections: I. Epidemiology and significance. Infection Control: IC, 1(1), 33-37). Identification of those patients in wards with a BaSIRS or VaSIRS, especially early in the course of infection when there are non-specific clinical signs, would assist clinicians and hospital staff in determining appropriate measures (e.g quarantine, hygiene methods) to be put in place to reduce the risk of spread of infection to other non-infected patients.

### Differentiating Patients with an Infection in Emergency Departments

In 2010, approximately 130 million people presented to emergency departments in the USA and the third most common primary reason for the visit was fever (5.6 million people had a fever (>38° C.) and for 5 million people it was the primary reason for the visit) (Niska R, Bhuiya F, Xu J (2010) National hospital ambulatory medical care survey: 2007 emergency department summary. Natl Health Stat Report 26: 1-31). Of those patients with a fever, 664,000 had a fever of unknown origin—that is, the cause of the fever was not obvious at presentation. As part of diagnosing the reason for the emergency department visit 48,614,000 complete blood counts (CBC) were performed and 5.3 million blood cultures were taken. In 3.65 million patients presenting the primary diagnosis was “infectious” and in approximately 25% of cases (32.4 million) antibiotics were administered. 13.5% of all people presenting to emergency were admitted to hospital. Clinicians in emergency need to determine the answer to a number of questions quickly, including: what is the reason for the visit, is the reason for the visit an infection, does the patient need to be admitted? The diagnosis, treatment and management of patients with a fever, InSIRS, VaSIRS or BaSIRS are different. By way of example, a patient with a fever without other SIRS clinical signs and no obvious source of viral, or bacterial infection may be sent home, or provided with other non-hospital services, without further hospital treatment. However, a patient with a fever may have early BaSIRS, and not admitting such a patient and aggressively treating with antibiotics may put their life at risk. Such a patient may also have VaSIRS and quickly deteriorate, or progress to BaSIRS without appropriate hospital care and/or the use of anti-viral agents. The difference in the number of patients presenting to emergency that are ultimately diagnosed with an “infection” (3.65 million) and the number treated with antibiotics (32.4 million) suggests the following; 1) diagnostic tools that determine the presence of an infection are not available, or are not being used, or are not accurate enough, or do not provide strong enough negative predictive value, or are not providing accurate information that can be acted on within a reasonable timeframe 2) when it comes to suspected infection, and because of the acute nature of infections, clinicians err on the side of caution by administering antibiotics. Further, in a study performed in the Netherlands on patients presenting to emergency with fever, 36.6% of patients admitted to hospital had a suspected bacterial infection (that is, it was not confirmed) (Limper M, Eeftinck Schattenkerk D, de Kruif M D, van Wissen M, Brandjes D P M, et al. (2011) One-year epidemiology of fever at the Emergency Department. Neth J Med 69: 124-128). This suggests that a large proportion of patients presenting to emergency are admitted to hospital without a diagnosis. The BaSIRS and VaSIRS biomarkers described herein can identify those patients with a BaSIRS or VaSIRS from those without a BaSIRS or VaSIRS, assisting medical practitioners in the USA in triaging patients with fever or SIRS. Such effective triage tools make best use of scarce hospital resources, including staff, equipment and therapies. Accurate triage decision-making also ensures that patients requiring hospital treatment are given it, and those that don't are provided with other appropriate services.

In a study performed in Argentina in patients presenting to emergency with influenza-like symptoms, only 37% of samples taken and analyzed for the presence of viruses (using immunofluorescence, RT-PCR and virus culture) were positive (Santamaria, C., Uruena, A., Videla, C., Suarez, A., Ganduglia, C., Carballal, G., et al. (2008). Epidemiological study of influenza virus infections in young adult outpatients from Buenos Aires, Argentina. Influenza and Other Respiratory Viruses, 2(4), 131-134). In a study based in Boston, USA, acute respiratory infections were a common reason children presented to emergency departments in Winter (Bourgeois, F. T., Valim, C., Wei, J. C., McAdam, A. J., & Mandl, K. D. (2006). Influenza and other respiratory virus-related emergency department visits among young children. Pediatrics, 118(1), e1-8). Using a respiratory classifier (based on clinical signs) these authors found that in children less than, or equal to, 7 years of age an acute respiratory infection was suspected in 39.8% of all emergency department visits (less at a whole city or state level). In this latter study only 55.5% of these patients had a virus isolated. Thus, a large percentage of patients with influenza-like symptoms presenting to emergency are likely not being diagnosed as having a viral infection using laboratory-based tests. The VaSIRS biomarkers outlined herein can identify those patients with a VaSIRS from those without a VaSIRS, assisting medical practitioners in making an accurate diagnosis of a viral infection in patients with influenza-like symptoms. Such patients can then be further tested to determine the presence of specific viruses amenable to anti-viral therapies. Accurate diagnosis of a VaSIRS also assists in ensuring that only those patients that need either anti-viral treatment or antibiotics receive them which may lead to fewer side effects and fewer days on antibiotics (Adcock, P. M., Stout, G. G., Hauck, M. A., & Marshall, G. S. (1997). Effect of rapid viral diagnosis on the management of children hospitalized with lower respiratory tract infection. The Pediatric Infectious Disease Journal, 16(9), 842-846).

In a study of febrile pediatric patients presenting to an emergency department in Tanzania, 56.7% had a positive urine test, 19.2% were HIV positive and 8.7% were positive for malaria. Clinical diagnoses included; malaria (24.3%), pneumonia (15.2%), sepsis (9.5%), urinary tract infection (7.6%) and sickle cell anemia (2.9%). A wide range of infections were diagnosed (Ringo, F H., et al., (2013). Clinical presentation, diagnostic evaluation, treatment and diagnoses of febrile children presenting to the emergency department at Muhimbili national hospital in Dar es Salaam, Tanzania. African Journal of Emergency Medicine, 3(4), S21-S22). In this population with systemic inflammation it would therefore be important to distinguish between bacterial, viral and protozoal infection to ensure appropriate treatment and management procedures were rapidly implemented. The biomarkers described in the present specification would assist clinicians in determining whether the cause of the presenting clinical signs of systemic inflammation were due to a bacterial, viral or protozoal infection.

### Differentiating Patients with a Systemic Inflammatory Response to Infection in Medical Clinics

Patients presenting to medical clinics as outpatients often have clinical signs of SIRS including abnormal temperature, heart rate or respiratory rate and there are many causes of these clinical signs. Such patients need to be assessed thoroughly to determine the cause of the clinical signs because in some instances it could be a medical emergency. By way of example, a patient with colic might present with clinical signs of increased heart rate. Differential diagnoses could be (but not limited to) appendicitis, urolithiasis, cholecystitis, pancreatitis, enterocolitis. In each of these conditions it would be important to determine if there was a non-infectious systemic inflammatory response (InSIRS) or whether an infection was contributing to the systemic response. The treatment and management of patients with non-infectious systemic inflammation and/or SIRS due to infectious causes are different. The BaSIRS, VaSIRS, PaSIRS and InSIRS biomarkers detailed herein can differentiate infectious causes of SIRS from other causes of SIRS so that a medical practitioner can either rule in or rule out a systemic inflammation of bacterial, viral or protozoal etiology. As a result medical practitioners can more easily determine the next medical actions and procedure(s) to perform to satisfactorily resolve the patient issue.

### Detection of Reactivation of Latent Viruses

Reactivation of latent viruses is common in patients that are immunocompromised, including those with prolonged sepsis and those on immunosuppressive therapy (Walton A H, Muenzer J T, Rasche D, Boomer J S, Sato B, et al. (2014) Reactivation of multiple viruses in patients with sepsis. PLoS ONE 9: e98819; Andersen, H. K., and E. S. Spencer. 1969. Cytomegalovirus infection among renal allograft recipients. Acta Med. Scand. 186:7-19; Bustamante C I, Wade J C (1991) Herpes simplex virus infection in the immunocompromised cancer patient. J Clin Oncol 9: 1903-1915). For patients with sepsis (Walton et al., 2014), cytomegalovirus (CMV), Epstein-Barr (EBV), herpes-simplex (HSV), human herpes virus-6 (HHV-6), and anellovirus TTV were all detectable in blood at higher rates compared to control patients, and those patients with detectable CMV had higher 90-day mortality. However, because these viruses have only been detected in sepsis patients it is not known whether reactivated latent viruses contribute to pathology, morbidity and mortality. The BaSIRS, VaSIRS, PaSIRS and InSIRS biomarkers detailed herein can differentiate infectious causes of SIRS, and the VaSIRS biomarkers can also detect systemic inflammation due to reactivation of latent herpes viruses. Patients with reactivated herpes virus infection could then be put on appropriate anti-viral therapies.

### Determining the Extent of Systemic Inflammation in Patients

Patients presenting to medical facilities often have any one of the four clinical signs of SIRS. However, many different conditions can present with one of the four clinical signs of SIRS and such patients need to be assessed to determine if they have InSIRS, and if so the extent of InSIRS, or BaSIRS, and if so the extent of BaSIRS, or VaSIRS, and if so the extent of VaSIRS, or PaSIRS, and if so the extent of PaSIRS, and to exclude other differential diagnoses.

By way of example, a patient with respiratory distress is likely to present with clinical signs of increased respiratory rate. Differential diagnoses could be (but not limited to) asthma, viral or bacterial pneumonia, respiratory distress due to malaria, congestive heart failure, physical blockage of airways, allergic reaction, collapsed lung, pneumothorax. In this instance it would be important to determine if there was a infection-negative systemic inflammatory response (InSIRS) or whether an infection (viral, bacterial, or protozoal) was contributing to the condition. The treatment and management of patients with and without systemic inflammation and/or viral, bacterial, protozoal infections are different. Because the biomarkers described herein can determine the degree of systemic involvement, the use of them will allow medical practitioners to determine the next medical procedure(s) to perform to satisfactorily resolve the patient issue. Patients with a collapsed lung, pneumothorax or a physical blockage are unlikely to have a systemic inflammatory response and patients with congestive heart failure, allergic reaction or asthma may have a large systemic inflammatory response but not due to infection. The extent of BaSIRS, VaSIRS, PaSIRS or InSIRS, as indicated by biomarkers presented herein, allows clinicians to determine a cause of the respiratory distress, to rule out other possible causes and provides them with information to assist in decision making on next treatment and management steps. For example, a patient with respiratory distress and a strong marker response indicating VaSIRS is likely to be hospitalized and specific viral diagnostic tests performed to ensure that appropriate anti-viral therapy is administered.

### Antibiotic Stewardship

In patients suspected of having a systemic infection (InSIRS, BaSIRS, VaSIRS, PaSIRS) a clinical diagnosis and treatment regimen is provided by the physician(s) at the time the patient presents and often in the absence of any results from diagnostic tests. This is done in the interests of rapid treatment and positive patient outcomes. However, such an approach leads to over-prescribing of antibiotics irrespective of whether the patient has a bacterial infection or not. Clinician diagnosis of BaSIRS is reasonably reliable (0.88) in children but only with respect to differentiating between patients ultimately shown to be blood culture positive and those that were judged to be unlikely to have an infection at the time antibiotics were administered (Fischer, J. E. et al. Quantifying uncertainty: physicians' estimates of infection in critically ill neonates and children. Clin. Infect. Dis. 38, 1383-1390 (2004)). In Fischer et al., (2004), 54% of critically ill children were put on antibiotics during their hospital stay, of which only 14% and 16% had proven systemic bacterial infection or localized infection respectively. In this study, 53% of antibiotic treatment courses for critically ill children were for those that had an unlikely infection and 38% were antibiotic treatment courses for critically ill children as a rule-out treatment episode. Clearly, pediatric physicians err on the side of caution with respect to treating critically ill patients by placing all patients suspected of an infection on antibiotics—38% of all antibiotics used in critically ill children are used on the basis of ruling out BaSIRS, that is, are used as a precaution. Antibiotics are also widely prescribed and overused in adult patients as reported in Braykov et al., 2014 (Braykov, N. P., Morgan, D. J., Schweizer, M. L., Uslan, D. Z., Kelesidis, T., Weisenberg, S. A., et al. (2014). Assessment of empirical antibiotic therapy optimisation in six hospitals: an observational cohort study. The Lancet Infectious Diseases, 14(12), 1220-1227). In this study, across six US hospitals over four days in 2009 and 2010, 60% of all patients admitted received antibiotics. Of those patients prescribed antibiotics 30% were afebrile and had a normal white blood cell count and where therefore prescribed antibiotics as a precaution. Further, in study of febrile children presenting to an African emergency department 70% were put on antibiotics despite approximately only 35% being diagnosed as having a bacterial infection (Ringo, F H., et al., (2013). Clinical presentation, diagnostic evaluation, treatment and diagnoses of febrile children presenting to the emergency department at Muhimbili national hospital in Dar es Salaam, Tanzania. African Journal of Emergency Medicine, 3(4), S21-S22). As such, an assay that can accurately diagnose BaSIRS, VaSIRS, PaSIRS or InSIRS in patients presenting with non-pathognomonic clinical signs of infection would be clinically useful and may lead to more appropriate use of antibiotics, anti-viral and anti-malarial therapies.

### Controlling the Spread of Infectious Agents

Often the best method of limiting infectious disease spread is through a combination of accurate diagnosis, surveillance, patient isolation and practical measures to prevent transmission (e.g., hand washing) (Sydnor, E. R. M., & Perl, T. M. (2011). Hospital Epidemiology and Infection Control in Acute-Care Settings. Clinical Microbiology Reviews, 24(1), 141-173; Chowell, G., Castillo-Chavez, C., Fenimore, P. W., Kribs-Zaleta, C. M., Arriola, L., & Hyman, J. M. (2004). Model parameters and outbreak control for SARS. Emerging Infectious Diseases, 10(7), 1258-1263.; Centers for Disease Control, Interim U.S. Guidance for Monitoring and Movement of Persons with Potential Ebola Virus Exposure, Dec. 24, 2014; Fletcher, S. M., Stark, D., Harkness, J., & Ellis, J. (2012). Enteric Protozoa in the Developed World: a Public Health Perspective. Clinical Microbiology Reviews, 25(3), 420-449). The BaSIRS, VaSIRS, PaSIRS and InSIRS biomarkers detailed herein can be used to identify those people with early clinical signs that actually have a BaSIRS, VaSIRS, PaSIRS or InSIRS. For those people identified as having a BaSIRS, VaSIRS, PaSIRS or InSIRS appropriate testing and procedures can then be performed to obtain an accurate and specific diagnosis and to limit infectious agent spread, if diagnosed, through isolation of patients and the use of appropriate protective measures.

### Example 10

### Example Applications of a Combination of Host Response Biomarker Profiles and/or Pathogen Specific Biomarkers

Combining host response biomarker profiles and pathogen specific biomarkers provides extra diagnostic power that is useful in a number of medical facility locations (e.g., clinics, emergency, ward, ICU) and infectious disease diagnostic situations. For the diagnosis of BaSIRS, typically blood and other body fluid samples are taken for culture. In comparison to a physician's retrospective diagnosis these culture results are often falsely positive or falsely negative. Possible causes of such false positive or negative results include: growth of a contaminant or commensal organism, overgrowth of a dominant non-pathogenic organism, organism not viable, organism will not grow in media, organism not present in the sample, not enough sample taken, antibiotics in the sample inhibit growth. TABLE 39 indicates possible interpretation of either positive or negative results using a combination of BaSIRS and BIP biomarkers.

For the diagnosis of VaSIRS, typically blood and other body fluid samples are taken for protein-based or molecular DNA testing (as either individual tests or a panel of tests). In comparison to a physician's retrospective diagnosis these test results are also often falsely positive or falsely negative. Possible causes of such false positive or negative results include; presence of a virus that is not contributing to pathology (latency, commensal), virus not present in the sample, not enough sample taken, assay not sensitive enough, wrong assay performed, specific antibodies have not yet been produced, residual antibodies from a previous non-relevant infection. TABLE 40 indicates possible interpretation of either positive or negative results using a combination of VaSIRS and VIP biomarkers.

For the diagnosis of PaSIRS, typically blood and other body fluid samples are taken for antibody or antigen testing (as either individual tests or a panel of tests). In comparison to a physician's retrospective diagnosis these test results are also often falsely positive or falsely negative. Possible causes of such false positive or negative results include; presence of a protozoan that is not contributing to pathology, protozoan not present in the sample, not enough sample taken, assay not sensitive enough, wrong assay performed, antibodies not yet produced, residual antibodies from a previous non-relevant infection. TABLE 41 indicates possible interpretation of either positive or negative results using a combination of PaSIRS and PIP biomarkers.

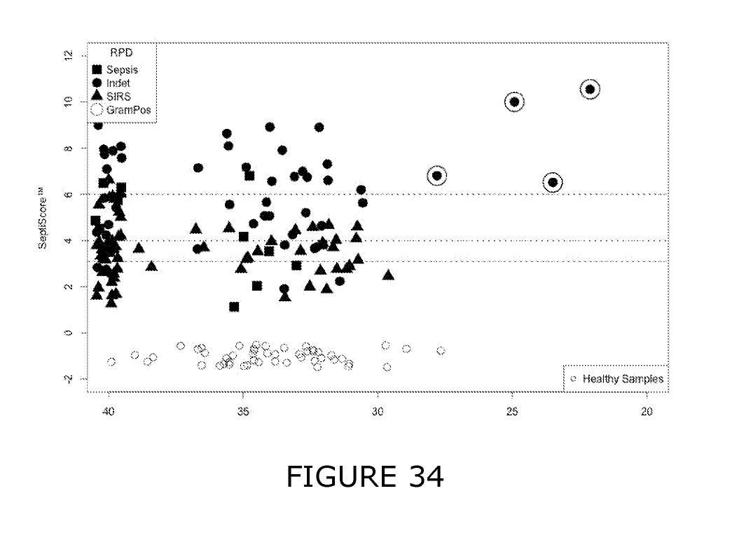

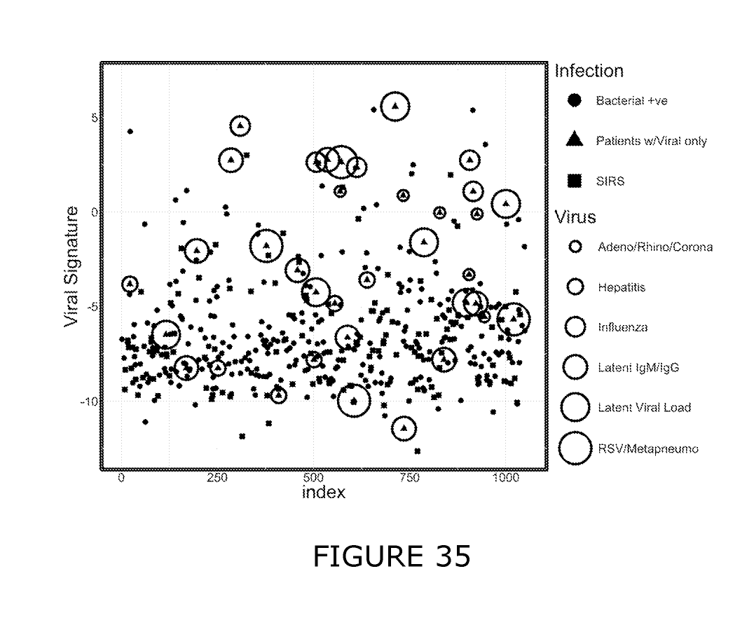

In some instances it would be useful to use BaSIRS and VaSIRS host response specific biomarkers in combination with bacterial and viral pathogen specific biomarkers. For example, children often present to first world emergency departments with fever. Interpretation of results would be along the same lines as described in the tables above. However, double positive results (for either bacterial or viral) would provide greater assurance to the clinician that a child had either a bacterial or viral infection. If all assays were positive then a mixed infection would be likely. If all assays were negative then it is likely the child has InSIRS. A positive BaSIRS host response in combination with a positive bacterial pathogen test would be the most life threatening and require immediate medical attention, administration of appropriate therapies (antibiotics) and appropriate interventions. A negative BaSIRS host response in combination with a negative bacterial pathogen test would provide clinicians with assurance that the cause of the fever was not bacterial. FIGS. 34 and 35 show the use of a combination of BaSIRS and bacterial pathogen detection, and VaSIRS and viral pathogen detection respectively when using in-house clinical samples (Venus A study and MARS study). TABLES 38 and 39 demonstrate how the results of the use of such combinations may be interpreted.

In some instances it would be useful to use BaSIRS, VaSIRS, PaSIRS and InSIRS host response biomarkers in combination with bacterial, viral and protozoal pathogen specific biomarkers. For example, children often present to third world emergency departments with fever. Interpretation of results would be along the same lines as described in the tables above. However, double positive results (for either bacterial or viral or protozoal) would provide greater assurance to the clinician that a child had either a bacterial or viral or protozoal infection. If two or more assays were positive then a mixed infection would be likely. If BaSIRS, VaSIRS and PaSIRS assays and pathogen assays were negative then it is likely the child has InSIRS. A positive BaSIRS host response in combination with a positive bacterial pathogen test would be the most life threatening and require immediate medical attention, administration of appropriate therapies (antibiotics) and appropriate interventions. A negative BaSIRS host response in combination with a positive InSIRS host response and a negative bacterial pathogen test would provide clinicians with assurance that the cause of the fever was not bacterial.

In some instances it would be useful to use just host response biomarkers (BaSIRS, VaSIRS, PaSIRS, InSIRS, alone or in combination), especially in instances where it is known that growth and isolation of a causative organism has a low positive rate (e.g. blood culture in patients in a setting with a low prevalence of sepsis).

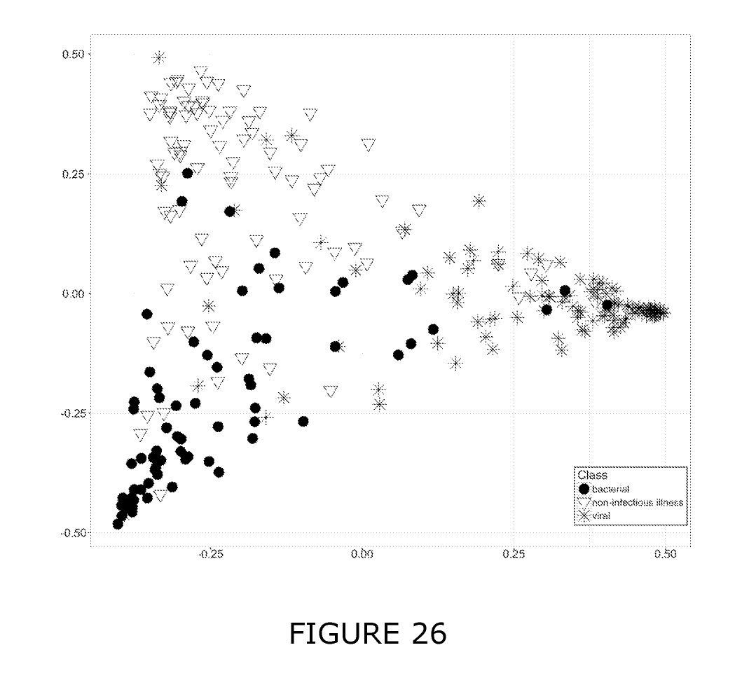

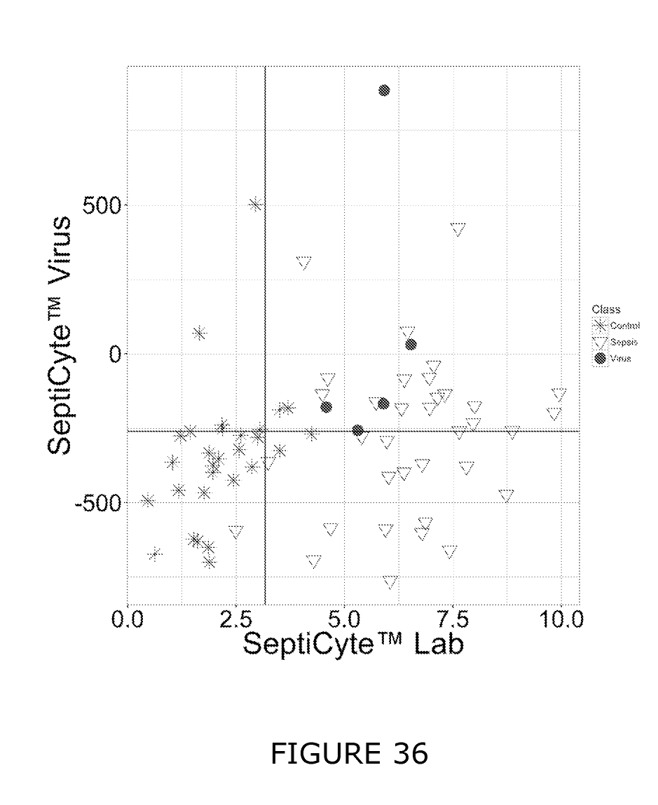

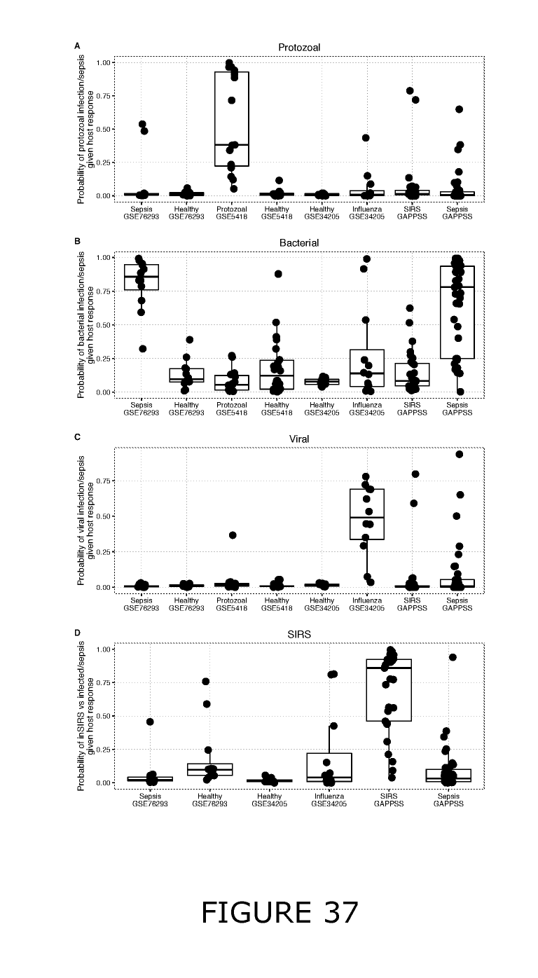

Examples of the use of multiple host response biomarkers are depicted in FIGS. 26, 36 and 37. FIG. 26 shows a multi-dimensional scaling plot using random forest and BaSIRS and VaSIRS derived biomarkers on data associated with GSE63990. In this dataset patients with acute respiratory inflammation were retrospectively categorized by a clinician into the cohorts of: bacterial, viral or non-infectious. Separation of such patients into these three cohorts using BaSIRS and VaSIRS derived biomarkers can be seen clearly. FIG. 36 shows the use of the BaSIRS and VaSIRS signature in a pediatric population with retrospectively diagnosed sepsis, InSIRS, viral infection and mixed infection. Some patients show host responses to both bacteria and viruses suggesting that co-infections can occur and/or one type of infection may predispose to another type of infection. FIG. 37 demonstrates the specificity of the BaSIRS, VaSIRS, PaSIRS and InSIRS signatures in a number of GEO datasets covering a variety of conditions including sepsis, malaria, SIRS and influenza, and in healthy subjects.

### Example 11

### First Example Workflow for Determining Host Response

A first example workflow for measuring host response to BaSIRS, VaSIRS, PaSIRS and InSIRS will now be described. The workflow involves a number of steps depending upon availability of automated platforms. The assay uses quantitative, real-time determination of the amount of each host immune cell RNA transcript in the sample based on the detection of fluorescence on a qRT-PCR instrument (e.g., Applied Biosystems 7500 Fast Dx Real-Time PCR Instrument, Applied Biosystems, Foster City, Calif., catalogue number 440685; K082562). Transcripts are each reverse-transcribed, amplified, detected, and quantified in a separate reaction well for each target gene using a probe that is visualized in the FAM channel (by example). Such reactions can be run as single-plexes (one probe for one transcript per tube), multiplexed (multiple probes for multiple transcripts in one tube), one-step (reverse transcription and PCR are performed in the same tube), or two-step (reverse transcription and PCR performed as two separate reactions in two tubes). A score is calculated for each set of BaSIRS, VaSIRS, PaSIRS and InSIRS host response biomarkers using interpretive software provided separately to the kit but designed to integrate with RT-PCR machines. It is contemplated that a separate score is calculated that combines the results of BaSIRS, VaSIRS, PaSIRS and InSIRS host response specific biomarkers using interpretive software provided separately to the kit but designed to integrate with RT-PCR machines. Such a combined score aims to provide clinicians with information regarding the type(s) and degree(s) of systemic inflammation for each of BaSIRS, VaSIRS, PaSIRS and InSIRS.

The workflow below describes the use of manual processing and a pre-prepared kit.

Pre-Analytical

Blood collection

Total RNA isolation

Analytical

Reverse transcription (generation of cDNA)

qPCR preparation

qPCR

Software, Interpretation of Results and Quality Control

Output.

Kit Contents

Diluent

RT Buffer

RT Enzyme Mix

qPCR Buffer

Primer/Probe Mixes

AmpliTaq Gold® (or similar)

High Positive Control (one for each of BaSIRS, VaSIRS, PaSIRS and InSIRS)

Low Positive Control (one for each of BaSIRS, VaSIRS, PaSIRS and InSIRS)

Negative Control

Blood Collection

The specimen used is a 2.5 mL sample of blood collected by venipuncture using the PAXgene® collection tubes within the PAXgene® Blood RNA System (Qiagen, kit catalogue #762164; Becton Dickinson, Collection Tubes catalogue number 762165; K042613). An alternate collection tube is Tempus® (Life Technologies).

Total RNA Isolation

Blood (2.5 mL) collected into a PAXgene RNA tube is processed according to the manufacturer's instructions. Briefly, 2.5 mL sample of blood collected by venipuncture using the PAXgene™ collection tubes within the PAXgene™ Blood RNA System (Qiagen, kit catalogue #762164; Becton Dickinson, Collection Tubes catalogue number 762165; K042613). Total RNA isolation is performed using the procedures specified in the PAXgene™ Blood RNA kit (a component of the PAXgene™ Blood RNA System). The extracted RNA is then tested for purity and yield (for example by running an A_{260/280} ratio using a Nanodrop® (Thermo Scientific)) for which a minimum quality must be (ratio >1.6). RNA should be adjusted in concentration to allow for a constant input volume to the reverse transcription reaction (below). RNA should be processed immediately or stored in single-use volumes at or below −70° C. for later processing.

Reverse Transcription

Determine the appropriate number of reaction equivalents to be prepared (master mix formulation) based on a plate map and the information provided directly below. Each clinical specimen is run in singleton.

Each batch run desirably includes the following specimens:
High Control (one for each of BaSIRS, VaSIRS, PaSIRS and InSIRS), Low Control (one for each of BaSIRS, VaSIRS, PaSIRS and InSIRS), Negative Control, and No Template Control (Test Diluent instead of sample) in singleton each

Program the ABI 7500 Fast Dx Instrument as detailed below.
Launch the software.
Click Create New Document
In the New Document Wizard, select the following options:
   i. Assay: Standard Curve (Absolute Quantitation)
   i. Container: 96-Well Clear
   iii. Template: Blank Document (or select a laboratory-defined template)
   iv. Run Mode: Standard 7500
   v. Operator: Enter operator's initials
   vi. Plate name: [default]
Click Finish
Select the Instrument tab in the upper left
In the Thermal Cycler Protocol area, Thermal Profile tab, enter the following times:
   i. 25° C. for 10 minutes
   ii. 45° C. for 45 minutes
   iii. 93° C. for 10 minutes
   iv. Hold at 25° C. for 60 minutes

In a template-free area, remove the test Diluent and RT-qPCR Test RT Buffer to room temperature to thaw. Leave the RT-qPCR Test RT Enzyme mix in the freezer and/or on a cold block.

In a template-free area, assemble the master mix in the order listed below.

### RT Master Mix—Calculation

| | Per well | ×N |
|---|---|---|
| RT-qPCR Test RT Buffer | 3.5 μL | 3.5 × N |
| RT-qPCR Test RT Enzyme mix | 1.5 μL | 1.5 × N |
| Total Volume | 5 μL | 5 × N |

Gently vortex the master mix then pulse spin. Add the appropriate volume (5 μL) of the RT Master Mix into each well at room temperature.

Remove clinical specimens and control RNAs to thaw. (If the specimens routinely take longer to thaw, this step may be moved upstream in the validated method.)

Vortex the clinical specimens and control RNAs, then pulse spin. Add 10 μL of control RNA or RT-qPCR Test Diluent to each respective control or negative well.

Add 10 μL of sample RNA to each respective sample well (150 ng total input for RT; OD₂₆₀/OD₂₈₀ ratio greater than 1.6). Add 10 μL of RT-qPCR Test Diluent to the respective NTC well.

Note: The final reaction volume per well is 15 μL.

| | Samples |
|---|---|
| RT Master Mix | 5 μL |
| RNA sample | 10 μL |
| Total Volume (per well) | 15 μL |

Mix by gentle pipetting. Avoid forming bubbles in the wells.

Cover wells with a seal.

Spin the plate to remove any bubbles (1 minute at 400×g).

Rapidly transfer to ABI 7500 Fast Dx Instrument pre-programmed as detailed above.

Click Start. Click Save and Continue. Before leaving the instrument, it is recommended to verify that the run started successfully by displaying a time under Estimated Time Remaining.

qPCR master mix may be prepared to coincide roughly with the end of the RT reaction. For example, start about 15 minutes before this time. See below.

When RT is complete (i.e. resting at 25° C.; stop the hold at any time before 60 minutes is complete), spin the plate to collect condensation (1 minute at 400×g).

qPCR Preparation

Determine the appropriate number of reaction equivalents to be prepared (master mix formulation) based on a plate map and the information provided in RT Preparation above.

Program the ABI 7500 Fast Dx with the settings below.
a) Launch the software.
b) Click Create New Document
c) In the New Document Wizard, select the following options:
   i. Assay: Standard Curve (Absolute Quantitation)
   ii. Container: 96-Well Clear
   iii. Template: Blank Document (or select a laboratory-defined template)
   iv. Run Mode: Standard 7500
   v. Operator: Enter operator's initials
   vi. Plate name: Enter desired file name
d) Click Next
e) In the Select Detectors dialog box:
   i. Select the detector for the first biomarker, and then click Add>>.
   ii. Select the detector second biomarker, and then click Add>>, etc.
   iii. Passive Reference: ROX
f) Click Next
g) Assign detectors to appropriate wells according to plate map.
   i. Highlight wells in which the first biomarker assay will be assigned
   ii. Click use for the first biomarker detector
   iii. Repeat the previous two steps for the other biomarkers
   iv. Click Finish
h) Ensure that the Setup and Plate tabs are selected
i) Select the Instrument tab in the upper left
j) In the Thermal Cycler Protocol area, Thermal Profile tab, perform the following actions:
   i. Delete Stage 1 (unless this was completed in a laboratory-defined template).
   ii. Enter sample volume of 25 μL.
   iii. 95° C. 10 minutes
   iv. 40 cycles of 95° C. for 15 seconds, 63° C. for 1 minute
   v. Run Mode: Standard 7500
   vi. Collect data using the “stage 2, step 2 (63.0@1:00)” setting
k) Label the wells as below using this process: Right click over the plate map, then select Well Inspector. With the Well Inspector open, select a well or wells. Click back into the Well Inspector and enter the Sample Name. Close the Well Inspector when completed.
   i. CONN for High Control
   ii. CONL for Low Control
   iii. CONN for Negative Control
   iv. NTC for No Template Control
   v. [Accession ID] for clinical specimens
l) Ensure that detectors and quenchers are selected as listed below (for singleplex reactions—one target per reaction).
   i. FAM for CEACAM4 biomarker 1; quencher=none
   ii. FAM for LAMP1 biomarker 2; quencher=none
   iii. FAM for PLAC8 biomarker 3; quencher=none
   iv. FAM for PLA2G7 biomarker 4; quencher=none
   v. FAM for ISG15 biomarker 1; quencher=none
   vi. FAM for IL16 biomarker 2; quencher=none
   vii. FAM for OASL; biomarker 3; quencher=none
   viii. FAM for ADGRE5; biomarker 4; quencher=none
   ix. FAM for TTC17 biomarker 1; quencher=none
   x. FAM for G6PD biomarker 2; quencher=none
   xi. FAM for HERC6 biomarker 3; quencher=none
   xii. FAM for LAP3 biomarker 4; quencher=none
   xiii. FAM for NUP160 biomarker 5; quencher=none
   xiv. FAM for TPP1 biomarker 6; quencher=none
   xv. FAM for ARL6IP5 biomarker 1; quencher=none
   xvi. FAM for ENTPD1 biomarker 2; quencher=none
   xvii. FAM for HEATR1 biomarker 3; quencher=none
   xviii. FAM for TNFSF8 biomarker 4; quencher=none
   xix. Select “ROX” for passive reference

qPCR

In a template-free area, remove the assay qPCR Buffer and assay Primer/Probe Mixes for each target to room temperature to thaw. Leave the assay AmpliTaq Gold in the freezer and/or on a cold block.

Still in a template-free area, prepare qPCR Master Mixes for each target in the listed order at room temperature.

qPCR Master Mixes - Calculation Per Sample

| | Per well | ×N |
|---|---|---|
| qPCR Buffer | 11 μL | 11 × N |
| Primer/Probe Mix | 3.4 μL | 3.4 × N |
| AmpliTaq Gold ® | 0.6 μL | 0.6 × N |
| Total Volume | 15 μL | 15 × N |

Example forward (F) and reverse (R) primers and probes (P) (in 5′-3′ orientation) and their final reaction concentration for measuring 14 host response transcripts to bacterial, viral and protozoal host response specific biomarkers are contained in TABLE H (F, forward; R, reverse; P, probe). The melting temperature for all primers and probes in this table is approximately 60° C. Primers are designed for best coverage of all transcripts and across an exon/intron border to reduce the likelihood of amplifying genomic DNA.

TABLE H

| Reagent | 5′-3′ Sequence | Reactionr mM | SEQ ID NO |
|---|---|---|---|
| OPLAH-F | GCTGGACATCAACACCGTGGC | 360 | 1666 |
| OPLAH-R | GTCCTGGGTGGGCTCCTGC | 360 | 1667 |
| OPLAH-P | GGGGTTCCCGCCTCTTCTTCAG | 50 | 1668 |
| ZHX2-F | GCGGCAGAAGGTGTGTCGGAA | 360 | 1669 |
| ZHX2-R | GTCCCGTTGATCAGCACAGCAG | 360 | 1670 |
| ZHX2-P | GCAGAGGCTGGCCAGGC | 50 | 1671 |
| TSPO-F | CTGAACTGGGCATGGCCCCC | 360 | 1672 |
| TSPO-R | CCCCACTGACCAGCAGGAGATC | 360 | 1673 |
| TSPO-P | GGTGCCCGACAAATGGGCTG | 50 | 1674 |
| HCLS1-F | GGTCGGTTTGGAGTAGAAAGAGACC | 360 | 1675 |
| HCLS1-R | CCCTCTCAAGTCCGTACTTGCC | 360 | 1676 |
| HCLS1-P | TGGGCCATGAGTATGTTGCC | 50 | 1677 |
| ISG15-F | CTTCGAGGGGAAGCCCCTGGAG | 360 | 1678 |
| ISG15-R | CCTGCTCGGATGCTGGTGGAGC | 360 | 1679 |
| ISG15-P | CATGAATCTGCGCCTGCGGGG | 50 | 1680 |
| IL16-F | GCCCAGTGACCCAAACATCCCC | 360 | 1681 |
| IL16-R | CAAAGCTATAGTCCATCCGAGCCTCG | 360 | 1682 |
| IL16-P | GATAAAACACCCACTGCTTAAG | 50 | 1683 |
| OASL-F | CCCTGGGGCCTTCTCTTCCCA | 360 | 1684 |
| OASL-R | CCGCAGGCCTTGATCAGGC | 360 | 1685 |
| OASL-P | CCCAGCCACCCCCTGAGGTC | 50 | 1686 |
| ADGRE5-F | CCATCCAGAATGTCATCAAATTGGTGGA | 360 | 1687 |
| ADGRE5-R | GGACAGGTGGCGCCAGGG | 360 | 1688 |
| ADGRE5-P | GAACTGATGGAAGCTCCTGGAGAC | 50 | 1689 |
| TTC17-F | GGACGGAAAATCCAGCAGC | 360 | 1690 |
| TTC17-R | CTTCTTGTCTCATTAATATGACTAGG | 360 | 1691 |
| TTC17-P | CACCAATGAACTTGAAGCATCC | 50 | 1692 |
| G6PD-F | GCGACGACGACGAAGCGC | 360 | 1693 |
| G6PD-R | CGCAGGATCCCGCACACC | 360 | 1694 |
| G6PD-P | GGCAGAGCAGGTGGCCCT | 50 | 1695 |
| HERC6-F | GTTTCCTGCCAAGCCTAAACC | 360 | 1696 |
| HERC6-R | GAGCCAGTGGGAAAGGAAGG | 360 | 1697 |
| HERC-P | GAATGCTGTGTGGACTCTCC | 50 | 1698 |
| LAP3-F | CTAGTAGTAAAACCGAGGTCCA | 360 | 1699 |
| LAP3-R | GTGAATTTCCAAGAAGACTGGG | 360 | 1700 |
| LAP3-P | GTCTTGGATTGAGGAAACAGGC | 50 | 1701 |
| NUP160-F | TGATGGAGAATGCACAGCTGC | 360 | 1702 |
| NUP160-R | ATGCGAGCCAAGGAACACTC | 360 | 1703 |
| NUP160-P | TCCTGGAACTGGAAGATCTGG | 50 | 1704 |
| TPP1-F | AATGTGTTCCCACGGCCTTC | 360 | 1705 |
| TPP1-R | GTAGGCACGGCCACTGGC | 360 | 1706 |
| TPP-P | GAGCTCTAGCCCCCACCT | 50 | 1707 |
| ARL6IP5-F | GGAGGAGTCATGGTCTTTGTGTTTGG | 360 | 1708 |
| ARL6IP5-R | ATGCCCATCGGTGTCCTCTTC | 360 | 1709 |
| ARL6IP5-P | TGATGTTTATCCATGCATCGTTGAGAC | 50 | 1710 |
| ENTPD1-F | GGAGCACATCCATTTCATTGGCA | 360 | 1711 |
| ENTPD1-R | GCTGGGATCATGTTGGTCAGG | 360 | 1712 |
| ENTPD1-P | ATCCAGGGCAGCGACGC | 50 | 1713 |
| HEATR1-F | CCCACTGCTACAAAGATCTTGGATTC | 360 | 1714 |
| HEATR1-R | CCAAGAGCACCCTCAACTGAG | 360 | 1715 |
| HEATR1-P | CTGAGTACCCGGGCAGCT | 50 | 1716 |
| TNFSF8-F | GGTGGCCACTATTATGGTGTTGG | 360 | 1717 |
| TNFSF8-R | GAGCAATTTCCTCCTTTGAGGGG | 360 | 1718 |
| TNFSF8-P | CATTCCCAACTCACCTGACAACG | 50 | 1719 |

Gently mix the master mixes by flicking or by vortexing, and then pulse spin. Add 15 μL of qPCR Master Mix to each well at room temperature.

In a template area, add 130 μL of Test Diluent to each cDNA product from the RT Reaction. Reseal the plate tightly and vortex the plate to mix thoroughly.

Add 10 μL of diluted cDNA product to each well according to the plate layout.

Mix by gentle pipetting. Avoid forming bubbles in the wells.

Cover wells with an optical seal.

Spin the plate to remove any bubbles (1 minute at 400×g).

Place on real-time thermal cycler pre-programmed with the settings above.

Click Start. Click Save and Continue. Before leaving the instrument, it is recommended to verify that the run started successfully by displaying a time under Estimated Time Remaining.

Note: Do not open the qPCR plate at any point after amplification has begun.

When amplification has completed, discard the unopened plate.

Software, Interpretation of Results and Quality Control

Software is specifically designed to integrate with the output of PCR machines and to apply an algorithm based on the use of multiple biomarkers. The software takes into account appropriate controls and reports results in a desired format.

When the run has completed on the ABI 7500 Fast Dx Instrument, complete the steps below in the application 7500 Fast System with 21 CFR Part 11 Software, ABI software SDS v1.4.

Click on the Results tab in the upper left corner.

Click on the Amplification Plot tab in the upper left corner.

In the Analysis Settings area, select an auto baseline and manual threshold for all targets. Enter 0.01 as the threshold.

Click on the Analyze button on the right in the Analysis Settings area.

From the menu bar in the upper left, select File then Close.

Complete the form in the dialog box that requests a reason for the change. Click

OK.

Transfer the data file (.sds) to a separate computer running the specific assay RT-qPCR Test Software.

Launch the assay RT-qPCR Test Software. Log in.

From the menu bar in the upper left, select File then Open.

Browse to the location of the transferred data file (.sds). Click OK.

The data file will then be analyzed using the assay's software application for interpretation of results.

Interpretation of Results and Quality Control

Results

Launch the interpretation software. Software application instructions are provided separately.

Following upload of the .sds file, the Software will automatically generate classifier scores for controls and clinical specimens.

Controls

The Software compares each CON (control) specimen (CONN, CONL, CONN) to its expected result. The controls are run in singleton.

| Control specimen | | |
|---|---|---|
| Designation | Name | Expected result |
| CONH | High Control | Score range |
| CONL | Low Control | Score range |
| CONN | Negative Control | Score range |
| NTC | No Template Control | Fail (no Ct for all targets) |

If CONN, CONL, and/or CONN fail the batch run is invalid and no data will be reported for the clinical specimens. This determination is made automatically by the interpretive software. The batch run should be repeated starting with either a new RNA preparation or starting at the RT reaction step.

If NTC yields a result other than Fail (no Ct for all targets), the batch run is invalid and no data may be reported for the clinical specimens. This determination is made by visual inspection of the run data. The batch run should be repeated starting with either a new RNA preparation or starting at the RT reaction step.

If a second batch run fails, please contact technical services. If both the calibrations and all controls are valid, then the batch run is valid and specimen results will be reported.

Specimens

Note that a valid batch run may contain both valid and invalid specimen results.

Analytical criteria (e.g., Ct values) that qualify each specimen as passing or failing (using pre-determined data) are called automatically by the software.

Scores out of range—reported.

Quality Control

Singletons each of the Negative Control, Low Positive Control, and High Positive Control must be included in each batch run. The batch is valid if no flags appear for any of these controls.

A singleton of the No Template Control is included in each batch run and Fail (no Ct for all targets) is a valid result indicating no amplifiable material was detectable in the well.

The negative control must yield a Negative result. If the negative control is flagged as Invalid, then the entire batch run is invalid.

The low positive and high positive controls must fall within the assigned ranges. If one or both of the positive controls are flagged as Invalid, then the entire batch run is invalid.

### Example 12

### Detection of Pathogen Specific Biomarkers

An example workflow for measuring pathogen (bacterial, viral, protozoal) nucleic acid in whole blood will now be described. The workflow is largely similar to that for detecting host response specific biomarkers but involves a number of unique steps. Specific enrichment of pathogens, especially from whole blood, may be required upstream of nucleic acid detection. Nucleic acid is amplified using specific or broad-range forward and reverse primers and the amplicon is detected using fluorescence-labelled probes and a qPCR instrument (e.g., Applied Biosystems 7500 Fast Dx Real-Time PCR Instrument, Applied Biosystems, Foster City, Calif., catalogue number 440685; K082562). Appropriate positive and negative controls need to be used to ensure that the assay has worked and that contamination has not occurred. In part, some steps depend upon availability of automated platforms and specific cartridges designed to enrich, isolate and amplify pathogen nucleic acids.

Bacterial DNA transcripts are each amplified, detected, and quantified in a single multiplexed reaction using a pair of forward and reverse primers and three probes. The forward and reverse primers are broad-range, designed to 16S rDNA and amplify a large number of bacterial species. The probes are designed to identify DNA sequences unique to Gram positive and Gram negative bacteria. Viral DNA transcripts are detected using assays designed specifically for viruses that cause a viremia and for which anti-viral medicines are available, including Influenza A and B, Hepatitis B virus, Hepatitis C virus, Human Immunodeficiency Virus 1 and 2 (HIV-1, -2), Cytomegalovirus (CMV), Varicella Zoster Virus (VZV), Herpes Simplex Virus 1 and 2 (HSV-1 and -2), Epstein Barr Virus (EBV). Alternatively, and for detection of such viruses, commercially available kits could be used, for example, HBV Digene Hybrid Capture II Microplate assay (Digene/Qiagen), Luminex (12212 Technology Blvd. Austin, Tex. 78727 United States), xTAG® Respiratory Viral Panel, Seegene (Washingtonian Blvd. Suite 290 Gaithersburg, Md. 20878 U.S.A.) Respiratory Virus Detection Assay. Protozoal DNA transcripts are each amplified, detected, and quantified in a single multiplexed reaction using three pairs of forward and reverse primers and four probes. The forward and reverse primers are designed to known common protozoal pathogens and the probes are designed to differentiate key protozoal species.

Blood (approximately 0.5 mL) collected into anti-coagulant is processed using a proprietary method, a commercially available kit, or a cartridge designed for use on a point-of-care instrument, and according to the manufacturer's instructions. Microbial DNA may need to be enriched from whole blood prior to performing PCR because the amount of background host DNA in blood reduces the effectiveness and sensitivity of downstream assays designed to detect bacterial DNA. Proprietary methods or commercially available kits or cartridges associated with a point-of-care instrument can be used. A proprietary method could involve the steps of: 1). lysis of microbes through chemical or mechanical means 2). proteolytic digestion in the presence of chaotropic agents and detergents 3).addition of magnetic silicon beads 4). isolation and washing of the beads 5). elution of nucleic acid from the beads. An example bacterial DNA enrichment kit for use on whole blood is MolYsis® Pathogen DNA Isolation (Molzym Life Science, GmbH & Co. KG Mary-Astell-Strasse 10 D-28359 Bremen, Germany) and an example automated machine is Polaris® by Biocartis (Biocartis N V, Generaal De Wittelaan 11 B3 2800 Mechelen Belgium). Other companies, such as Curetis AG and Enigma Limited provide sample preparation methodologies upstream of their proprietary testing cartridges. Kits and automated machines that enrich bacterial DNA from whole blood generally rely on selective lysis of mammalian host cells, digestion of host cell DNA using DNAse enzymes, and filtration and lysis of microbial cells. European patent 2333185 entitled “Selective Lysis of Cells” describes the general procedure. Example commercial kits that enrich for microbial and viral DNAs from whole blood are ApoH Captovir® and ApoH Captobac® (ApoH Technologies, 94, Allée des fauvettes 34 280 La Grande Motte FRANCE). Virus-specific DNA or RNA can be detected in plasma (HIV-1, -2, HBV, HCV, Influenza A and B), whole blood (HCV), or white-blood-cell-enriched fractions (HBV, HCV, herpes viruses). In some instances protozoan DNA needs to be enriched from whole blood (Plasmodium, Babesia), red blood cells (Plasmodium, Babesia), plasma (Trypanosoma), or white blood cells (Toxoplasma, Leishmania) so that it can be sensitively detected in the host DNA milieu. Example methods that enrich for malarial protozoa from whole blood are described in: Venkatesan M, Amaratunga C, Campino S, Auburn S, Koch O, et al. (2012) Using CF11 cellulose columns to inexpensively and effectively remove human DNA from Plasmodium falciparum-infected whole blood samples. Malaria journal 11: 41 and; Trang D T X, Huy N T, Kariu T, Tajima K, Kamei K (2004) One-step concentration of malarial parasite-infected red blood cells and removal of contaminating white blood cells. Malar J 3: 7. An example method that enriches for Trypanosoma from plasma is described in: Nagarkatti R, Bist V, Sun S, Fortes de Araujo F, Nakhasi H L, et al. (2012) Development of an Aptamer-Based Concentration Method for the Detection of Trypanosoma cruzi in Blood. PLoS ONE 7: e43533. An example method that enriches for Leishmania from white blood cells in whole blood is described in: Mathis A, Deplazes P (1995) PCR and in vitro cultivation for detection of Leishmania spp. in diagnostic samples from humans and dogs. Journal of Clinical Microbiology 33: 1145-1149. An example method that enriches for Toxoplasma from white blood cells in whole blood is described in: Colombo F A, Vidal J E, Oliveira A C P D, Hernandez A V, Bonasser-Filho F, et al. (2005) Diagnosis of Cerebral Toxoplasmosis in AIDS Patients in Brazil: Importance of Molecular and Immunological Methods Using Peripheral Blood Samples. Journal of Clinical Microbiology 43: 5044-5047. An example method that enriches for Babesia from red blood cells in whole blood is described in: Persing D H, Mathiesen D, Marshall W F, Telford S R, Spielman A, et al. (1992) Detection of Babesia microti by polymerase chain reaction. Journal of Clinical Microbiology 30: 2097-2103. Once enriched, microbial, viral or protozoan DNA should be processed immediately or stored in single-use volumes at or below −70° C. for later processing.

The downstream amplification, detection and interpretation of qPCR for bacterial DNA is similar to that described in the first example host response workflow but without the need for reverse transcription. Some viruses (RNA viruses, e.g., Influenza) require a reverse transcription step prior to performing qPCR.

Example forward (F) and reverse (R) primers and probes (P) and their final reaction concentration for detecting bacterial DNA are contained in TABLE I.

TABLE I

| | | SEQ | Reaction |
|---|---|---|---|
| Reagent | 5′-3′ Sequence | ID NO. | nM |
| Bacterial-F | ACTCCTACGGGAGGCAGCAGT | 1720 | 800 nM |
| Bacterial-R | GTATTACCGCGGCTGCTGGCA | 1721 | 800 nM |
| G+/−P1 | AGCAACGCCGCGT | 1722 | 250 nM |
| G+/−P2 | AGCGACGCCGCGT | 1723 | 100 nM |
| G+/−P | AGCCATGCCGCGT | 1724 | 200 nM |

Example forward (F) and reverse (R) primers and probes (P) and the protozoan parasitic DNA detected are contained in TABLE G supra.

Example forward (F) and reverse (R) primers and probes for common human pathogenic viruses that cause systemic inflammation and viremia are listed in TABLE F supra, which are disclosed for example in the following references: Watzinger, F., Suda, M., Preuner, S., Baumgartinger, R., Ebner, K., Baskova, L., et al. (2004). Real-time quantitative PCR assays for detection and monitoring of pathogenic human viruses in immunosuppressed pediatric patients. Journal of Clinical Microbiology, 42(11), 5189-5198; Pripuzova N, Wang R, Tsai S, Li B, Hung G-C, et al. (2012) Development of Real-Time PCR Array for Simultaneous Detection of Eight Human Blood-Borne Viral Pathogens. PLoS ONE 7: e43246; van Elden L J R, Nijhuis M, Schipper P, Schuurman R, van Loon A M (2001) Simultaneous Detection of Influenza Viruses A and B Using Real-Time Quantitative PCR. Journal of Clinical Microbiology 39: 196-200; U.S. Pat. No. 5,962,665 (application Ser. No. 08/876,546); Pas S D, Fries E, De Man R A, Osterhaus A D, Niesters H G (2000) Development of a quantitative real-time detection assay for hepatitis B virus DNA and comparison with two commercial assays. Journal of Clinical Microbiology 38: 2897-2901; Namvar L, Olofsson S, Bergstrom T, Lindh M (2005) Detection and Typing of Herpes Simplex Virus (HSV) in Mucocutaneous Samples by TaqMan PCR Targeting a gB Segment Homologous for HSV Types 1 and 2. Journal of Clinical Microbiology 43: 2058-2064; Mentel, R. (2003). Real-time PCR to improve the diagnosis of respiratory syncytial virus infection. Journal of Medical Microbiology, 52(10), 893-896; Do, D. H., Laus, S., Leber, A., Marcon, M. J., Jordan, J. A., Martin, J. M., & Wadowsky, R. M. (2010). A One-Step, Real-Time PCR Assay for Rapid Detection of Rhinovirus. The Journal of Molecular Diagnostics, 12(1), 102-108; Fellner, M. D., Durand, K., Rodriguez, M., Irazu, L., Alonio, V., & Picconi, M. A. (2014). Duplex realtime PCR method for Epstein-Barr virus and human DNA quantification: its application for post-transplant lymphoproliferative disorders detection. The Brazilian Journal of Infectious Diseases, 18(3), 271-280; Sanchez, J. L., & Storch, G. A. (2002). Multiplex, Quantitative, Real-Time PCR Assay for Cytomegalovirus and Human DNA. Journal of Clinical Microbiology, 40(7), 2381-2386; Collot, S., Petit, B., Bordessoule, D., Alain, S., Touati, M., Denis, F., & Ranger-Rogez, S. (2002). Real-Time PCR for Quantification of Human Herpesvirus 6 DNA from Lymph Nodes and Saliva. Journal of Clinical Microbiology, 40(7), 2445-2451; Akiyama, M., Kimura, H., Tsukagoshi, H., Taira, K., Mizuta, K., Saitoh, M., et al. (2009). Development of an assay for the detection and quantification of the measles virus nucleoprotein (N) gene using real-time reverse transcriptase PCR. Journal of Medical Microbiology, 58(5), 638-643; Lanciotti, R. S., Kerst, A. J., Nasci, R. S., Godsey, M. S., Mitchell, C. J., Savage, H. M., et al. (2000). Rapid detection of west nile virus from human clinical specimens, field-collected mosquitoes, and avian samples by a TaqMan reverse transcriptase-PCR assay. Journal of Clinical Microbiology, 38(11), 4066-4071; Moës, E., Vijgen, L., Keyaerts, E., Zlateva, K., Li, S., Maes, P., et al. (2005). BMC Infectious Diseases. BMC Infectious Diseases, 5(1), 6-10; Neske, F., Blessing, K., Tollmann, F., Schubert, J., Rethwilm, A., Kreth, H. W., & Weissbrich, B. (2007). Real-time PCR for diagnosis of human bocavirus infections and phylogenetic analysis. Journal of Clinical Microbiology, 45(7), 2116-2122; Verstrepen, W. A., Kuhn, S., Kockx, M. M., Van De Vyvere, M. E., & Mertens, A. H. (2001). Rapid Detection of Enterovirus RNA in Cerebrospinal Fluid Specimens with a Novel Single-Tube Real-Time Reverse Transcription-PCR Assay. Journal of Clinical Microbiology, 39(11), 4093-4096; Logan, C., O'Leary, J. J., & O'Sullivan, N. (2006). Real-Time Reverse Transcription-PCR for Detection of Rotavirus and Adenovirus as Causative Agents of Acute Viral Gastroenteritis in Children. Journal of Clinical Microbiology, 44(9), 3189-3195; Chigor, V., & Okoh, A. (2012). Quantitative RT-PCR Detection of Hepatitis A Virus, Rotaviruses and Enteroviruses in the Buffalo River and Source Water Dams in the Eastern Cape Province of South Africa. International Journal of Environmental Research and Public Health, 9(12), 4017-4032; Ito, M., Takasaki, T., Yamada, K. I., Nerome, R., Tajima, S., & Kurane, I. (2004). Development and Evaluation of Fluorogenic TaqMan Reverse Transcriptase PCR Assays for Detection of Dengue Virus Types 1 to 4. Journal of Clinical Microbiology, 42(12), 5935-5937; Nix, W. A., Maher, K., Johansson, E. S., Niklasson, B., Lindberg, A. M., Pallansch, M. A., & Oberste, M. S. (2008). Detection of all known parechoviruses by real-time PCR. Journal of Clinical Microbiology, 46(8), 2519-2524; McQuaig, S. M., Scott, T. M., Lukasik, J. O., Paul, J. H., & Harwood, V. J. (2009). Quantification of Human Polyomaviruses JC Virus and BK Virus by TaqMan Quantitative PCR and Comparison to Other Water Quality Indicators in Water and Fecal Samples. Applied and Environmental Microbiology, 75(11), 3379-3388; Raymond, F., Carbonneau, J., Boucher, N., Robitaille, L., Boisvert, S., Wu, W. K., et al. (2009). Comparison of Automated Microarray Detection with Real-Time PCR Assays for Detection of Respiratory Viruses in Specimens Obtained from Children. Journal of Clinical Microbiology, 47(3), 743-750; Kato, T., Mizokami, M., Mukaide, M., Orito, E., Ohno, T., Nakano, T., et al. (2000). Development of a TT virus DNA quantification system using real-time detection PCR. Journal of Clinical Microbiology, 38(1), 94-98; Xiao, X.-L., He, Y.-Q., Yu, Y.-G., Yang, H., Chen, G., Li, H.-F., et al. (2008). Simultaneous detection of human enterovirus 71 and coxsackievirus A16 in clinical specimens by multiplex real-time PCR with an internal amplification control. Archives of Virology, 154(1), 121-125.

Important controls in pathogen detection assays, especially broad-range PCR assays, include the use of 1). a process control 2). a no-template control 3). internal amplification control. A process control added to the clinical sample and detection demonstrates successful pathogen enrichment, isolation and amplification. For the bacterial and protozoal assays described here an appropriate process control is Stenotrophomonas nitritireducens, since it is a harmless soil organism and its 16S rDNA is not amplified by the described broad range forward and reverse primers. Specific forward and reverse primers and a probe are required to detect this organism. Armored RNA (Life Technologies) is an example of a process control that could be used in the viral assays described herein, and again, specific forward and reverse primers and a probe are required to detect this control. A no-template control (e.g., nucleic-acid-free phospate buffered saline) run in parallel demonstrates the level of contamination or background nucleic acid. Broad-range PCR detects many microorganisms commonly found in and on water, soil, human skin, material surfaces, reagents, Taq polymerase, blood collection tubes and chemical preparations. As such, it is almost impossible to eliminate contaminating bacterial nucleic acid. A known level of contaminating or background nucleic acid, determined by running a no-template control, can be subtracted from the results obtained for a clinical sample. An internal amplification control run as part of a PCR demonstrate successful amplification. A synthetic DNA (with no known homology to natural DNA sequence), specific primers and a probe spiked into the PCR reaction are required to detect this control.

### Example 13

### Host Response Example Outputs (BaSIRS, VaSIRS, PaSIRS)

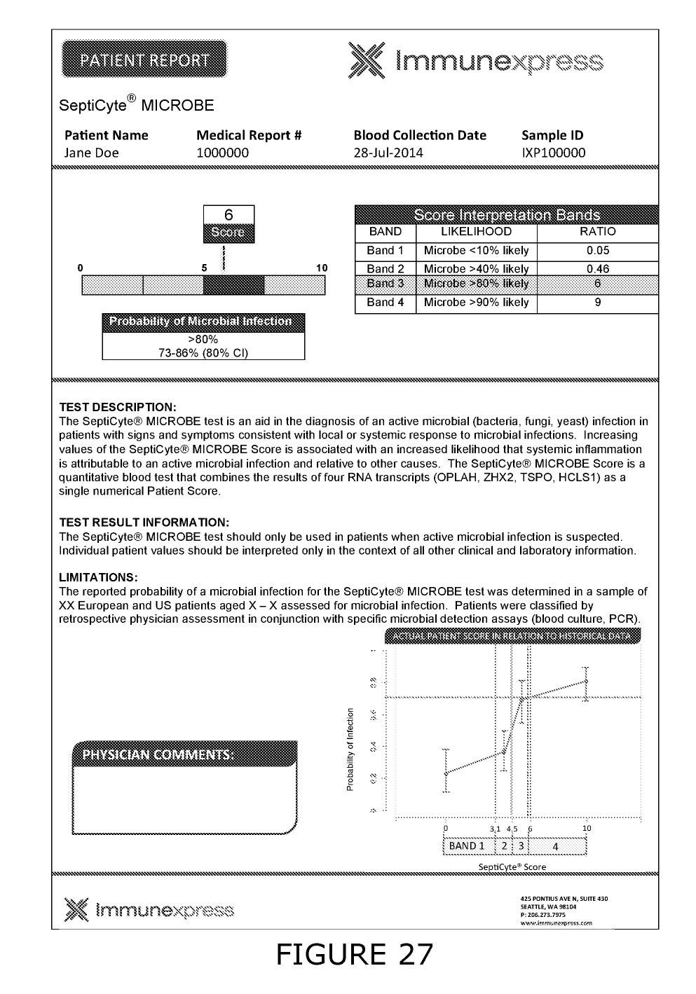

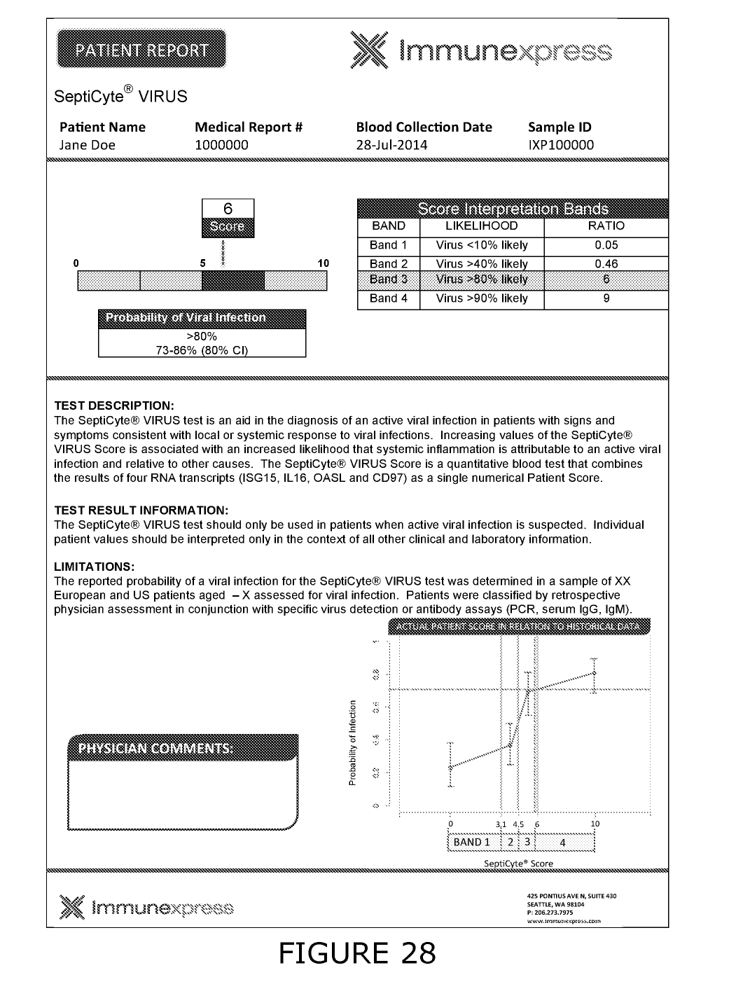

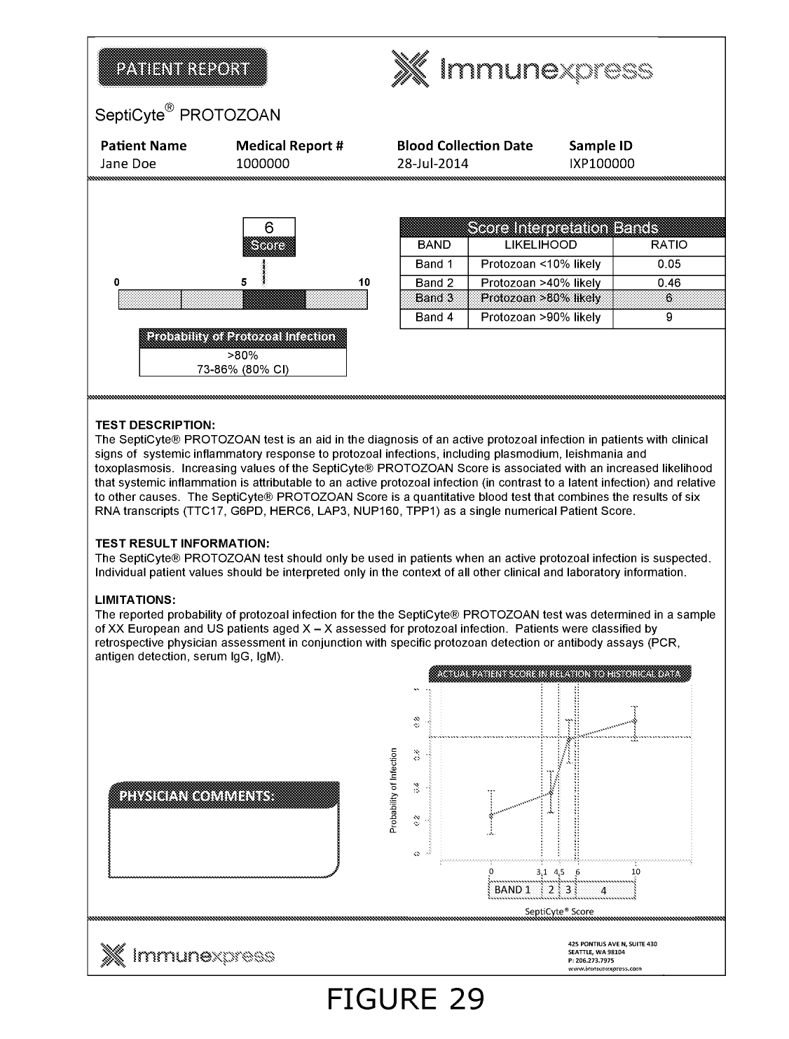

Possible example outputs from the software for BaSIRS, VaSIRS, PaSIRS assays run and analyzed individually are presented in FIGS. 27, 28 and 29. The format of such reports depends on many factors including; quality control, regulatory authorities, cut-off values, the algorithm used, laboratory and clinician requirements, likelihood of misinterpretation.

The host response assays are called “SeptiCyte MICROBE”, “SeptiCyte VIRUS” and “SeptiCyte PROTOZOAN”. The results are reported as a number representing a position on a linear scale, and a probability of the patient having BaSIRS, VaSIRS or PaSIRS based on historical results and the use of pre-determined cut-offs (using results from clinical studies). Results of controls within the assays may also be reported. Other information that could be reported might include: previous results and date and time of such results, a prognosis, a scale that provides cut-off values for historical testing results that separate the conditions of healthy, BaSIRS, VaSIRS, PaSIRS and InSIRS such that those patients with higher scores are considered to have more severe BaSIRS, VaSIRS or PaSIRS.

### Example 14

### Combining Host Response Signatures and Example Outputs

One method of combining the four host response signatures is to calculate a probability of a subject, or subjects, having each of the conditions, as described below.

Additional datasets independent of the discovery process were used including; GSE70311 (Trauma patients that developed bacterial sepsis), GSE34205 (Influenza), GSE5418 (Malaria-infection) and GSE76293 (Bacterial). These datasets included at least one clinical group from each of the pathologies of interest, i.e. bacterial, protozoal and viral infections and a similar control group (InSIRS).

Each of the datasets was log₂ transformed and then the final score was linearly shifted to align each of the control groups across all of the datasets. This latter approach was required because the data were produced on different machines under different study conditions. Because the discovery process for each of the signatures (BaSIRS, VaSIRS, PaSIRS, InSIRS) involved a subtraction step to ensure specificity (signal for conditions other than the one of interest were subtracted), displacing the score in this manner controlled for this variability without losing biological signal.

Probabilities were then calculated by mapping the raw scores through a logit function via a logistic regression model. A one-vs-all response label was set because each of the signatures (BaSIRS, VaSIRS, PaSIRS, InSIRS) had been developed and designed to force non-specific infections into the control group (e.g., for the BaSIRS all non-BaSIRS conditions (VaSIRS, PaSIRS, InSIRS) were treated as controls). Each of the signatures were then applied to each sample and probabilities for each individual sample were calculated using a leave-one-out cross validation (LOO-CV). FIG. 37 demonstrates the use of this approach, through box and whisker plots, for the four host response signatures when using various datasets representing the four conditions.

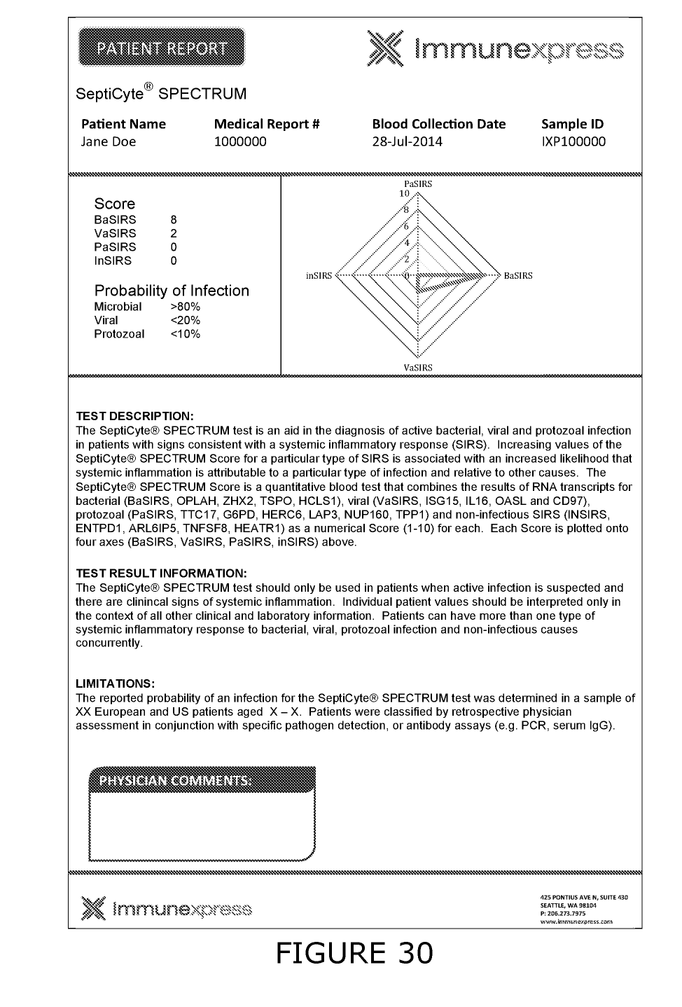

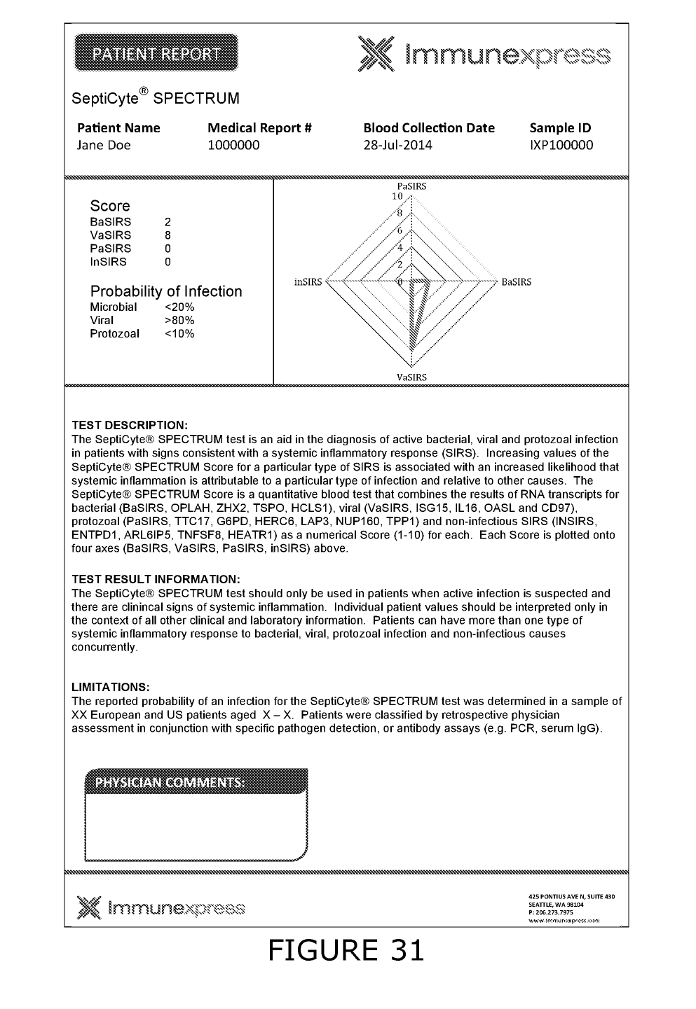

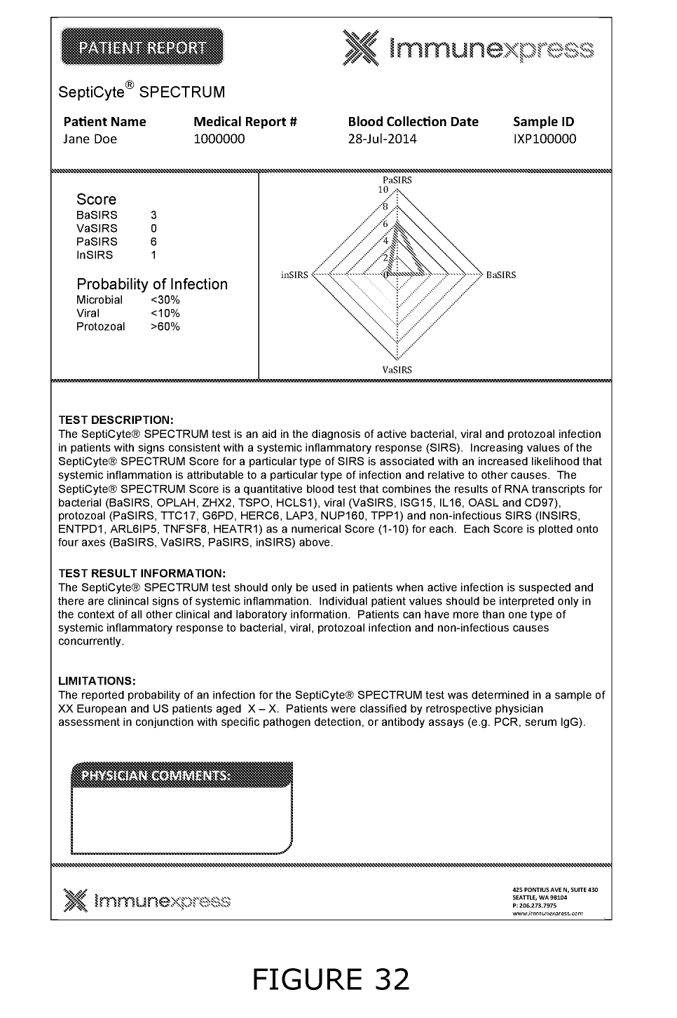

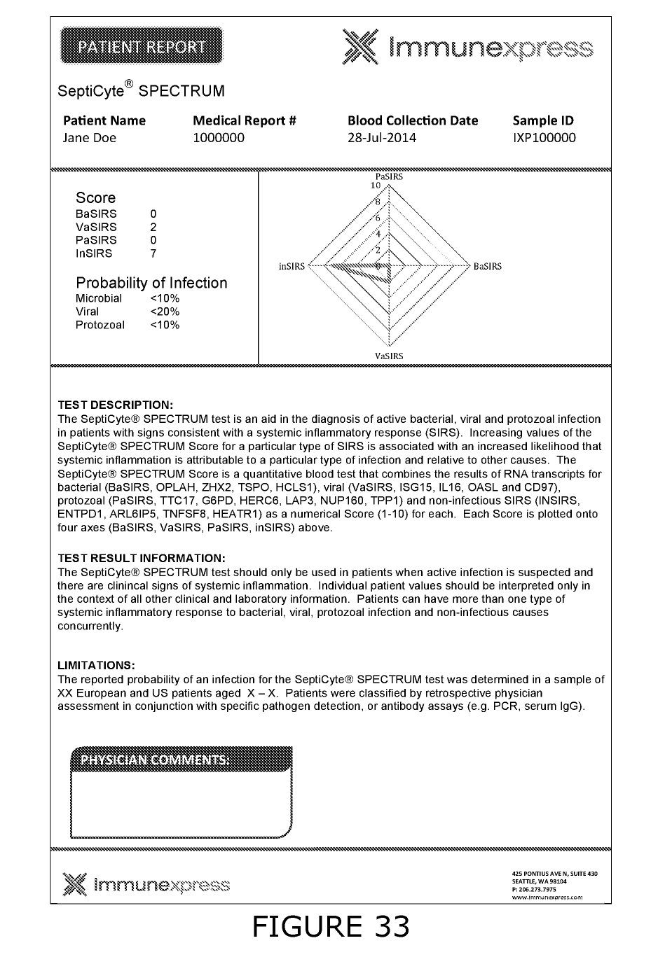

Possible example patient report outputs from the software for BaSIRS, VaSIRS, PaSIRS and InSIRS assays combined are presented in FIGS. 30, 31, 32 and 33. The format of such reports depends on many factors including; quality control, regulatory authorities, cut-off values, the algorithm used, laboratory and clinician requirements, likelihood of misinterpretation.

The combined host response assay is called “SeptiCyte SPECTRUM”. The result is reported as numbers representing positions on linear scales, and a probability of the patient having BaSIRS, VaSIRS, PaSIRS or InSIRS based on historical results and the use of pre-determined cut-offs (using results from clinical studies). Results of controls within the assays may also be reported. Other information that could be reported might include: previous results and date and time of such results, a prognosis, a scale that provides cut-off values for historical testing results that separate the conditions of healthy, InSIRS, BaSIRS, VaSIRS, PaSIRS and InSIRS such that those patients with higher scores are considered to have more severe BaSIRS, VaSIRS, PaSIRS or InSIRS.

### Example 15

### Combination of Host Response Specific Biomarkers Assay Output and Pathogen Specific Biomarkers Assay Output—Example Output (BaSIRS and BIP Combined)

Possible example output from software that combines the results for a host response specific biomarker assay (e.g., BaSIRS) and a pathogen specific biomarker assay (e.g., BIP) for over 50 patients suspected of sepsis and over 50 healthy volunteers is presented in FIG. 34. A similar output is envisaged for a single patient. In this instance, SeptiScore (results of a BaSIRS host response specific biomarker assay) on a scale of −2-12 are plotted on the Y axis, and SeptID (results of a BIP pathogen specific biomarker assay on a reverse scale of 40-20, representing the output of a real-time PCR assay in Ct values) are plotted on the X axis. The higher the SeptiScore the higher the likelihood that a particular patient has BaSIRS. The lower the SeptID score the higher the concentration of bacterial DNA in the sample taken from a patient. Thus, patients with a high SeptiScore and a low SeptID score have a higher probability (or “likelihood”) of BaSIRS compared to patients with a low SeptiScore and a high SeptID score. In FIG. 34, those patients that were ultimately shown to be blood culture positive are circled in the top right of the plot—that is, such patients had a high SeptiScore and low SeptID score. Healthy volunteers had low SeptiScore values and a range (27->40) of SeptID scores.

In this instance the value of combining host response specific biomarkers with pathogen specific biomarkers is; 1) increased positive predictive value in those samples that are positive for both assays, 2) increased negative predictive value in those samples that are negative for both assays, 3) capturing those patients that were retrospectively diagnosed as sepsis and had high SeptiScores, but were blood culture negative, 4) indicating which samples might be contaminated (low SeptiScore, high pathogen detection), and 5) confirmation of blood culture results in a shorter time frame.

Similar outputs are envisaged for: the combination of VaSIRS biomarker assay results and VIP biomarker assay results, and the combination of PaSIRS biomarker assay results and PIP biomarker assay results. A report may contain individual plots for each of the conditions (bacterial, viral and protozoal) or a plot that combines the results for each of these conditions. The format of such reports therefore depends on many factors including; the suspected conditions that the patient has (e.g., bacterial, viral, protozoal), the number and type of assays that are run, quality control, regulatory authority requirements, pre-determined cut-off values, the algorithm used, laboratory and clinician requirements, likelihood of misinterpretation.

In a patient report other information could be conveyed, including: probability of a patient having a particular condition based on historical results, results of controls run, previous results and date and time of such results, a prognosis, a scale that provides cut-off values for historical testing results that separate the conditions of healthy, BaSIRS, VaSIRS, PaSIRS and InSIRS such that those patients with higher scores are considered to have more severe BaSIRS, VaSIRS, PaSIRS or InSIRS.

### Example 16

### Combination of Host Response Specific Biomarkers Assay Output and Pathogen Specific Biomarkers Assay Output—Example Output (VaSIRS and VIP Combined)

Possible example output from software that combines the results for a host response specific biomarker assay (e.g., VaSIRS) and a pathogen specific biomarker assay (e.g., VIP) for over 200 patients suspected of sepsis for which some were concurrently tested for the presence of virus antigen is shown in FIG. 35. A similar output is envisaged for a single patient. In this instance, the VaSIRS signature result is plotted on the Y axis and patients with positive viral pathogen results are circled (with varying sized circles for different virus types). In particular, those patients positive for influenza and RSV virus antigens are also strongly positive for VaSIRS signature. The value of combining host response specific biomarkers (VaSIRS signature) with pathogen specific biomarkers is; 1) increased positive predictive value in those samples that are positive for both assays, 2) increased negative predictive value in those samples that are negative for both assays, and 3) confirmation of virus pathogen detection assay results (not an incidental finding or commensal virus).

### Example 17

### Example Workflow on Automated Machines

A second example automated workflow will now be described. Machines have been, and are being, developed that are capable of processing a patient sample at point-of-care, or near point-of-care. Such machines require few molecular biology skills to run and are aimed at non-technical users. The idea is that the sample would be pipetted directly into a disposable cartridge(s) that is/are then inserted into the machine. One cartridge may be able to run a host response assay and pathogen assay in combination, or separate cartridges may be required to run each assay separately. In both instances the results of each assay will be combined algorithmically following completion of the assay. For determining host response specific biomarkers the cartridge will need to extract high quality RNA from the host cells in the sample for use in reverse transcription followed by RT-PCR. For determining pathogen specific biomarkers the cartridge will need to extract high quality pathogen nucleic acid from the cells in the sample, and away from potentially interfering host nucleic acid, for use in RT-PCR, or reverse transcription followed by RT-PCR. The machines are designed for minimum user interaction such that the user presses “Start” and within 1-3 hours results are generated. The cartridges contains all of the required reagents to perform host cell and pathogen nucleic acid extraction (RNA and/or DNA), reverse transcription, and qRT-PCR, and the machine has appropriate software incorporated to allow use of algorithms to interpret each result and combine results, and final interpretation and printing of results.

Fresh, whole, anti-coagulated blood can be pipetted into a specialized cartridge (e.g., cartridges designed for Enigma ML machine by Enigma Diagnostics Limited (Enigma Diagnostics Limited, Building 224, Tetricus Science Park, DstI, Porton Down, Salisbury, Wiltshire SP4 0JQ) or similar (Unyvero, Curetis A G, Max-Eyth-Str. 42 71088 Holzgerlingen, Germany) (Biocartis N V, Generaal De Wittelaan 11 B3, 2800 Mechelen, Belgium)), and on-screen instructions followed to test for differentiating a BaSIRS, VaSIRS, PaSIRS or InSIRS. For determining host response specific biomarkers, inside the machine RNA is first extracted from the whole blood and is then converted into cDNA. The cDNA is then used in qRT-PCR reactions. For determining pathogen specific biomarkers, inside the machine pathogen nucleic acid is first extracted (possibly selectively) from the whole blood and is then used directly in qRT-PCR reactions, or converted into cDNA and then used in qRT-PCR reactions. The reactions are followed in real time and Ct values calculated. On-board software generates a result output (see, FIGS. 30-33). Appropriate quality control measures for RNA and DNA quality, a process control, no template controls, high and low template controls and expected Ct ranges ensure that results are not reported erroneously.

### Example 18

### Example Algorithms Combining Derived Biomarkers for Assessing SIRS

Derived biomarkers can be used in combination to increase the diagnostic power for separating various conditions. Determining which markers to use, and how many, for separating various conditions can be achieved by calculating Area Under Curve (AUC).

As such, and by example, immune host response biomarker profiles using four to six biomarkers can offer the appropriate balance between simplicity, practicality and commercial risk for diagnosing BaSIRS, VaSIRS, PaSIRS or InSIRS. Further, equations using four to six biomarkers weighs each biomarker equally which provides robustness in cases of analytical or clinical variability.

One example equation (amongst others) that provides good diagnostic power for diagnosing a BaSIRS is:

Diagnostic Score=(TSPO−HCLS1)+(OPLAH−ZHX2)
Note: each marker in the Diagnostic Score above is the Log 2 transformed concentration of the marker in the sample.

One example equation (amongst others) that provides good diagnostic power for diagnosing a VaSIRS is:

Diagnostic Score=(IL16−ISG15)+(ADGRE5−OASL)
Note: each marker in the Diagnostic Score above is the Log 2 transformed concentration of the marker in the sample.

One example equation (amongst others) that provides good diagnostic power for diagnosing a PaSIRS is:

Diagnostic Score=(TTC17−G6PD)+(HERC6−LAP3)+(NUP160−TPP1)
Note: each marker in the Diagnostic Score above is the Log 2 transformed concentration of the marker in the sample.

One example equation (amongst others) that provides good diagnostic power for diagnosing a INSIRS is:

Diagnostic Score=(ARL6IIP5−ENTPD1)+(HEATR1−TNFSF8)
Note: each marker in the Diagnostic Score above is the Log 2 transformed concentration of the marker in the sample.

### Example 19

### Validation of Derived Biomarkers for BaSIRS and VaSIRS on a Pediatric Patient Sample Set

The best performing pairs of host response derived biomarkers for BaSIRS and VaSIRS (TSPO/HCLS1+OPLAH/ZHX2 and IL16/ISG15+ADGRE5/OASL) were further validated on an independent pediatric patient sample set. In this study, samples were collected from three groups of patients including 1). SIRS following cardiopulmonary bypass surgery (n=12) (“Control” in FIG. 36), 2). Sepsis (SIRS+confirmed or strongly suspected bacterial infection) (n=28) (“Sepsis” in FIG. 36), 3). Severe respiratory virus-infected (n=6) (“Virus” in FIG. 36). For SIRS patients, samples were taken within the first 24 hours following surgery and when the patient had at least two clinical signs of SIRS. Sepsis patients were retrospectively diagnosed by a panel of clinicians using all available clinical and diagnostic data. Virus-infected patients were also retrospectively diagnosed by a panel of clinicians using all available clinical and diagnostic data including the use of a viral PCR panel used on nasal or nasal/pharyngeal swabs (Biofire, FilmArray, Respiratory Panel, Biomerieux, 390 Wakara Way Salt Lake City, Utah 84108 USA). The respiratory viruses detected in these patients were: rhinovirus/enterovirus, parainfluenza 3, respiratory syncytial virus and coronavirus HKU1. Three of the six patients with a confirmed virus infection also had a confirmed or suspected bacterial infection. It should be noted that sepsis patients that were not suspected of having a viral infection were also tested with the Biofire FilmArray and nine of the 28 sepsis patients had a positive viral PCR. Thus, there is some overlapping etiologies/pathologies in the sepsis and viral groups which is illustrated in FIG. 36.

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

### TABLES

TABLE 1
NON-LIMITING HUMAN PATHOGENS THAT ARE KNOWN
TO CAUSE SYSTEMIC INFLAMMATION AND BACTEREMIA,
VIREMIA OR PROTOZOAN PARASITEMIA

| Bacteria/Fungi | Viruses | Protozoans |
|---|---|---|
| Coagulase-negative | Respiratory Clinical Signs | Plasmodium falciparum |
| Staphylococcus (CoNS consist | Respiratory Syncytial Virus (RSV) | Plasmodium ovale |
| mainly of S. epidemidis, | Influenza A and B | Plasmodium malariae |
| saprophyticus and hominus) | Adenovirus | Plasmodium vivax |
| Staphylococcus, aureus | Parainfluenza virus 1, 2, 3 and 4 | Leishmania donovani |
| Enterococcus faecalis | Human Coronavirus types 229e, | Trypanosoma brucei |
| Escherichia coli | OC43, HKU1, NL-63 | Trypanosoma cruzi |
| Klebsiella pneumoniae | Rhinovirus | Toxoplasma gondii |
| Enterococcus faecium | SARS Coronavirus | Babesia microti |
| Streptococcus viridans group | Enterovirus | |
| (Streptococcus viridans group | BK virus | |
| includes: mitis, mutans, | Respiratory/Gastrointestinal | |
| oralis, sanginus, sobrinus and | Bocavirus | |
| milleri (anginosus, | Fever/Rash/Aches/Generalised | |
| constellatus, intermedius) | Measles | |
| Pseudomonas aeruginosa | Hantavirus | |
| Streptococcus pneumoniae | Cytomegalovirus | |
| Enterobacter cloacae | Varicella Zoster Virus | |
| Serratia marcescens | Herpes Simplex Virus | |
| Acinetobacter baumammii | Epstein Barr Virus | |
| Proteus mirabilis | Parechovirus | |
| Streptococcus agalactiae | Human immunodeficiency virus | |
| Klebsiella oxytoca | Hepatitis B virus | |
| Enterobacter aerogenes | HTLV1 and 2 | |
| Stenotrophomonas | Vaccinia virus | |
| maltophilia | West Nile Virus | |
| Citrobacter freundii | Coxsackie virus | |
| Streptococcus pyogenes | Parvovirus B19 | |
| Enterococcus avium | Dengue | |
| Bacteroides fragilis | Few Clinical Signs | |
| Bacteroides vulgatus | TTV (torque teno virus) | |
| | Hepatitis C virus | |

TABLE 2
COMMON HUMAN VIRUSES THAT CAUSE SIRS AS
PART OF THEIR PATHOGENESIS AND FOR WHICH
THERE ARE SPECIFIC ANTI-VIRAL TREATMENTS

| Virus | Reference |
|---|---|
| Influenza A and B | Wootton SH, Aguilera EA, Wanger A, |
| | Jewell A, Patel K, et al. (2014) |
| | Detection of NH1N1 influenza virus in |
| | nonrespiratory sites among children. |
| | Pediatr Infect Dis J 33: 95-96. |
| Hepatitis B virus | Pripuzova N, Wang R, Tsai S, Li B, |
| Hepatitis C virus | Hung G-C, et al. (2012) Development |
| Human immunodeficiency | of Real-Time PCR Array for Simulta- |
| virus 1 and 2 | neous Detection of Eight Human Blood- |
| | Borne Viral Pathogens. PLoS ONE 7: |
| | e43246. |
| Cytomegalovirus | Johnson G, Nelson S, Petric M, Tellier |
| Varicella Zoster Virus | R (2000) Comprehensive PCR-based assay |
| Herpes Simplex Virus | for detection and species identification |
| Epstein Barr Virus | of human herpesviruses. Journal of |
| | Clinical Microbiology 38: 3274-3279. |
| Respiratory Syncytial | Najarro, P., Angell, R., & Powell, K. |
| Virus | (2012). The prophylaxis and treatment |
| | with antiviral agents of respiratory |
| | syncytial virus infections. Antiviral |
| | Chemistry & Chemotherapy, 22(4), |
| | 139-150. |

TABLE 3
BASIRS BIOMARKER DETAILS INCLUDING;
SEQUENCE IDENTIFICATION NUMBER,
GENE SYMBOL, ENSEMBL TRANSCRIPT
ID AND DNA SEQUENCE

| SEQ ID | Gene | Ensembl Transcript |
|---|---|---|
| # DNA | Symbol | ID |
| 1 | ADAM19 | ENST00000257527 |
| 2 | ADM | ENST00000278175 |
| 3 | ALPL | ENST00000374840 |
| 4 | CAMK1D | ENST00000378845 |
| 5 | CASS4 | ENST00000360314 |
| 6 | CBLL1 | ENST00000440859 |
| 7 | CCNK | ENST00000389879 |
| 8 | CD82 | ENST00000227155 |
| 9 | CLEC7A | ENST00000353231 |
| 10 | CNNM3 | ENST00000305510 |
| 11 | COX15 | ENST00000370483 |
| 12 | CR1 | ENST00000400960 |
| 13 | DENND3 | ENST00000262585 |
| 14 | DOCK5 | ENST00000276440 |
| 15 | ENTPD7 | ENST00000370489 |
| 16 | EPHB4 | ENST00000358173 |
| 17 | EXTL3 | ENST00000220562 |
| 18 | FAM129A | ENST00000367511 |
| 19 | FBXO28 | ENST00000366862 |
| 20 | FIG4 | ENST00000230124 |
| 21 | FOXJ3 | ENST00000361346 |
| 22 | GAB2 | ENST00000340149 |
| 23 | GALNT2 | ENST00000366672 |
| 24 | GAS7 | ENST00000580865 |
| 25 | GCC2 | ENST00000309863 |
| 26 | GRK5 | ENST00000392870 |
| 27 | HAL | ENST00000261208 |
| 28 | HCLS1 | ENST00000314583 |
| 29 | HK3 | ENST00000292432 |
| 30 | ICK | ENST00000350082 |
| 31 | IGFBP7 | ENST00000295666 |
| 32 | IK | ENST00000417647 |
| 33 | IKZF5 | ENST00000617859 |
| 34 | IL2RB | ENST00000216223 |
| 35 | IMPDH1 | ENST00000338791 |
| 36 | INPP5D | ENST00000359570 |
| 37 | ITGA7 | ENST00000257879 |
| 38 | JARID2 | ENST00000341776 |
| 39 | KIAA0101 | ENST00000300035 |
| 40 | KIAA0355 | ENST00000299505 |
| 41 | KIAA0907 | ENST00000368321 |
| 42 | KLRD1 | ENST00000336164 |
| 43 | KLRF1 | ENST00000617889 |
| 44 | LAG3 | ENST00000203629 |
| 45 | LEPROTL1 | ENST00000321250 |
| 46 | LPIN2 | ENST00000261596 |
| 47 | MBIP | ENST00000416007 |
| 48 | MCTP1 | ENST00000515393 |
| 49 | MGAM | ENST00000549489 |
| 50 | MME | ENST00000460393 |
| 51 | NCOA6 | ENST00000359003 |
| 52 | NFIC | ENST00000341919 |
| 53 | NLRP1 | ENST00000269280 |
| 54 | NMUR1 | ENST00000305141 |
| 55 | NOV | ENST00000259526 |
| 56 | NPAT | ENST00000278612 |
| 57 | OPLAH | ENST00000618853 |
| 58 | PARP8 | ENST00000281631 |
| 59 | PCOLCE2 | ENST00000295992 |
| 60 | PDGFC | ENST00000502773 |
| 61 | PDS5B | ENST00000315596 |
| 62 | PHF3 | ENST00000393387 |
| 63 | PIK3C2A | ENST00000265970 |
| 64 | PLA2G7 | ENST00000274793 |
| 65 | POGZ | ENST00000271715 |
| 66 | PRKD2 | ENST00000433867 |
| 67 | PRKDC | ENST00000314191 |
| 68 | PRPF38B | ENST00000370025 |
| 69 | PRSS23 | ENST00000280258 |
| 70 | PYHIN1 | ENST00000368140 |
| 71 | QRICH1 | ENST00000357496 |
| 72 | RAB32 | ENST00000367495 |
| 73 | RBM15 | ENST00000618772 |
| 74 | RBM23 | ENST00000399922 |
| 75 | RFC1 | ENST00000349703 |
| 76 | RNASE6 | ENST00000304677 |
| 77 | RUNX2 | ENST00000371432 |
| 78 | RYK | ENST00000623711 |
| 79 | SAP130 | ENST00000259235 |
| 80 | SEMA4D | ENST00000438547 |
| 81 | SIDT1 | ENST00000264852 |
| 82 | SMPDL3A | ENST00000368440 |
| 83 | SPIN1 | ENST00000375859 |
| 84 | ST3GAL2 | ENST00000342907 |
| 85 | SYTL2 | ENST00000389960 |
| 86 | TGFBR3 | ENST00000212355 |
| 87 | TLE3 | ENST00000558939 |
| 88 | TLR5 | ENST00000366881 |
| 89 | TMEM165 | ENST00000381334 |
| 90 | TSPO | ENST00000337554 |
| 91 | UTRN | ENST00000367545 |
| 92 | YPEL1 | ENST00000339468 |
| 93 | ZFP36L2 | ENST00000282388 |
| 94 | ZHX2 | ENST00000314393 |

TABLE 4
BASIRS BIOMARKER DETAILS INCLUDING;
SEQUENCE IDENTIFICATION NUMBER,
GENE SYMBOL, GENBANK ACCESSION
AND AMINO ACID SEQUENCE

| SEQ ID | Gene | GenBank |
|---|---|---|
| # AA | Symbol | Accession |
| 95 | ADAM19 | NP_150377 |
| 96 | ADM | NP_001115 |
| 97 | ALPL | NP_000469 |
| 98 | CAMK1D | NP_065130 |
| 99 | CASS4 | NP_065089 |
| 100 | CBLL1 | NP_079090 |
| 101 | CCNK | NP_001092872 |
| 102 | CD82 | NP_002222 |
| 103 | CLEC7A | NP_072092 |
| 104 | CNNM3 | NP_060093 |
| 105 | COX15 | NP_004367 |
| 106 | CR1 | NP_000564 |
| 107 | DENND3 | NP_055772 |
| 108 | DOCK5 | NP_079216 |
| 109 | ENTPD7 | NP_065087 |
| 110 | EPHB4 | NP_004435 |
| 111 | EXTL3 | NP_001431 |
| 112 | FAM129A | NP_443198 |
| 113 | FBXO28 | NP_055991 |
| 114 | FIG4 | NP_055660 |
| 115 | FOXJ3 | NP_055762 |
| 116 | GAB2 | NP_036428 |
| 117 | GALNT2 | NP_004472 |
| 118 | GAS7 | NP_003635 |
| 119 | GCC2 | NP_852118 |
| 120 | GRK5 | NP_005299 |
| 121 | HAL | NP_002099 |
| 122 | HCLS1 | NP_005326 |
| 123 | HK3 | NP_002106 |
| 124 | ICK | NP_055735 |
| 125 | IGFBP7 | NP_001544 |
| 126 | IK | NP_006074 |
| 127 | IKZF5 | NP_001258769 |
| 128 | IL2RB | NP_000869 |
| 129 | IMPDH1 | NP_000874 |
| 130 | INPP5D | NP_005532 |
| 131 | ITGA7 | NP_002197 |
| 132 | JARID2 | NP_004964 |
| 133 | KIAA0101 | NP_055551 |
| 134 | KIAA0355 | NP_055501 |
| 135 | KIAA0907 | NP_055764 |
| 136 | KLRD1 | NP_002253 |
| 137 | KLRF1 | NP_057607 |
| 138 | LAG3 | NP_002277 |
| 139 | LEPROTL1 | NP_056159 |
| 140 | LPIN2 | NP_055461 |
| 141 | MBIP | NP_057670 |
| 142 | MCTP1 | NP_078993 |
| 143 | MGAM | NP_004659 |
| 144 | MME | NP_000893 |
| 145 | NCOA6 | NP_054790 |
| 146 | NFIC | NP_005588 |
| 147 | NLRP1 | NP_055737 |
| 148 | NMUR1 | NP_006047 |
| 149 | NOV | NP_002505 |
| 150 | NPAT | NP_002510 |
| 151 | OPLAH | NP_060040 |
| 152 | PARP8 | NP_078891 |
| 153 | PCOLCE2 | NP_037495 |
| 154 | PDGFC | NP_057289 |
| 155 | PDS5B | NP_055847 |
| 156 | PHF3 | NP_055968 |
| 157 | PIK3C2A | NP_002636 |
| 158 | PLA2G7 | NP_005075 |
| 159 | POGZ | NP_055915 |
| 160 | PRKD2 | NP_057541 |
| 161 | PRKDC | NP_008835 |
| 162 | PRPF38B | NP_060531 |
| 163 | PRSS23 | NP_009104 |
| 164 | PYHIN1 | NP_689714 |
| 165 | QRICH1 | NP_060200 |
| 166 | RAB32 | NP_006825 |
| 167 | RBM15 | NP_073605 |
| 168 | RBM23 | NP_060577 |
| 169 | RFC1 | NP_002904 |
| 170 | RNASE6 | NP_005606 |
| 171 | RUNX2 | NP_001015051 |
| 172 | RYK | NP_002949 |
| 173 | SAP130 | NP_078821 |
| 174 | SEMA4D | NP_006369 |
| 175 | SIDT1 | NP_060169 |
| 176 | SMPDL3A | NP_006705 |
| 177 | SPIN1 | NP_006708 |
| 178 | ST3GAL2 | NP_008858 |
| 179 | SYTL2 | NP_116561 |
| 180 | TGFBR3 | NP_003234 |
| 181 | TLE3 | NP_005069 |
| 182 | TLR5 | NP_003259 |
| 183 | TMEM165 | NP_060945 |
| 184 | TSPO | NP_000705 |
| 185 | UTRN | NP_009055 |
| 186 | YPEL1 | NP_037445 |
| 187 | ZFP36L2 | NP_008818 |
| 188 | ZHX2 | NP_055758 |

TABLE 5
VASIRS BIOMARKER DETAILS INCLUDING;
SEQUENCE IDENTIFICATION NUMBER,
GENE SYMBOL, ENSEMBL TRANSCRIPT
ID AND DNA SEQUENCE

| SEQ ID | Gene | Ensembl Transcript |
|---|---|---|
| # (DNA) | Symbol | ID |
| 189 | ABAT | ENST00000396600 |
| 190 | ABHD2 | ENST00000565973 |
| 191 | ABI1 | ENST00000376142 |
| 192 | ABLIM1 | ENST00000277895 |
| 193 | ACAA1 | ENST00000333167 |
| 194 | ACAP2 | ENST00000326793 |
| 195 | ACVR1B | ENST00000257963 |
| 196 | AIF1 | ENST00000413349 |
| 197 | ALDH3A2 | ENST00000579855 |
| 198 | ANKRD49 | ENST00000544612 |
| 199 | AOAH | ENST00000617537 |
| 200 | APBB1IP | ENST00000376236 |
| 201 | APLP2 | ENST00000263574 |
| 202 | ARAP1 | ENST00000334211 |
| 203 | ARHGAP15 | ENST00000295095 |
| 204 | ARHGAP25 | ENST00000409030 |
| 205 | ARHGAP26 | ENST00000274498 |
| 206 | ARHGEF2 | ENST00000313695 |
| 207 | ARRB1 | ENST00000420843 |
| 208 | ARRB2 | ENST00000269260 |
| 209 | ASAP1 | ENST00000518721 |
| 210 | ATAD2B | ENST00000238789 |
| 211 | ATF7IP2 | ENST00000396560 |
| 212 | ATM | ENST00000278616 |
| 213 | ATP6V1B2 | ENST00000276390 |
| 214 | BACH1 | ENST00000286800 |
| 215 | BANP | ENST00000355022 |
| 216 | BAZ2B | ENST00000392783 |
| 217 | BCL2 | ENST00000398117 |
| 218 | BEX4 | ENST00000372695 |
| 219 | BMP2K | ENST00000502871 |
| 220 | BRD1 | ENST00000216267 |
| 221 | BRD4 | ENST00000371835 |
| 222 | BTG1 | ENST00000256015 |
| 223 | C19orf66 | ENST00000253110 |
| 224 | C2orf68 | ENST00000306336 |
| 225 | CAMK1D | ENST00000378845 |
| 226 | CAMK2G | ENST00000351293 |
| 227 | CAP1 | ENST00000372797 |
| 228 | CASC3 | ENST00000264645 |
| 229 | CASP8 | ENST00000264275 |
| 230 | CBX7 | ENST00000216133 |
| 231 | CCND3 | ENST00000372991 |
| 232 | CCNG2 | ENST00000316355 |
| 233 | CCNT2 | ENST00000295238 |
| 234 | CCR7 | ENST00000246657 |
| 235 | CD37 | ENST00000323906 |
| 236 | CD93 | ENST00000246006 |
| 237 | ADGRE5 (CD97) | ENST00000358600 |
| 238 | CDIPT | ENST00000219789 |
| 239 | CEP170 | ENST00000612450 |
| 240 | CEP68 | ENST00000377990 |
| 241 | CHD3 | ENST00000358181 |
| 242 | CHMP1B | ENST00000526991 |
| 243 | CHMP7 | ENST00000397677 |
| 244 | CHST11 | ENST00000303694 |
| 245 | CIAPIN1 | ENST00000394391 |
| 246 | CLEC4A | ENST00000229332 |
| 247 | CLK4 | ENST00000316308 |
| 248 | CNPY3 | ENST00000372836 |
| 249 | CREB1 | ENST00000353267 |
| 250 | CREBBP | ENST00000262367 |
| 251 | CRLF3 | ENST00000324238 |
| 252 | CRTC3 | ENST00000268184 |
| 253 | CSAD | ENST00000267085 |
| 254 | CSF2RB | ENST00000403662 |
| 255 | CSNK1D | ENST00000314028 |
| 256 | CST3 | ENST00000376925 |
| 257 | CTBP2 | ENST00000337195 |
| 258 | CTDSP2 | ENST00000398073 |
| 259 | CUL1 | ENST00000325222 |
| 260 | CYLD | ENST00000311559 |
| 261 | CYTH4 | ENST00000248901 |
| 262 | DCP2 | ENST00000389063 |
| 263 | DDX60 | ENST00000393743 |
| 264 | DGCR2 | ENST00000263196 |
| 265 | DGKA | ENST00000331886 |
| 266 | DHX58 | ENST00000251642 |
| 267 | DIDO1 | ENST00000370371 |
| 268 | DOCK9 | ENST00000376460 |
| 269 | DOK3 | ENST00000357198 |
| 270 | DPEP2 | ENST00000393847 |
| 271 | DPF2 | ENST00000528416 |
| 272 | EIF2AK2 | ENST00000395127 |
| 273 | EIF3H | ENST00000521861 |
| 274 | EMR2 | ENST00000315576 |
| 275 | ERBB2IP | ENST00000380943 |
| 276 | ETS2 | ENST00000360938 |
| 277 | FAIM3 | ENST00000367091 |
| 278 | FAM134A | ENST00000430297 |
| 279 | FAM65B | ENST00000259698 |
| 280 | FBXO11 | ENST00000402508 |
| 281 | FBXO9 | ENST00000244426 |
| 282 | FCGRT | ENST00000426395 |
| 283 | FES | ENST00000328850 |
| 284 | FGR | ENST00000374005 |
| 285 | FLOT2 | ENST00000394908 |
| 286 | FNBP1 | ENST00000446176 |
| 287 | FOXJ2 | ENST00000162391 |
| 288 | FOXO1 | ENST00000379561 |
| 289 | FOXO3 | ENST00000406360 |
| 290 | FRY | ENST00000542859 |
| 291 | FYB | ENST00000505428 |
| 292 | GABARAP | ENST00000302386 |
| 293 | GCC2 | ENST00000309863 |
| 294 | GMIP | ENST00000203556 |
| 295 | GNA12 | ENST00000275364 |
| 296 | GNAQ | ENST00000286548 |
| 297 | GOLGA7 | ENST00000520817 |
| 298 | GPBP1L1 | ENST00000355105 |
| 299 | GPR97 | ENST00000333493 |
| 300 | GPS2 | ENST00000389167 |
| 301 | GPSM3 | ENST00000383269 |
| 302 | GRB2 | ENST00000316804 |
| 303 | GSK3B | ENST00000316626 |
| 304 | GYPC | ENST00000259254 |
| 305 | HAL | ENST00000261208 |
| 306 | HCK | ENST00000534862 |
| 307 | HERC5 | ENST00000264350 |
| 308 | HERC6 | ENST00000264346 |
| 309 | HGSNAT | ENST00000379644 |
| 310 | HHEX | ENST00000282728 |
| 311 | HIP1 | ENST00000336926 |
| 312 | HPCAL1 | ENST00000307845 |
| 313 | HPS1 | ENST00000325103 |
| 314 | ICAM3 | EN5T00000160262 |
| 315 | IFI44 | ENST00000370747 |
| 316 | IFI6 | ENST00000361157 |
| 317 | IFIH1 | ENST00000263642 |
| 318 | IGSF6 | ENST00000268389 |
| 319 | IKBKB | ENST00000520810 |
| 320 | IL10RB | ENST00000290200 |
| 321 | IL13RA1 | ENST00000371666 |
| 322 | IL16 | ENST00000394652 |
| 323 | IL1RAP | ENST00000447382 |
| 324 | IL27RA | ENST00000263379 |
| 325 | IL4R | ENST00000395762 |
| 326 | IL6R | ENST00000368485 |
| 327 | IL6ST | ENST00000381298 |
| 328 | INPP5D | ENST00000359570 |
| 329 | IQSEC1 | ENST00000273221 |
| 330 | ISG15 | ENST00000379389 |
| 331 | ITGAX | ENST00000268296 |
| 332 | ITGB2 | ENST00000302347 |
| 333 | ITPKB | ENST00000429204 |
| 334 | ITSN2 | ENST00000355123 |
| 335 | JAK1 | ENST00000342505 |
| 336 | KBTBD2 | ENST00000304056 |
| 337 | KIAA0232 | ENST00000307659 |
| 338 | KIAA0247 | ENST00000342745 |
| 339 | KIAA0513 | ENST00000258180 |
| 340 | KLF3 | ENST00000261438 |
| 341 | KLF6 | ENST00000497571 |
| 342 | KLF7 | ENST00000309446 |
| 343 | KLHL2 | ENST00000226725 |
| 344 | LAP3 | ENST00000618908 |
| 345 | LAPTM5 | ENST00000294507 |
| 346 | LAT2 | ENST00000344995 |
| 347 | LCP2 | ENST00000046794 |
| 348 | LDLRAP1 | ENST00000374338 |
| 349 | LEF1 | ENST00000265165 |
| 350 | LILRA2 | ENST00000251376 |
| 351 | LILRB3 | ENST00000617251 |
| 352 | LIMK2 | ENST00000331728 |
| 353 | LPAR2 | ENST00000407877 |
| 354 | LPIN2 | ENST00000261596 |
| 355 | LRMP | ENST00000354454 |
| 356 | LRP10 | ENST00000359591 |
| 357 | LST1 | ENST00000376093 |
| 358 | LTB | ENST00000429299 |
| 359 | LYL1 | ENST00000264824 |
| 360 | LYN | ENST00000519728 |
| 361 | LYST | ENST00000389793 |
| 362 | MAML1 | ENST00000292599 |
| 363 | MANSC1 | ENST00000535902 |
| 364 | MAP1LC3B | ENST00000268607 |
| 365 | MAP3K11 | ENST00000309100 |
| 366 | MAP3K3 | ENST00000361733 |
| 367 | MAP3K5 | ENST00000359015 |
| 368 | MAP4K4 | ENST00000350198 |
| 369 | MAPK1 | ENST00000215832 |
| 370 | MAPK14 | ENST00000229795 |
| 371 | MAPRE2 | ENST00000300249 |
| 372 | MARCH7 | ENST00000259050 |
| 373 | MARCH8 | ENST00000319836 |
| 374 | MARK3 | ENST00000303622 |
| 375 | MAST3 | ENST00000262811 |
| 376 | MAX | ENST00000358664 |
| 377 | MBP | ENST00000359645 |
| 378 | MCTP2 | ENST00000357742 |
| 379 | MED13 | ENST00000397786 |
| 380 | MEF2A | ENST00000354410 |
| 381 | METTL3 | ENST00000298717 |
| 382 | MKLN1 | ENST00000352689 |
| 383 | MKRN1 | ENST00000255977 |
| 384 | MMP25 | ENST00000336577 |
| 385 | MORC3 | ENST00000400485 |
| 386 | MOSPD2 | ENST00000380492 |
| 387 | MPPE1 | ENST00000588072 |
| 388 | MSL1 | ENST00000579565 |
| 389 | MTMR3 | ENST00000401950 |
| 390 | MX1 | ENST00000398598 |
| 391 | MXI1 | ENST00000239007 |
| 392 | MYC | ENST00000613283 |
| 393 | N4BP1 | ENST00000262384 |
| 394 | NAB1 | ENST00000337386 |
| 395 | NACA | ENST00000356769 |
| 396 | NCBP2 | ENST00000321256 |
| 397 | NCOA1 | ENST00000348332 |
| 398 | NCOA4 | ENST00000585132 |
| 399 | NDE1 | ENST00000396354 |
| 400 | NDEL1 | ENST00000334527 |
| 401 | NDFIP1 | ENST00000253814 |
| 402 | NECAP2 | ENST00000337132 |
| 403 | NEK7 | ENST00000367385 |
| 404 | NFKB1 | ENST00000226574 |
| 405 | NFYA | ENST00000341376 |
| 406 | NLRP1 | ENST00000269280 |
| 407 | NOD2 | ENST00000300589 |
| 408 | NOSIP | ENST00000596358 |
| 409 | NPL | ENST00000367553 |
| 410 | NR3C1 | ENST00000394464 |
| 411 | NRBF2 | ENST00000277746 |
| 412 | NSUN3 | ENST00000314622 |
| 413 | NUMB | ENST00000557597 |
| 414 | OAS2 | ENST00000392583 |
| 415 | OASL | ENST00000257570 |
| 416 | OGFRL1 | ENST00000370435 |
| 417 | OSBPL11 | ENST00000296220 |
| 418 | OSBPL2 | ENST00000358053 |
| 419 | PACSIN2 | ENST00000403744 |
| 420 | PAFAH1B1 | ENST00000397195 |
| 421 | PARP12 | ENST00000263549 |
| 422 | PBX3 | ENST00000373489 |
| 423 | PCBP2 | ENST00000359462 |
| 424 | PCF11 | ENST00000298281 |
| 425 | PCNX | ENST00000304743 |
| 426 | PDCD6IP | ENST00000307296 |
| 427 | PDE3B | ENST00000282096 |
| 428 | PECAM1 | ENST00000563924 |
| 429 | PFDN5 | ENST00000551018 |
| 430 | PGS1 | ENST00000262764 |
| 431 | PHC2 | ENST00000373418 |
| 432 | PHF11 | ENST00000378319 |
| 433 | PHF2 | ENST00000359246 |
| 434 | PHF20 | ENST00000374012 |
| 435 | PHF20L1 | ENST00000395386 |
| 436 | PHF3 | ENST00000393387 |
| 437 | PIAS1 | ENST00000249636 |
| 438 | PIK3IP1 | ENST00000215912 |
| 439 | PINK1 | ENST00000321556 |
| 440 | PISD | ENST00000266095 |
| 441 | PITPNA | ENST00000313486 |
| 442 | PLEKHO1 | ENST00000369124 |
| 443 | PLEKHO2 | ENST00000323544 |
| 444 | PLXNC1 | ENST00000258526 |
| 445 | POLB | ENST00000265421 |
| 446 | POLD4 | ENST00000312419 |
| 447 | POLR1D | ENST00000302979 |
| 448 | PPARD | ENST00000360694 |
| 449 | PPM1F | ENST00000263212 |
| 450 | PPP1R11 | ENST00000448378 |
| 451 | PPP1R2 | ENST00000618156 |
| 452 | PPP2R5A | ENST00000261461 |
| 453 | PPP3R1 | ENST00000234310 |
| 454 | PPP4R1 | ENST00000400555 |
| 455 | PRKAA1 | ENST00000397128 |
| 456 | PRKAG2 | ENST00000287878 |
| 457 | PRKCD | ENST00000330452 |
| 458 | PRMT2 | ENST00000397638 |
| 459 | PRUNE | ENST00000271620 |
| 460 | PSAP | ENST00000394936 |
| 461 | PSEN1 | ENST00000324501 |
| 462 | PSTPIP1 | ENST00000558012 |
| 463 | PTAFR | ENST00000373857 |
| 464 | PTEN | ENST00000371953 |
| 465 | PTGER4 | ENST00000302472 |
| 466 | PTPN6 | ENST00000318974 |
| 467 | PTPRE | ENST00000254667 |
| 468 | PUM2 | ENST00000338086 |
| 469 | R3HDM2 | ENST00000358907 |
| 470 | RAB11FIP1 | ENST00000287263 |
| 471 | RAB14 | ENST00000373840 |
| 472 | RAB31 | ENST00000578921 |
| 473 | RAB4B | ENST00000357052 |
| 474 | RAB7A | ENST00000265062 |
| 475 | RAF1 | ENST00000251849 |
| 476 | RALB | ENST00000272519 |
| 477 | RARA | ENST00000254066 |
| 478 | RASSF2 | ENST00000379400 |
| 479 | RBM23 | ENST00000399922 |
| 480 | RBMS1 | ENST00000348849 |
| 481 | RC3H2 | ENST00000423239 |
| 482 | RERE | ENST00000337907 |
| 483 | RGS14 | ENST00000408923 |
| 484 | RGS19 | ENST00000395042 |
| 485 | RHOG | ENST00000351018 |
| 486 | RIN3 | ENST00000216487 |
| 487 | RNASET2 | ENST00000508775 |
| 488 | RNF130 | EN5T00000521389 |
| 489 | RNF141 | ENST00000265981 |
| 490 | RNF146 | ENST00000608991 |
| 491 | RNF19B | ENST00000373456 |
| 492 | RPL10A | ENST00000322203 |
| 493 | RPL22 | ENST00000234875 |
| 494 | RPS6KA1 | ENST00000374168 |
| 495 | RPS6KA3 | ENST00000379565 |
| 496 | RSAD2 | ENST00000382040 |
| 497 | RTN3 | ENST00000537981 |
| 498 | RTP4 | ENST00000259030 |
| 499 | RXRA | ENST00000481739 |
| 500 | RYBP | ENST00000477973 |
| 501 | SAFB2 | ENST00000252542 |
| 502 | SATB1 | ENST00000338745 |
| 503 | SEC62 | ENST00000337002 |
| 504 | SEMA4D | ENST00000438547 |
| 505 | SERINC3 | ENST00000342374 |
| 506 | SERINC5 | ENST00000509193 |
| 507 | SERTAD2 | ENST00000313349 |
| 508 | SESN1 | ENST00000436639 |
| 509 | SETD2 | ENST00000409792 |
| 510 | SH2B3 | ENST00000341259 |
| 511 | SH2D3C | ENST00000373277 |
| 512 | SIRPA | ENST00000356025 |
| 513 | SIRPB1 | ENST00000381605 |
| 514 | SLCO3A1 | ENST00000318445 |
| 515 | SMAD4 | ENST00000342988 |
| 516 | SNN | ENST00000329565 |
| 517 | SNRK | ENST00000296088 |
| 518 | SNX27 | ENST00000368843 |
| 519 | SOATI | ENST00000367619 |
| 520 | SORL1 | ENST00000260197 |
| 521 | SOS2 | ENST00000216373 |
| 522 | SP3 | ENST00000310015 |
| 523 | SSBP2 | ENST00000320672 |
| 524 | SSFA2 | ENST00000320370 |
| 525 | ST13 | ENST00000216218 |
| 526 | ST3GAL1 | ENST00000521180 |
| 527 | STAM2 | ENST00000263904 |
| 528 | STAT1 | ENST00000361099 |
| 529 | STAT5A | ENST00000345506 |
| 530 | STAT5B | ENST00000293328 |
| 531 | STK38L | ENST00000389032 |
| 532 | STX10 | ENST00000587230 |
| 533 | STX3 | ENST00000337979 |
| 534 | STX6 | ENST00000258301 |
| 535 | SYPL1 | ENST00000011473 |
| 536 | TAP1 | ENST00000428324 |
| 537 | TFE3 | ENST00000315869 |
| 538 | TFEB | ENST00000230323 |
| 539 | TGFBI | ENST00000442011 |
| 540 | TGFBR2 | ENST00000295754 |
| 541 | TGOLN2 | ENST00000377386 |
| 542 | TIAM1 | ENST00000286827 |
| 543 | TLE3 | ENST00000558939 |
| 544 | TLE4 | ENST00000376552 |
| 545 | TLR2 | ENST00000260010 |
| 546 | TM2D3 | ENST00000347970 |
| 547 | TMBIM1 | ENST00000258412 |
| 548 | TMEM127 | ENST00000258439 |
| 549 | TMEM204 | ENST00000566264 |
| 550 | TNFRSF1A | ENST00000162749 |
| 551 | TNFSF13 | ENST00000338784 |
| 552 | TNIP1 | ENST00000521591 |
| 553 | TNK2 | ENST00000333602 |
| 554 | TNRC6B | ENST00000335727 |
| 555 | TOPORS | ENST00000360538 |
| 556 | TRAK1 | ENST00000341421 |
| 557 | TREM1 | ENST00000244709 |
| 558 | TRIB2 | ENST00000155926 |
| 559 | TRIM8 | ENST00000302424 |
| 560 | TRIOBP | ENST00000403663 |
| 561 | TSC22D3 | ENST00000372397 |
| 562 | TYK2 | ENST00000525621 |
| 563 | TYROBP | ENST00000262629 |
| 564 | UBE2D2 | ENST00000398733 |
| 565 | UBE2L6 | ENST00000287156 |
| 566 | UBN1 | ENST00000262376 |
| 567 | UBQLN2 | ENST00000338222 |
| 568 | UBXN2B | ENST00000399598 |
| 569 | USP10 | ENST00000219473 |
| 570 | USP15 | ENST00000353364 |
| 571 | USP18 | ENST00000215794 |
| 572 | USP4 | ENST00000265560 |
| 573 | UTP14A | ENST00000394422 |
| 574 | VAMP3 | ENST00000054666 |
| 575 | VAV3 | EN5T00000370056 |
| 576 | VEZF1 | ENST00000581208 |
| 577 | VPS8 | ENST00000436792 |
| 578 | WASF2 | ENST00000618852 |
| 579 | WBP2 | ENST00000254806 |
| 580 | WDR37 | ENST00000263150 |
| 581 | WDR47 | ENST00000369965 |
| 582 | XAF1 | ENST00000361842 |
| 583 | XPC | ENST00000285021 |
| 584 | XPO6 | ENST00000304658 |
| 585 | YPEL5 | ENST00000261353 |
| 586 | YTHDF3 | ENST00000539294 |
| 588 | ZBTB18 | ENST00000622512 |
| 589 | ZC3HAV1 | ENST00000242351 |
| 590 | ZDHHC17 | ENST00000426126 |
| 591 | ZDHHC18 | ENST00000374142 |
| 592 | ZFAND5 | ENST00000376960 |
| 593 | ZFC3H1 | ENST00000378743 |
| 594 | ZFYVE16 | ENST00000338008 |
| 595 | ZMIZ1 | ENST00000334512 |
| 596 | ZNF143 | ENST00000396602 |
| 597 | ZNF148 | ENST00000360647 |
| 598 | ZNF274 | ENST00000424679 |
| 599 | ZNF292 | ENST00000369577 |
| 600 | ZXDC | ENST00000389709 |
| 601 | ZYX | ENST00000322764 |

TABLE 6
VASIRS BIOMARKER DETAILS INCLUDING;
SEQUENCE IDENTIFICATION NUMBER,
GENE SYMBOL, GENBANK ACCESSION
AND AMINO ACID SEQUENCE

| SEQ ID | Gene | GenBank |
|---|---|---|
| # (AA) | Symbol | Accession |
| 602 | ABAT | NP_000654 |
| 603 | ABHD2 | NP_008942 |
| 604 | ABI1 | NP_005461 |
| 605 | ABLIM1 | NP_002304 |
| 606 | ACAA1 | NP_001598 |
| 607 | ACAP2 | NP_036419 |
| 608 | ACVR1B | NP_004293 |
| 609 | AIF1 | NP_001614 |
| 610 | ALDH3A2 | NP_000373 |
| 611 | ANKRD49 | NP_060174 |
| 612 | AOAH | NP_001628 |
| 613 | APBB1IP | NP_061916 |
| 614 | APLP2 | NP_001633 |
| 615 | ARAP1 | NP_056057 |
| 616 | ARHGAP15 | NP_060930 |
| 617 | ARHGAP25 | NP_055697 |
| 618 | ARHGAP26 | NP_055886 |
| 619 | ARHGEF2 | NP_004714 |
| 620 | ARRB1 | NP_004032 |
| 621 | ARRB2 | NP_004304 |
| 622 | ASAP1 | NP_060952 |
| 623 | ATAD2B | NP_060022 |
| 624 | ATF7IP2 | NP_079273 |
| 625 | ATM | NP_000042 |
| 626 | ATP6V1B2 | NP_001684 |
| 627 | BACH1 | NP_001177 |
| 628 | BANP | NP_060339 |
| 629 | BAZ2B | NP_038478 |
| 630 | BCL2 | NP_000624 |
| 631 | BEX4 | NP_001073894 |
| 632 | BMP2K | NP_060063 |
| 633 | BRD1 | NP_055392 |
| 634 | BRD4 | NP_055114 |
| 635 | BTG1 | NP_001722 |
| 636 | C19orf66 | NP_060851 |
| 637 | C2orf68 | NP_001013671 |
| 638 | CAMK1D | NP_065130 |
| 639 | CAMK2G | NP_001213 |
| 640 | CAP1 | NP_006358 |
| 641 | CASC3 | NP_031385 |
| 642 | CASP8 | NP_001219 |
| 643 | CBX7 | NP_783640 |
| 644 | CCND3 | NP_001751 |
| 645 | CCNG2 | NP_004345 |
| 646 | CCNT2 | NP_001232 |
| 647 | CCR7 | NP_001829 |
| 648 | CD37 | NP_001765 |
| 649 | CD93 | NP_036204 |
| 650 | ADGRE5 (CD97) | NP_001775 |
| 651 | CDIPT | NP_006310 |
| 652 | CEP170 | NP_055627 |
| 653 | CEP68 | NP_055962 |
| 654 | CHD3 | NP_005843 |
| 655 | CHMP1B | NP_065145 |
| 656 | CHMP7 | NP_689485 |
| 657 | CHST11 | NP_060883 |
| 658 | CIAPIN1 | NP_064709 |
| 659 | CLEC4A | NP_057268 |
| 660 | CLK4 | NP_065717 |
| 661 | CNPY3 | NP_006577 |
| 662 | CREB1 | NP_004370 |
| 663 | CREBBP | NP_004371 |
| 664 | CRLF3 | NP_057070 |
| 665 | CRTC3 | NP_073606 |
| 666 | CSAD | NP_057073 |
| 667 | CSF2RB | NP_000386 |
| 668 | CSNK1D | NP_001884 |
| 669 | CST3 | NP_000090 |
| 670 | CTBP2 | NP_001320 |
| 671 | CTDSP2 | NP_005721 |
| 672 | CUL1 | NP_003583 |
| 673 | CYLD | NP_056062 |
| 674 | CYTH4 | NP_037517 |
| 675 | DCP2 | NP_689837 |
| 676 | DDX60 | NP_060101 |
| 677 | DGCR2 | NP_005128 |
| 678 | DGKA | NP_001336 |
| 679 | DHX58 | NP_077024 |
| 680 | DIDO1 | NP_071388 |
| 681 | DOCK9 | NP_056111 |
| 682 | DOK3 | NP_079148 |
| 683 | DPEP2 | NP_071750 |
| 684 | DPF2 | NP_006259 |
| 685 | EIF2AK2 | NP_002750 |
| 686 | EIF3H | NP_003747 |
| 687 | EMR2 | NP_038475 |
| 688 | ERBB2IP | NP_061165 |
| 689 | ETS2 | NP_005230 |
| 690 | FAIM3 | NP_005440 |
| 691 | FAM134A | NP_077269 |
| 692 | FAM65B | NP_055537 |
| 693 | FBXO11 | NP_079409 |
| 694 | FBXO9 | NP_036479 |
| 695 | FCGRT | NP_004098 |
| 696 | FES | NP_001996 |
| 697 | FGR | NP_005239 |
| 698 | FLOT2 | NP_004466 |
| 699 | FNBP1 | NP_055848 |
| 700 | FOXJ2 | NP_060886 |
| 701 | FOXO1 | NP_002006 |
| 702 | FOXO3 | NP_001446 |
| 703 | FRY | NP_075463 |
| 704 | FYB | NP_001456 |
| 705 | GABARAP | NP_009209 |
| 706 | GCC2 | NP_852118 |
| 707 | GMIP | NP_057657 |
| 708 | GNA12 | NP_031379 |
| 709 | GNAQ | NP_002063 |
| 710 | GOLGA7 | NP_057183 |
| 711 | GPBP1L1 | NP_067652 |
| 712 | GPR97 | NP_740746 |
| 713 | GPS2 | NP_004480 |
| 714 | GPSM3 | NP_071390 |
| 715 | GRB2 | NP_002077 |
| 716 | GSK3B | NP_002084 |
| 717 | GYPC | NP_002092 |
| 718 | HAL | NP_002099 |
| 719 | HCK | NP_002101 |
| 720 | HERC5 | NP_057407 |
| 721 | HERC6 | NP_060382 |
| 722 | HGSNAT | NP_689632 |
| 723 | HHEX | NP_002720 |
| 724 | HIP1 | NP_005329 |
| 725 | HPCAL1 | NP_002140 |
| 726 | HPS1 | NP_000186 |
| 727 | ICAM3 | NP_002153 |
| 728 | IFI44 | NP_006408 |
| 729 | IFI6 | NP_002029 |
| 730 | IFIH1 | NP_071451 |
| 731 | IGSF6 | NP_005840 |
| 732 | IKBKB | NP_001547 |
| 733 | IL10RB | NP_000619 |
| 734 | IL13RA1 | NP_001551 |
| 735 | IL16 | NP_004504 |
| 736 | IL1RAP | NP_002173 |
| 737 | IL27RA | NP_004834 |
| 738 | IL4R | NP_000409 |
| 739 | IL6R | NP_000556 |
| 740 | IL6ST | NP_002175 |
| 741 | INPP5D | NP_005532 |
| 742 | IQSEC1 | NP_055684 |
| 743 | ISG15 | NP_005092 |
| 744 | ITGAX | NP_000878 |
| 745 | ITGB2 | NP_000202 |
| 746 | ITPKB | NP_002212 |
| 747 | ITSN2 | NP_006268 |
| 748 | JAK1 | NP_002218 |
| 749 | KBTBD2 | NP_056298 |
| 750 | KIAA0232 | NP_055558 |
| 751 | KIAA0247 | NP_055549 |
| 752 | KIAA0513 | NP_055547 |
| 753 | KLF3 | NP_057615 |
| 754 | KLF6 | NP_001291 |
| 755 | KLF7 | NP_003700 |
| 756 | KLHL2 | NP_009177 |
| 757 | LAP3 | NP_056991 |
| 758 | LAPTM5 | NP_006753 |
| 759 | LAT2 | NP_054865 |
| 760 | LCP2 | NP_005556 |
| 761 | LDLRAP1 | NP_056442 |
| 762 | LEF1 | NP_057353 |
| 763 | LILRA2 | NP_006857 |
| 764 | LILRB3 | NP_006855 |
| 765 | LIMK2 | NP_005560 |
| 766 | LPAR2 | NP_004711 |
| 767 | LPIN2 | NP_055461 |
| 768 | LRMP | NP_006143 |
| 769 | LRP10 | NP_054764 |
| 770 | LST1 | NP_009092 |
| 771 | LTB | NP_002332 |
| 772 | LYL1 | NP_005574 |
| 773 | LYN | NP_002341 |
| 774 | LYST | NP_000072 |
| 775 | MAML1 | NP_055572 |
| 776 | MANSC1 | NP_060520 |
| 777 | MAP1LC3B | NP_073729 |
| 778 | MAP3K11 | NP_002410 |
| 779 | MAP3K3 | NP_002392 |
| 780 | MAP3K5 | NP_005914 |
| 781 | MAP4K4 | NP_004825 |
| 782 | MAPK1 | NP_002736 |
| 783 | MAPK14 | NP_001306 |
| 784 | MAPRE2 | NP_055083 |
| 785 | MARCH7 | NP_073737 |
| 786 | MARCH8 | NP_659458 |
| 787 | MARK3 | NP_002367 |
| 788 | MAST3 | NP_055831 |
| 789 | MAX | NP_002373 |
| 790 | MBP | NP_002376 |
| 791 | MCTP2 | NP_060819 |
| 792 | MED13 | NP_005112 |
| 793 | MEF2A | NP_005578 |
| 794 | METTL3 | NP_062826 |
| 795 | MKLN1 | NP_037387 |
| 796 | MKRN1 | NP_038474 |
| 797 | MMP25 | NP_071913 |
| 798 | MORC3 | NP_056173 |
| 799 | MOSPD2 | NP_689794 |
| 800 | MPPE1 | NP_075563 |
| 801 | MSL1 | NP_001012241 |
| 802 | MTMR3 | NP_066576 |
| 803 | MX1 | NP_002453 |
| 804 | MXI1 | NP_005953 |
| 805 | MYC | NP_002458 |
| 806 | N4BP1 | NP_694574 |
| 807 | NAB1 | NP_005957 |
| 808 | NACA | NP_001106673 |
| 809 | NCBP2 | NP_031388 |
| 810 | NCOA1 | NP_003734 |
| 811 | NCOA4 | NP_005428 |
| 812 | NDE1 | NP_060138 |
| 813 | NDEL1 | NP_110435 |
| 814 | NDFIP1 | NP_085048 |
| 815 | NECAP2 | NP_060560 |
| 816 | NEK7 | NP_598001 |
| 817 | NFKB1 | NP_003989 |
| 818 | NFYA | NP_002496 |
| 819 | NLRP1 | NP_055737 |
| 820 | NOD2 | NP_071445 |
| 821 | NOSIP | NP_057037 |
| 822 | NPL | NP_110396 |
| 823 | NR3C1 | NP_000167 |
| 824 | NRBF2 | NP_110386 |
| 825 | NSUN3 | NP_071355 |
| 826 | NUMB | NP_003735 |
| 827 | OAS2 | NP_002526 |
| 828 | OASL | NP_003724 |
| 829 | OGFRL1 | NP_078852 |
| 830 | OSBPL11 | NP_073613 |
| 831 | OSBPL2 | NP_055650 |
| 832 | PACSIN2 | NP_009160 |
| 833 | PAFAH1B1 | NP_000421 |
| 834 | PARP12 | NP_073587 |
| 835 | PBX3 | NP_006186 |
| 836 | PCBP2 | NP_005007 |
| 837 | PCF11 | NP_056969 |
| 838 | PCNX | NP_055797 |
| 839 | PDCD6IP | NP_037506 |
| 840 | PDE3B | NP_000913 |
| 841 | PECAM1 | NP_000433 |
| 842 | PFDN5 | NP_002615 |
| 843 | PGS1 | NP_077733 |
| 844 | PHC2 | NP_004418 |
| 845 | PHF11 | NP_001035533 |
| 846 | PHF2 | NP_005383 |
| 847 | PHF20 | NP_057520 |
| 848 | PHF20L1 | NP_057102 |
| 849 | PHF3 | NP_055968 |
| 850 | PIAS1 | NP_057250 |
| 851 | PIK3IP1 | NP_443112 |
| 852 | PINK1 | NP_115785 |
| 853 | PISD | NP_055153 |
| 854 | PITPNA | NP_006215 |
| 855 | PLEKHO1 | NP_057358 |
| 856 | PLEKHO2 | NP_079477 |
| 857 | PLXNC1 | NP_005752 |
| 858 | POLB | NP_002681 |
| 859 | POLD4 | NP_066996 |
| 860 | POLR1D | NP_057056 |
| 861 | PPARD | NP_006229 |
| 862 | PPM1F | NP_055449 |
| 863 | PPP1R11 | NP_068778 |
| 864 | PPP1R2 | NP_006232 |
| 865 | PPP2R5A | NP_006234 |
| 866 | PPP3R1 | NP_000936 |
| 867 | PPP4R1 | NP_005125 |
| 868 | PRKAA1 | NP_006242 |
| 869 | PRKAG2 | NP_057287 |
| 870 | PRKCD | NP_006245 |
| 871 | PRMT2 | NP_001526 |
| 872 | PRUNE | NP_067045 |
| 873 | PSAP | NP_002769 |
| 874 | PSEN1 | NP_000012 |
| 875 | PSTPIP1 | NP_003969 |
| 876 | PTAFR | NP_000943 |
| 877 | PTEN | NP_000305 |
| 878 | PTGER4 | NP_000949 |
| 879 | PTPN6 | NP_002822 |
| 880 | PTPRE | NP_006495 |
| 881 | PUM2 | NP_056132 |
| 882 | R3HDM2 | NP_055740 |
| 883 | RAB11FIP1 | NP_079427 |
| 884 | RAB14 | NP_057406 |
| 885 | RAB31 | NP_006859 |
| 886 | RAB4B | NP_057238 |
| 887 | RAB7A | NP_004628 |
| 888 | RAF1 | NP_002871 |
| 889 | RALB | NP_002872 |
| 890 | RARA | NP_000955 |
| 891 | RASSF2 | NP_055552 |
| 892 | RBM23 | NP_060577 |
| 893 | RBMS1 | NP_002888 |
| 894 | RC3H2 | NP_061323 |
| 895 | RERE | NP_036234 |
| 896 | RGS14 | NP_006471 |
| 897 | RGS19 | NP_005864 |
| 898 | RHOG | NP_001656 |
| 899 | RIN3 | NP_079108 |
| 900 | RNASET2 | NP_003721 |
| 901 | RNF130 | NP_060904 |
| 902 | RNF141 | NP_057506 |
| 903 | RNF146 | NP_112225 |
| 904 | RNF19B | NP_699172 |
| 905 | RPL10A | NP_009035 |
| 906 | RPL22 | NP_000974 |
| 907 | RPS6KA1 | NP_002944 |
| 908 | RPS6KA3 | NP_004577 |
| 909 | RSAD2 | NP_542388 |
| 910 | RTN3 | NP_006045 |
| 911 | RTP4 | NP_071430 |
| 912 | RXRA | NP_002948 |
| 913 | RYBP | NP_036366 |
| 914 | SAFB2 | NP_055464 |
| 915 | SATB1 | NP_002962 |
| 916 | SEC62 | NP_003253 |
| 917 | SEMA4D | NP_006369 |
| 918 | SERINC3 | NP_006802 |
| 919 | SERINC5 | NP_840060 |
| 920 | SERTAD2 | NP_055570 |
| 921 | SESN1 | NP_055269 |
| 922 | SETD2 | NP_054878 |
| 923 | SH2B3 | NP_005466 |
| 924 | SH2D3C | NP_005480 |
| 925 | SIRPA | NP_542970 |
| 926 | SIRPB1 | NP_006056 |
| 927 | SLCO3A1 | NP_037404 |
| 928 | SMAD4 | NP_005350 |
| 929 | SNN | NP_003489 |
| 930 | SNRK | NP_060189 |
| 931 | SNX27 | NP_112180 |
| 932 | SOAT1 | NP_003092 |
| 933 | SORL1 | NP_003096 |
| 934 | SOS2 | NP_008870 |
| 935 | SP3 | NP_003102 |
| 936 | SSBP2 | NP_036578 |
| 937 | SSFA2 | NP_006742 |
| 938 | ST13 | NP_003923 |
| 939 | ST3GAL1 | NP_003024 |
| 940 | STAM2 | NP_005834 |
| 941 | STAT1 | NP_009330 |
| 942 | STAT5A | NP_003143 |
| 943 | STAT5B | NP_036580 |
| 944 | STK38L | NP_055815 |
| 945 | STX10 | NP_003756 |
| 946 | STX3 | NP_004168 |
| 947 | STX6 | NP_005810 |
| 948 | SYPL1 | NP_006745 |
| 949 | TAP1 | NP_000584 |
| 950 | TFE3 | NP_006512 |
| 951 | TFEB | NP_009093 |
| 952 | TGFBI | NP_000349 |
| 953 | TGFBR2 | NP_003233 |
| 954 | TGOLN2 | NP_006455 |
| 955 | TIAM1 | NP_003244 |
| 956 | TLE3 | NP_005069 |
| 957 | TLE4 | NP_008936 |
| 958 | TLR2 | NP_003255 |
| 959 | TM2D3 | NP_079417 |
| 960 | TMBIM1 | NP_071435 |
| 961 | TMEM127 | NP_060319 |
| 962 | TMEM204 | NP_078876 |
| 963 | TNFRSF1A | NP_001056 |
| 964 | TNFSF13 | NP_003799 |
| 965 | TNIP1 | NP_006049 |
| 966 | TNK2 | NP_005772 |
| 967 | TNRC6B | NP_055903 |
| 968 | TOPORS | NP_005793 |
| 969 | TRAK1 | NP_055780 |
| 970 | TREM1 | NP_061113 |
| 971 | TRIB2 | NP_067675 |
| 972 | TRIM8 | NP_112174 |
| 973 | TRIOBP | NP_008963 |
| 974 | TSC22D3 | NP_004080 |
| 975 | TYK2 | NP_003322 |
| 976 | TYROBP | NP_003323 |
| 977 | UBE2D2 | NP_003330 |
| 978 | UBE2L6 | NP_004214 |
| 979 | UBN1 | NP_001072982 |
| 980 | UBQLN2 | NP_038472 |
| 981 | UBXN2B | NP_001071087 |
| 982 | USP10 | NP_005144 |
| 983 | USP15 | NP_006304 |
| 984 | USP18 | NP_059110 |
| 985 | USP4 | NP_003354 |
| 986 | UTP14A | NP_006640 |
| 987 | VAMP3 | NP_004772 |
| 988 | VAV3 | NP_006104 |
| 989 | VEZF1 | NP_009077 |
| 990 | VPS8 | NP_056118 |
| 991 | WASF2 | NP_008921 |
| 992 | WBP2 | NP_036610 |
| 993 | WDR37 | NP_054742 |
| 994 | WDR47 | NP_055784 |
| 995 | XAF1 | NP_059993 |
| 996 | XPC | NP_004619 |
| 997 | XPO6 | NP_055986 |
| 998 | YPEL5 | NP_057145 |
| 999 | YTHDF3 | NP_689971 |
| 1000 | ZBTB18 | NP_006343 |
| 1001 | ZC3HAV1 | NP_064504 |
| 1002 | ZDHHC17 | NP_056151 |
| 1003 | ZDHHC18 | NP_115659 |
| 1004 | ZFAND5 | NP_005998 |
| 1005 | ZFC3H1 | NP_659419 |
| 1006 | ZFYVE16 | NP_055548 |
| 1007 | ZMIZ1 | NP_065071 |
| 1008 | ZNF143 | NP_003433 |
| 1009 | ZNF148 | NP_068799 |
| 1010 | ZNF274 | NP_057408 |
| 1011 | ZNF292 | NP_055836 |
| 1012 | ZXDC | NP_079388 |
| 1013 | ZVX | NP_003452 |

TABLE 7
PASIRS BIOMARKER DETAILS
INCLUDING; SEQUENCE IDENTIFICATION
NUMBER, GENE SYMBOL, ENSEMBL
TRANSCRIPT ID AND DNA SEQUENCE

| Seq ID | Gene | Ensembl Transcript |
|---|---|---|
| # DNA | Symbol | ID |
| 1014 | ACSL4 | ENST00000348502 |
| 1015 | ADK | ENST00000372734 |
| 1016 | ADSL | ENST00000623063 |
| 1017 | AHCTF1 | ENST00000326225 |
| 1018 | APEX1 | ENST00000216714 |
| 1019 | ARHGAP17 | ENST00000303665 |
| 1020 | ARID1A | ENST00000324856 |
| 1021 | ARIH2 | ENST00000356401 |
| 1022 | ASXL2 | ENST00000435504 |
| 1023 | ATOX1 | ENST00000313115 |
| 1024 | ATP2A2 | ENST00000308664 |
| 1025 | ATP6V1B2 | ENST00000276390 |
| 1026 | BCL11A | ENST00000356842 |
| 1027 | BCL3 | ENST00000164227 |
| 1028 | BCL6 | ENST00000406870 |
| 1029 | C3AR1 | ENST00000307637 |
| 1030 | CAMK2G | ENST00000351293 |
| 1031 | CCND3 | ENST00000372991 |
| 1032 | CCR7 | ENST00000246657 |
| 1033 | CD52 | ENST00000374213 |
| 1034 | CD55 | ENST00000367064 |
| 1035 | CD63 | ENST00000257857 |
| 1036 | CEBPB | ENST00000303004 |
| 1037 | CEP192 | ENST00000506447 |
| 1038 | CHN2 | ENST00000222792 |
| 1039 | CLIP4 | ENST00000320081 |
| 1040 | CNOT7 | ENST00000361272 |
| 1041 | CSNK1G2 | ENST00000255641 |
| 1042 | CSTB | ENST00000291568 |
| 1043 | DNAJC10 | ENST00000264065 |
| 1044 | ENO1 | ENST00000234590 |
| 1045 | ERLIN1 | ENST00000421367 |
| 1046 | ETV6 | ENST00000396373 |
| 1047 | EXOSC10 | ENST00000304457 |
| 1048 | EXOSC2 | ENST00000372358 |
| 1049 | EXOSC9 | ENST00000243498 |
| 1050 | FBL | ENST00000221801 |
| 1051 | FBXO11 | ENST00000402508 |
| 1052 | FCER1G | ENST00000289902 |
| 1053 | FGR | ENST00000374005 |
| 1054 | FLII | ENST00000327031 |
| 1055 | FLOT1 | ENST00000383382 |
| 1056 | FNTA | ENST00000302279 |
| 1057 | G6PD | ENST00000393562 |
| 1058 | GLG1 | ENST00000205061 |
| 1059 | GNG5 | ENST00000370645 |
| 1060 | GPI | ENST00000356487 |
| 1061 | GRINA | ENST00000313269 |
| 1062 | HCK | ENST00000534862 |
| 1063 | HERC6 | ENST00000264346 |
| 1064 | HLA-DPA1 | ENST00000383224 |
| 1065 | IL10RA | ENST00000227752 |
| 1066 | IMP3 | ENST00000403490 |
| 1067 | IRF1 | ENST00000245414 |
| 1068 | IRF8 | ENST00000268638 |
| 1069 | JUNB | ENST00000302754 |
| 1070 | KIF1B | ENST00000263934 |
| 1071 | LAP3 | ENST00000618908 |
| 1072 | LDHA | ENST00000422447 |
| 1073 | LY9 | ENST00000263285 |
| 1074 | METAP1 | ENST00000296411 |
| 1075 | MGEA5 | ENST00000361464 |
| 1076 | MLLT10 | ENST00000377072 |
| 1077 | MYD88 | ENST00000396334 |
| 1078 | NFIL3 | ENST00000297689 |
| 1079 | NFKBIA | ENST00000216797 |
| 1080 | NOSIP | ENST00000596358 |
| 1081 | NUMB | ENST00000557597 |
| 1082 | NUP160 | ENST00000378460 |
| 1083 | PCBP1 | ENST00000303577 |
| 1084 | PCID2 | ENST00000375479 |
| 1085 | PCMT1 | ENST00000464889 |
| 1086 | PGD | ENST00000270776 |
| 1087 | PLAUR | ENST00000340093 |
| 1088 | PLSCR1 | ENST00000342435 |
| 1089 | POMP | ENST00000380842 |
| 1090 | PREPL | ENST00000260648 |
| 1091 | PRKCD | ENST00000330452 |
| 1092 | RAB27A | ENST00000396307 |
| 1093 | RAB7A | ENST00000265062 |
| 1094 | RALB | ENST00000272519 |
| 1095 | RBMS1 | ENST00000348849 |
| 1096 | RIT1 | ENST00000368323 |
| 1097 | RPL15 | ENST00000611050 |
| 1098 | RPL22 | ENST00000234875 |
| 1099 | RPL9 | ENST00000295955 |
| 1100 | RPS14 | ENST00000407193 |
| 1101 | RP54X | ENST00000316084 |
| 1102 | RTN4 | ENST00000394609 |
| 1103 | SEH1L | ENST00000262124 |
| 1104 | SERBP1 | ENST00000370994 |
| 1105 | SERPINB1 | ENST00000380739 |
| 1106 | SERTAD2 | ENST00000313349 |
| 1107 | SETX | ENST00000224140 |
| 1108 | SH3GLB1 | ENST00000370558 |
| 1109 | SLAMF7 | ENST00000368043 |
| 1110 | SOCS3 | ENST00000330871 |
| 1111 | SORT1 | ENST00000256637 |
| 1112 | SPI1 | ENST00000378538 |
| 1113 | SQRDL | ENST00000260324 |
| 1114 | STAT3 | ENST00000404395 |
| 1115 | SUCLG2 | ENST00000307227 |
| 1116 | TANK | ENST00000259075 |
| 1117 | TAPI | ENST00000424897 |
| 1118 | TCF4 | ENST00000356073 |
| 1119 | TCIRG1 | ENST00000265686 |
| 1120 | TIMP2 | ENST00000262768 |
| 1121 | TMEM106B | ENST00000396667 |
| 1122 | TMEM50B | ENST00000573374 |
| 1123 | TNIP1 | ENST00000521591 |
| 1124 | TOP2B | ENST00000435706 |
| 1125 | TPP1 | ENST00000299427 |
| 1126 | TRAF3IP3 | ENST00000367025 |
| 1127 | TRIB1 | ENST00000311922 |
| 1128 | TRIT1 | ENST00000316891 |
| 1129 | TROVE2 | ENST00000367446 |
| 1130 | TRPC4AP | ENST00000252015 |
| 1131 | TSPO | ENST00000337554 |
| 1132 | TTC17 | ENST00000039989 |
| 1133 | TUBA1B | ENST00000336023 |
| 1134 | UBE2L6 | ENST00000287156 |
| 1135 | UFM1 | ENST00000239878 |
| 1136 | UPP1 | ENST00000395564 |
| 1137 | USP34 | ENST00000398571 |
| 1138 | VAMP3 | ENST00000054666 |
| 1139 | WARS | ENST00000392882 |
| 1140 | WAS | ENST00000376701 |
| 1141 | ZBED5 | ENST00000432999 |
| 1142 | ZMYND11 | ENST00000397962 |
| 1143 | ZNF266 | ENST00000590306 |

TABLE 8
PASIRS BIOMARKER DETAILS
INCLUDING; SEQUENCE IDENTIFICATION
NUMBER, GENE SYMBOL, GENBANK
ACCESSION AND AMINO ACID SEQUENCE

| Seq ID | Gene | GenBank |
|---|---|---|
| # AA | Symbol | Accession |
| 1144 | ACSL4 | NP_004449 |
| 1145 | ADK | NP_001114 |
| 1146 | ADSL | NP_000017 |
| 1147 | AHCTF1 | NP_056261 |
| 1148 | APEX1 | NP_001632 |
| 1149 | ARHGAP17 | NP_060524 |
| 1150 | ARID1A | NP_006006 |
| 1151 | ARIH2 | NP_006312 |
| 1152 | ASXL2 | NP_060733 |
| 1153 | ATOX1 | NP_004036 |
| 1154 | ATP2A2 | NP_001672 |
| 1155 | ATP6V1B2 | NP_001684 |
| 1156 | BCL11A | NP_060484 |
| 1157 | BCL3 | NP_005169 |
| 1158 | BCL6 | NP_001697 |
| 1159 | C3AR1 | NP_004045 |
| 1160 | CAMK2G | NP_001213 |
| 1161 | CCND3 | NP_001751 |
| 1162 | CCR7 | NP_001829 |
| 1163 | CD52 | NP_001794 |
| 1164 | CD55 | NP_000565 |
| 1165 | CD63 | NP_001771 |
| 1166 | CEBPB | NP_005185 |
| 1167 | CEP192 | NP_115518 |
| 1168 | CHN2 | NP_004058 |
| 1169 | CLIP4 | NP_078968 |
| 1170 | CNOT7 | NP_037486 |
| 1171 | CSNK1G2 | NP_001310 |
| 1172 | CSTB | NP_000091 |
| 1173 | DNAJC10 | NP_061854 |
| 1174 | ENO1 | NP_001419 |
| 1175 | ERLIN1 | NP_006450 |
| 1176 | ETV6 | NP_001978 |
| 1177 | EXOSC1O | NP_002676 |
| 1178 | EXOSC2 | NP_055100 |
| 1179 | EXOSC9 | NP_005024 |
| 1180 | FBL | NP_001427 |
| 1181 | FBXO11 | NP_079409 |
| 1182 | FCER1G | NP_004097 |
| 1183 | FGR | NP_005239 |
| 1184 | FLII | NP_002009 |
| 1185 | FLOT1 | NP_005794 |
| 1186 | FNTA | NP_002018 |
| 1187 | G6PD | NP_000393 |
| 1188 | GLG1 | NP_036333 |
| 1189 | GNG5 | NP_005265 |
| 1190 | GPI | NP_000166 |
| 1191 | GRINA | NP_000828 |
| 1192 | HCK | NP_002101 |
| 1193 | HERC6 | NP_060382 |
| 1194 | HLA-DPA1 | NP_291032 |
| 1195 | IL10RA | NP_001549 |
| 1196 | IMP3 | NP_060755 |
| 1197 | IRF1 | NP_002189 |
| 1198 | IRF8 | NP_002154 |
| 1199 | JUNB | NP_002220 |
| 1200 | KIF1B | NP_055889 |
| 1201 | LAP3 | NP_056991 |
| 1202 | LDHA | NP_005557 |
| 1203 | LY9 | NP_002339 |
| 1204 | METAP1 | NP_055958 |
| 1205 | MGEA5 | NP_036347 |
| 1206 | MLLT10 | NP_004632 |
| 1207 | MYD88 | NP_002459 |
| 1208 | NFIL3 | NP_005375 |
| 1209 | NFKBIA | NP_065390 |
| 1210 | NOSIP | NP_057037 |
| 1211 | NUMB | NP_003735 |
| 1212 | NUP160 | NP_056046 |
| 1213 | PCBP1 | NP_006187 |
| 1214 | PCID2 | NP_060856 |
| 1215 | PCMT1 | NP_005380 |
| 1216 | PGD | NP_002622 |
| 1217 | PLAUR | NP_002650 |
| 1218 | PLSCR1 | NP_066928 |
| 1219 | POMP | NP_057016 |
| 1220 | PREPL | NP_006027 |
| 1221 | PRKCD | NP_006245 |
| 1222 | RAB27A | NP_004571 |
| 1223 | RAB7A | NP_004628 |
| 1224 | RALB | NP_002872 |
| 1225 | RBMS1 | NP_002888 |
| 1226 | RIT1 | NP_008843 |
| 1227 | RPL15 | NP_002939 |
| 1228 | RPL22 | NP_000974 |
| 1229 | RPL9 | NP_000652 |
| 1230 | RPS14 | NP_005608 |
| 1231 | RPS4X | NP_000998 |
| 1232 | RTN4 | NP_008939 |
| 1233 | SEH1L | NP_112493 |
| 1234 | SERBP1 | NP_056455 |
| 1235 | SERPINB1 | NP_109591 |
| 1236 | SERTAD2 | NP_055570 |
| 1237 | SETX | NP_055861 |
| 1238 | SH3GLB1 | NP_057093 |
| 1239 | SLAMF7 | NP_067004 |
| 1240 | SOCS3 | NP_003946 |
| 1241 | SORT1 | NP_002950 |
| 1242 | SPI1 | NP_003111 |
| 1243 | SQRDL | NP_057022 |
| 1244 | STAT3 | NP_003141 |
| 1245 | SUCLG2 | NP_003839 |
| 1245 | TANK | NP_004171 |
| 1247 | TAP1 | NP_000584 |
| 1248 | TCF4 | NP_003190 |
| 1249 | TCIRG1 | NP_006010 |
| 1250 | TIMP2 | NP_003246 |
| 1251 | TMEM106B | NP_060844 |
| 1252 | TMEM50B | NP_006125 |
| 1253 | TNIP1 | NP_006049 |
| 1254 | TOP2B | NP_001059 |
| 1255 | TPP1 | NP_000382 |
| 1256 | TRAF3IP3 | NP_079504 |
| 1257 | TRIB1 | NP_079471 |
| 1258 | TRIT1 | NP_060116 |
| 1259 | TROVE2 | NP_004591 |
| 1260 | TRPC4AP | NP_056453 |
| 1261 | TSPO | NP_000705 |
| 1262 | TTC17 | NP_060729 |
| 1263 | TUBA1B | NP_006073 |
| 1264 | UBE2L6 | NP_004214 |
| 1265 | UFM1 | NP_057701 |
| 1266 | UPP1 | NP_003355 |
| 1267 | USP34 | NP_055524 |
| 1268 | VAMP3 | NP_004772 |
| 1269 | WARS | NP_004175 |
| 1270 | WAS | NP_000368 |
| 1271 | ZBED5 | NP_067034 |
| 1272 | ZMYND11 | NP_006615 |
| 1273 | ZNF266 | NP_006622 |

TABLE 9
INSIRS BIOMARKER DETAILS INCLUDING;
SEQUENCE IDENTIFICATION NUMBER,
GENE SYMBOL, ENSEMBL TRANSCRIPT
ID AND DNA SEQUENCE

| Seq ID | Gene | Ensembl Transcript |
|---|---|---|
| # DNA | Symbol | ID |
| 1274 | ADAM19 | ENST00000257527 |
| 1275 | ADRBK2 | ENST00000324198 |
| 1276 | ADSL | ENST00000623063 |
| 1277 | AGA | ENST00000264595 |
| 1278 | AGPAT5 | ENST00000285518 |
| 1279 | ANK3 | ENST00000355288 |
| 1280 | ARHGAP5 | ENST00000556611 |
| 1281 | ARHGEF6 | ENST00000250617 |
| 1282 | ARL6IP5 | ENST00000273258 |
| 1283 | ASCC3 | ENST00000369162 |
| 1284 | ATP8A1 | ENST00000381668 |
| 1285 | ATXN3 | ENST00000558190 |
| 1286 | BCKDHB | ENST00000356489 |
| 1287 | BRCC3 | ENST00000369462 |
| 1288 | BTN2A1 | ENST00000312541 |
| 1289 | BZW2 | ENST00000258761 |
| 1290 | C14orf1 | ENST00000256319 |
| 1291 | CD28 | ENST00000324106 |
| 1292 | CD40LG | ENST00000370629 |
| 1293 | CD84 | ENST00000368054 |
| 1294 | CDA | ENST00000375071 |
| 1295 | CDK6 | ENST00000265734 |
| 1296 | CDKN1B | ENST00000228872 |
| 1297 | CKAP2 | ENST00000258607 |
| 1298 | CLEC4E | ENST00000299663 |
| 1299 | CLOCK | ENST00000309964 |
| 1300 | CLUAP1 | ENST00000576634 |
| 1301 | CPA3 | ENST00000296046 |
| 1302 | CREB1 | ENST00000353267 |
| 1303 | CYP4F3 | ENST00000221307 |
| 1304 | CYSLTR1 | ENST00000373304 |
| 1305 | DIAPH2 | ENST00000324765 |
| 1306 | EFHD2 | ENST00000375980 |
| 1307 | EFTUD1 | ENST00000268206 |
| 1308 | EIF5B | ENST00000289371 |
| 1309 | ENOSF1 | ENST00000251101 |
| 1310 | ENTPD1 | ENST00000371205 |
| 1311 | ERCC4 | ENST00000311895 |
| 1312 | ESF1 | ENST00000202816 |
| 1313 | EXOC7 | ENST00000332065 |
| 1314 | EXTL3 | ENST00000220562 |
| 1315 | FASTKD2 | ENST00000236980 |
| 1316 | FCF1 | ENST00000341162 |
| 1317 | FUT8 | ENST00000557164 |
| 1318 | G3BP1 | ENST00000356245 |
| 1319 | GAB2 | ENST00000340149 |
| 1320 | GGPS1 | ENST00000358966 |
| 1321 | GOLPH3L | ENST00000271732 |
| 1322 | HAL | ENST00000261208 |
| 1323 | HEATR1 | ENST00000366582 |
| 1324 | HEBP2 | ENST00000607197 |
| 1325 | HIBCH | ENST00000359678 |
| 1326 | HLTF | ENST00000310053 |
| 1327 | HRH4 | ENST00000256906 |
| 1328 | IDE | ENST00000265986 |
| 1329 | IGF2R | ENST00000356956 |
| 1330 | IKBKAP | ENST00000374647 |
| 1331 | IPO7 | ENST00000379719 |
| 1332 | IQCB1 | ENST00000310864 |
| 1333 | IQSEC1 | ENST00000273221 |
| 1334 | KCMF1 | ENST00000409785 |
| 1335 | KIAA0391 | ENST00000534898 |
| 1336 | KLHL20 | ENST00000209884 |
| 1337 | KLHL24 | ENST00000454652 |
| 1338 | KRIT1 | ENST00000340022 |
| 1339 | LANCL1 | ENST00000450366 |
| 1340 | LARP1 | ENST00000336314 |
| 1341 | LARP4 | ENST00000398473 |
| 1342 | LRRC8D | ENST00000394593 |
| 1343 | MACF1 | ENST00000361689 |
| 1344 | MANEA | ENST00000358812 |
| 1345 | MDH1 | ENST00000233114 |
| 1346 | METTL5 | ENST00000260953 |
| 1347 | MLLT10 | ENST00000377072 |
| 1348 | MRPS10 | ENST00000053468 |
| 1349 | MTO1 | ENST00000498286 |
| 1350 | MTRR | ENST00000440940 |
| 1351 | MXD1 | ENST00000264444 |
| 1352 | MYH9 | ENST00000216181 |
| 1353 | MYO9A | ENST00000356056 |
| 1354 | NCBP1 | ENST00000375147 |
| 1355 | NEK1 | ENST00000439128 |
| 1356 | NFX1 | ENST00000379540 |
| 1357 | NGDN | ENST00000397154 |
| 1358 | NIP7 | ENST00000254940 |
| 1359 | NOL10 | ENST00000381685 |
| 1360 | NOL8 | ENST00000442668 |
| 1361 | NOTCH2 | ENST00000256646 |
| 1362 | NR2C1 | ENST00000333003 |
| 1363 | PELI1 | ENST00000358912 |
| 1364 | PEX1 | ENST00000248633 |
| 1365 | PHC3 | ENST00000495893 |
| 1366 | PLCL2 | ENST00000432376 |
| 1367 | POLR2A | ENST00000621442 |
| 1368 | PRKAB2 | ENST00000254101 |
| 1369 | PRPF39 | ENST00000355765 |
| 1370 | PRUNE | ENST00000271620 |
| 1371 | PSMD5 | ENST00000210313 |
| 1372 | PTGS1 | ENST00000362012 |
| 1373 | PWP1 | ENST00000412830 |
| 1374 | RAB11FIP2 | ENST00000355624 |
| 1375 | RABGAP1L | ENST00000251507 |
| 1376 | RAD50 | ENST00000378823 |
| 1377 | RBM26 | ENST00000267229 |
| 1378 | RCBTB2 | ENST00000344532 |
| 1379 | RDX | ENST00000343115 |
| 1380 | REPS1 | ENST00000258062 |
| 1381 | RFC1 | ENST00000349703 |
| 1382 | RGS2 | ENST00000235382 |
| 1383 | RIOK2 | ENST00000283109 |
| 1384 | RMND1 | ENST00000367303 |
| 1385 | RNF170 | ENST00000527424 |
| 1386 | RNMT | ENST00000383314 |
| 1387 | RRAGC | ENST00000373001 |
| 1388 | S100PBP | ENST00000373475 |
| 1389 | SIDT2 | ENST00000324225 |
| 1390 | SLC35A3 | ENST00000370155 |
| 1391 | SLC35D1 | ENST00000235345 |
| 1392 | SLCO3A1 | ENST00000318445 |
| 1393 | SMC3 | ENST00000361804 |
| 1394 | SMC6 | ENST00000351948 |
| 1395 | STK17B | ENST00000263955 |
| 1396 | SUPT7L | ENST00000337768 |
| 1397 | SYNE2 | ENST00000344113 |
| 1398 | SYT11 | ENST00000368324 |
| 1399 | TBCE | ENST00000366601 |
| 1400 | TCF12 | ENST00000267811 |
| 1401 | TCF7L2 | ENST00000369397 |
| 1402 | TFIP11 | ENST00000407690 |
| 1403 | TGS1 | ENST00000260129 |
| 1404 | THOC2 | ENST00000245838 |
| 1405 | TIA1 | ENST00000415783 |
| 1406 | TLK1 | ENST00000431350 |
| 1407 | TMEM87A | ENST00000389834 |
| 1408 | TNFSF8 | ENST00000223795 |
| 1409 | TRAPPC2 | ENST00000359680 |
| 1410 | TRIP11 | ENST00000267622 |
| 1411 | TTC17 | ENST00000039989 |
| 1412 | TTC27 | ENST00000317907 |
| 1413 | VEZT | ENST00000436874 |
| 1414 | VNN3 | ENST00000207771 |
| 1415 | VPS13A | ENST00000357409 |
| 1416 | VPS13B | ENST00000355155 |
| 1417 | VPS13C | ENST00000249837 |
| 1418 | WDR70 | ENST00000265107 |
| 1419 | XPO4 | ENST00000255305 |
| 1420 | YEATS4 | ENST00000247843 |
| 1421 | YTHDC2 | ENST00000161863 |
| 1422 | ZMYND11 | ENST00000397962 |
| 1423 | ZNF507 | ENST00000311921 |
| 1424 | ZNF562 | ENST00000293648 |

TABLE 10
INSIRS BIOMARKER DETAILS INCLUDING;
SEQUENCE IDENTIFICATION NUMBER,
GENE SYMBOL, GENBANK ACCESSION
AND AMINO ACID SEQUENCE

| SEQ ID | Gene | GenBank |
|---|---|---|
| # AA | Symbol | Accession |
| 1425 | ADAM19 | NP_150377 |
| 1426 | ADRBK2 | NP_005151 |
| 1427 | ADSL | NP_000017 |
| 1428 | AGA | NP_000018 |
| 1429 | AGPAT5 | NP_060831 |
| 1430 | ANK3 | NP_001140 |
| 1431 | ARHGAP5 | NP_001164 |
| 1432 | ARHGEF6 | NP_004831 |
| 1433 | ARL6IP5 | NP_006398 |
| 1434 | ASCC3 | NP_006819 |
| 1435 | ATP8A1 | NP_006086 |
| 1436 | ATXN3 | NP_004984 |
| 1437 | BCKDHB | NP_000047 |
| 1438 | BRCC3 | NP_077308 |
| 1439 | BTN2A1 | NP_008980 |
| 1440 | BZW2 | NP_054757 |
| 1441 | C14orf1 | NP_009107 |
| 1442 | CD28 | NP_006130 |
| 1443 | CD40LG | NP_000065 |
| 1444 | CD84 | NP_003865 |
| 1445 | CDA | NP_001776 |
| 1446 | CDK6 | NP_001250 |
| 1447 | CDKN1B | NP_004055 |
| 1448 | CKAP2 | NP_060674 |
| 1449 | CLEC4E | NP_055173 |
| 1450 | CLOCK | NP_004889 |
| 1451 | CLUAP1 | NP_055856 |
| 1452 | CPA3 | NP_001861 |
| 1453 | CREB1 | NP_004370 |
| 1454 | CYP4F3 | NP_000887 |
| 1455 | CYSLTR1 | NP_006630 |
| 1456 | DIAPH2 | NP_006720 |
| 1457 | EFHD2 | NP_077305 |
| 1458 | EFTUD1 | NP_078856 |
| 1459 | EIF5B | NP_056988 |
| 1460 | ENOSF1 | NP_059982 |
| 1461 | ENTPD1 | NP_001767 |
| 1462 | ERCC4 | NP_005227 |
| 1463 | ESF1 | NP_057733 |
| 1464 | EXOC7 | NP_056034 |
| 1465 | EXTL3 | NP_001431 |
| 1466 | FASTKD2 | NP_055744 |
| 1467 | FCF1 | NP_057046 |
| 1468 | FUT8 | NP_004471 |
| 1469 | G3BP1 | NP_005745 |
| 1470 | GAB2 | NP_036428 |
| 1471 | GGPS1 | NP_001032354 |
| 1472 | GOLPH3L | NP_060648 |
| 1473 | HAL | NP_002099 |
| 1474 | HEATR1 | NP_060542 |
| 1475 | HEBP2 | NP_055135 |
| 1476 | HIBCH | NP_055177 |
| 1477 | HLTF | NP_003062 |
| 1478 | HRH4 | NP_067637 |
| 1479 | IDE | NP_004960 |
| 1480 | IGF2R | NP_000867 |
| 1481 | IKBKAP | NP_003631 |
| 1482 | IPO7 | NP_006382 |
| 1483 | IQCB1 | NP_001018864 |
| 1484 | IQSEC1 | NP_055684 |
| 1485 | KCMF1 | NP_064507 |
| 1486 | KIAA0391 | NP_055487 |
| 1487 | KLHL20 | NP_055273 |
| 1488 | KLHL24 | NP_060114 |
| 1489 | KRIT1 | NP_004903 |
| 1490 | LANCL1 | NP_006046 |
| 1491 | LARP1 | NP_056130 |
| 1492 | LARP4 | NP_443111 |
| 1493 | LRRC8D | NP_060573 |
| 1494 | MACF1 | NP_036222 |
| 1495 | MANEA | NP_078917 |
| 1496 | MDH1 | NP_005908 |
| 1497 | METTL5 | NP_054887 |
| 1498 | MLLT10 | NP_004632 |
| 1499 | MRPS10 | NP_060611 |
| 1500 | MTO1 | NP_036255 |
| 1501 | MTRR | NP_002445 |
| 1502 | MXD1 | NP_002348 |
| 1503 | MYH9 | NP_002464 |
| 1504 | MYO9A | NP_008832 |
| 1505 | NCBP1 | NP_002477 |
| 1506 | NEK1 | NP_036356 |
| 1507 | NFX1 | NP_002495 |
| 1508 | NGDN | NP_056329 |
| 1509 | NIP7 | NP_057185 |
| 1510 | NOL10 | NP_079170 |
| 1511 | NOL8 | NP_060418 |
| 1512 | NOTCH2 | NP_077719 |
| 1513 | NR2C1 | NP_003288 |
| 1514 | PELI1 | NP_065702 |
| 1515 | PEX1 | NP_000457 |
| 1516 | PHC3 | NP_079223 |
| 1517 | PLCL2 | NP_055999 |
| 1518 | POLR2A | NP_000928 |
| 1519 | PRKAB2 | NP_005390 |
| 1520 | PRPF39 | NP_060392 |
| 1521 | PRUNE | NP_067045 |
| 1522 | PSMD5 | NP_005038 |
| 1523 | PTGS1 | NP_000953 |
| 1524 | PWP1 | NP_008993 |
| 1525 | RAB11FIP2 | NP_055719 |
| 1526 | RABGAP1L | NP_055672 |
| 1527 | RAD50 | NP_005723 |
| 1528 | RBM26 | NP_071401 |
| 1529 | RCBTB2 | NP_001259 |
| 1530 | RDX | NP_002897 |
| 1531 | REPS1 | NP_114128 |
| 1532 | RFC1 | NP_002904 |
| 1533 | RGS2 | NP_002914 |
| 1534 | RIOK2 | NP_060813 |
| 1535 | RMND1 | NP_060379 |
| 1536 | RNF170 | NP_112216 |
| 1537 | RNMT | NP_003790 |
| 1538 | RRAGC | NP_071440 |
| 1539 | S100PBP | NP_073590 |
| 1540 | SIDT2 | NP_001035545 |
| 1541 | SLC35A3 | NP_036375 |
| 1542 | SLC35D1 | NP_055954 |
| 1543 | SLCO3A1 | NP_037404 |
| 1544 | SMC3 | NP_005436 |
| 1545 | SMC6 | NP_078900 |
| 1546 | STK17B | NP_004217 |
| 1547 | SUPT7L | NP_055675 |
| 1548 | SYNE2 | NP_055995 |
| 1549 | SYT11 | NP_689493 |
| 1550 | TBCE | NP_003184 |
| 1551 | TCF12 | NP_003196 |
| 1552 | TCF7L2 | NP_110383 |
| 1553 | TFIP11 | NP_036275 |
| 1554 | TGS1 | NP_079107 |
| 1555 | THOC2 | NP_001075019 |
| 1556 | TIA1 | NP_071320 |
| 1557 | TLK1 | NP_036422 |
| 1558 | TMEM87A | NP_056312 |
| 1559 | TNFSF8 | NP_001235 |
| 1560 | TRAPPC2 | NP_055378 |
| 1561 | TRIP11 | NP_004230 |
| 1562 | TTC17 | NP_060729 |
| 1563 | TTC27 | NP_060205 |
| 1564 | VEZT | NP_060069 |
| 1565 | VNN3 | NP_001278631 |
| 1566 | VPS13A | NP_056001 |
| 1567 | VPS13B | NP_056058 |
| 1568 | VPS13C | NP_060154 |
| 1569 | WDR70 | NP_060504 |
| 1570 | XPO4 | NP_071904 |
| 1571 | YEATS4 | NP_006521 |
| 1572 | YTHDC2 | NP_073739 |
| 1573 | ZMYND11 | NP_006615 |
| 1574 | ZNF507 | NP_055725 |
| 1575 | ZNF562 | NP_060126 |

TABLE 11
EXEMPLARY ESCHERICHIA COLI DNA SEQUENCE
INCLUDING SINGLE NUCLEOTIDE POLYMORPHISMS
(SNPS) AT POSITIONS 396 AND 398 (BOLDED)

| SEQ ID # DNA | Organism | GenBank Accession |
|---|---|---|
| 1576 | Escherichia coli | NR_074891 |

TABLE 12
DESCRIPTION OF DATASETS AND NUMBER OF
SAMPLES USED AS PART OF DISCOVERY OF
DERIVED BIOMARKERS FOR BASIRS
The total number of genes that were able to be used across all of these
datasets was 3698. All useable samples in these datasets were randomly
divided into BaSIRS discovery and validation (see Table 13) sets.

| Dataset Identifier | Source | # Samples | Control | Case |
|---|---|---|---|---|
| FEVER | In-house | 30 | 8 | 22 |
| FEVER (Bact vs Viral) | In-house | 34 | 7 | 27 |
| GAPPSS | In-house | 32 | 15 | 17 |
| MARS (Healthy) | In-house | 40 | 20 | 20 |
| MARS (Healthy vs All) | In-house | 459 | 21 | 438 |
| MARS (SIRS) | In-house | 73 | 57 | 16 |
| GSE16129 | GEO | 19 | 6 | 13 |
| GSE30119 | GEO | 40 | 13 | 27 |
| GSE40396 | GEO | 16 | 10 | 6 |
| GSE6269 | GEO | 16 | 6 | 10 |
| GSE63990 | GEO | 79 | 44 | 35 |
| GSE63990 (Bact vs Viral) | GEO | 93 | 58 | 35 |
| GSE74224 | GEO | 53 | 16 | 37 |

| | | |
|---|---|---|
| 984 | 281 | 703 |

TABLE 13
DESCRIPTION OF DATASETS AND NUMBER
OF SAMPLES USED AS PART OF VALIDATION
OF DERIVED BIOMARKERS FOR BASIRS

| Dataset Identifier | Source | # Samples | Control | Case |
|---|---|---|---|---|
| FEVER | In-house | 30 | 8 | 22 |
| FEVER (Bact vs Viral) | In-house | 34 | 7 | 27 |
| GAPPSS | In-house | 31 | 14 | 17 |
| MARS (Healthy) | In-house | 40 | 20 | 20 |
| MARS (Healthy vs All) | In-house | 459 | 21 | 438 |
| MARS (SIRS) | In-house | 71 | 56 | 15 |
| GSE16129 | GEO | 39 | 10 | 29 |
| GSE30119 | GEO | 73 | 31 | 42 |
| GSE40396 | GEO | 20 | 12 | 8 |
| GSE6269 | GEO | 25 | 6 | 19 |
| GSE63990 | GEO | 79 | 44 | 35 |
| GSE63990 (Bact vs Viral) | GEO | 92 | 57 | 35 |
| GSE74224 | GEO | 52 | 15 | 37 |

| | | |
|---|---|---|
| 1045 | 301 | 744 |

TABLE 14
DESCRIPTION OF CONTROL DATASETS AND
NUMBER OF SAMPLES USED FOR SUBTRACTION
FROM THE DERIVED BIOMARKERS FOR BASIRS
The subtraction process ensured that the
BaSIRS derived biomarkers were specific.

| Dataset | Numbers | Comments | Case | Control | Total |
|---|---|---|---|---|---|
| GSE11908 | 58 sterile inflammation; | General inflammation: | 6 | 58 | 64 |
| | 6 Staph or E. coli infection | inSIRS = SLE, Diabetes, | | | |
| | | Melanoma | | | |
| GSE52428 | 39 healthy; 41 Influenza A | Influenza virus | 41 | 39 | 80 |
| GSE19301 | 394 healthy; 166 Asthma | Asthma | 166 | 394 | 560 |
| GSE38485 | 96 healthy; 106 schizophrenia | Schizophrenia | 106 | 96 | 202 |
| GSE29532 | 6 Healthy; 25 acute coronary | Coronary artery disease | 25 | 6 | 31 |
| | syndrome | | | | |
| GSE46743 | 160 baseline; 160 stress | Depression/stress | 160 | 160 | 320 |
| GSE64813 | 141 Pre-deployment; 47 post- | PTSD | 47 | 141 | 188 |
| | deployment Post- Traumatic | | | | |
| | Stress Disorder | | | | |
| GSE51808 | 9 healthy; 28 infected | Dengue Virus | 28 | 9 | 37 |
| GSE41752 | 11 controls; 19 cases | Lassa Virus | 19 | 11 | 30 |
| GSE42834 | 198 healthy; 46 controls | Patients with tuberculosis, | 198 | 35 | 233 |
| | | sarcoidosis, and lung | | | |
| | | cancer (pneumonia patients | | | |
| | | removed) | | | |
| | | | 796 | 949 | 1745 |

TABLE 15
PERFORMANCE (AS MEASURED BY AUC) OF THE
FINAL BASIRS SIGNATURE IN EACH OF THE DISCOVERY,
VALIDATION AND CONTROL DATASETS

| Dataset | AUC | Analysis |
|---|---|---|
| FEVER | 0.858 | Discovery |
| FEVER (Bact vs Viral) | 0.910 | Discovery |
| GAPPSS | 0.925 | Discovery |
| GSE16129 | 1.000 | Discovery |
| GSE30119 | 0.892 | Discovery |
| GSE36809 | 0.899 | Discovery |
| GSE40012 (Healthy) | 0.834 | Discovery |
| GSE40012 (SIRS) | 0.834 | Discovery |
| GSE40396 | 1.000 | Discovery |
| GSE6269 | 1.000 | Discovery |
| GSE63990 | 0.890 | Discovery |
| GSE63990 (Bact vs Viral) | 0.940 | Discovery |
| GSE74224 | 0.856 | Discovery |
| MARS (Healthy) | 1.000 | Discovery |
| MARS (Healthy vs All) | 0.987 | Discovery |
| MARS (SIRS) | 0.935 | Discovery |
| FEVER | 0.926 | Validation |
| FEVER (Bact vs Viral) | 0.799 | Validation |
| GAPPSS | 0.916 | Validation |
| GSE16129 | 0.928 | Validation |
| GSE30119 | 0.856 | Validation |
| GSE36809 | 0.729 | Validation |
| GSE40012 (Healthy) | 0.910 | Validation |
| GSE40012 (SIRS) | 0.551 | Validation |
| GSE40396 | 0.979 | Validation |
| GSE6269 | 0.965 | Validation |
| GSE63990 | 0.795 | Validation |
| GSE63990 (Bact vs Viral) | 0.873 | Validation |
| GSE74224 | 0.942 | Validation |
| MARS (Healthy) | 1.000 | Validation |
| MARS (Healthy vs All) | 0.986 | Validation |
| MARS_SIRS | 0.927 | Validation |
| GSE19301 | 0.573 | Non-BaSIRS |
| GSE29532 | 0.807 | Non-BaSIRS |
| GSE38485 | 0.569 | Non-BaSIRS |
| GSE64813 | 0.674 | Non-BaSIRS |
| GSE46743 | 0.517 | Non-BaSIRS |
| GSE11908 | 0.546 | Non-BaSIRS |
| GSE42834 | 0.647 | Non-BaSIRS |
| GSE52428 | 0.633 | Non-BaSIRS |
| GSE41752 | 0.694 | Non-BaSIRS |
| GSE51808 | 0.484 | Non-BaSIRS |

TABLE 16
PERFORMANCE (AS MEASURED BY AUC) OF THE TOP 102 BASIRS DERIVED
BIOMARKERS IN EACH OF THE BASIRS VALIDATION DATASETS.
Only those derived biomarkers with a mean AUC >0.85 were used in a greedy
search to identify the best combination of derived biomarkers

| | | | | | | FEVER | GSE63990 | |
|---|---|---|---|---|---|---|---|---|
| | | | | | MARS | (Bact vs | (Bact vs | |
| Derived Biomarker | FEVER | GAPPSS | GSE63990 | GSE74224 | (SIRS) | Viral) | Viral) | Mean |
| PDGFC_KLRF1 | 0.972 | 0.882 | 0.823 | 0.923 | 0.933 | 0.947 | 0.899 | 0.911 |
| TMEM165_PARP8 | 0.960 | 0.950 | 0.863 | 0.796 | 0.865 | 0.931 | 0.954 | 0.903 |
| ITGA7_KLRF1 | 0.966 | 0.916 | 0.767 | 0.888 | 0.955 | 0.963 | 0.828 | 0.897 |
| CR1_GAB2 | 0.983 | 0.878 | 0.891 | 0.829 | 0.854 | 0.937 | 0.903 | 0.896 |
| PCOLCE2_KLRF1 | 0.966 | 0.891 | 0.826 | 0.897 | 0.894 | 0.947 | 0.850 | 0.896 |
| ITGA7_INPP5D | 0.938 | 0.903 | 0.792 | 0.850 | 0.971 | 0.915 | 0.894 | 0.895 |
| GALNT2_CCNK | 0.920 | 0.916 | 0.847 | 0.872 | 0.889 | 0.963 | 0.842 | 0.893 |
| PDGFC_KLRD1 | 0.920 | 0.882 | 0.830 | 0.877 | 0.889 | 0.937 | 0.911 | 0.892 |
| PDGFC_CCNK | 0.949 | 0.916 | 0.821 | 0.899 | 0.860 | 0.910 | 0.886 | 0.891 |
| CR1_ADAM19 | 0.983 | 0.954 | 0.825 | 0.802 | 0.842 | 0.947 | 0.882 | 0.891 |
| ITGA7_CCNK | 0.949 | 0.941 | 0.859 | 0.816 | 0.869 | 0.905 | 0.889 | 0.890 |
| PCOLCE2_PRSS23 | 0.920 | 0.916 | 0.827 | 0.886 | 0.876 | 0.915 | 0.877 | 0.888 |
| TMEM165_PRPF38B | 0.977 | 0.933 | 0.852 | 0.840 | 0.743 | 0.937 | 0.935 | 0.888 |
| PDGFC_PHF3 | 0.926 | 0.924 | 0.814 | 0.825 | 0.918 | 0.889 | 0.911 | 0.887 |
| GAS7_NLRP1 | 0.909 | 0.958 | 0.857 | 0.766 | 0.965 | 0.831 | 0.912 | 0.885 |
| PCOLCE2_KLRD1 | 0.903 | 0.908 | 0.860 | 0.867 | 0.846 | 0.921 | 0.891 | 0.885 |
| GALNT2_KLRD1 | 0.926 | 0.853 | 0.841 | 0.852 | 0.899 | 0.947 | 0.871 | 0.884 |
| KIAA0101_IL2RB | 0.949 | 0.845 | 0.853 | 0.829 | 0.855 | 0.958 | 0.897 | 0.884 |
| CR1_HAL | 1.000 | 0.815 | 0.869 | 0.791 | 0.950 | 0.926 | 0.834 | 0.884 |
| PDGFC_RFC1 | 0.989 | 0.723 | 0.825 | 0.892 | 0.893 | 0.947 | 0.911 | 0.883 |
| ENTPD7_KLRF1 | 0.977 | 0.874 | 0.792 | 0.854 | 0.931 | 0.952 | 0.795 | 0.882 |
| PDGFC_GRK5 | 0.864 | 0.958 | 0.827 | 0.877 | 0.890 | 0.857 | 0.901 | 0.882 |
| PCOLCE2_PYHIN1 | 0.920 | 0.866 | 0.844 | 0.798 | 0.902 | 0.931 | 0.910 | 0.882 |
| GAS7_PRKDC | 0.903 | 0.950 | 0.807 | 0.654 | 0.974 | 0.963 | 0.914 | 0.881 |
| GAS7_CAMK1D | 0.892 | 0.899 | 0.867 | 0.755 | 0.908 | 0.931 | 0.910 | 0.880 |
| MGAM_MME | 0.977 | 0.920 | 0.836 | 0.744 | 0.957 | 0.841 | 0.884 | 0.880 |
| GAS7_GAB2 | 0.841 | 0.975 | 0.824 | 0.773 | 0.933 | 0.889 | 0.911 | 0.878 |
| PDGFC_INPP5D | 0.884 | 0.903 | 0.793 | 0.894 | 0.858 | 0.921 | 0.883 | 0.876 |
| ST3GAL2_PRKD2 | 0.795 | 0.958 | 0.846 | 0.921 | 0.771 | 0.952 | 0.889 | 0.876 |
| HK3_INPP5D | 0.966 | 0.815 | 0.761 | 0.874 | 0.917 | 0.942 | 0.856 | 0.876 |
| ENTPD7_KLRD1 | 0.926 | 0.830 | 0.840 | 0.823 | 0.895 | 0.947 | 0.865 | 0.875 |
| PDGFC_SIDT1 | 1.000 | 0.794 | 0.805 | 0.773 | 0.856 | 0.963 | 0.936 | 0.875 |
| PDGFC_SPIN1 | 0.955 | 0.737 | 0.805 | 0.845 | 0.914 | 0.974 | 0.893 | 0.875 |
| PCOLCE2_YPEL1 | 0.966 | 0.916 | 0.866 | 0.849 | 0.787 | 0.862 | 0.869 | 0.873 |
| PDGFC_SYTL2 | 0.972 | 0.807 | 0.808 | 0.852 | 0.860 | 0.921 | 0.894 | 0.873 |
| PDGFC_TGFBR3 | 0.938 | 0.790 | 0.817 | 0.802 | 0.889 | 0.963 | 0.914 | 0.873 |
| IGFBP7_KLRF1 | 0.841 | 1.000 | 0.832 | 0.890 | 0.813 | 0.889 | 0.846 | 0.873 |
| PCOLCE2_RUNX2 | 0.909 | 0.916 | 0.806 | 0.917 | 0.962 | 0.770 | 0.832 | 0.873 |
| SMPDL3A_KLRD1 | 0.881 | 0.777 | 0.798 | 0.877 | 0.932 | 0.958 | 0.888 | 0.873 |
| GALNT2_KLRF1 | 0.943 | 0.786 | 0.812 | 0.872 | 0.927 | 0.942 | 0.826 | 0.873 |
| PDGFC_YPEL1 | 0.977 | 0.819 | 0.818 | 0.863 | 0.835 | 0.910 | 0.886 | 0.873 |
| HK3_DENND3 | 0.920 | 0.824 | 0.778 | 0.960 | 0.967 | 0.820 | 0.836 | 0.872 |
| PDGFC_CBLL1 | 0.989 | 0.815 | 0.782 | 0.825 | 0.911 | 0.873 | 0.910 | 0.872 |
| OPLAH_KLRD1 | 0.943 | 0.723 | 0.830 | 0.838 | 0.924 | 0.952 | 0.892 | 0.872 |
| OPLAH_ZHX2 | 0.972 | 0.777 | 0.812 | 0.849 | 0.914 | 0.915 | 0.860 | 0.871 |
| PDGFC_RYK | 0.994 | 0.723 | 0.799 | 0.845 | 0.913 | 0.923 | 0.900 | 0.871 |
| PDGFC_IKZF5 | 0.932 | 0.765 | 0.809 | 0.933 | 0.915 | 0.825 | 0.913 | 0.870 |
| GALNT2_INPP5D | 0.926 | 0.912 | 0.819 | 0.771 | 0.874 | 0.931 | 0.858 | 0.870 |
| PDGFC_GCC2 | 0.915 | 0.782 | 0.805 | 0.872 | 0.898 | 0.915 | 0.905 | 0.870 |
| PDGFC_MBIP | 0.977 | 0.693 | 0.835 | 0.915 | 0.870 | 0.899 | 0.891 | 0.869 |
| COX15_UTRN | 0.943 | 0.899 | 0.858 | 0.796 | 0.814 | 0.926 | 0.837 | 0.868 |
| SMPDL3A_QRICH1 | 0.966 | 0.777 | 0.792 | 0.825 | 0.890 | 0.947 | 0.874 | 0.867 |
| PDGFC_LPIN2 | 0.841 | 0.861 | 0.836 | 0.849 | 0.808 | 0.974 | 0.901 | 0.867 |
| TSPO_NLRP1 | 0.903 | 0.676 | 0.832 | 0.899 | 0.975 | 0.915 | 0.867 | 0.867 |
| PCOLCE2_NMUR1 | 0.920 | 0.895 | 0.826 | 0.926 | 0.836 | 0.804 | 0.860 | 0.867 |
| FAM129A_GAB2 | 0.943 | 0.832 | 0.771 | 0.845 | 0.925 | 0.952 | 0.794 | 0.866 |
| ALPL_NLRP1 | 0.920 | 0.803 | 0.778 | 0.872 | 0.973 | 0.894 | 0.821 | 0.866 |
| TSPO_ZFP36L2 | 0.966 | 0.651 | 0.820 | 0.856 | 0.919 | 0.958 | 0.891 | 0.866 |
| ALPL_ZFP36L2 | 0.943 | 0.773 | 0.790 | 0.818 | 0.980 | 0.915 | 0.841 | 0.866 |
| PCOLCE2_FOXJ3 | 0.972 | 0.903 | 0.818 | 0.822 | 0.870 | 0.825 | 0.849 | 0.866 |
| PDGFC_KIAA0355 | 0.864 | 0.845 | 0.839 | 0.899 | 0.885 | 0.810 | 0.917 | 0.865 |
| PDGFC_KIAA0907 | 0.966 | 0.723 | 0.800 | 0.856 | 0.895 | 0.905 | 0.906 | 0.864 |
| GAS7_DOCK5 | 0.886 | 0.945 | 0.834 | 0.654 | 0.902 | 0.905 | 0.922 | 0.864 |
| CD82_CNNM3 | 0.960 | 0.714 | 0.882 | 0.859 | 0.871 | 0.889 | 0.872 | 0.864 |
| GAS7_EXTL3 | 0.949 | 0.941 | 0.856 | 0.598 | 0.871 | 0.937 | 0.895 | 0.864 |
| TSPO_RNASE6 | 0.938 | 0.685 | 0.806 | 0.829 | 0.974 | 0.937 | 0.874 | 0.863 |
| ALPL_MME | 0.920 | 0.857 | 0.779 | 0.814 | 0.979 | 0.878 | 0.807 | 0.862 |
| HK3_TLE3 | 0.892 | 0.853 | 0.724 | 0.957 | 0.954 | 0.831 | 0.822 | 0.862 |
| MCTP1_PARP8 | 0.830 | 0.866 | 0.833 | 0.923 | 0.880 | 0.847 | 0.854 | 0.862 |
| TSPO_HCLS1 | 0.920 | 0.912 | 0.783 | 0.894 | 0.860 | 0.788 | 0.872 | 0.861 |
| TSPO_CASS4 | 0.909 | 0.647 | 0.851 | 0.876 | 0.938 | 0.942 | 0.863 | 0.861 |
| GAS7_RBM23 | 0.903 | 0.983 | 0.740 | 0.650 | 0.902 | 0.931 | 0.910 | 0.860 |
| GAS7_EPHB4 | 0.929 | 0.941 | 0.842 | 0.591 | 0.871 | 0.952 | 0.893 | 0.860 |
| PDGFC_RBM15 | 0.955 | 0.756 | 0.769 | 0.858 | 0.921 | 0.942 | 0.810 | 0.859 |
| ADM_CLEC7A | 0.955 | 0.798 | 0.810 | 0.881 | 0.917 | 0.831 | 0.818 | 0.858 |
| PDGFC_LEPROTL1 | 0.943 | 0.815 | 0.733 | 0.811 | 0.898 | 0.963 | 0.842 | 0.858 |
| PDGFC_NPAT | 1.000 | 0.807 | 0.801 | 0.764 | 0.844 | 0.884 | 0.905 | 0.858 |
| TSPO_PLA2G7 | 0.909 | 0.643 | 0.822 | 0.872 | 0.930 | 0.947 | 0.877 | 0.857 |
| GALNT2_IK | 0.955 | 0.782 | 0.812 | 0.823 | 0.908 | 0.873 | 0.842 | 0.856 |
| CD82_JARID2 | 0.972 | 0.744 | 0.810 | 0.836 | 0.942 | 0.878 | 0.812 | 0.856 |
| PDGFC_ICK | 0.938 | 0.660 | 0.823 | 0.883 | 0.899 | 0.897 | 0.892 | 0.856 |
| GALNT2_SAP130 | 0.920 | 0.714 | 0.836 | 0.856 | 0.920 | 0.926 | 0.816 | 0.856 |
| PDGFC_FBXO28 | 0.895 | 0.769 | 0.780 | 0.868 | 0.930 | 0.865 | 0.881 | 0.855 |
| TSPO_GAB2 | 0.898 | 0.668 | 0.792 | 0.930 | 0.924 | 0.937 | 0.838 | 0.855 |
| COX15_INPP5D | 0.881 | 0.899 | 0.801 | 0.877 | 0.826 | 0.884 | 0.817 | 0.855 |
| ITGA7_LAG3 | 0.943 | 0.769 | 0.759 | 0.796 | 0.888 | 0.921 | 0.908 | 0.855 |
| TSPO_CAMK1D | 0.909 | 0.639 | 0.840 | 0.865 | 0.931 | 0.942 | 0.859 | 0.855 |
| OPLAH_POGZ | 0.955 | 0.761 | 0.758 | 0.865 | 0.877 | 0.905 | 0.864 | 0.855 |
| ALPL_RNASE6 | 0.920 | 0.794 | 0.753 | 0.852 | 0.962 | 0.894 | 0.804 | 0.854 |
| RAB32_NLRP1 | 0.926 | 0.866 | 0.779 | 0.897 | 0.939 | 0.746 | 0.826 | 0.854 |
| TLR5_SEMA4D | 0.886 | 0.845 | 0.812 | 0.741 | 0.902 | 0.915 | 0.876 | 0.854 |
| IMPDH1_NLRP1 | 0.875 | 0.697 | 0.810 | 0.901 | 0.943 | 0.921 | 0.830 | 0.854 |
| ALPL_CAMK1D | 0.915 | 0.756 | 0.779 | 0.879 | 0.938 | 0.905 | 0.800 | 0.853 |
| TSPO_NFIC | 0.915 | 0.639 | 0.815 | 0.892 | 0.910 | 0.947 | 0.854 | 0.853 |
| GAS7_HAL | 0.869 | 0.924 | 0.778 | 0.757 | 0.977 | 0.836 | 0.829 | 0.853 |
| PDGFC_NCOA6 | 0.821 | 0.769 | 0.841 | 0.928 | 0.907 | 0.825 | 0.877 | 0.853 |
| PDGFC_PIK3C2A | 0.886 | 0.824 | 0.756 | 0.793 | 0.926 | 0.921 | 0.861 | 0.852 |
| TSPO_ADAM19 | 0.881 | 0.685 | 0.758 | 0.941 | 0.915 | 0.942 | 0.844 | 0.852 |
| CD82_NOV | 0.943 | 0.718 | 0.813 | 0.903 | 0.937 | 0.878 | 0.768 | 0.851 |
| PDGFC_PDS5B | 0.892 | 0.744 | 0.799 | 0.861 | 0.918 | 0.868 | 0.872 | 0.850 |
| FIG4_INPP5D | 0.869 | 0.908 | 0.724 | 0.818 | 0.981 | 0.878 | 0.773 | 0.850 |
| TSPO_NOV | 0.909 | 0.643 | 0.804 | 0.883 | 0.915 | 0.947 | 0.850 | 0.850 |

TABLE 17
DETAILS OF GENE EXPRESSION OMNIBUS
(GEO) DATASETS USED FOR DISCOVERY
OF VIRAL DERIVED BIOMARKERS

| Dataset | Description and Comparison Made |
|---|---|
| GSE40336 | Cytomegalovirus in humans (natural infection) |
| | Comparison of nonagenarians with a titer of 0 |
| | (n = 6) vs >20,000 (n = 67) Herpesviridae; |
| | Baltimore Group I |
| GSE41752 | Lassa virus in macaques (time course, challenge) |
| | Comparison of samples collected pre-challenge |
| | (n = 11) to those collected on Days 2, 3, and |
| | 6, post-challenge (n = 9) Arenaviridae; |
| | Baltimore Group V |
| GSE51808 | Dengue virus in humans (natural infection) |
| | Comparison of healthy controls (n = 9) vs |
| | samples collected at acute infection (n = 28) |
| | Flaviviridae; Baltimore Group IV |
| GSE52428 | Influenza virus (H1N1 & H3N2) (time course, |
| | challenge) Comparison of samples collected |
| | pre-challenge (n = 20) vs samples collected |
| | in the early stages of symptom development |
| | (before peak) (n = 62) Orthomyxoviridae; |
| | Baltimore Group V |

TABLE 18
DETAILS OF GENE EXPRESSION OMNIBUS
(GEO) DATASETS USED FOR VALIDATION
OF VIRAL DERIVED BIOMARKERS

| Dataset | Description and Comparison Made |
|---|---|
| GSE6269 | Influenza infection in humans (naturally acquired) |
| | Comparison of influenza A/B (n = 30/6) vs healthy |
| | (n = 6) Orthomyxoviridae; Baltimore Group V |
| GSE40396 | Adenovirus, Human Herpes Virus 6, Enterovirus and |
| | Rhinovirus in humans (pediatric, naturally |
| | acquired) Comparison of virus-infected (n = 35) vs |
| | virus-negative afebrile controls (n = 19) Adenoviridae; |
| | Baltimore Group I |
| GSE40012 | Influenza A infection in humans (naturally acquired) |
| | Comparison of influenza A infected (n = 39, up to five |
| | time points, 9 subjects) vs healthy controls (n = 36, |
| | two time points, 18 subjects) Orthomyxoviridae; |
| | Baltimore Group V |
| GSE18090 | Dengue fever in humans (naturally acquired) |
| | Comparison of febrile dengue fever (n = 18) vs |
| | febrile patients without dengue fever (n = 8) |
| | Flaviviridae; Baltimore Group IV |
| GSE30550 | Influenza A (H3N2) infection in humans (challenged) |
| | Comparison of all samples where symptoms reported |
| | (17 subjects) vs samples where no symptoms were |
| | reported Orthomyxoviridae; Baltimore Group V |
| GSE40224 | Hepatitis C virus infection in humans (naturally |
| | acquired) Comparison of infected (n = 10) vs |
| | healthy (n = 8) Flaviviridae; Baltimore Group IV |
| GSE5790 | Lymphocytic choriomeningitis virus infection in |
| | macaques (challenged) Comparison of samples taken |
| | pre-infection (n = 11) vs samples taken pre- and |
| | post-viremia with lethal dose (n = 8) |
| | Arenaviridae; Baltimore Group V |
| GSE34205 | Influenza A and Respiratory Syncytial Virus |
| | in humans (pediatric, naturally acquired) |
| | Comparison of infected (Influenza, n = 28; |
| | RSV, n = 51) vs healthy controls (n = 22) |
| | Orthomyxoviridae; Baltimore Group V |
| | Paramyxoviridae; Baltimore Group V |
| GSE5808 | Measles virus in humans (naturally acquired) |
| | Comparison of infected at hospital entry |
| | (n = 5) and healthy controls (n = 3) |
| | Paramyxoviridae; Baltimore Group V |
| GSE2729 | Rotavirus infection in humans (naturally |
| | acquired) Comparison of acute infection |
| | (n = 10) vs healthy controls (n = 8) |
| | Reoviridae; Baltimore Group III |
| GSE29429 | Human immunodeficiency virus infection |
| | in humans (naturally acquired) |
| | Comparison of acute infection at enrolment |
| | (African cohort, n = 17) vs matched |
| | uninfected controls (n = 30) |
| | Retroviridae; Baltimore Group VI |
| GSE14790 | Porcine circovirus in pigs (challenged) |
| | Comparison of pre-challenge (Day 0, n = 4) |
| | to post-challenge on Days 7, 14, 21 |
| | and 28 (n = 15) For performance |
| | calculations in this dataset N4BP1 was |
| | substituted for OASL since it is known |
| | that OASL does not exist in pigs Circoviridae; |
| | Baltimore Group II |
| GSE22160 | Hepatitis C and E in chimpanzees (challenged, |
| | liver biopsies) Comparison of pre-challenge |
| | samples (HCV, n = 3) (HEV, n = 4) to post- |
| | challenge samples over time. HCV: |
| | Flaviviridae, Baltimore Group IV HEV: |
| | Hepeviridae, Baltimore Group IV |
| GSE69606 | Respiratory Syncytial Virus in children |
| | Comparison of mild (n = 9), moderate (n = 9) and |
| | severe (n = 8) cases at presentation to |
| | recovery samples 4-6 weeks later (in |
| | moderate and severe cases). |
| | Paramyxoviridae, Baltimore Group V |
| GSE67059 | Rhinovirus in children |
| | Comparison of HRV− (n = 37) versus HRV+ |
| | asymptomatic (n = 14), HRV+ outpatients |
| | (n = 30), HRV+ inpatients (n = 70). |
| | Picornaviridae, Baltimore Group IV |
| GSE58287 | Marburg virus in Macaques |
| | Comparison of pre-inoculation samples (n = 3) |
| | to samples taken over five time |
| | points. Total samples = 15. |
| | Filoviridae, Baltimore Group V |

TABLE 19
DESCRIPTION OF CONTROL DATASETS
USED FOR SUBTRACTION FROM THE
DERIVED BIOMARKERS FOR VASIRS
The subtraction process ensured that the
VaSIRS derived biomarkers were specific.

| Dataset | Description and Comparison Made |
|---|---|
| GSE33341 | Bacterial sepsis in humans (natural infection) |
| | Comparison of healthy (n = 43) vs bacteremia (Staphylococcus aureus (n = 34), |
| | Escherichia coli (n = 15)) |
| GSE40366 | Age in humans (CMV titer = 0) |
| | Comparison of nonagenerians (n = 6) vs young (n = 11) (<28 years old) |
| GSE42834 | Multiple conditions in humans (naturally acquired) |
| | Comparison of controls (healthy, n = 147) vs tuberculosis (n = 66); sarcoidosis |
| | (active, n = 68; non-active n = 22); lung cancer (n = 16); bacterial pneumonia |
| | (treated and untreated with antibiotics) (n = 16) |
| GSE25504 | Bacterial sepsis in humans (neonates, naturally acquired). |
| | Comparison of controls (healthy and blood taken for other clinical reasons, |
| | n = 35) vs sepsis (n = 28) |
| GSE30119 | Bacterial sepsis in humans (naturally acquired) |
| | Comparison of controls (healthy, n = 44) vs sepsis (Staphylococcus aureus |
| | infection including bacteremia, n = 99) |
| GSE17755 | Autoimmune disease in humans |
| | Comparison of healthy (n = 53) vs autoimmune disease (rheumatoid arthritis, |
| | n = 112; Systemic Lupus Erythematosis, n = 22; Poly juvenile idiopathic arthritis, |
| | n = 6; Systemic juvenile idiopathic arthritis, n = 51) |
| GSE19301 | Asthma in humans (n = 117) |
| | Comparison of “quiet” (n = 292) vs “exacerbation” (n = 117) |
| GSE47655 | Anaphylaxis in humans (naturally acquired) |
| | Comparison of healthy (6 subjects, three time points, n = 18) vs anaphylaxis (6 |
| | patients, three time points, n = 18) |
| GSE38485 | Schizophrenia in humans |
| | Comparison of healthy (n = 22) vs schizophrenia (n = 15) |
| GSE36809 | Blunt trauma in humans |
| | Comparison of healthy (n = 37) vs trauma within 12 hours (n = 167) |
| GSE29532 | Coronary artery disease in humans, multiple time points. |
| | Comparison of controls (n = 6) vs CAD upon admission to ER (n = 49) |
| GSE46743 | Dexamethasone in human subjects (oral dose, induced) |
| | Comparison of pre-dose (n = 160) vs 3 hours post-dose (n = 160) |
| GSE61672 | Generalised anxiety disorder in humans |
| | Comparison of controls (n = 179) vs Patients on first visit (n = 157) |
| GSE64813 | Post-traumatic stress disorder in humans |
| | Comparison of 94 pre-deployment with 47 post-deployment with PTSD |
| GSE11908 | Multiple conditions in humans (naturally acquired) |
| | Comparison of healthy controls (n = 10) vs systemic juvenile idiopathic arthritis |
| | (n = 47); systemic lupus erythematosus (n = 40); type I diabetes (n = 20); |
| | metastatic melanoma (n = 39); Escherichia coli (n = 22); Staphylococcus aureus |
| | (n = 18); Liver-transplant recipients undergoing immunosuppressive therapy |
| | (n = 37) |
| GSE16129 | Staphylococcus aureus infection in humans (naturally acquired) |
| | Comparison of healthy control (n = 29) vs Staphylococcus aureus: (n = 97) |
| GSE40012 | SIRS in humans (naturally acquired) |
| | Comparison of healthy controls (n = 36, two time points, 18 subjects) vs SIRS |
| | (n = 40, multiple time points, 13 subjects) |
| GSE40396 | Bacterial infection in human (pediatric) (naturally acquired) |
| | Comparison of virus-negative afebrile controls (n = 19) vs culture positive |
| | Escherichia coli (n = 2) and Staphylococcus aureus (n = 4) |
| GSE6269 | Bacterial infection in human (pediatric) (naturally acquired) |
| | Comparison of healthy control (n = 6) vs Staphylococcus aureus (n = 50); |
| | Escherichia coli (n = 29); Streptococcus pneumoniae (n = 22) |
| GSE35846 | Race, gender and obesity in human subjects (mean age 51) |
| | Comparison of men (n = 69) vs women (n = 124) |
| | Comparison across race (Caucasian, n = 140; African American, n = 37; Asian, |
| | n = 11; American Indian, n = 1) |
| | Comparison across percentage body fat (9-53%) |

TABLE 20
LIST OF DERIVED VASIRS BIOMARKERS
WITH AN OF AUC >0.8 IN AT LEAST
11 OF 14 VIRAL DATASETS.

| Derived | Mean |
|---|---|
| Biomarker | AUC |
| IFI6:IL16 | 0.916 |
| OASL:NR3C1 | 0.915 |
| OASL:EMR2 | 0.914 |
| OASL:SORL1 | 0.908 |
| OASL:SERTAD2 | 0.907 |
| OASL:LPAR2 | 0.904 |
| OASL:ITGAX | 0.902 |
| OASL:TGFBR2 | 0.901 |
| OASL:KIAA0247 | 0.9 |
| OASL:ARHGAP26 | 0.899 |
| OASL:LYN | 0.899 |
| OASL:PCBP2 | 0.898 |
| OASL:TOPORS | 0.898 |
| EIF2AK2:IL16 | 0.896 |
| OASL:NCOA1 | 0.896 |
| OASL:PTGER4 | 0.896 |
| OASL:TLR2 | 0.895 |
| OASL:PACSIN2 | 0.894 |
| OASL:LILRA2 | 0.893 |
| OASL:PTPRE | 0.893 |
| OASL:RPS6KA1 | 0.893 |
| OASL:CASC3 | 0.892 |
| OASL:VEZF1 | 0.892 |
| OASL:CRLF3 | 0.891 |
| OASL:NDEL1 | 0.891 |
| OASL:RASSF2 | 0.891 |
| OASL:TLE4 | 0.891 |
| OASL:ADGRE5 | 0.89 |
| OASL:CEP68 | 0.89 |
| OASL:RXRA | 0.89 |
| OASL:SP3 | 0.89 |
| OASL:ABLIM1 | 0.889 |
| OASL:AOAH | 0.889 |
| OASL:MBP | 0.889 |
| OASL:NLRP1 | 0.889 |
| OASL:PBX3 | 0.889 |
| OASL:PTPN6 | 0.889 |
| OASL:RYBP | 0.889 |
| OASL:IL13RA1 | 0.888 |
| OASL:LCP2 | 0.888 |
| OASL:LRP10 | 0.888 |
| OASL:SYPL1 | 0.888 |
| OASL:VAMP3 | 0.888 |
| IFI44:LTB | 0.887 |
| OASL:ARHGEF2 | 0.887 |
| OASL:CTDSP2 | 0.887 |
| OASL:LST1 | 0.887 |
| OASL:MAPK1 | 0.887 |
| OASL:N4BP1 | 0.887 |
| OASL:STAT5B | 0.887 |
| IFI44:ABLIM1 | 0.886 |
| IFI44:IL6ST | 0.886 |
| OASL:BACH1 | 0.886 |
| OASL:KLF7 | 0.886 |
| OASL:PRMT2 | 0.886 |
| OASL:HCK | 0.885 |
| OASL:ITPKB | 0.885 |
| OASL:MAP4K4 | 0.885 |
| OASL:PPM1F | 0.885 |
| OASL:RAB14 | 0.885 |
| IFI6:ABLIM1 | 0.884 |
| OAS2:FAIM3 | 0.884 |
| OASL:ARHGAP25 | 0.884 |
| OASL:GNA12 | 0.884 |
| OASL:NUMB | 0.884 |
| OASL:CREBBP | 0.883 |
| OASL:PINK1 | 0.883 |
| OASL:PITPNA | 0.883 |
| OASL:SEMA4D | 0.883 |
| OASL:TGFBI | 0.883 |
| OASL:APLP2 | 0.882 |
| OASL:CCNG2 | 0.882 |
| OASL:MKRN1 | 0.882 |
| OASL:RGS14 | 0.882 |
| OASL:LYST | 0.881 |
| OASL:TNRC6B | 0.881 |
| OASL:TYROBP | 0.881 |
| OASL:WDR37 | 0.881 |
| OASL:WDR47 | 0.881 |
| UBE2L6:IL16 | 0.881 |
| OASL:BTG1 | 0.88 |
| OASL:CD93 | 0.88 |
| OASL:DCP2 | 0.88 |
| OASL:FYB | 0.88 |
| OASL:MAML1 | 0.88 |
| OASL:SNRK | 0.88 |
| OASL:USP4 | 0.88 |
| OASL:YTHDF3 | 0.88 |
| OASL:CEP170 | 0.879 |
| OASL:PLEKHO2 | 0.879 |
| OASL:SMAD4 | 0.879 |
| OASL:ST3GAL1 | 0.879 |
| OASL:ZNF292 | 0.879 |
| IFI44:IL4R | 0.878 |
| OASL:HPCAL1 | 0.878 |
| OASL:IGSF6 | 0.878 |
| OASL:MTMR3 | 0.878 |
| OASL:PHF20 | 0.878 |
| OASL:PPARD | 0.878 |
| OASL:PPP4R1 | 0.878 |
| OASL:RBMS1 | 0.878 |
| OASL:RHOG | 0.878 |
| OASL:TIAM1 | 0.878 |
| USP18:IL16 | 0.878 |
| OASL:CBX7 | 0.877 |
| OASL:RAF1 | 0.877 |
| OASL:SERINC5 | 0.877 |
| OASL:UBQLN2 | 0.877 |
| OASL:XPO6 | 0.877 |
| OASL:ATP6V1B2 | 0.876 |
| OASL:CSF2RB | 0.876 |
| OASL:GYPC | 0.876 |
| OASL:IL4R | 0.876 |
| OASL:MMP25 | 0.876 |
| OASL:PSEN1 | 0.876 |
| OASL:SH2B3 | 0.876 |
| OASL:STAT5A | 0.876 |
| ISG15:IL16 | 0.875 |
| MX1:LEF1 | 0.875 |
| OASL:CAMK2G | 0.875 |
| OASL:ETS2 | 0.875 |
| OASL:POLB | 0.875 |
| OASL:STK38L | 0.875 |
| OASL:TFE3 | 0.875 |
| OASL:ICAM3 | 0.874 |
| OASL:ITGB2 | 0.874 |
| OASL:PISD | 0.874 |
| OASL:PLXNC1 | 0.874 |
| OASL:SNX27 | 0.874 |
| OASL:TNIP1 | 0.874 |
| OASL:ZMIZ1 | 0.874 |
| OASL:FOXO3 | 0.873 |
| OASL:IL10RB | 0.873 |
| OASL:MAP3K5 | 0.873 |
| OASL:POLD4 | 0.873 |
| OASL:ARAP1 | 0.872 |
| OASL:CTBP2 | 0.872 |
| OASL:DGKA | 0.872 |
| OASL:NFYA | 0.872 |
| OASL:PCNX | 0.872 |
| OASL:PFDN5 | 0.872 |
| OASL:R3HDM2 | 0.872 |
| OASL:STX6 | 0.872 |
| EIF2AK2:SYPL1 | 0.871 |
| ISG15:ABLIM1 | 0.871 |
| OASL:FOXJ2 | 0.871 |
| OASL:IQSEC1 | 0.871 |
| OASL:LRMP | 0.871 |
| OASL:NAB1 | 0.871 |
| OASL:RAB31 | 0.871 |
| OASL:WASF2 | 0.871 |
| OASL:ZNF274 | 0.871 |
| OAS2:LEF1 | 0.87 |
| OASL:BRD1 | 0.87 |
| OASL:GNAQ | 0.87 |
| OASL:GSK3B | 0.87 |
| OASL:IL6R | 0.87 |
| OASL:MAPK14 | 0.87 |
| USP18:TGFBR2 | 0.87 |
| ISG15:LTB | 0.869 |
| OASL:INPP5D | 0.869 |
| OASL:MED13 | 0.869 |
| OASL:MORC3 | 0.869 |
| OASL:PTAFR | 0.869 |
| OASL:RBM23 | 0.869 |
| OASL:SNN | 0.869 |
| OASL:ST13 | 0.869 |
| OASL:TFEB | 0.869 |
| OASL:ZFYVE16 | 0.869 |
| EIF2AK2:SATB1 | 0.868 |
| OASL:ABAT | 0.868 |
| OASL:ABI1 | 0.868 |
| OASL:ACVR1B | 0.868 |
| OASL:GPSM3 | 0.868 |
| OASL:MPPE1 | 0.868 |
| OASL:PTEN | 0.868 |
| OASL:SEC62 | 0.868 |
| IFI6:MYC | 0.867 |
| IFI6:PCF11 | 0.867 |
| OASL:AIF1 | 0.867 |
| OASL:CSNK1D | 0.867 |
| OASL:GABARAP | 0.867 |
| OASL:HAL | 0.867 |
| OASL:LAPTM5 | 0.867 |
| OASL:XPC | 0.867 |
| USP18:NFKB1 | 0.867 |
| OASL:ACAP2 | 0.866 |
| OASL:CLEC4A | 0.866 |
| OASL:HIP1 | 0.866 |
| OASL:PIAS1 | 0.866 |
| OASL:PPP3R1 | 0.866 |
| OASL:RALB | 0.866 |
| OASL:RGS19 | 0.866 |
| OASL:TRIOBP | 0.866 |
| EIF2AK2:PDE3B | 0.865 |
| OASL:NCOA4 | 0.865 |
| OASL:RARA | 0.865 |
| OASL:RPS6KA3 | 0.865 |
| OASL:SIRPA | 0.865 |
| OASL:TLE3 | 0.865 |
| OASL:TNFRSF1A | 0.865 |
| DDX60:TGFBR2 | 0.864 |
| OASL:FLOT2 | 0.864 |
| OASL:FNBP1 | 0.864 |
| OASL:MAP3K3 | 0.864 |
| OASL:STX10 | 0.864 |
| OASL:ZDHHC18 | 0.864 |
| OASL:ZNF143 | 0.864 |
| TAP1:TGFBR2 | 0.864 |
| OAS2:ABLIM1 | 0.863 |
| OASL:ARRB2 | 0.863 |
| OASL:IKBKB | 0.863 |
| OASL:KBTBD2 | 0.863 |
| OASL:PHC2 | 0.863 |
| OASL:PUM2 | 0.863 |
| OASL:SSFA2 | 0.863 |
| IFI44:MYC | 0.862 |
| OASL:ABHD2 | 0.862 |
| OASL:CYLD | 0.862 |
| OASL:MAST3 | 0.862 |
| OASL:UBN1 | 0.862 |
| IFI6:IL6ST | 0.861 |
| IFIH1:TGFBR2 | 0.861 |
| OASL:CNPY3 | 0.861 |
| OASL:KIAA0232 | 0.861 |
| USP18:CHMP7 | 0.861 |
| USP18:NECAP2 | 0.861 |
| OASL:CAP1 | 0.86 |
| OASL:HPS1 | 0.86 |
| OASL:IL1RAP | 0.86 |
| OASL:MEF2A | 0.86 |
| OASL:RNF19B | 0.86 |
| OASL:TMEM127 | 0.86 |
| USP18:IL27RA | 0.86 |
| OASL:CDIPT | 0.859 |
| OASL:CREB1 | 0.859 |
| OASL:GPS2 | 0.859 |
| OASL:NDE1 | 0.859 |
| OASL:RAB11FIP1 | 0.859 |
| USP18:ABLIM1 | 0.859 |
| EIF2AK2:TNRC6B | 0.858 |
| OASL:FAM134A | 0.858 |
| OASL:FCGRT | 0.858 |
| OASL:LPIN2 | 0.858 |
| OASL:PECAM1 | 0.858 |
| OASL:WBP2 | 0.858 |
| OASL:ZNF148 | 0.858 |
| OASL:RTN3 | 0.857 |
| OASL:TYK2 | 0.857 |
| USP18:LTB | 0.857 |
| DHX58:IL16 | 0.856 |
| ISG15:IL4R | 0.856 |
| OASL:BRD4 | 0.856 |
| OASL:CCNT2 | 0.856 |
| OASL:FGR | 0.856 |
| OASL:ITSN2 | 0.856 |
| OASL:LYL1 | 0.856 |
| OASL:PHF3 | 0.856 |
| OASL:PSAP | 0.856 |
| OASL:STX3 | 0.856 |
| OASL:TNK2 | 0.856 |
| EIF2AK2:ZNF274 | 0.855 |
| OASL:ACAA1 | 0.855 |
| OASL:CHD3 | 0.855 |
| OASL:FRY | 0.855 |
| OASL:GRB2 | 0.855 |
| OASL:MAP3K11 | 0.855 |
| OASL:NEK7 | 0.855 |
| OASL:PPP2R5A | 0.855 |
| USP18:ST13 | 0.855 |
| XAF1:LEF1 | 0.855 |
| OASL:CASP8 | 0.854 |
| OASL:PCF11 | 0.854 |
| OASL:PRKCD | 0.854 |
| OASL:PSTPIP1 | 0.854 |
| OASL:SLCO3A1 | 0.854 |
| OASL:ZDHHC17 | 0.854 |
| USP18:FOXO1 | 0.854 |
| OASL:ASAP1 | 0.853 |
| OASL:BAZ2B | 0.853 |
| OASL:FAM65B | 0.853 |
| OASL:HHEX | 0.853 |
| OASL:MAX | 0.853 |
| OASL:PHF2 | 0.853 |
| OASL:RNF130 | 0.853 |
| OASL:SOS2 | 0.853 |
| OASL:STAM2 | 0.853 |
| OASL:ZFC3H1 | 0.853 |
| IFI44:CYLD | 0.852 |
| IFIH1:CRLF3 | 0.852 |
| OASL:BANP | 0.852 |
| OASL:CCND3 | 0.852 |
| OASL:DGCR2 | 0.852 |
| OASL:USP15 | 0.852 |
| USP18:EIF3H | 0.852 |
| OASL:LAT2 | 0.851 |
| OASL:ZYX | 0.851 |
| USP18:CAMK1D | 0.851 |
| ZBP1:NDE1 | 0.851 |
| EIF2AK2:IL4R | 0.85 |
| IFI44:SESN1 | 0.85 |
| OASL:CD37 | 0.85 |
| OASL:CST3 | 0.85 |
| OASL:DPEP2 | 0.85 |
| OASL:MYC | 0.85 |
| OASL:RERE | 0.85 |
| OASL:USP10 | 0.85 |
| USP18:LEF1 | 0.85 |
| OASL:MXI1 | 0.849 |
| OASL:PRUNE | 0.849 |
| OASL:VPS8 | 0.849 |
| OASL:CYTH4 | 0.848 |
| OASL:FBXO11 | 0.848 |
| OASL:PRKAA1 | 0.848 |
| OASL:SERINC3 | 0.848 |
| OASL:UBXN2B | 0.848 |
| USP18:DPF2 | 0.848 |
| USP18:NACA | 0.848 |
| USP18:SYPL1 | 0.848 |
| ISG15:DGKA | 0.847 |
| OASL:MARK3 | 0.847 |
| USP18:DIDO1 | 0.846 |
| CUL1:IL16 | 0.845 |
| OASL:DOCK9 | 0.845 |
| USP18:PIK3IP1 | 0.845 |
| OASL:FBXO9 | 0.844 |
| OASL:MKLN1 | 0.844 |
| OASL:PPP1R11 | 0.844 |
| USP18:DGKA | 0.844 |
| USP18:ZNF274 | 0.844 |
| OASL:POLR1D | 0.843 |
| OASL:SETD2 | 0.843 |
| DDX60:ABLIM1 | 0.842 |
| OASL:ARHGAP15 | 0.842 |
| OASL:BCL2 | 0.842 |
| OASL:GOLGA7 | 0.842 |
| OASL:KIAA0513 | 0.842 |
| OASL:MARCH7 | 0.842 |
| USP18:LDLRAP1 | 0.842 |
| C19orf66:IL16 | 0.841 |
| OASL:ARRB1 | 0.841 |
| OASL:BMP2K | 0.841 |
| OASL:LIMK2 | 0.841 |
| OASL:RNASET2 | 0.841 |
| USP18:ATM | 0.841 |
| USP18:CYLD | 0.841 |
| USP18:NOSIP | 0.841 |
| OASL:TNFSF13 | 0.84 |
| OASL:TRIM8 | 0.84 |
| XAF1:IL4R | 0.84 |
| DHX58:ABLIM1 | 0.839 |
| OASL:MANSC1 | 0.839 |
| OASL:MAP1LC3B | 0.839 |
| OASL:OSBPL2 | 0.839 |
| OASL:RAB7A | 0.839 |
| EIF2AK2:ZFC3H1 | 0.838 |
| IFIH1:LTB | 0.838 |
| OASL:FES | 0.838 |
| OASL:HGSNAT | 0.838 |
| OASL:KLF6 | 0.838 |
| OASL:TM2D3 | 0.838 |
| OASL:KLHL2 | 0.837 |
| OASL:MAPRE2 | 0.837 |
| OASL:RNF146 | 0.837 |
| USP18:RPL22 | 0.837 |
| DHX58:LTB | 0.836 |
| OASL:GMIP | 0.836 |
| DDX60:SYPL1 | 0.835 |
| EIF2AK2:IL6ST | 0.835 |
| EIF2AK2:PCF11 | 0.835 |
| ISG15:NOSIP | 0.835 |
| OASL:NRBF2 | 0.835 |
| OASL:RNF141 | 0.835 |
| OASL:VAV3 | 0.835 |
| OASL:ZFAND5 | 0.835 |
| USP18:NDFIP1 | 0.835 |
| USP18:TMEM204 | 0.835 |
| USP18:UBE2D2 | 0.835 |
| OASL:CAMK1D | 0.834 |
| OASL:CLK4 | 0.834 |
| OASL:MCTP2 | 0.834 |
| OASL:MOSPD2 | 0.834 |
| OASL:TSC22D3 | 0.834 |
| USP18:CRLF3 | 0.834 |
| USP18:SESN1 | 0.834 |
| USP18:ZC3HAV1 | 0.834 |
| OASL:MSL1 | 0.833 |
| OASL:TREM1 | 0.833 |
| OASL:YPEL5 | 0.833 |
| USP18:CIAPIN1 | 0.833 |
| USP18:PDCD6IP | 0.833 |
| HERC5:ABLIM1 | 0.832 |
| OASL:OSBPL11 | 0.832 |
| OASL:PLEKHO1 | 0.832 |
| USP18:CRTC3 | 0.832 |
| HERC6:ATM | 0.831 |
| ISG15:SESN1 | 0.831 |
| OAS2:MYC | 0.831 |
| OASL:OGFRL1 | 0.831 |
| OASL:ZXDC | 0.831 |
| USP18:CCR7 | 0.831 |
| OASL:APBB1IP | 0.83 |
| OASL:CHST11 | 0.83 |
| OASL:GPBP1L1 | 0.83 |
| USP18:SSBP2 | 0.83 |
| OASL:RC3H2 | 0.829 |
| USP18:UTP14A | 0.829 |
| OASL:GCC2 | 0.828 |
| USP18:LRMP | 0.828 |
| USP18:TRIB2 | 0.828 |
| OASL:GPR97 | 0.827 |
| EIF2AK2:BTG1 | 0.826 |
| EIF2AK2:CYLD | 0.826 |
| OASL:PAFAH1B1 | 0.826 |
| USP18:BTG1 | 0.826 |
| USP18:NCBP2 | 0.826 |
| USP18:PPP1R2 | 0.826 |
| LAP3:MAP4K4 | 0.825 |
| OASL:ERBB2IP | 0.825 |
| OASL:NOD2 | 0.825 |
| OASL:RIN3 | 0.825 |
| OASL:TMBIM1 | 0.825 |
| ZBP1:XPO6 | 0.825 |
| ISG15:LDLRAP1 | 0.824 |
| OASL:CHMP1B | 0.824 |
| OASL:LILRB3 | 0.824 |
| OASL:PHF20L1 | 0.823 |
| USP18:PCF11 | 0.823 |
| OASL:ANKRD49 | 0.822 |
| OASL:DOK3 | 0.822 |
| OASL:PRKAG2 | 0.822 |
| OASL:SOAT1 | 0.822 |
| USP18:IL6ST | 0.822 |
| USP18:RPL10A | 0.822 |
| LAP3:SYPL1 | 0.82 |
| OASL:MARCH8 | 0.819 |
| TAP1:TNRC6B | 0.819 |
| OASL:KLF3 | 0.818 |
| PHF11:ZNF274 | 0.818 |
| OASL:PGS1 | 0.817 |
| OASL:ZNF238 | 0.817 |
| STAT1:PCBP2 | 0.817 |
| OASL:SH2D3C | 0.816 |
| USP18:SAFB2 | 0.816 |
| EIF2AK2:CAMK1D | 0.815 |
| LAP3:CNPY3 | 0.815 |
| LAP3:NDFIP1 | 0.815 |
| LAP3:TRAK1 | 0.815 |
| OASL:NPL | 0.815 |
| OASL:NSUN3 | 0.815 |
| OASL:ATAD2B | 0.814 |
| ZBP1:KLF7 | 0.813 |
| ZBP1:PCF11 | 0.813 |
| LAP3:ABLIM1 | 0.812 |
| OASL:CSAD | 0.812 |
| PHF11:IL16 | 0.812 |
| USP18:BEX4 | 0.812 |
| USP18:METTL3 | 0.812 |
| RTP4:ABLIM1 | 0.811 |
| HERC6:MYC | 0.81 |
| USP18:ALDH3A2 | 0.81 |
| OASL:RAB4B | 0.809 |
| USP18:ATF7IP2 | 0.809 |
| TAP1:TGOLN2 | 0.807 |
| PARP12:ABLIM1 | 0.806 |
| RSAD2:CAMK1D | 0.806 |
| ZBP1:CYLD | 0.806 |
| STAT1:FBXO11 | 0.805 |
| ZBP1:ZFC3H1 | 0.805 |
| OASL:SIRPB1 | 0.804 |
| OASL:C2orf68 | 0.802 |
| RTP4:SYPL1 | 0.802 |
| LAP3:JAK1 | 0.801 |

TABLE 21
DETAILS OF GENE EXPRESSION OMNIBUS
(GEO) DATASETS USED FOR DISCOVERY
OF PROTOZOAL DERIVED BIOMARKER

| Dataset | Group | Case | Controls | Total # | Genes |
|---|---|---|---|---|---|
| GSE34404 | Malaria | 42 | 61 | 103 | 21511 |
| GSE64610 | Leishmania | 10 | 5 | 15 | 6805 |
| GSE15221 | Malaria | 14 | 14 | 28 | 36292 |
| GSE5418 | Malaria | 15 | 22 | 37 | 12439 |
| Merged Data | All | 86 | 107 | 193 | 4421 |

TABLE 22
DESCRIPTION OF THE GEO DATASETS USED FOR VALIDATION
OF THE PROTOZOAL DERIVED BIOMARKERS

| GEO | | | |
|---|---|---|---|
| Dataset | Organism | Tissue | Study Description |
| GSE43661 | Leishmania | Macrophages, | 3 donors, cultured cells either infected with |
| | major | in vitro | Leishmania or not. Samples taken at 0, 3, |
| | | | 6, 12 and 24 hours |
| GSE23750 | Entamoeba | Intestinal | 8 donors, samples taken on Day 1 and 60, |
| | histolytica | biopsies | pre- and post-treatment |
| GSE7047 | Trypanosoma | HeLa cells, | 3 replicates of cells either infected or not |
| | cruzi | in vitro | |
| GSE50957 | Plasmodium | Peripheral | Pilot study: 5 donors, samples taken pre- |
| | falciparum | blood | and post- being bitten by infected mosquitos. |
| | | | All donors were on chloroquin treatment |
| GSE52166 | Plasmodium | Peripheral | Large study, as per GSE50957 |
| | falciparum | blood | |

TABLE 23
DESCRIPTION OF CONTROL DATASETS
USED FOR SUBTRACTION FROM THE
DERIVED BIOMARKERS FOR PASIRS
The subtraction process ensured that the
PaSIRS derived biomarkers were specific.

| Dataset | Case | Controls | Total # | Genes | Response |
|---|---|---|---|---|---|
| GSE40366 | 69 | 17 | 86 | 20293 | Viral |
| GSE38485 | 106 | 96 | 202 | 19206 | SIRS |
| GSE46743 | 160 | 160 | 320 | 9595 | SIRS |
| GSE64813 | 47 | 141 | 188 | 10146 | SIRS |
| GSE17755 | 191 | 53 | 244 | 6620 | SIRS |
| GSE41752 | 19 | 11 | 30 | 18515 | Viral |
| GSE29532 | 25 | 6 | 31 | 14332 | SIRS |
| GSE51808 | 28 | 9 | 37 | 18353 | Viral |
| GSE19301 | 166 | 394 | 560 | 12631 | SIRS |
| GSE52428 | 41 | 39 | 80 | 12631 | Viral |
| GSE11908 | 40 | 196 | 236 | 12631 | Bacterial |
| GSE47655 | 1 | 35 | 36 | 5196 | SIRS |
| GSE25504 | 26 | 37 | 63 | 13510 | Bacterial |
| GSE61672 | 157 | 179 | 336 | 9291 | SIRS |
| GSE35846 | 124 | 65 | 189 | 10330 | Gender |
| GSE33341 | 51 | 43 | 94 | 12631 | Bacterial |

TABLE 24
DESCRIPTION OF DATASETS USED FOR
DISCOVERY, VALIDATION AND SUBTRACTION
FROM THE DERIVED BIOMARKERS FOR INSIRS.
The subtraction process ensured that the
InSIRS derived biomarkers were specific.

| Dataset | Description | How Used |
|---|---|---|
| GAPPSS | In-house clinical trial. Pediatric patients | Discovery/Validation |
| | in ICU. Post-surgical vs confirmed sepsis | |
| GSE17755 | Autoimmune disease vs infected | Discovery/Validation |
| GSE36809 | Trauma (non-infected early stage vs | Discovery/Validation |
| | infected) | |
| GSE47655 | Anaphylaxis (presentation vs resolved) | Discovery/Validation |
| GSE63990 | Acute respiratory inflammation | Discovery/Validation |
| | (infected vs non-infected) | |
| GSE74224 | Sepsis vs SIRS (in-house data) | Discovery/Validation |
| GSE11908 | Autoimmune disease, cancer, liver | Control/Subtraction |
| | cirrhosis vs infected | |
| GSE19301 | Asthma (exacerbation vs quiescent) | Control/Subtraction |
| GSE38485 | Schizophrenia vs healthy control | Control/Subtraction |
| GSE41752 | Lassa virus infection vs healthy | Control/Subtraction |
| GSE42834 | Tuberculosis vs sarcoidosis | Control/Subtraction |
| GSE51808 | Dengue virus vs healthy control | Control/Subtraction |
| GSE52428 | Influenza virus vs healthy control | Control/Subtraction |
| GSE61672 | Anxiety vs healthy control | Control/Subtraction |
| GSE64813 | Post-traumatic stress disorder vs | Control/Subtraction |
| | pre-stress | |

TABLE 25
DERIVED BIOMARKERS GROUPED (A, B, C, D) BASED ON
CORRELATION TO EACH OF THE BIOMARKERS IN THE
FINAL BASIRS SIGNATURE (OPLAH, ZHX2, TSPO, HCLS1)

| Group A | | | Group B | | | Group C | | | Group D | | |
|---|---|---|---|---|---|---|---|---|---|---|---|
| Corre- | | | Corre- | | | Corre- | | | Corre- | | |
| lation | HUGO | DNA | lation | HUGO | DNA | lation | HUGO | DNA | lation | HUGO | DNA |
| to | Gene | SEQ | to | Gene | SEQ | to | Gene | SEQ | to | Gene | SEQ |
| OPLAH | Symbol | ID | ZHX2 | Symbol | ID | TSPO | Symbol | ID | HCLS1 | Symbol | ID |
| 0.703 | ENTPD7 | 15 | 0.474 | SAP130 | 79 | 0.789 | HK3 | 29 | 0.490 | SEMA4D | 80 |
| 0.678 | PCOLCE2 | 59 | 0.468 | NLRP1 | 53 | 0.770 | RAB32 | 72 | 0.488 | INPP5D | 36 |
| 0.656 | ITGA7 | 37 | 0.440 | CNNM3 | 10 | 0.763 | IMPDH1 | 35 | 0.381 | ZFP36L2 | 93 |
| 0.611 | PDGFC | 60 | 0.427 | POGZ | 65 | 0.703 | FAM129A | 18 | 0.337 | CLEC7A | 9 |
| 0.590 | SMPDL3A | 82 | 0.387 | GRK5 | 26 | 0.661 | GAS7 | 24 | 0.321 | RNASE6 | 76 |
| 0.531 | GALNT2 | 23 | 0.375 | IL2RB | 34 | 0.622 | CD82 | 8 | 0.292 | MME | 50 |
| 0.465 | CR1 | 12 | 0.357 | NPAT | 56 | 0.599 | ADM | 2 | 0.291 | PARP8 | 58 |
| 0.458 | IGFBP7 | 31 | 0.352 | PRKD2 | 66 | 0.563 | IK | 32 | 0.242 | NCOA6 | 51 |
| 0.441 | FIG4 | 20 | 0.350 | FOXJ3 | 21 | 0.525 | ALPL | 3 | 0.127 | LPIN2 | 46 |
| 0.404 | COX15 | 11 | 0.335 | JARID2 | 38 | 0.519 | TLR5 | 88 | | | |
| 0.386 | NMUR1 | 54 | 0.323 | RBM23 | 74 | 0.484 | DENND3 | 13 | | | |
| 0.364 | MCTP1 | 48 | 0.318 | PRKDC | 67 | 0.470 | MGAM | 49 | | | |
| 0.348 | EPHB4 | 16 | 0.310 | TGFBR3 | 86 | 0.435 | TMEM165 | 89 | | | |
| 0.302 | ICK | 30 | 0.308 | SIDT1 | 81 | 0.409 | PRPF38B | 68 | | | |
| 0.218 | MBIP | 47 | 0.305 | TLE3 | 87 | 0.402 | EXTL3 | 17 | | | |
| 0.155 | PDS5B | 61 | 0.296 | QRICH1 | 71 | 0.352 | ADAM19 | 1 | | | |
| 0.082 | IKZF5 | 33 | 0.286 | KLRD1 | 42 | 0.335 | ST3GAL2 | 84 | | | |
| 0.046 | KIAA0101 | 39 | 0.267 | NFIC | 52 | 0.330 | CAMK1D | 4 | | | |
| | | | 0.265 | HAL | 27 | 0.310 | DOCK5 | 14 | | | |
| | | | 0.260 | RBM15 | 73 | 0.290 | GAB2 | 22 | | | |
| | | | 0.258 | KIAA0355 | 40 | 0.283 | CCNK | 7 | | | |
| | | | 0.255 | CBLL1 | 6 | | | | | | |
| | | | 0.251 | PYHIN1 | 70 | | | | | | |
| | | | 0.249 | KIAA0907 | 41 | | | | | | |
| | | | 0.217 | RFC1 | 75 | | | | | | |
| | | | 0.210 | KLRF1 | 43 | | | | | | |
| | | | 0.205 | SPIN1 | 83 | | | | | | |
| | | | 0.198 | PHF3 | 62 | | | | | | |
| | | | 0.196 | UTRN | 91 | | | | | | |
| | | | 0.190 | RUNX2 | 77 | | | | | | |
| | | | 0.184 | PRSS23 | 69 | | | | | | |
| | | | 0.178 | NOV | 55 | | | | | | |
| | | | 0.177 | RYK | 78 | | | | | | |
| | | | 0.176 | LEPROTL1 | 45 | | | | | | |
| | | | 0.174 | CASS4 | 5 | | | | | | |
| | | | 0.164 | GCC2 | 25 | | | | | | |
| | | | 0.146 | PLA2G7 | 64 | | | | | | |
| | | | 0.141 | FBXO28 | 19 | | | | | | |
| | | | 0.140 | YPEL1 | 92 | | | | | | |
| | | | 0.134 | PIK3C2A | 63 | | | | | | |
| | | | 0.133 | LAG3 | 44 | | | | | | |
| | | | 0.120 | SYTL2 | 85 | | | | | | |

TABLE 26
DERIVED BIOMARKERS GROUPED (A, B, C, D) BASED ON CORRELATION TO EACH OF THE
BIOMARKErS IN THE FINAL VASIRS SIGNATURE (ISG15, IL16, OASL ADGRE5)

| Group A | | | Group B | | | Group C | | | Group D | | |
|---|---|---|---|---|---|---|---|---|---|---|---|
| Corre- | | | Corre- | | | Corre- | | | Corre- | | |
| lation | HUGO | | lation | HUGO | | lation | HUGO | | lation | HUGO | |
| to | Gene | SEQ | to | Gene | SEQ | to | Gene | | to | Gene | |
| ISG15 | Name | ID | IL16 | Name | ID | OASL | Name | SEQID | ADGRE5 | Name | SEQID |
| 1.000 | ISG15 | 330 | 1.000 | IL16 | 322 | 1.000 | OASL | 415 | 1.000 | ADGRE5 | 237 |
| 0.915 | IFI44 | 315 | 0.661 | ITPKB | 333 | 0.529 | N4BP1 | 393 | 0.634 | CYTH4 | 261 |
| 0.915 | RSAD2 | 496 | 0.600 | CAMK2G | 226 | 0.460 | NOD2 | 407 | 0.599 | HCK | 306 |
| 0.913 | HERC5 | 307 | 0.567 | CTDSP2 | 258 | 0.455 | RNF19B | 491 | 0.582 | ARHGAP26 | 205 |
| 0.906 | MX1 | 390 | 0.555 | DPEP2 | 270 | 0.437 | PRKAG2 | 456 | 0.555 | RARA | 477 |
| 0.895 | HERC6 | 308 | 0.551 | LTB | 358 | 0.413 | IGSF6 | 318 | 0.547 | XPO6 | 584 |
| 0.894 | OA52 | 414 | 0.549 | CBX7 | 230 | 0.352 | MEF2A | 380 | 0.534 | TNFRSF1A | 550 |
| 0.894 | XAF1 | 582 | 0.535 | FNBP1 | 286 | 0.343 | LPIN2 | 354 | 0.527 | SLCO3A1 | 514 |
| 0.892 | IFI6 | 316 | 0.534 | FOXO1 | 288 | 0.341 | PPP1R11 | 450 | 0.526 | ICAM3 | 314 |
| 0.873 | PARP12 | 421 | 0.531 | MAST3 | 375 | 0.316 | USP15 | 570 | 0.521 | PTPN6 | 466 |
| 0.872 | EIF2AK2 | 272 | 0.529 | LDLRAP1 | 348 | 0.308 | BACH1 | 214 | 0.519 | PRKCD | 457 |
| 0.849 | DHX58 | 266 | 0.529 | TMEM204 | 549 | 0.307 | SSFA2 | 524 | 0.518 | RAB11FIP1 | 470 |
| 0.844 | UBE2L6 | 565 | 0.526 | FAIM3 | 277 | 0.299 | MKLN1 | 382 | 0.514 | CSF2RB | 254 |
| 0.838 | DDX60 | 263 | 0.516 | RGS14 | 483 | 0.270 | FYB | 291 | 0.512 | LCP2 | 347 |
| 0.819 | USP18 | 571 | 0.516 | IKBKB | 319 | 0.250 | NSUN3 | 412 | 0.509 | TYROBP | 563 |
| 0.817 | RTP4 | 498 | 0.516 | ZXDC | 600 | 0.232 | MAX | 376 | 0.499 | PHC2 | 431 |
| 0.815 | PHF11 | 432 | 0.508 | PHF20 | 434 | 0.218 | STAM2 | 527 | 0.496 | RHOG | 485 |
| 0.812 | IFIH1 | 317 | 0.507 | DGKA | 265 | 0.209 | HHEX | 310 | 0.496 | PSAP | 460 |
| 0.792 | ZBP1 | 587 | 0.501 | XPC | 583 | 0.207 | CLEC4A | 246 | 0.496 | LYN | 360 |
| 0.766 | STAT1 | 528 | 0.499 | PPARD | 448 | 0.197 | ZFAND5 | 592 | 0.494 | TMEM127 | 548 |
| 0.765 | LAP3 | 344 | 0.499 | C2orf68 | 224 | 0.188 | ABI1 | 191 | 0.492 | LILRA2 | 350 |
| 0.755 | TAP1 | 536 | 0.494 | NLRP1 | 406 | 0.144 | MORC3 | 385 | 0.485 | AOAH | 199 |
| 0.741 | C19orf66 | 223 | 0.488 | IL27RA | 324 | 0.142 | RC3H2 | 481 | 0.476 | FGR | 284 |
| 0.617 | CUL1 | 259 | 0.480 | ABLIM1 | 192 | 0.137 | MAP1LC3B | 364 | 0.470 | PLEKHO2 | 443 |
| 0.453 | POLB | 445 | 0.477 | JAK1 | 335 | 0.120 | TM2D3 | 546 | 0.470 | ARAP1 | 202 |
| 0.395 | ZC3HAV1 | 589 | 0.475 | METTL3 | 381 | 0.100 | CHST11 | 244 | 0.468 | RBM23 | 479 |
| | | | 0.474 | SAFB2 | 501 | 0.097 | NAB1 | 394 | 0.462 | PTPRE | 467 |
| | | | 0.474 | PPM1F | 449 | 0.014 | KLF3 | 340 | 0.459 | KLF6 | 341 |
| | | | 0.473 | TYK2 | 562 | −0.030 | YPEL5 | 585 | 0.458 | LIMK2 | 352 |
| | | | 0.471 | BANP | 215 | −0.063 | MXI1 | 391 | 0.456 | LILRB3 | 351 |
| | | | 0.470 | CRTC3 | 252 | | | | 0.454 | TLR2 | 545 |
| | | | 0.468 | ATM | 212 | | | | 0.451 | GPR97 | 299 |
| | | | 0.453 | PAFAH1B1 | 420 | | | | 0.451 | GMIP | 294 |
| | | | 0.447 | PIK3IP1 | 438 | | | | 0.446 | SIRPA | 512 |
| | | | 0.445 | WDR37 | 580 | | | | 0.444 | LRP10 | 356 |
| | | | 0.444 | TGFBR2 | 540 | | | | 0.444 | LPAR2 | 353 |
| | | | 0.442 | ZNF274 | 598 | | | | 0.442 | TREM1 | 557 |
| | | | 0.429 | STAT5B | 530 | | | | 0.441 | IL13RA1 | 321 |
| | | | 0.427 | MAML1 | 362 | | | | 0.439 | ITGAX | 331 |
| | | | 0.420 | SATB1 | 502 | | | | 0.435 | ARHGAP25 | 204 |
| | | | 0.419 | DOCK9 | 268 | | | | 0.433 | SIRPB1 | 513 |
| | | | 0.417 | CHMP7 | 243 | | | | 0.433 | ZDHHC18 | 591 |
| | | | 0.413 | BRD1 | 220 | | | | 0.433 | TLE3 | 543 |
| | | | 0.410 | BTG1 | 222 | | | | 0.432 | ITGB2 | 332 |
| | | | 0.408 | ATF7IP2 | 211 | | | | 0.432 | SNX27 | 518 |
| | | | 0.408 | DIDO1 | 267 | | | | 0.431 | PGS1 | 430 |
| | | | 0.407 | LEF1 | 349 | | | | 0.429 | ATP6V1B2 | 213 |
| | | | 0.407 | TNRC6B | 554 | | | | 0.428 | RAB31 | 472 |
| | | | 0.405 | SERTAD2 | 507 | | | | 0.427 | MAP3K11 | 365 |
| | | | 0.405 | CEP68 | 240 | | | | 0.427 | PACSIN2 | 419 |
| | | | 0.398 | BCL2 | 217 | | | | 0.427 | KIAA0513 | 339 |
| | | | 0.397 | VPS8 | 577 | | | | 0.426 | EMR2 | 274 |
| | | | 0.396 | CHD3 | 241 | | | | 0.426 | RERE | 482 |
| | | | 0.393 | PUM2 | 468 | | | | 0.426 | NUMB | 413 |
| | | | 0.390 | TGOLN2 | 541 | | | | 0.425 | RALB | 476 |
| | | | 0.383 | NDE1 | 399 | | | | 0.425 | ETS2 | 276 |
| | | | 0.382 | CCR7 | 234 | | | | 0.422 | STAT5A | 529 |
| | | | 0.381 | PSTPIP1 | 462 | | | | 0.421 | LST1 | 357 |
| | | | 0.379 | TIAM1 | 542 | | | | 0.417 | RIN3 | 486 |
| | | | 0.376 | PECAM1 | 428 | | | | 0.417 | TNK2 | 553 |
| | | | 0.374 | PDE3B | 427 | | | | 0.416 | IQSEC1 | 329 |
| | | | 0.374 | MYC | 392 | | | | 0.413 | PISD | 440 |
| | | | 0.371 | FOXJ2 | 287 | | | | 0.412 | SORL1 | 520 |
| | | | 0.370 | PRMT2 | 458 | | | | 0.412 | FES | 283 |
| | | | 0.370 | CSNK1D | 255 | | | | 0.411 | K1AA0247 | 338 |
| | | | 0.357 | RPL10A | 492 | | | | 0.404 | IL6R | 326 |
| | | | 0.356 | SERINC5 | 506 | | | | 0.404 | LAPTM5 | 345 |
| | | | 0.354 | ARHGEF2 | 206 | | | | 0.402 | VAMP3 | 574 |
| | | | 0.352 | HGSNAT | 309 | | | | 0.400 | FAM65B | 279 |
| | | | 0.350 | TRAK1 | 556 | | | | 0.398 | MAP3K5 | 367 |
| | | | 0.350 | PHF2 | 433 | | | | 0.396 | TRIM8 | 559 |
| | | | 0.349 | PBX3 | 422 | | | | 0.396 | ZYX | 601 |
| | | | 0.349 | SESN1 | 508 | | | | 0.388 | MAPK14 | 370 |
| | | | 0.341 | DPF2 | 271 | | | | 0.387 | PLEKHO1 | 442 |
| | | | 0.338 | IL4R | 325 | | | | 0.387 | NCOA1 | 397 |
| | | | 0.334 | NOSIP | 408 | | | | 0.384 | RNASET2 | 487 |
| | | | 0.331 | MPPE1 | 387 | | | | 0.383 | APBB1IP | 200 |
| | | | 0.321 | NR3C1 | 410 | | | | 0.381 | RXRA | 499 |
| | | | 0.320 | ABAT | 189 | | | | 0.375 | PTAFR | 463 |
| | | | 0.320 | GCC2 | 293 | | | | 0.373 | CNPY3 | 248 |
| | | | 0.316 | ZFC3H1 | 593 | | | | 0.373 | TNFSF13 | 551 |
| | | | 0.311 | SETD2 | 509 | | | | 0.368 | RPS6KA1 | 494 |
| | | | 0.308 | ITSN2 | 334 | | | | 0.367 | OSBPL2 | 418 |
| | | | 0.306 | R3HDM2 | 469 | | | | 0.367 | MTMR3 | 389 |
| | | | 0.302 | ARHGAP15 | 203 | | | | 0.362 | TMBIM1 | 547 |
| | | | 0.301 | PCF11 | 424 | | | | 0.359 | TFEB | 538 |
| | | | 0.301 | MAPRE2 | 371 | | | | 0.359 | TFE3 | 537 |
| | | | 0.299 | ST3GAL1 | 526 | | | | 0.358 | RAF1 | 475 |
| | | | 0.299 | NACA | 395 | | | | 0.357 | STX3 | 533 |
| | | | 0.299 | WDR47 | 581 | | | | 0.357 | LAT2 | 346 |
| | | | 0.298 | SSBP2 | 523 | | | | 0.356 | GRB2 | 302 |
| | | | 0.293 | CLK4 | 247 | | | | 0.355 | NDEL1 | 400 |
| | | | 0.289 | EIF3H | 273 | | | | 0.355 | SEMA4D | 504 |
| | | | 0.287 | FRY | 290 | | | | 0.353 | FCGRT | 282 |
| | | | 0.286 | ZNF238 | 597 | | | | 0.353 | DOK3 | 269 |
| | | | 0.286 | PTGER4 | 465 | | | | 0.353 | HIP1 | 311 |
| | | | 0.285 | PCNX | 425 | | | | 0.353 | UBN1 | 566 |
| | | | 0.283 | NECAP2 | 402 | | | | 0.352 | PLXNC1 | 444 |
| | | | 0.279 | CASC3 | 228 | | | | 0.351 | NRBF2 | 411 |
| | | | 0.279 | MSL1 | 388 | | | | 0.348 | INPP5D | 328 |
| | | | 0.278 | VEZF1 | 576 | | | | 0.347 | SH2D3C | 511 |
| | | | 0.275 | K1AA0232 | 337 | | | | 0.347 | MMP25 | 384 |
| | | | 0.274 | RASSF2 | 478 | | | | 0.342 | IL10RB | 320 |
| | | | 0.268 | RPL22 | 493 | | | | 0.340 | FLOT2 | 285 |
| | | | 0.265 | ACAA1 | 193 | | | | 0.339 | PIAS1 | 437 |
| | | | 0.263 | MAP4K4 | 368 | | | | 0.338 | PITPNA | 441 |
| | | | 0.263 | BEX4 | 218 | | | | 0.334 | APLP2 | 201 |
| | | | 0.263 | NCBP2 | 396 | | | | 0.333 | CTBP2 | 257 |
| | | | 0.262 | LRMP | 355 | | | | 0.332 | GPSM3 | 301 |
| | | | 0.259 | CAMK1D | 225 | | | | 0.331 | RNF130 | 488 |
| | | | 0.257 | UTP14A | 573 | | | | 0.326 | DGCR2 | 264 |
| | | | 0.253 | STX6 | 534 | | | | 0.326 | ZMIZ1 | 595 |
| | | | 0.253 | RPS6KA3 | 495 | | | | 0.320 | CAP1 | 227 |
| | | | 0.249 | PRKAA1 | 455 | | | | 0.319 | GSK3B | 303 |
| | | | 0.240 | GOLGA7 | 297 | | | | 0.318 | RGS19 | 484 |
| | | | 0.239 | ZNF143 | 596 | | | | 0.317 | RAB7A | 474 |
| | | | 0.237 | SNRK | 517 | | | | 0.316 | CREBBP | 250 |
| | | | 0.233 | SYPL1 | 535 | | | | 0.313 | RBMS1 | 480 |
| | | | 0.229 | CYLD | 260 | | | | 0.310 | IL1RAP | 323 |
| | | | 0.228 | PRUNE | 459 | | | | 0.308 | RTN3 | 497 |
| | | | 0.224 | CRLF3 | 251 | | | | 0.308 | PPP4R1 | 454 |
| | | | 0.223 | CD93 | 236 | | | | 0.307 | TRIOBP | 560 |
| | | | 0.223 | GPS2 | 300 | | | | 0.306 | GABARAP | 292 |
| | | | 0.221 | FBXO11 | 280 | | | | 0.305 | MCTP2 | 378 |
| | | | 0.217 | UBE2D2 | 564 | | | | 0.304 | NFKB1 | 404 |
| | | | 0.217 | USP10 | 569 | | | | 0.303 | CST3 | 256 |
| | | | 0.216 | CCNG2 | 232 | | | | 0.292 | ABHD2 | 190 |
| | | | 0.212 | S0S2 | 521 | | | | 0.285 | SH2B3 | 510 |
| | | | 0.211 | ARRB1 | 207 | | | | 0.284 | STX10 | 532 |
| | | | 0.207 | CEP170 | 239 | | | | 0.282 | TSC22D3 | 561 |
| | | | 0.206 | SMAD4 | 515 | | | | 0.280 | TLE4 | 544 |
| | | | 0.205 | CIAPIN1 | 245 | | | | 0.277 | HAL | 305 |
| | | | 0.204 | KLF7 | 342 | | | | 0.277 | ARRB2 | 208 |
| | | | 0.198 | PHF20L1 | 435 | | | | 0.276 | MAP3K3 | 366 |
| | | | 0.194 | ALDH3A2 | 197 | | | | 0.274 | NPL | 409 |
| | | | 0.193 | PDCD6IP | 426 | | | | 0.265 | CCND3 | 231 |
| | | | 0.185 | WASF2 | 578 | | | | 0.265 | SERINC3 | 505 |
| | | | 0.184 | TGFBI | 539 | | | | 0.263 | GNAQ | 296 |
| | | | 0.175 | GPBP1L1 | 298 | | | | 0.262 | USP4 | 572 |
| | | | 0.174 | PCBP2 | 423 | | | | 0.261 | PSEN1 | 461 |
| | | | 0.166 | DCP2 | 262 | | | | 0.256 | KBTBD2 | 336 |
| | | | 0.165 | LYST | 361 | | | | 0.254 | LYL1 | 359 |
| | | | 0.154 | ERBB2IP | 275 | | | | 0.241 | AIF1 | 196 |
| | | | 0.146 | ANKRD49 | 198 | | | | 0.239 | MBP | 377 |
| | | | 0.145 | NDFIP1 | 401 | | | | 0.238 | ACVR1B | 195 |
| | | | 0.141 | ATAD2B | 210 | | | | 0.238 | RAB4B | 473 |
| | | | 0.138 | ZNF292 | 599 | | | | 0.232 | PTEN | 464 |
| | | | 0.137 | CCNT2 | 233 | | | | 0.231 | ASAP1 | 209 |
| | | | 0.134 | MARCH7 | 372 | | | | 0.231 | MANSC1 | 363 |
| | | | 0.133 | ACAP2 | 194 | | | | 0.228 | RYBP | 500 |
| | | | 0.132 | MED13 | 379 | | | | 0.225 | CSAD | 253 |
| | | | 0.131 | IL6ST | 327 | | | | 0.223 | UBXN2B | 568 |
| | | | 0.131 | PHF3 | 436 | | | | 0.223 | TNIP1 | 552 |
| | | | 0.129 | SP3 | 522 | | | | 0.222 | WBP2 | 579 |
| | | | 0.110 | SEC62 | 503 | | | | 0.211 | OGFRL1 | 416 |
| | | | 0.098 | ZFYVE16 | 594 | | | | 0.209 | SNN | 516 |
| | | | 0.095 | NEK7 | 403 | | | | 0.205 | HPCAL1 | 312 |
| | | | 0.094 | POLD4 | 446 | | | | 0.196 | CD37 | 235 |
| | | | 0.091 | GNA12 | 295 | | | | 0.194 | RNF146 | 490 |
| | | | 0.087 | TRIB2 | 558 | | | | 0.184 | RAB14 | 471 |
| | | | 0.086 | YTHDF3 | 586 | | | | 0.177 | TOPORS | 555 |
| | | | 0.082 | PPP2R5A | 452 | | | | 0.176 | NFYA | 405 |
| | | | 0.081 | PPP1R2 | 451 | | | | 0.172 | FOXO3 | 289 |
| | | | 0.076 | ZDHHC17 | 590 | | | | 0.171 | CREB1 | 249 |
| | | | 0.060 | STK38L | 531 | | | | 0.170 | MAPK1 | 369 |
| | | | 0.057 | ST13 | 525 | | | | 0.170 | SOAT1 | 519 |
| | | | 0.046 | FAM134A | 278 | | | | 0.168 | UBQLN2 | 567 |
| | | | 0.022 | PFDN5 | 429 | | | | 0.166 | OSBPL11 | 417 |
| | | | 0.015 | MARCH8 | 373 | | | | 0.165 | KLHL2 | 343 |
| | | | −0.041 | POLR1D | 447 | | | | 0.153 | VAV3 | 575 |
| | | | | | | | | | 0.135 | BRD4 | 221 |
| | | | | | | | | | 0.130 | MARK3 | 374 |
| | | | | | | | | | 0.114 | BAZ2B | 216 |
| | | | | | | | | | 0.112 | ZNF148 | 597 |
| | | | | | | | | | 0.110 | CASP8 | 229 |
| | | | | | | | | | 0.108 | CHMP1B | 242 |
| | | | | | | | | | 0.105 | HPS1 | 313 |
| | | | | | | | | | 0.099 | RNF141 | 489 |
| | | | | | | | | | 0.096 | MOSPD2 | 386 |
| | | | | | | | | | 0.081 | PINK1 | 439 |
| | | | | | | | | | 0.080 | CDIPT | 238 |
| | | | | | | | | | 0.060 | NCOA4 | 398 |
| | | | | | | | | | 0.059 | PPP3R1 | 453 |
| | | | | | | | | | 0.014 | MKRN1 | 383 |
| | | | | | | | | | 0.005 | GYPC | 304 |
| | | | | | | | | | −0.021 | BMP2K | 219 |
| | | | | | | | | | −0.058 | FBXO9 | 281 |

TABLE 27
DERIVED BIOMARKERS GROUPED (A, B, C, D) BASED ON CORRELATION TO EACH OF THE BIOMARKERS IN THE FINAL PASIRS SIGNATURE (TTC17, G6PD, HERC6, LAP3, NUP160, TPP1)

| Group A | | | Group B | | | Group C | | | Group D | | | Group E | | | Group F | | |
|---|---|---|---|---|---|---|---|---|---|---|---|---|---|---|---|---|---|
| Correlation | HUGO | DNA | Correlation | HUGO | DNA | Correlation | HUGO | DNA | Correlation | HUGO | DNA | Correlation | HUGO | DNA | Correlation | HUGO | DNA |
| to | Gene | SEQ | to | Gene | SEQ | to | Gene | SEQ | to | Gene | SEQ | to | Gene | SEQ | to | Gene | SEQ |
| TTC17 | Symbol | ID | G6PD | Symbol | ID | HERC6 | Symbol | ID | LAP3 | Symbol | ID | NUP160 | Symbol | ID | TPP1 | Symbol | ID |
| 1 | TTC17 | 1132 | 1 | G6PD | 1057 | 1 | HERC6 | 1063 | 1 | LAP3 | 1071 | 1 | NUP160 | 1082 | 1 | TPP1 | 1125 |
| 0.647 | ZMYND11 | 1142 | 0.723 | SP11 | 1112 | 0.408 | SETX | 1107 | 0.889 | WARS | 1139 | 0.736 | TOP2B | 1124 | 0.634 | WAS | 1140 |
| 0.574 | ASXL2 | 1022 | 0.692 | PGD | 1086 | 0.241 | HLA- | 1064 | 0.845 | UBE2L6 | 1134 | 0.686 | METAP1 | 1074 | 0.630 | RTN4 | 1102 |
| | | | | | | | DPA1 | | | | | | | | | | |
| 0.570 | ARID1A | 1020 | 0.682 | GRINA | 1061 | | | | 0.801 | TAP1 | 1117 | 0.653 | ZBED5 | 1141 | 0.562 | ATP6V1B2 | 1025 |
| 0.560 | CEP192 | 1037 | 0.679 | CD63 | 1035 | | | | 0.797 | SQRDL | 1113 | 0.626 | FNTA | 1056 | 0.528 | TIMP2 | 1120 |
| 0.551 | PCID2 | 1084 | 0.663 | FGR | 1053 | | | | 0.782 | POMP | 1089 | 0.624 | TRIT1 | 1128 | 0.482 | FLII | 1054 |
| 0.534 | BCL11A | 1026 | 0.657 | TCIRG1 | 1119 | | | | 0.781 | PLSCR1 | 1088 | 0.611 | ZNF266 | 1143 | 0.481 | RAB7A | 1093 |
| 0.534 | ARIH2 | 1021 | 0.642 | NUMB | 1081 | | | | 0.706 | MYD88 | 1077 | 0.604 | EXOSC10 | 1047 | | | |
| 0.524 | ARHGAP17 | 1019 | 0.618 | PRKCD | 1091 | | | | 0.701 | ATOX1 | 1023 | 0.598 | APEX1 | 1018 | | | |
| 0.504 | EXOSC2 | 1048 | 0.607 | TSPO | 1131 | | | | 0.694 | SH3GLB1 | 1108 | 0.595 | SERBP1 | 1104 | | | |
| 0.465 | TCF4 | 1118 | 0.570 | BCL3 | 1027 | | | | 0.678 | CEBPB | 1036 | 0.555 | TRAF3IP3 | 1126 | | | |
| 0.460 | RPL15 | 1097 | 0.569 | JUNB | 1069 | | | | 0.675 | SERPINB1 | 1105 | 0.552 | MLLT10 | 1076 | | | |
| 0.459 | GLG1 | 1058 | 0.553 | BCL6 | 1028 | | | | 0.674 | FCER1G | 1052 | 0.537 | IMP3 | 1066 | | | |
| 0.452 | CSNK1G2 | 1041 | 0.551 | TNIP1 | 1123 | | | | 0.671 | RALB | 1094 | 0.533 | ADSL | 1016 | | | |
| 0.445 | SUCLG2 | 1115 | 0.550 | ENO1 | 1044 | | | | 0.661 | IRF1 | 1067 | 0.533 | MGEA5 | 1075 | | | |
| 0.444 | LY9 | 1073 | 0.536 | FLOT1 | 1055 | | | | 0.658 | GNG5 | 1059 | 0.525 | NOSIP | 1080 | | | |
| 0.442 | USP34 | 1137 | 0.514 | PLAUR | 1087 | | | | 0.655 | TANK | 1116 | 0.522 | SEH1L | 1103 | | | |
| 0.416 | ADK | 1015 | 0.491 | PCBP1 | 1083 | | | | 0.651 | VAMP3 | 1138 | 0.521 | PREPL | 1090 | | | |
| 0.411 | CNOT7 | 1040 | 0.476 | GPI | 1060 | | | | 0.626 | LDHA | 1072 | 0.478 | FBXO11 | 1051 | | | |
| 0.410 | UFM1 | 1135 | 0.424 | NFKBIA | 1079 | | | | 0.617 | UPP1 | 1136 | 0.477 | CAMK2G | 1030 | | | |
| 0.403 | AHCTF1 | 1017 | 0.422 | CCND3 | 1031 | | | | 0.612 | HCK | 1062 | 0.476 | TMEM50B | 1122 | | | |
| 0.372 | TROVE2 | 1129 | | | | | | | 0.607 | NFIL3 | 1078 | 0.470 | RPS4X | 1101 | | | |
| 0.364 | CLIP4 | 1039 | | | | | | | 0.605 | SLAMF7 | 1109 | 0.466 | RPL9 | 1099 | | | |
| 0.350 | CD52 | 1033 | | | | | | | 0.593 | ACSL4 | 1014 | 0.463 | RPL22 | 1098 | | | |
| 0.326 | SERTAD2 | 1106 | | | | | | | 0.592 | ERLIN1 | 1045 | 0.457 | FBL | 1050 | | | |
| 0.323 | IRF8 | 1068 | | | | | | | 0.584 | RBMS1 | 1095 | 0.438 | CCR7 | 1032 | | | |
| 0.234 | CHN2 | 1038 | | | | | | | 0.581 | STAT3 | 1114 | 0.412 | IL10RA | 1065 | | | |
| | | | | | | | | | 0.572 | TRIB1 | 1127 | 0.403 | DNAJC10 | 1043 | | | |
| | | | | | | | | | 0.570 | C3AR1 | 1029 | 0.384 | RPS14 | 1100 | | | |
| | | | | | | | | | 0.560 | ATP2A2 | 1024 | 0.371 | EXOSC9 | 1049 | | | |
| | | | | | | | | | 0.546 | SOCS3 | 1110 | 0.367 | TMEM106B | 1121 | | | |
| | | | | | | | | | 0.545 | RIT1 | 1096 | | | | | | |
| | | | | | | | | | 0.538 | SORT1 | 1111 | | | | | | |
| | | | | | | | | | 0.538 | RAB27A | 1092 | | | | | | |
| | | | | | | | | | 0.536 | ETV6 | 1046 | | | | | | |
| | | | | | | | | | 0.529 | TUBA1B | 1133 | | | | | | |
| | | | | | | | | | 0.499 | PCMT1 | 1085 | | | | | | |
| | | | | | | | | | 0.486 | CD55 | 1034 | | | | | | |
| | | | | | | | | | 0.476 | CSTB | 1042 | | | | | | |
| | | | | | | | | | 0.424 | TRPC4AP | 1130 | | | | | | |
| | | | | | | | | | 0.389 | KIF1B | 1070 | | | | | | |

TABLE 28
DERIVED BIOMARKERS GROUPED (A, B, C, D) BASED ON CORRELATION TO EACH OF THE BIOMARKERS
IN THE FINAL INSIRS SIGNATURE (ARL6IP5, ENTPD1, HEATR1, TNFSF8

| Group A | | | Group B | | | Group C | | | Group D | | |
|---|---|---|---|---|---|---|---|---|---|---|---|
| Correlation | HUGO | DNA | Correlation | HUGO | DNA | Correlation | HUGO | DNA | Correlation | HUGO | DNA |
| to | Gene | SEQ | to | Gene | SEQ | to | Gene | SEQ | to | Gene | SEQ |
| ARL6IP5 | Name | ID | ENTPD1 | Name | ID | HEATR1 | Name | ID | TNFSF8 | Name | ID |
| 0.902 | MACF1 | 1343 | 0.957 | KCMF1 | 1334 | 0.974 | BCKDHB | 1286 | 0.867 | KLHL24 | 1337 |
| 0.884 | EFHD2 | 1306 | 0.949 | IQSEC1 | 1333 | 0.974 | CLOCK | 1299 | 0.858 | RBM26 | 1377 |
| 0.850 | TIA1 | 1405 | 0.943 | SLCO3A1 | 1392 | 0.972 | MY09A | 1353 | 0.832 | SUPT7L | 1396 |
| 0.847 | FCF1 | 1316 | 0.930 | GAB2 | 1319 | 0.971 | XPO4 | 1419 | 0.829 | SYNE2 | 1397 |
| 0.831 | THOC2 | 1404 | 0.925 | STK17B | 1395 | 0.965 | HLTF | 1326 | 0.826 | RABGAP1L | 1375 |
| 0.814 | MDH1 | 1345 | 0.919 | HEBP2 | 1324 | 0.964 | SLC35D1 | 1391 | 0.825 | PLCL2 | 1366 |
| 0.775 | ADSL | 1276 | 0.916 | BTN2A1 | 1288 | 0.961 | CDK6 | 1295 | 0.822 | ATXN3 | 1285 |
| 0.704 | SIDT2 | 1389 | 0.916 | CDKN1B | 1296 | 0.961 | VPS13A | 1415 | 0.807 | KIAA0391 | 1335 |
| | | | 0.910 | EXOC7 | 1313 | 0.960 | ANK3 | 1279 | 0.783 | NGDN | 1357 |
| | | | 0.904 | MXD1 | 1351 | 0.960 | PRKAB2 | 1368 | 0.764 | TRAPPC2 | 1409 |
| | | | 0.891 | IGF2R | 1329 | 0.957 | LANCL1 | 1339 | 0.763 | FUT8 | 1317 |
| | | | 0.888 | ADAM19 | 1274 | 0.956 | IDE | 1328 | 0.761 | G3BP1 | 1318 |
| | | | 0.887 | VNN3 | 1414 | 0.955 | LARP4 | 1341 | 0.757 | VPS13C | 1417 |
| | | | 0.882 | TFIP11 | 1402 | 0.955 | NEK1 | 1355 | 0.754 | TMEM87A | 1407 |
| | | | 0.880 | POLR2A | 1367 | 0.953 | SLC35A3 | 1390 | 0.740 | PWP1 | 1373 |
| | | | 0.876 | HAL | 1322 | 0.951 | RAB11FIP2 | 1374 | 0.732 | CD28 | 1291 |
| | | | 0.869 | MYH9 | 1352 | 0.951 | DIAPH2 | 1305 | | | |
| | | | 0.850 | PELI1 | 1363 | 0.945 | KLHL20 | 1336 | | | |
| | | | 0.839 | ARHGEF6 | 1281 | 0.944 | TBCE | 1399 | | | |
| | | | 0.827 | CLEC4E | 1298 | 0.944 | TGS1 | 1403 | | | |
| | | | 0.822 | TTC17 | 1411 | 0.942 | ADRBK2 | 1275 | | | |
| | | | 0.819 | RGS2 | 1382 | 0.942 | TTC27 | 1412 | | | |
| | | | 0.811 | EXTL3 | 1314 | 0.942 | AGPAT5 | 1278 | | | |
| | | | 0.809 | CDA | 1294 | 0.941 | TCF12 | 1400 | | | |
| | | | 0.805 | NOTCH2 | 1361 | 0.939 | BRCC3 | 1287 | | | |
| | | | 0.804 | RCBTB2 | 1378 | 0.935 | YTHDC2 | 1421 | | | |
| | | | 0.799 | CYP4F3 | 1303 | 0.934 | ZMYND11 | 1422 | | | |
| | | | 0.786 | RRAGC | 1387 | 0.934 | NOL10 | 1359 | | | |
| | | | | | | 0.932 | C14orf1 | 1290 | | | |
| | | | | | | 0.932 | EFTUD1 | 1307 | | | |
| | | | | | | 0.932 | ZNF507 | 1423 | | | |
| | | | | | | 0.932 | TRIP11 | 1410 | | | |
| | | | | | | 0.931 | ASCC3 | 1283 | | | |
| | | | | | | 0.931 | ERCC4 | 1311 | | | |
| | | | | | | 0.930 | CD84 | 1293 | | | |
| | | | | | | 0.930 | RAD50 | 1376 | | | |
| | | | | | | 0.927 | CLUAP1 | 1300 | | | |
| | | | | | | 0.927 | FASTKD2 | 1315 | | | |
| | | | | | | 0.923 | TCF7L2 | 1401 | | | |
| | | | | | | 0.922 | CKAP2 | 1297 | | | |
| | | | | | | 0.921 | ESF1 | 1312 | | | |
| | | | | | | 0.921 | VPS13B | 1416 | | | |
| | | | | | | 0.919 | RMND1 | 1384 | | | |
| | | | | | | 0.916 | PHC3 | 1365 | | | |
| | | | | | | 0.916 | ARHGAP5 | 1280 | | | |
| | | | | | | 0.913 | MLLT10 | 1347 | | | |
| | | | | | | 0.913 | CPA3 | 1301 | | | |
| | | | | | | 0.911 | NCBP1 | 1354 | | | |
| | | | | | | 0.911 | MANEA | 1344 | | | |
| | | | | | | 0.908 | RDX | 1379 | | | |
| | | | | | | 0.906 | RIOK2 | 1383 | | | |
| | | | | | | 0.900 | IP07 | 1331 | | | |
| | | | | | | 0.900 | SYT11 | 1398 | | | |
| | | | | | | 0.898 | RNF170 | 1385 | | | |
| | | | | | | 0.896 | SMC6 | 1394 | | | |
| | | | | | | 0.893 | PEX1 | 1364 | | | |
| | | | | | | 0.891 | ATP8A1 | 1284 | | | |
| | | | | | | 0.888 | HIBCH | 1325 | | | |
| | | | | | | 0.887 | GOLPH3L | 1321 | | | |
| | | | | | | 0.882 | ZNF562 | 1424 | | | |
| | | | | | | 0.874 | HRH4 | 1327 | | | |
| | | | | | | 0.864 | KRIT1 | 1338 | | | |
| | | | | | | 0.860 | IKBKAP | 1330 | | | |
| | | | | | | 0.857 | YEATS4 | 1420 | | | |
| | | | | | | 0.854 | CREB1 | 1302 | | | |
| | | | | | | 0.854 | VEZT | 1413 | | | |
| | | | | | | 0.851 | PSMD5 | 1371 | | | |
| | | | | | | 0.849 | LRRC8D | 1342 | | | |
| | | | | | | 0.848 | PRPF39 | 1369 | | | |
| | | | | | | 0.839 | NR2C1 | 1362 | | | |
| | | | | | | 0.838 | CD40LG | 1292 | | | |
| | | | | | | 0.835 | ENOSF1 | 1309 | | | |
| | | | | | | 0.831 | TLK1 | 1406 | | | |
| | | | | | | 0.825 | RFC1 | 1381 | | | |
| | | | | | | 0.823 | NIP7 | 1358 | | | |
| | | | | | | 0.822 | MTRR | 1350 | | | |
| | | | | | | 0.819 | MTO1 | 1349 | | | |
| | | | | | | 0.819 | METTL5 | 1346 | | | |
| | | | | | | 0.814 | RNMT | 1386 | | | |
| | | | | | | 0.813 | MRPS10 | 1348 | | | |
| | | | | | | 0.812 | WDR70 | 1418 | | | |
| | | | | | | 0.809 | IQCB1 | 1332 | | | |
| | | | | | | 0.809 | REPS1 | 1380 | | | |
| | | | | | | 0.806 | PRUNE | 1370 | | | |
| | | | | | | 0.806 | NFX1 | 1356 | | | |
| | | | | | | 0.801 | AGA | 1277 | | | |
| | | | | | | 0.796 | EIF5B | 1308 | | | |
| | | | | | | 0.791 | NOL8 | 1360 | | | |
| | | | | | | 0.789 | SMC3 | 1393 | | | |
| | | | | | | 0.786 | S100PBP | 1388 | | | |
| | | | | | | 0.779 | BZW2 | 1289 | | | |
| | | | | | | 0.761 | CYSLTR1 | 1304 | | | |
| | | | | | | 0.748 | LARP1 | 1340 | | | |
| | | | | | | 0.734 | GGPS1 | 1320 | | | |
| | | | | | | 0.599 | PTGS1 | 1372 | | | |

TABLE 29
TOP PERFORMING (BASED ON AUC) BASIRS DERIVED
BIOMARKERS FOLLOWING A GREEDY SEARCH ON A
COMBINED DATASET
The top derived biomarker was TSPO:HCLS1 with an AUC of 0.838.
Incremental AUC increases can be made with the addition of further
derived biomarkers as indicated.

| Greedy Addition | AUC | AUC_{SD} |
|---|---|---|
| TSPO_HCLS1 | 0.838 | 0.0083 |
| OPLAH_ZHX2 | 0.863 | 0.0061 |
| TSPO_RNASE6 | 0.881 | 0.0055 |
| GAS7_CAMK1D | 0.891 | 0.0044 |
| ST3GAL2_PRKD2 | 0.897 | 0.0032 |
| PCOLCE2_NMUR1 | 0.901 | 0.0031 |
| CR1_HAL | 0.901 | 0.0040 |

TABLE 30
BASIRS NUMERATORS AND DENOMINATORS APPEARING MORE
THAN ONCE IN DERIVED BIOMARKERS WITH A MEAN AUC >
0.85 IN THE VALIDATION DATASETS
BaSIRS numerators and denominators appearing more than once in
derived biomarkers with an AUC > 0.85

| Numerator | # | Denominator | # |
|---|---|---|---|
| PDGFC | 28 | INPP5D | 6 |
| TSPO | 11 | KLRD1 | 6 |
| GAS7 | 9 | KLRF1 | 6 |
| PCOLCE2 | 8 | NLRP1 | 5 |
| GALNT2 | 6 | GAB2 | 4 |
| ALPL | 5 | CAMK1D | 3 |
| ITGA7 | 4 | CCNK | 3 |
| CD82 | 3 | ADAM19 | 2 |
| CR1 | 3 | HAL | 2 |
| HK3 | 3 | MME | 2 |
| OPLAH | 3 | NOV | 2 |
| COX15 | 2 | PARP8 | 2 |
| ENTPD7 | 2 | RNASE6 | 2 |
| SMPDL3A | 2 | YPEL1 | 2 |
| TMEM165 | 2 | ZFP36L2 | 2 |

TABLE 31
TOP PERFORMING (BASED ON AUC) VASIRS DERIVED
BIOMARKERS FOLLOWING A GREEDY SEARCH ON A
COMBINED DATASET
The top derived biomarker was ISG15:IL16 with an AUC of 0.92.
Incremental AUC increases can be made with the addition of further
derived biomarkers as indicated.

| Greedy Addition | Individual AUC | Combined AUC |
|---|---|---|
| ISG15:IL16 | 0.92 | 0.92 |
| OASL:ADGRE5 | 0.865 | 0.936 |
| TAP1:TGFBR2 | 0.879 | 0.945 |
| IFIH1:CRLF3 | 0.873 | 0.946 |
| IFI44:IL4R | 0.867 | 0.947 |
| EIF2AK2:SYPL1 | 0.859 | 0.947 |
| OAS2:LEF1 | 0.875 | 0.946 |
| STAT1:PCBP2 | 0.844 | 0.944 |
| IFI6:IL6ST | 0.821 | 0.942 |

TABLE 32
VASIRS NUMERATORS AND DENOMINATORS APPEARING
MORE THAN TWICE IN THE 473 DERIVED BIOMARKERS WITH A
MEAN AUC > 0.80 IN AT LEAST 11 OF 14 VIRAL DATASETS.
VaSIRS numerators and denominators appearing more than once in
derived biomarkers with an AUC > 0.80

| Numerator | # | Denominator | # |
|---|---|---|---|
| OASL | 344 | ABLIM1 | 12 |
| USP18 | 50 | IL16 | 9 |
| EIF2AK2 | 13 | SYPL1 | 6 |
| ISG15 | 8 | CYLD | 5 |
| IFI44 | 7 | IL4R | 5 |
| LAP3 | 7 | LTB | 5 |
| ZBP1 | 6 | MYC | 5 |
| IFI6 | 5 | PCF11 | 5 |
| OAS2 | 4 | TGFBR2 | 5 |
| DDX60 | 3 | CAMK1D | 4 |
| DHX58 | 3 | IL6ST | 4 |
| IFIH1 | 3 | LEF1 | 4 |
| TAP1 | 3 | ZNF274 | 4 |
| | | BTG1 | 3 |
| | | CRLF3 | 3 |
| | | DGKA | 3 |
| | | SESN1 | 3 |
| | | TNRC6B | 3 |
| | | ZFC3H1 | 3 |

TABLE 33
TOP PERFORMING (BASED ON AUC) PASIRS DERIVED
BIOMARKERS FOLLOWING A GREEDY SEARCH ON A
COMBINED DATASET
The top derived biomarker was TTC17:G6PD with an AUC of 0.96.
Incremental AUC increases can be made with the addition of further
derived biomarkers as indicated.

| Greedy Addition | Individual AUC | Combined AUC |
|---|---|---|
| TTC17_G6PD | 0.96 | 0.96 |
| HERC6_LAP3 | 0.84 | 0.99 |
| NUP160_TPP1 | 0.847 | 0.99 |

TABLE 34
PASIRS NUMERATORS AND DENOMINATORS APPEARING MORE
THAN TWICE IN THE 523 DERIVED BIOMARKERS WITH A MEAN
AUC > 0.75 IN THE VALIDATION DATASETS.
PaSIRS numerators and denominators appearing more than once in
derived biomarkers with an AUC > 0.75

| Numerator | # | Denominator | # |
|---|---|---|---|
| ARID1A | 62 | SQRDL | 45 |
| CEP192 | 35 | CEBPB | 40 |
| EXOSC10 | 33 | WARS | 39 |
| IMP3 | 33 | CD63 | 38 |
| RPL9 | 24 | SH3GLB1 | 31 |
| TTC17 | 24 | POMP | 23 |
| BCL11A | 22 | PGD | 21 |
| TCF4 | 21 | FCER1G | 17 |
| ASXL2 | 18 | MYD88 | 15 |
| RPS4X | 15 | UPP1 | 15 |
| ZMYND11 | 13 | G6PD | 13 |
| AHCTF1 | 12 | GNG5 | 13 |
| LY9 | 12 | LAP3 | 12 |
| FBXO11 | 11 | TCIRG1 | 12 |
| FNTA | 11 | SERPINB1 | 11 |
| ARIH2 | 10 | ATOX1 | 10 |
| EXOSC2 | 9 | TANK | 10 |
| NUP160 | 8 | TSPO | 10 |
| ZBED5 | 8 | TNIP1 | 9 |
| CAMK2G | 7 | CSTB | 8 |
| CNOT7 | 7 | ENO1 | 8 |
| TOP2B | 7 | RALB | 8 |
| ARHGAP17 | 6 | VAMP3 | 7 |
| HLA-DPA1 | 6 | BCL6 | 6 |
| IRF8 | 6 | LDHA | 6 |
| PCID2 | 6 | FGR | 5 |
| RPL15 | 6 | IRF1 | 5 |
| RPL22 | 6 | ERLIN1 | 4 |
| ADSL | 5 | PCMT1 | 4 |
| IL10RA | 5 | PRKCD | 4 |
| NOSIP | 5 | RTN4 | 4 |
| SETX | 5 | SPI1 | 4 |
| SUCLG2 | 5 | TAP1 | 4 |
| CSNK1G2 | 4 | UBE2L6 | 4 |
| PREPL | 4 | C3AR1 | 3 |
| RPS14 | 4 | FLII | 3 |
| TMEM50B | 4 | NFIL3 | 3 |
| TROVE2 | 4 | PLAUR | 3 |
| CHN2 | 3 | SLAMF7 | 3 |
| METAP1 | 3 | WAS | 3 |
| MLLT10 | 3 | ATP2A2 | 2 |
| SERBP1 | 3 | ETV6 | 2 |
| SERTAD2 | 3 | GPI | 2 |
| CCR7 | 2 | HCK | 2 |
| CLIP4 | 2 | PCBP1 | 2 |
| SEH1L | 2 | PLSCR1 | 2 |
| TRAF3IP3 | 2 | RAB27A | 2 |
| UFM1 | 2 | STAT3 | 2 |
| USP34 | 2 | TIMP2 | 2 |
| ZNF266 | 2 | TPP1 | 2 |
| | | TUBA1B | 2 |

TABLE 35
TABLE OF INDIVIDUAL PERFORMANCE, IN DESCENDING AUC, OF THE 523 PASIRS DERIVED
BIOMARKERS WITH AN AVERAGE AUC >0.75 ACROSS EACH OF FIVE PROTOZOAL DATASETS.

| | | | Severe vs | | | |
|---|---|---|---|---|---|---|
| | | | Mild | | | |
| | Malaria | Leishmania | Malaria | Malaria | Malaria | |
| Derived Biomarker | GSE34404 | G5E64610 | G5E33811 | G5E15221 | G5E5418 | Mean |
| RPL9_WARS | 0.935 | 0.920 | 1.000 | 0.852 | 0.964 | 0.934 |
| RPL9_CSTB | 0.895 | 0.900 | 1.000 | 0.888 | 0.982 | 0.933 |
| NUP160_WARS | 0.915 | 0.980 | 0.920 | 0.898 | 0.948 | 0.932 |
| IMP3_ATOX1 | 0.950 | 0.900 | 0.880 | 0.974 | 0.955 | 0.932 |
| RPS4X_WARS | 0.937 | 1.000 | 0.840 | 0.944 | 0.933 | 0.931 |
| TCF4_CEBPB | 0.984 | 0.960 | 0.840 | 0.959 | 0.909 | 0.930 |
| IMP3_LAP3 | 0.937 | 0.900 | 0.920 | 0.929 | 0.952 | 0.927 |
| EXOSC10_WARS | 0.960 | 1.000 | 0.840 | 0.939 | 0.891 | 0.926 |
| TTC17_WARS | 0.954 | 1.000 | 0.800 | 0.990 | 0.885 | 0.926 |
| TCF4_WARS | 0.955 | 0.960 | 0.960 | 0.903 | 0.848 | 0.925 |
| METAP1_WARS | 0.912 | 0.940 | 0.880 | 0.913 | 0.979 | 0.925 |
| FNTA_POMP | 0.966 | 0.920 | 0.840 | 0.923 | 0.970 | 0.924 |
| TCF4_TANK | 0.975 | 0.980 | 0.960 | 0.781 | 0.921 | 0.923 |
| TOP2B_CEBPB | 0.936 | 1.000 | 0.760 | 0.934 | 0.979 | 0.922 |
| AHCTF1_CEBPB | 0.977 | 0.820 | 0.840 | 0.980 | 0.991 | 0.921 |
| RPS4X_MYD88 | 0.935 | 0.980 | 0.800 | 0.929 | 0.964 | 0.921 |
| IMP3_CEBPB | 0.976 | 0.880 | 0.840 | 0.923 | 0.985 | 0.921 |
| RPL9_CEBPB | 0.952 | 1.000 | 0.800 | 0.852 | 1.000 | 0.921 |
| RPS4X_CEBPB | 0.949 | 1.000 | 0.720 | 0.949 | 0.985 | 0.921 |
| TTC17_CEBPB | 0.980 | 1.000 | 0.640 | 0.990 | 0.991 | 0.920 |
| PREPL_WARS | 0.911 | 1.000 | 0.920 | 0.791 | 0.979 | 0.920 |
| TCF4_LAP3 | 0.944 | 0.980 | 0.880 | 0.918 | 0.876 | 0.920 |
| ZBED5_WARS | 0.940 | 0.940 | 0.880 | 0.974 | 0.864 | 0.920 |
| TCF4_POMP | 0.952 | 0.900 | 0.880 | 0.954 | 0.909 | 0.919 |
| NUP160_SQRDL | 0.899 | 0.960 | 0.800 | 0.959 | 0.973 | 0.918 |
| TRIT1_WARS | 0.908 | 1.000 | 0.800 | 0.903 | 0.976 | 0.917 |
| ZBED5_CEBPB | 0.965 | 0.940 | 0.720 | 0.990 | 0.964 | 0.916 |
| IMP3_WARS | 0.964 | 0.880 | 0.920 | 0.908 | 0.906 | 0.916 |
| RPS4X_SQRDL | 0.934 | 1.000 | 0.720 | 0.980 | 0.942 | 0.915 |
| NUP160_POMP | 0.923 | 0.880 | 0.840 | 0.954 | 0.979 | 0.915 |
| EXOSC10_LAP3 | 0.946 | 1.000 | 0.760 | 0.939 | 0.927 | 0.914 |
| RPS4X_GNG5 | 0.965 | 0.960 | 0.760 | 0.898 | 0.988 | 0.914 |
| TOP2B_WARS | 0.930 | 1.000 | 0.840 | 0.923 | 0.876 | 0.914 |
| RPL9_POMP | 0.959 | 0.840 | 0.880 | 0.918 | 0.970 | 0.913 |
| EXOSC10_ATOX1 | 0.959 | 1.000 | 0.680 | 1.000 | 0.927 | 0.913 |
| TTC17_TANK | 0.958 | 1.000 | 0.720 | 0.923 | 0.964 | 0.913 |
| EXOSC10_CEBPB | 0.977 | 1.000 | 0.680 | 0.929 | 0.979 | 0.913 |
| NOSIP_CEBPB | 0.963 | 0.900 | 0.840 | 0.949 | 0.912 | 0.913 |
| RPL22_CEBPB | 0.950 | 1.000 | 0.720 | 0.959 | 0.933 | 0.913 |
| TTC17_ATP2A2 | 0.941 | 0.940 | 0.760 | 0.939 | 0.982 | 0.912 |
| SEH1L_WARS | 0.955 | 0.980 | 0.840 | 0.837 | 0.945 | 0.911 |
| EXOSC10_UBE2L6 | 0.932 | 1.000 | 0.800 | 0.969 | 0.852 | 0.911 |
| TTC17_LAP3 | 0.919 | 1.000 | 0.680 | 1.000 | 0.948 | 0.910 |
| SUCLG2_CEBPB | 0.976 | 1.000 | 0.800 | 0.959 | 0.812 | 0.909 |
| EXOSC10_G6PD | 0.982 | 1.000 | 0.800 | 0.898 | 0.864 | 0.909 |
| CEP192_WARS | 0.945 | 0.840 | 0.920 | 0.934 | 0.903 | 0.908 |
| NUP160_CD63 | 0.951 | 0.940 | 0.760 | 0.923 | 0.964 | 0.908 |
| TMEM50B_WARS | 0.959 | 0.980 | 0.840 | 0.964 | 0.794 | 0.908 |
| EXOSC10_LDHA | 0.980 | 0.900 | 0.760 | 0.954 | 0.942 | 0.907 |
| ARID1A_CSTB | 0.944 | 0.860 | 0.880 | 0.913 | 0.939 | 0.907 |
| SUCLG2_WARS | 0.963 | 1.000 | 0.920 | 0.969 | 0.682 | 0.907 |
| ARID1A_CEBPB | 0.976 | 0.940 | 0.680 | 0.954 | 0.982 | 0.906 |
| FBXO11_TANK | 0.908 | 0.940 | 0.760 | 0.918 | 1.000 | 0.905 |
| SUCLG2_SH3GLB1 | 0.976 | 1.000 | 0.800 | 0.923 | 0.827 | 0.905 |
| TTC17_G6PD | 0.986 | 0.880 | 0.760 | 1.000 | 0.900 | 0.905 |
| IMP3_PCMT1 | 0.962 | 0.900 | 0.840 | 0.974 | 0.848 | 0.905 |
| ARID1A_LAP3 | 0.909 | 0.980 | 0.760 | 0.939 | 0.936 | 0.905 |
| IMP3_SQRDL | 0.966 | 0.820 | 0.840 | 0.959 | 0.936 | 0.904 |
| TCF4_ATOX1 | 0.948 | 0.980 | 0.760 | 0.923 | 0.909 | 0.904 |
| IMP3_SH3GLB1 | 0.970 | 0.780 | 0.840 | 0.934 | 0.994 | 0.904 |
| EXOSC10_MYD88 | 0.957 | 1.000 | 0.680 | 0.929 | 0.952 | 0.904 |
| LY9_WARS | 0.949 | 0.820 | 0.960 | 0.964 | 0.824 | 0.903 |
| IMP3_CSTB | 0.985 | 0.780 | 0.920 | 0.908 | 0.921 | 0.903 |
| RPL15_CEBPB | 0.968 | 1.000 | 0.800 | 0.985 | 0.761 | 0.903 |
| ARHGAP17_ATOX1 | 0.983 | 0.980 | 0.800 | 0.872 | 0.876 | 0.902 |
| TTC17_MYD88 | 0.968 | 0.960 | 0.680 | 0.944 | 0.958 | 0.902 |
| EXOSC10_TCIRG1 | 0.977 | 1.000 | 0.680 | 0.934 | 0.918 | 0.902 |
| ZMYND11_CEBPB | 0.938 | 1.000 | 0.600 | 0.980 | 0.991 | 0.902 |
| CEP192_TANK | 0.959 | 0.860 | 0.840 | 0.872 | 0.976 | 0.901 |
| IMP3_UBE2L6 | 0.918 | 0.900 | 0.840 | 0.954 | 0.894 | 0.901 |
| RPS4X_CD63 | 0.973 | 1.000 | 0.640 | 0.974 | 0.918 | 0.901 |
| RPL9_CD63 | 0.984 | 0.920 | 0.720 | 0.908 | 0.973 | 0.901 |
| ARID1A_UBE2L6 | 0.887 | 0.960 | 0.760 | 0.959 | 0.933 | 0.900 |
| TCF4_UBE2L6 | 0.923 | 0.960 | 0.920 | 0.888 | 0.806 | 0.899 |
| ARID1A_WARS | 0.938 | 0.920 | 0.800 | 0.918 | 0.918 | 0.899 |
| CAMK2G_G6PD | 0.925 | 0.980 | 0.720 | 0.944 | 0.924 | 0.899 |
| RPS4X_SH3GLB1 | 0.941 | 0.940 | 0.680 | 0.954 | 0.979 | 0.899 |
| RPL9_TANK | 0.929 | 0.960 | 0.880 | 0.730 | 0.994 | 0.898 |
| IMP3_TANK | 0.942 | 0.840 | 0.880 | 0.842 | 0.988 | 0.898 |
| ZBED5_SH3GLB1 | 0.959 | 0.880 | 0.720 | 0.990 | 0.942 | 0.898 |
| TMEM50B_CEBPB | 0.963 | 1.000 | 0.680 | 0.964 | 0.882 | 0.898 |
| RPS4X_POMP | 0.953 | 0.940 | 0.680 | 0.969 | 0.945 | 0.898 |
| TOP2B_POMP | 0.948 | 0.980 | 0.640 | 0.949 | 0.970 | 0.897 |
| METAP1_POMP | 0.921 | 0.880 | 0.760 | 0.934 | 0.991 | 0.897 |
| EXOSC10_CSTB | 0.964 | 0.940 | 0.760 | 0.918 | 0.903 | 0.897 |
| ZNF266_CEBPB | 0.948 | 0.920 | 0.720 | 0.985 | 0.912 | 0.897 |
| TTC17_ATOX1 | 0.914 | 1.000 | 0.600 | 0.995 | 0.976 | 0.897 |
| CSNK1G2_G6PD | 0.978 | 1.000 | 0.680 | 0.923 | 0.900 | 0.896 |
| SETX_CEBPB | 0.983 | 0.960 | 0.680 | 0.893 | 0.964 | 0.896 |
| ARHGAP17_CEBPB | 0.986 | 1.000 | 0.800 | 0.791 | 0.900 | 0.895 |
| ZMYND11_WARS | 0.919 | 1.000 | 0.680 | 0.974 | 0.903 | 0.895 |
| IMP3_UPP1 | 0.982 | 0.880 | 0.800 | 0.934 | 0.879 | 0.895 |
| EXOSC10_IRF1 | 0.961 | 1.000 | 0.760 | 0.821 | 0.930 | 0.895 |
| UFM1_CEBPB | 0.948 | 0.920 | 0.640 | 1.000 | 0.964 | 0.894 |
| ARID1A_LDHA | 0.956 | 0.800 | 0.800 | 0.954 | 0.961 | 0.894 |
| RPL9_ATOX1 | 0.906 | 0.960 | 0.680 | 0.934 | 0.991 | 0.894 |
| TTC17_GNG5 | 0.972 | 0.840 | 0.680 | 0.990 | 0.988 | 0.894 |
| EXOSC10_POMP | 0.979 | 0.980 | 0.600 | 0.959 | 0.948 | 0.893 |
| ARID1A_ATOX1 | 0.904 | 0.980 | 0.600 | 0.995 | 0.988 | 0.893 |
| RPL9_SH3GLB1 | 0.951 | 0.900 | 0.680 | 0.934 | 1.000 | 0.893 |
| LY9_CEBPB | 0.971 | 0.760 | 0.800 | 0.969 | 0.964 | 0.893 |
| RP514_WARS | 0.942 | 0.980 | 0.840 | 0.883 | 0.818 | 0.893 |
| FNTA_SQRDL | 0.960 | 0.900 | 0.720 | 0.964 | 0.918 | 0.893 |
| APEX1_CD63 | 0.965 | 1.000 | 0.720 | 0.964 | 0.812 | 0.892 |
| SETX_WARS | 0.950 | 0.940 | 0.760 | 0.939 | 0.870 | 0.892 |
| IMP3_TNIP1 | 0.971 | 0.860 | 0.840 | 0.872 | 0.915 | 0.892 |
| FNTA_CD63 | 0.995 | 0.900 | 0.760 | 0.923 | 0.879 | 0.891 |
| TTC17_TCIRG1 | 0.988 | 0.920 | 0.680 | 0.995 | 0.873 | 0.891 |
| EXOSC10_SH3GLB1 | 0.981 | 0.960 | 0.600 | 0.913 | 1.000 | 0.891 |
| RPS4X_FCER1G | 0.979 | 0.880 | 0.640 | 0.969 | 0.985 | 0.891 |
| RPS4X_PGD | 0.970 | 1.000 | 0.680 | 0.980 | 0.824 | 0.891 |
| CAMK2G_CEBPB | 0.926 | 1.000 | 0.600 | 0.944 | 0.982 | 0.890 |
| ZMYND11_G6PD | 0.968 | 0.880 | 0.640 | 1.000 | 0.964 | 0.890 |
| FNTA_CEBPB | 0.977 | 1.000 | 0.600 | 0.898 | 0.976 | 0.890 |
| ZMYND11_CD63 | 0.968 | 0.980 | 0.560 | 1.000 | 0.942 | 0.890 |
| TCF4_RALB | 0.980 | 0.980 | 0.800 | 0.929 | 0.761 | 0.890 |
| ARHGAP17_LAP3 | 0.959 | 0.980 | 0.880 | 0.776 | 0.855 | 0.890 |
| IMP3_CD63 | 0.994 | 0.720 | 0.840 | 0.964 | 0.930 | 0.890 |
| ZMYND11_C3AR1 | 0.978 | 0.840 | 0.720 | 0.964 | 0.945 | 0.890 |
| AHCTF1_WARS | 0.943 | 0.800 | 0.840 | 0.929 | 0.936 | 0.890 |
| RPS4X_ENO1 | 0.937 | 0.920 | 0.720 | 0.995 | 0.873 | 0.889 |
| CEP192_PLSCR1 | 0.950 | 0.960 | 0.760 | 0.913 | 0.861 | 0.889 |
| EXOSC9_POMP | 0.977 | 0.940 | 0.760 | 0.969 | 0.797 | 0.889 |
| FNTA_GNG5 | 0.968 | 0.960 | 0.640 | 0.898 | 0.976 | 0.888 |
| CEP192_IRF1 | 0.945 | 0.980 | 0.800 | 0.765 | 0.952 | 0.888 |
| CEP192_CEBPB | 0.989 | 0.860 | 0.680 | 0.923 | 0.988 | 0.888 |
| ZMYND11_CSTB | 0.907 | 0.960 | 0.640 | 0.980 | 0.952 | 0.888 |
| FNTA_SH3GLB1 | 0.966 | 0.880 | 0.720 | 0.893 | 0.979 | 0.888 |
| ARID1A_TAP1 | 0.937 | 0.980 | 0.640 | 0.944 | 0.936 | 0.887 |
| NOSIP_WARS | 0.944 | 0.860 | 0.880 | 0.944 | 0.809 | 0.887 |
| RPS4X_UPP1 | 0.945 | 1.000 | 0.600 | 0.949 | 0.942 | 0.887 |
| CNOT7_CEBPB | 0.984 | 1.000 | 0.720 | 0.852 | 0.879 | 0.887 |
| ARHGAP17_WARS | 0.978 | 1.000 | 0.880 | 0.801 | 0.776 | 0.887 |
| UFM1_WARS | 0.923 | 0.880 | 0.760 | 0.980 | 0.891 | 0.887 |
| PREPL_SQRDL | 0.905 | 0.980 | 0.680 | 0.867 | 1.000 | 0.886 |
| IMP3_TAP1 | 0.953 | 0.920 | 0.800 | 0.944 | 0.815 | 0.886 |
| ARID1A_PCMT1 | 0.960 | 0.980 | 0.680 | 0.985 | 0.827 | 0.886 |
| SUCLG2_SQRDL | 0.977 | 1.000 | 0.760 | 0.959 | 0.733 | 0.886 |
| RPL22_SH3GLB1 | 0.941 | 0.940 | 0.680 | 0.959 | 0.909 | 0.886 |
| BCL11A_WARS | 0.960 | 0.660 | 0.960 | 0.980 | 0.870 | 0.886 |
| CNOT7_WARS | 0.969 | 1.000 | 0.840 | 0.832 | 0.788 | 0.886 |
| ZBED5_TCIRG1 | 0.964 | 0.820 | 0.760 | 0.985 | 0.900 | 0.886 |
| EXOSC10_SQRDL | 0.974 | 1.000 | 0.560 | 0.985 | 0.909 | 0.886 |
| AHCTF1_GNG5 | 0.970 | 0.640 | 0.880 | 0.964 | 0.973 | 0.885 |
| ZMYND11_FCER1G | 0.959 | 0.940 | 0.600 | 0.980 | 0.948 | 0.885 |
| TOP2B_ENO1 | 0.966 | 0.980 | 0.680 | 0.964 | 0.836 | 0.885 |
| IMP3_IRF1 | 0.956 | 0.940 | 0.840 | 0.750 | 0.939 | 0.885 |
| CEP192_TAP1 | 0.950 | 0.920 | 0.760 | 0.929 | 0.867 | 0.885 |
| RPL9_MYD88 | 0.943 | 0.820 | 0.840 | 0.847 | 0.973 | 0.885 |
| RPL22_GNG5 | 0.956 | 0.860 | 0.760 | 0.929 | 0.918 | 0.885 |
| FNTA_MYD88 | 0.968 | 0.940 | 0.640 | 0.903 | 0.970 | 0.884 |
| TCF4_GNG5 | 0.975 | 0.800 | 0.800 | 0.918 | 0.927 | 0.884 |
| EXOSC10_TANK | 0.959 | 0.920 | 0.720 | 0.862 | 0.955 | 0.883 |
| MLLT10_WARS | 0.908 | 0.840 | 0.760 | 0.944 | 0.964 | 0.883 |
| TTC17_POMP | 0.932 | 0.920 | 0.640 | 0.969 | 0.955 | 0.883 |
| TCF4_MYD88 | 0.972 | 0.860 | 0.800 | 0.888 | 0.894 | 0.883 |
| IMP3_MYD88 | 0.958 | 0.820 | 0.800 | 0.908 | 0.927 | 0.883 |
| TOP2B_CD63 | 0.980 | 1.000 | 0.600 | 0.929 | 0.903 | 0.882 |
| CEP192_RALB | 0.982 | 0.840 | 0.760 | 0.959 | 0.870 | 0.882 |
| NUP160_PGD | 0.950 | 0.960 | 0.720 | 0.944 | 0.833 | 0.882 |
| RPL9_SQRDL | 0.938 | 0.840 | 0.720 | 0.918 | 0.991 | 0.881 |
| CEP192_PCMT1 | 0.965 | 0.920 | 0.840 | 0.893 | 0.788 | 0.881 |
| TCF4_SQRDL | 0.976 | 0.900 | 0.720 | 0.939 | 0.870 | 0.881 |
| RPL9_GNG5 | 0.962 | 0.760 | 0.800 | 0.878 | 1.000 | 0.880 |
| EXOSC10_CD63 | 0.997 | 1.000 | 0.560 | 0.954 | 0.888 | 0.880 |
| TCF4_SH3GLB1 | 0.979 | 0.820 | 0.760 | 0.913 | 0.927 | 0.880 |
| ADSL_WARS | 0.955 | 0.980 | 0.840 | 0.760 | 0.864 | 0.880 |
| TTC17_SH3GLB1 | 0.972 | 0.920 | 0.560 | 0.969 | 0.976 | 0.879 |
| ARID1A_SQRDL | 0.953 | 0.940 | 0.560 | 0.974 | 0.970 | 0.879 |
| ARID1A_G6PD | 0.972 | 0.780 | 0.760 | 0.893 | 0.988 | 0.879 |
| AHCTF1_TANK | 0.947 | 0.700 | 0.840 | 0.923 | 0.982 | 0.878 |
| EXOSC2_CEBPB | 0.950 | 1.000 | 0.600 | 0.923 | 0.918 | 0.878 |
| RPS4X_SERPINB1 | 0.953 | 0.980 | 0.640 | 0.939 | 0.879 | 0.878 |
| FBXO11_RALB | 0.946 | 0.840 | 0.720 | 0.939 | 0.945 | 0.878 |
| TMEM50B_SQRDL | 0.968 | 0.900 | 0.680 | 0.990 | 0.852 | 0.878 |
| CSNK1G2_CEBPB | 0.959 | 1.000 | 0.560 | 0.878 | 0.988 | 0.877 |
| RPL15_SH3GLB1 | 0.964 | 0.980 | 0.720 | 0.990 | 0.730 | 0.877 |
| BCL11A_G6PD | 0.979 | 0.620 | 0.960 | 0.954 | 0.870 | 0.876 |
| ZBED5_SQRDL | 0.963 | 0.860 | 0.680 | 0.995 | 0.885 | 0.876 |
| ARID1A_SERPINB1 | 0.977 | 0.880 | 0.640 | 0.949 | 0.936 | 0.876 |
| RPS14_SH3GLB1 | 0.954 | 0.940 | 0.640 | 0.908 | 0.939 | 0.876 |
| EXOSC10_TAP1 | 0.969 | 1.000 | 0.600 | 0.949 | 0.864 | 0.876 |
| BCL11A_CEBPB | 0.978 | 0.720 | 0.720 | 1.000 | 0.961 | 0.876 |
| ADSL_ATOX1 | 0.928 | 1.000 | 0.600 | 0.893 | 0.958 | 0.876 |
| TCF4_FCER1G | 0.992 | 0.780 | 0.760 | 0.923 | 0.921 | 0.875 |
| LY9_SH3GLB1 | 0.961 | 0.720 | 0.760 | 0.964 | 0.970 | 0.875 |
| IMP3_GNG5 | 0.979 | 0.700 | 0.800 | 0.918 | 0.976 | 0.875 |
| SERTAD2_CEBPB | 0.979 | 0.820 | 0.760 | 0.908 | 0.906 | 0.875 |
| AHCTF1_MYD88 | 0.962 | 0.640 | 0.840 | 0.964 | 0.967 | 0.875 |
| ARID1A_ENO1 | 0.949 | 0.800 | 0.640 | 0.995 | 0.988 | 0.874 |
| EXOSC10_UPP1 | 0.990 | 1.000 | 0.560 | 0.923 | 0.897 | 0.874 |
| CEP192_CSTB | 0.939 | 0.760 | 0.880 | 0.872 | 0.918 | 0.874 |
| LY9_SQRDL | 0.967 | 0.720 | 0.800 | 1.000 | 0.882 | 0.874 |
| LY9_TNIP1 | 0.982 | 0.660 | 0.920 | 0.903 | 0.903 | 0.874 |
| CNOT7_G6PD | 0.966 | 0.980 | 0.760 | 0.857 | 0.803 | 0.873 |
| ARID1A_PLSCR1 | 0.946 | 0.960 | 0.640 | 0.949 | 0.870 | 0.873 |
| CEP192_ATOX1 | 0.920 | 0.920 | 0.600 | 0.974 | 0.948 | 0.873 |
| IMP3_ENO1 | 0.983 | 0.720 | 0.800 | 0.985 | 0.876 | 0.873 |
| ARID1A_IRF1 | 0.923 | 1.000 | 0.640 | 0.811 | 0.988 | 0.872 |
| EXOSC10_GNG5 | 0.978 | 0.840 | 0.680 | 0.903 | 0.961 | 0.872 |
| LY9_ATOX1 | 0.953 | 0.700 | 0.760 | 1.000 | 0.948 | 0.872 |
| FBXO11_CEBPB | 0.932 | 0.880 | 0.600 | 0.944 | 1.000 | 0.871 |
| RPL9_SLAMF7 | 0.926 | 0.920 | 0.760 | 0.903 | 0.845 | 0.871 |
| RPL9_TNIP1 | 0.946 | 0.880 | 0.800 | 0.755 | 0.973 | 0.871 |
| PREPL_CD63 | 0.946 | 1.000 | 0.560 | 0.847 | 1.000 | 0.871 |
| ARHGAP17_SQRDL | 0.984 | 0.960 | 0.720 | 0.837 | 0.852 | 0.871 |
| ZBED5_POMP | 0.953 | 0.780 | 0.680 | 1.000 | 0.939 | 0.871 |
| RPS4X_TSPO | 0.944 | 0.820 | 0.720 | 0.944 | 0.924 | 0.870 |
| IMP3_G6PD | 0.989 | 0.680 | 0.840 | 0.939 | 0.903 | 0.870 |
| CEP192_POMP | 0.932 | 0.780 | 0.720 | 0.964 | 0.955 | 0.870 |
| TMEM5OB_CD63 | 0.988 | 0.860 | 0.680 | 0.995 | 0.827 | 0.870 |
| ZMYND11_ENO1 | 0.931 | 0.880 | 0.600 | 1.000 | 0.936 | 0.870 |
| CEP192_LAP3 | 0.920 | 0.860 | 0.680 | 0.923 | 0.964 | 0.869 |
| RPL9_UPP1 | 0.948 | 0.960 | 0.640 | 0.842 | 0.958 | 0.869 |
| TCF4_SERPINB1 | 0.984 | 0.920 | 0.760 | 0.883 | 0.800 | 0.869 |
| AHCTF1_PLAUR | 0.973 | 0.720 | 0.800 | 0.857 | 0.994 | 0.869 |
| RPL22_WARS | 0.932 | 1.000 | 0.760 | 0.903 | 0.748 | 0.869 |
| EXOSC2_POMP | 0.924 | 0.900 | 0.640 | 0.934 | 0.945 | 0.869 |
| ZMYND11_SH3GLB1 | 0.919 | 0.920 | 0.520 | 0.990 | 0.994 | 0.869 |
| RPS14_CD63 | 0.983 | 0.960 | 0.600 | 0.949 | 0.848 | 0.868 |
| CAMK2G_SQRDL | 0.882 | 1.000 | 0.520 | 0.990 | 0.948 | 0.868 |
| ARIH2_CEBPB | 0.959 | 0.780 | 0.680 | 0.980 | 0.939 | 0.868 |
| ARID1A_NFIL3 | 0.975 | 0.980 | 0.600 | 0.791 | 0.988 | 0.867 |
| IMP3_POMP | 0.968 | 0.760 | 0.720 | 0.944 | 0.942 | 0.867 |
| EXOSC10_ENO1 | 0.979 | 1.000 | 0.560 | 0.995 | 0.800 | 0.867 |
| PREPL_SH3GLB1 | 0.922 | 0.960 | 0.600 | 0.852 | 1.000 | 0.867 |
| TTC17_BCL6 | 0.991 | 0.920 | 0.600 | 0.903 | 0.918 | 0.867 |
| ZMYND11_POMP | 0.911 | 0.980 | 0.480 | 1.000 | 0.958 | 0.866 |
| IMP3_RIT1 | 0.967 | 0.880 | 0.760 | 0.939 | 0.782 | 0.866 |
| CAMK2G_CD63 | 0.961 | 1.000 | 0.480 | 0.980 | 0.906 | 0.865 |
| IL10RA_CEBPB | 0.976 | 0.800 | 0.680 | 0.985 | 0.885 | 0.865 |
| FNTA_TCIRG1 | 0.951 | 0.860 | 0.640 | 0.913 | 0.961 | 0.865 |
| CAMK2G_TCIRG1 | 0.912 | 0.980 | 0.560 | 0.959 | 0.912 | 0.865 |
| EXOSC10_PCMT1 | 0.982 | 0.980 | 0.760 | 0.918 | 0.682 | 0.865 |
| RPS14_SQRDL | 0.956 | 0.940 | 0.600 | 0.929 | 0.897 | 0.864 |
| IMP3_PGD | 0.994 | 0.720 | 0.840 | 0.949 | 0.818 | 0.864 |
| ZBED5_TNIP1 | 0.987 | 0.860 | 0.720 | 0.898 | 0.855 | 0.864 |
| CHN2_WARS | 0.950 | 1.000 | 0.640 | 0.786 | 0.942 | 0.864 |
| IMP3_TCIRG1 | 0.970 | 0.800 | 0.800 | 0.908 | 0.839 | 0.863 |
| AHCTF1_SQRDL | 0.957 | 0.660 | 0.760 | 0.985 | 0.955 | 0.863 |
| CLIP4_WARS | 0.927 | 0.740 | 0.760 | 0.944 | 0.942 | 0.863 |
| NOSIP_POMP | 0.950 | 0.800 | 0.680 | 0.980 | 0.903 | 0.862 |
| RPL22_SQRDL | 0.933 | 0.920 | 0.640 | 0.985 | 0.833 | 0.862 |
| IMP3_VAMP3 | 0.966 | 0.620 | 0.840 | 0.934 | 0.952 | 0.862 |
| TTC17_TIMP2 | 0.971 | 0.780 | 0.640 | 0.990 | 0.930 | 0.862 |
| TTC17_SQRDL | 0.956 | 0.980 | 0.440 | 0.995 | 0.939 | 0.862 |
| ARID1A_CD63 | 0.985 | 0.860 | 0.520 | 0.985 | 0.961 | 0.862 |
| FNTA_LAP3 | 0.923 | 0.960 | 0.560 | 0.918 | 0.948 | 0.862 |
| BCL11A_LAP3 | 0.931 | 0.680 | 0.800 | 0.974 | 0.924 | 0.862 |
| IMP3_FCER1G | 0.988 | 0.680 | 0.760 | 0.934 | 0.945 | 0.861 |
| CEP192_TNIP1 | 0.964 | 0.860 | 0.680 | 0.878 | 0.924 | 0.861 |
| ZMYND11_SQRDL | 0.910 | 0.920 | 0.520 | 0.995 | 0.961 | 0.861 |
| ZMYND11_GNG5 | 0.935 | 0.960 | 0.440 | 0.985 | 0.985 | 0.861 |
| ARID1A_SLAMF7 | 0.953 | 0.980 | 0.640 | 0.903 | 0.827 | 0.861 |
| ARID1A_TCIRG1 | 0.964 | 0.820 | 0.680 | 0.913 | 0.924 | 0.860 |
| ARID1A_TNIP1 | 0.951 | 1.000 | 0.520 | 0.872 | 0.958 | 0.860 |
| ZMYND11_PGD | 0.971 | 0.940 | 0.560 | 0.990 | 0.839 | 0.860 |
| CSNK1G2_TCIRG1 | 0.969 | 1.000 | 0.520 | 0.908 | 0.900 | 0.859 |
| TTC17_CD63 | 0.986 | 0.980 | 0.440 | 0.969 | 0.921 | 0.859 |
| NUP160_RTN4 | 0.978 | 0.840 | 0.720 | 0.944 | 0.812 | 0.859 |
| RPL15_SQRDL | 0.956 | 1.000 | 0.760 | 0.995 | 0.582 | 0.859 |
| TTC17_UPP1 | 0.981 | 0.940 | 0.520 | 0.939 | 0.912 | 0.858 |
| CAMK2G_FCER1G | 0.941 | 0.940 | 0.520 | 0.954 | 0.936 | 0.858 |
| CEP192_TCIRG1 | 0.966 | 0.740 | 0.760 | 0.913 | 0.912 | 0.858 |
| IRF8_CEBPB | 0.984 | 0.600 | 0.920 | 0.857 | 0.930 | 0.858 |
| CEP192_G6PD | 0.980 | 0.660 | 0.880 | 0.898 | 0.873 | 0.858 |
| FBXO11_UPP1 | 0.942 | 0.840 | 0.600 | 0.929 | 0.979 | 0.858 |
| ARIH2_TCIRG1 | 0.971 | 0.700 | 0.720 | 0.964 | 0.933 | 0.858 |
| PCID2_WARS | 0.948 | 0.640 | 0.800 | 0.923 | 0.976 | 0.858 |
| CAMK2G_PGD | 0.962 | 0.980 | 0.560 | 0.985 | 0.800 | 0.857 |
| EXOSC10_FLII | 0.954 | 0.840 | 0.680 | 0.934 | 0.879 | 0.857 |
| RPL15_CD63 | 0.991 | 1.000 | 0.720 | 0.990 | 0.585 | 0.857 |
| RPL22_CD63 | 0.978 | 0.960 | 0.600 | 0.959 | 0.788 | 0.857 |
| CNOT7_SQRDL | 0.956 | 1.000 | 0.600 | 0.862 | 0.867 | 0.857 |
| FBXO11_SQRDL | 0.914 | 0.860 | 0.520 | 0.990 | 1.000 | 0.857 |
| TCF4_UPP1 | 0.988 | 0.900 | 0.760 | 0.827 | 0.809 | 0.857 |
| PCID2_CEBPB | 0.953 | 0.660 | 0.720 | 0.949 | 1.000 | 0.856 |
| CNOT7_CSTB | 0.953 | 0.940 | 0.760 | 0.816 | 0.812 | 0.856 |
| ARID1A_PGD | 0.991 | 0.880 | 0.560 | 0.964 | 0.885 | 0.856 |
| ARID1A_STAT3 | 0.956 | 0.960 | 0.560 | 0.913 | 0.891 | 0.856 |
| NOSIP_TCIRG1 | 0.954 | 0.720 | 0.800 | 0.944 | 0.861 | 0.856 |
| RPL9_FCER1G | 0.979 | 0.740 | 0.680 | 0.888 | 0.991 | 0.856 |
| ARID1A_TRPC4AP | 0.946 | 0.920 | 0.600 | 0.811 | 1.000 | 0.855 |
| ARID1A_SH3GLB1 | 0.964 | 0.820 | 0.560 | 0.944 | 0.988 | 0.855 |
| CEP192_RAB27A | 0.972 | 0.840 | 0.720 | 0.832 | 0.912 | 0.855 |
| EXOSC10_FCER1G | 0.992 | 0.880 | 0.520 | 0.944 | 0.939 | 0.855 |
| SETX_SQRDL | 0.965 | 0.940 | 0.520 | 0.913 | 0.936 | 0.855 |
| CEP192_MYD88 | 0.959 | 0.780 | 0.680 | 0.883 | 0.973 | 0.855 |
| ARID1A_BCL6 | 0.987 | 0.920 | 0.560 | 0.888 | 0.918 | 0.855 |
| EXOSC2_CD63 | 0.965 | 0.920 | 0.560 | 0.949 | 0.879 | 0.855 |
| AHCTF1_UPP1 | 0.974 | 0.760 | 0.640 | 0.974 | 0.924 | 0.855 |
| IMP3_RALB | 0.965 | 0.700 | 0.840 | 0.939 | 0.827 | 0.854 |
| ADK_SH3GLB1 | 0.979 | 1.000 | 0.760 | 0.878 | 0.655 | 0.854 |
| SUCLG2_CD63 | 0.995 | 0.960 | 0.680 | 0.923 | 0.712 | 0.854 |
| FNTA_WARS | 0.950 | 0.960 | 0.560 | 0.918 | 0.882 | 0.854 |
| EXOSC10_TUBA1B | 0.981 | 0.640 | 0.760 | 1.000 | 0.888 | 0.854 |
| IMP3_PCBP1 | 0.975 | 0.600 | 0.920 | 0.878 | 0.894 | 0.853 |
| ARID1A_GRINA | 0.941 | 0.940 | 0.520 | 0.929 | 0.936 | 0.853 |
| TTC17_PGD | 0.993 | 1.000 | 0.480 | 0.995 | 0.797 | 0.853 |
| ARID1A_TANK | 0.948 | 1.000 | 0.440 | 0.898 | 0.979 | 0.853 |
| CSNK1G2_FLII | 0.929 | 0.920 | 0.640 | 0.883 | 0.894 | 0.853 |
| CEP192_STAT3 | 0.973 | 0.900 | 0.640 | 0.939 | 0.812 | 0.853 |
| AHCTF1_SH3GLB1 | 0.956 | 0.620 | 0.720 | 0.980 | 0.985 | 0.852 |
| TTC17_SERPINB1 | 0.975 | 0.900 | 0.520 | 0.959 | 0.906 | 0.852 |
| EXOSC2_UPP1 | 0.957 | 0.980 | 0.560 | 0.888 | 0.876 | 0.852 |
| IMP3_TSPO | 0.980 | 0.520 | 0.880 | 0.985 | 0.894 | 0.852 |
| BCL11A_TNIP1 | 0.986 | 0.640 | 0.840 | 0.878 | 0.915 | 0.852 |
| ADSL_ENO1 | 0.988 | 0.920 | 0.640 | 0.862 | 0.848 | 0.852 |
| NOSIP_SQRDL | 0.948 | 0.800 | 0.680 | 0.990 | 0.839 | 0.851 |
| SERBP1_SH3GLB1 | 0.971 | 0.920 | 0.600 | 0.888 | 0.879 | 0.851 |
| ARID1A_NFKBIA | 0.993 | 0.940 | 0.680 | 0.791 | 0.852 | 0.851 |
| RPL9_ENO1 | 0.948 | 0.780 | 0.720 | 0.888 | 0.918 | 0.851 |
| ARID1A_RAB27A | 0.960 | 0.880 | 0.600 | 0.862 | 0.952 | 0.851 |
| RPL15_WARS | 0.950 | 1.000 | 0.840 | 0.974 | 0.488 | 0.851 |
| BCL11A_CSTB | 0.939 | 0.500 | 1.000 | 0.929 | 0.885 | 0.851 |
| ARID1A_SOCS3 | 0.964 | 0.980 | 0.600 | 0.760 | 0.948 | 0.850 |
| ARID1A_C3AR1 | 0.993 | 0.720 | 0.680 | 0.913 | 0.945 | 0.850 |
| RPL15_GPI | 0.980 | 0.780 | 0.800 | 0.990 | 0.700 | 0.850 |
| ARIH2_TNIP1 | 0.978 | 0.800 | 0.600 | 0.908 | 0.964 | 0.850 |
| TOP2B_TUBA1B | 0.957 | 0.680 | 0.720 | 0.985 | 0.906 | 0.849 |
| ZBED5_CD63 | 0.988 | 0.820 | 0.600 | 0.995 | 0.842 | 0.849 |
| TCF4_PGD | 0.993 | 0.840 | 0.760 | 0.918 | 0.733 | 0.849 |
| ARID1A_MYD88 | 0.950 | 0.860 | 0.560 | 0.929 | 0.945 | 0.849 |
| TTC17_FCER1G | 0.983 | 0.800 | 0.560 | 0.985 | 0.915 | 0.849 |
| BCL11A_POMP | 0.953 | 0.540 | 0.840 | 0.990 | 0.918 | 0.848 |
| ARID1A_UPP1 | 0.974 | 0.860 | 0.560 | 0.934 | 0.912 | 0.848 |
| ARID1A_ERLIN1 | 0.949 | 0.900 | 0.600 | 0.990 | 0.797 | 0.847 |
| MGEA5_SQRDL | 0.877 | 0.940 | 0.440 | 0.995 | 0.982 | 0.847 |
| NUP160_TPP1 | 0.990 | 0.500 | 0.880 | 0.903 | 0.961 | 0.847 |
| HLA-DPA1_CEBPB | 0.986 | 0.720 | 0.800 | 0.872 | 0.855 | 0.847 |
| RPL9_SERPINB1 | 0.952 | 0.840 | 0.640 | 0.857 | 0.942 | 0.846 |
| SETX_CD63 | 0.973 | 0.820 | 0.600 | 0.898 | 0.939 | 0.846 |
| RPL9_LDHA | 0.960 | 0.780 | 0.600 | 0.908 | 0.982 | 0.846 |
| EXOSC10_SERPINB1 | 0.982 | 0.960 | 0.520 | 0.929 | 0.839 | 0.846 |
| EXOSC10_PGD | 0.995 | 1.000 | 0.560 | 0.964 | 0.709 | 0.846 |
| EXOSC10_RALB | 0.969 | 0.900 | 0.600 | 0.944 | 0.815 | 0.846 |
| EXOSC10_TSPO | 0.981 | 0.880 | 0.560 | 0.969 | 0.836 | 0.845 |
| ARID1A_CD55 | 0.976 | 0.820 | 0.640 | 0.821 | 0.970 | 0.845 |
| CHN2_FCER1G | 0.972 | 0.920 | 0.520 | 0.827 | 0.988 | 0.845 |
| LY9_MYD88 | 0.958 | 0.620 | 0.800 | 0.944 | 0.903 | 0.845 |
| ARID1A_BCL3 | 0.979 | 0.940 | 0.600 | 0.745 | 0.961 | 0.845 |
| ARID1A_ETV6 | 0.944 | 0.880 | 0.560 | 0.918 | 0.921 | 0.845 |
| IRF8_LAP3 | 0.933 | 0.660 | 0.960 | 0.791 | 0.879 | 0.844 |
| TCF4_CD63 | 0.986 | 0.840 | 0.680 | 0.883 | 0.833 | 0.844 |
| FBXO11_MYD88 | 0.915 | 0.780 | 0.600 | 0.934 | 0.994 | 0.844 |
| TMEM106B_CEBPB | 0.974 | 0.960 | 0.640 | 0.765 | 0.882 | 0.844 |
| RPL9_PGD | 0.979 | 0.820 | 0.680 | 0.872 | 0.870 | 0.844 |
| ZNF266_CD63 | 0.976 | 0.820 | 0.640 | 0.995 | 0.788 | 0.844 |
| CCR7_CEBPB | 0.945 | 0.620 | 0.760 | 0.923 | 0.970 | 0.844 |
| CEP192_SQRDL | 0.965 | 0.760 | 0.600 | 0.974 | 0.918 | 0.844 |
| ARID1A_PRKCD | 0.982 | 0.760 | 0.600 | 0.990 | 0.885 | 0.843 |
| FBXO11_SH3GLB1 | 0.926 | 0.760 | 0.560 | 0.969 | 1.000 | 0.843 |
| IMP3_PRKCD | 0.972 | 0.700 | 0.800 | 0.959 | 0.785 | 0.843 |
| EXOSC10_GPI | 0.964 | 0.760 | 0.600 | 0.985 | 0.906 | 0.843 |
| CEP192_UPP1 | 0.988 | 0.840 | 0.560 | 0.888 | 0.939 | 0.843 |
| BCL11A_GNG5 | 0.975 | 0.640 | 0.680 | 0.969 | 0.948 | 0.843 |
| ARIH2_SH3GLB1 | 0.947 | 0.680 | 0.640 | 0.985 | 0.961 | 0.843 |
| TOP2B_TPP1 | 0.992 | 0.640 | 0.680 | 0.985 | 0.915 | 0.842 |
| SEH1L_SQRDL | 0.961 | 0.820 | 0.600 | 0.883 | 0.942 | 0.841 |
| ARID1A_FCER1G | 0.980 | 0.780 | 0.520 | 0.959 | 0.967 | 0.841 |
| EXOSC10_ERLIN1 | 0.986 | 1.000 | 0.520 | 0.980 | 0.715 | 0.840 |
| ARID1A_RTN4 | 0.988 | 0.800 | 0.600 | 0.954 | 0.858 | 0.840 |
| HERC6_LAP3 | 0.907 | 0.840 | 0.560 | 0.913 | 0.979 | 0.840 |
| ARID1A_FLOT1 | 0.980 | 0.920 | 0.560 | 0.913 | 0.824 | 0.839 |
| TCF4_PRKCD | 0.993 | 0.760 | 0.800 | 0.898 | 0.742 | 0.839 |
| LY9_PLAUR | 0.971 | 0.640 | 0.760 | 0.852 | 0.970 | 0.839 |
| ARID1A_NUMB | 0.965 | 0.860 | 0.520 | 0.929 | 0.918 | 0.838 |
| TRAF3IP3_WARS | 0.949 | 0.760 | 0.600 | 0.929 | 0.955 | 0.838 |
| CEP192_SH3GLB1 | 0.965 | 0.720 | 0.600 | 0.918 | 0.988 | 0.838 |
| AHCTF1_PGD | 0.991 | 0.680 | 0.720 | 0.974 | 0.824 | 0.838 |
| EXOSC2_SQRDL | 0.924 | 0.880 | 0.520 | 0.974 | 0.891 | 0.838 |
| FBXO11_CD63 | 0.950 | 0.760 | 0.520 | 0.964 | 0.994 | 0.838 |
| PCID2_POMP | 0.957 | 0.520 | 0.760 | 0.954 | 0.997 | 0.838 |
| TTC17_FGR | 0.992 | 0.920 | 0.520 | 1.000 | 0.755 | 0.837 |
| TROVE2_CEBPB | 0.979 | 0.980 | 0.600 | 0.765 | 0.861 | 0.837 |
| ARID1A_RALB | 0.965 | 0.840 | 0.560 | 0.944 | 0.876 | 0.837 |
| BCL11A_SQRDL | 0.973 | 0.620 | 0.680 | 0.969 | 0.939 | 0.836 |
| IMP3_SERPINB1 | 0.975 | 0.720 | 0.640 | 0.918 | 0.927 | 0.836 |
| LY9_POMP | 0.966 | 0.540 | 0.760 | 0.985 | 0.930 | 0.836 |
| CLIP4_CEBPB | 0.960 | 0.660 | 0.600 | 0.959 | 1.000 | 0.836 |
| RPS4X_SPI1 | 0.952 | 0.680 | 0.720 | 0.923 | 0.903 | 0.836 |
| BCL11A_FCER1G | 0.990 | 0.620 | 0.640 | 0.995 | 0.930 | 0.835 |
| EXOSC2_FCER1G | 0.963 | 0.800 | 0.600 | 0.918 | 0.891 | 0.835 |
| AHCTF1_CD63 | 0.987 | 0.520 | 0.760 | 0.974 | 0.930 | 0.834 |
| ARIH2_SQRDL | 0.943 | 0.740 | 0.600 | 0.985 | 0.900 | 0.834 |
| CEP192_LDHA | 0.952 | 0.680 | 0.640 | 0.918 | 0.976 | 0.833 |
| FBXO11_SERPINB1 | 0.943 | 0.820 | 0.480 | 0.944 | 0.976 | 0.832 |
| IL10RA_TNIP1 | 0.980 | 0.840 | 0.640 | 0.923 | 0.779 | 0.832 |
| CEP192_GNG5 | 0.964 | 0.660 | 0.680 | 0.872 | 0.985 | 0.832 |
| ARHGAP17_CD63 | 0.998 | 0.960 | 0.640 | 0.745 | 0.818 | 0.832 |
| CNOT7_FCER1G | 0.996 | 0.920 | 0.520 | 0.867 | 0.858 | 0.832 |
| ARIH2_G6PD | 0.980 | 0.600 | 0.680 | 0.995 | 0.906 | 0.832 |
| NUP160_WAS | 0.960 | 0.540 | 0.800 | 0.908 | 0.952 | 0.832 |
| LY9_CD63 | 0.989 | 0.500 | 0.800 | 0.980 | 0.891 | 0.832 |
| BCL11A_SH3GLB1 | 0.980 | 0.540 | 0.720 | 0.959 | 0.961 | 0.832 |
| ASXL2_WARS | 0.920 | 0.680 | 0.680 | 0.888 | 0.991 | 0.832 |
| FBL_SQRDL | 0.959 | 1.000 | 0.520 | 0.995 | 0.685 | 0.832 |
| CD52_CD63 | 0.993 | 0.800 | 0.680 | 0.929 | 0.758 | 0.832 |
| ADSL_POMP | 0.972 | 0.900 | 0.520 | 0.796 | 0.970 | 0.832 |
| SERBP1_CD63 | 0.991 | 0.980 | 0.520 | 0.888 | 0.779 | 0.831 |
| ARID1A_POMP | 0.933 | 0.800 | 0.520 | 0.969 | 0.933 | 0.831 |
| CHN2_SQRDL | 0.929 | 1.000 | 0.480 | 0.770 | 0.976 | 0.831 |
| ARIH2_UPP1 | 0.970 | 0.800 | 0.480 | 0.990 | 0.915 | 0.831 |
| CEP192_VAMP3 | 0.972 | 0.680 | 0.640 | 0.908 | 0.955 | 0.831 |
| BCL11A_TANK | 0.979 | 0.420 | 0.880 | 0.908 | 0.967 | 0.831 |
| GLG1_SQRDL | 0.961 | 0.880 | 0.560 | 0.913 | 0.839 | 0.831 |
| IRF8_WARS | 0.966 | 0.580 | 0.960 | 0.862 | 0.785 | 0.831 |
| HLA-DPA1_WARS | 0.983 | 0.720 | 0.840 | 0.918 | 0.691 | 0.830 |
| DNAJC10_SQRDL | 0.952 | 0.940 | 0.560 | 0.781 | 0.918 | 0.830 |
| ARID1A_FGR | 0.989 | 0.820 | 0.560 | 0.944 | 0.836 | 0.830 |
| RPL9_TRIB1 | 0.952 | 0.780 | 0.680 | 0.806 | 0.927 | 0.829 |
| LY9_UPP1 | 0.979 | 0.600 | 0.720 | 0.964 | 0.882 | 0.829 |
| IL10RA_MYD88 | 0.963 | 0.740 | 0.640 | 0.985 | 0.815 | 0.829 |
| METAP1_RTN4 | 0.965 | 0.680 | 0.640 | 0.959 | 0.891 | 0.827 |
| BCL11A_RALB | 0.982 | 0.560 | 0.760 | 0.990 | 0.842 | 0.827 |
| ARID1A_ATP2A2 | 0.911 | 0.860 | 0.440 | 0.923 | 0.997 | 0.826 |
| SERBP1_SQRDL | 0.967 | 0.900 | 0.560 | 0.918 | 0.785 | 0.826 |
| MLLT10_CD63 | 0.964 | 0.720 | 0.480 | 0.974 | 0.988 | 0.825 |
| PCID2_CD63 | 0.975 | 0.600 | 0.600 | 0.954 | 0.997 | 0.825 |
| ARID1A_FLII | 0.936 | 0.600 | 0.760 | 0.903 | 0.924 | 0.825 |
| EXOSC10_VAMP3 | 0.969 | 0.640 | 0.640 | 0.939 | 0.924 | 0.822 |
| CEP192_NFIL3 | 0.980 | 0.920 | 0.440 | 0.801 | 0.970 | 0.822 |
| ARID1A_HCK | 0.961 | 0.700 | 0.560 | 0.964 | 0.924 | 0.822 |
| IMP3_SPI1 | 0.986 | 0.480 | 0.840 | 0.934 | 0.870 | 0.822 |
| PCID2_SQRDL | 0.934 | 0.560 | 0.640 | 0.974 | 1.000 | 0.822 |
| MLLT10_PGD | 0.957 | 0.760 | 0.480 | 0.969 | 0.942 | 0.822 |
| ARID1A_PLAUR | 0.956 | 0.860 | 0.560 | 0.745 | 0.988 | 0.822 |
| TCF4_HCK | 0.970 | 0.600 | 0.800 | 0.944 | 0.794 | 0.821 |
| TCF4_VAMP3 | 0.977 | 0.580 | 0.800 | 0.913 | 0.836 | 0.821 |
| EXOSC10_FGR | 0.991 | 0.980 | 0.520 | 0.934 | 0.682 | 0.821 |
| ADSL_CD63 | 0.990 | 0.940 | 0.480 | 0.801 | 0.894 | 0.821 |
| CEP192_BCL6 | 0.995 | 0.780 | 0.520 | 0.867 | 0.942 | 0.821 |
| RPL9_TSPO | 0.950 | 0.660 | 0.720 | 0.816 | 0.958 | 0.821 |
| FBXO11_FCER1G | 0.950 | 0.720 | 0.520 | 0.944 | 0.970 | 0.821 |
| HLA-DPA1_MYD88 | 0.980 | 0.660 | 0.800 | 0.898 | 0.764 | 0.820 |
| CEP192_CD63 | 0.991 | 0.700 | 0.560 | 0.929 | 0.921 | 0.820 |
| EXOSC2_PGD | 0.971 | 0.940 | 0.480 | 0.944 | 0.764 | 0.820 |
| EXOSC2_SH3GLB1 | 0.938 | 0.880 | 0.480 | 0.888 | 0.912 | 0.820 |
| EXOSC10_SLAMF7 | 0.979 | 1.000 | 0.480 | 0.908 | 0.730 | 0.819 |
| ARID1A_GNG5 | 0.960 | 0.820 | 0.440 | 0.903 | 0.973 | 0.819 |
| CEP192_FCER1G | 0.989 | 0.720 | 0.520 | 0.934 | 0.927 | 0.818 |
| ARID1A_CCND3 | 0.962 | 0.600 | 0.600 | 0.954 | 0.973 | 0.818 |
| CEP192_PGD | 0.995 | 0.720 | 0.600 | 0.969 | 0.803 | 0.818 |
| SERTAD2_SQRDL | 0.969 | 0.640 | 0.760 | 0.878 | 0.839 | 0.817 |
| ASXL2_SH3GLB1 | 0.943 | 0.600 | 0.640 | 0.903 | 1.000 | 0.817 |
| BCL11A_UPP1 | 0.989 | 0.640 | 0.640 | 0.934 | 0.882 | 0.817 |
| ARID1A_TSPO | 0.973 | 0.660 | 0.520 | 1.000 | 0.930 | 0.817 |
| ASXL2_IRF1 | 0.912 | 0.860 | 0.560 | 0.750 | 1.000 | 0.816 |
| TROVE2_SQRDL | 0.962 | 0.940 | 0.600 | 0.816 | 0.764 | 0.816 |
| IMP3_C3AR1 | 0.994 | 0.580 | 0.680 | 0.867 | 0.961 | 0.816 |
| BCL11A_NFIL3 | 0.982 | 0.620 | 0.640 | 0.903 | 0.936 | 0.816 |
| TROVE2_SH3GLB1 | 0.977 | 0.900 | 0.520 | 0.837 | 0.845 | 0.816 |
| RPL9_RTN4 | 0.994 | 0.760 | 0.760 | 0.867 | 0.697 | 0.816 |
| SERTAD2_SH3GLB1 | 0.970 | 0.640 | 0.680 | 0.903 | 0.885 | 0.816 |
| ASXL2_BCL6 | 0.975 | 0.580 | 0.680 | 0.852 | 0.991 | 0.816 |
| ASXL2_CEBPB | 0.964 | 0.680 | 0.560 | 0.872 | 1.000 | 0.815 |
| HLA-DPA1_LAP3 | 0.950 | 0.760 | 0.800 | 0.786 | 0.779 | 0.815 |
| CEP192_SERPINB1 | 0.973 | 0.740 | 0.560 | 0.908 | 0.891 | 0.814 |
| SETX_SH3GLB1 | 0.963 | 0.800 | 0.480 | 0.872 | 0.955 | 0.814 |
| IL10RA_SH3GLB1 | 0.963 | 0.680 | 0.560 | 0.980 | 0.882 | 0.813 |
| RPL9_VAMP3 | 0.959 | 0.520 | 0.760 | 0.847 | 0.976 | 0.812 |
| TROVE2_CD63 | 0.985 | 0.940 | 0.560 | 0.821 | 0.755 | 0.812 |
| CEP192_ACSL4 | 0.983 | 0.740 | 0.560 | 0.939 | 0.833 | 0.811 |
| ASXL2_CD63 | 0.975 | 0.580 | 0.600 | 0.903 | 0.997 | 0.811 |
| USP34_CD63 | 0.947 | 0.560 | 0.560 | 0.980 | 1.000 | 0.809 |
| ASXL2_UPP1 | 0.964 | 0.680 | 0.560 | 0.857 | 0.985 | 0.809 |
| ASXL2_TSPO | 0.962 | 0.660 | 0.520 | 0.908 | 0.994 | 0.809 |
| CEP192_ERLIN1 | 0.946 | 0.800 | 0.560 | 0.959 | 0.779 | 0.809 |
| TCF4_TSPO | 0.983 | 0.600 | 0.800 | 0.842 | 0.818 | 0.809 |
| ARIH2_FCER1G | 0.974 | 0.540 | 0.600 | 0.969 | 0.958 | 0.808 |
| ARID1A_SORT1 | 0.973 | 0.760 | 0.560 | 0.959 | 0.788 | 0.808 |
| FNTA_G6PD | 0.967 | 0.780 | 0.520 | 0.913 | 0.855 | 0.807 |
| RPL9_SPI1 | 0.967 | 0.500 | 0.720 | 0.888 | 0.958 | 0.806 |
| ARIH2_PGD | 0.982 | 0.680 | 0.640 | 0.985 | 0.742 | 0.806 |
| TRAF3IP3_SQRDL | 0.958 | 0.560 | 0.560 | 0.969 | 0.982 | 0.806 |
| TTC17_TSPO | 0.975 | 0.780 | 0.400 | 1.000 | 0.873 | 0.806 |
| ASXL2_FGR | 0.975 | 0.660 | 0.560 | 0.908 | 0.924 | 0.805 |
| LY9_PGD | 0.988 | 0.500 | 0.760 | 0.990 | 0.788 | 0.805 |
| ARID1A_TIMP2 | 0.963 | 0.720 | 0.480 | 0.913 | 0.948 | 0.805 |
| PCID2_SH3GLB1 | 0.951 | 0.540 | 0.600 | 0.934 | 1.000 | 0.805 |
| ARID1A_ATP6V1B2 | 0.976 | 0.720 | 0.520 | 0.959 | 0.848 | 0.805 |
| BCL11A_CD63 | 0.994 | 0.520 | 0.640 | 0.954 | 0.912 | 0.804 |
| ARID1A_RBMS1 | 0.974 | 0.740 | 0.480 | 0.878 | 0.942 | 0.803 |
| HLA-DPA1_SQRDL | 0.976 | 0.700 | 0.760 | 0.862 | 0.715 | 0.803 |
| IMP3_LDHA | 0.970 | 0.700 | 0.400 | 0.964 | 0.976 | 0.802 |
| CNOT7_PGD | 0.999 | 0.980 | 0.480 | 0.867 | 0.679 | 0.801 |
| IRF8_SQRDL | 0.972 | 0.620 | 0.720 | 0.852 | 0.839 | 0.801 |
| ASXL2_TCIRG1 | 0.954 | 0.600 | 0.600 | 0.883 | 0.967 | 0.801 |
| BCL11A_MYD88 | 0.966 | 0.420 | 0.680 | 0.985 | 0.942 | 0.799 |
| CCR7_SQRDL | 0.942 | 0.440 | 0.680 | 0.985 | 0.942 | 0.798 |
| ASXL2_G6PD | 0.956 | 0.520 | 0.680 | 0.832 | 1.000 | 0.798 |
| ARID1A_SPI1 | 0.978 | 0.580 | 0.560 | 0.918 | 0.948 | 0.797 |
| ARID1A_VAMP3 | 0.959 | 0.540 | 0.600 | 0.934 | 0.945 | 0.796 |
| AHCTF1_BCL6 | 0.995 | 0.580 | 0.480 | 0.969 | 0.952 | 0.795 |
| ASXL2_KIF1B | 0.975 | 0.680 | 0.600 | 0.929 | 0.791 | 0.795 |
| ASXL2_FCER1G | 0.972 | 0.540 | 0.560 | 0.903 | 1.000 | 0.795 |
| IL10RA_SQRDL | 0.959 | 0.680 | 0.560 | 0.980 | 0.791 | 0.794 |
| ASXL2_PGD | 0.979 | 0.540 | 0.560 | 0.923 | 0.964 | 0.793 |
| CEP192_FGR | 0.993 | 0.700 | 0.600 | 0.923 | 0.748 | 0.793 |
| ASXL2_SQRDL | 0.934 | 0.660 | 0.440 | 0.923 | 1.000 | 0.792 |
| BCL11A_LDHA | 0.969 | 0.560 | 0.520 | 0.959 | 0.942 | 0.790 |
| IRF8_POMP | 0.963 | 0.560 | 0.680 | 0.847 | 0.897 | 0.789 |
| BCL11A_PGD | 0.994 | 0.520 | 0.680 | 0.969 | 0.782 | 0.789 |
| ARID1A_JUNB | 0.981 | 0.800 | 0.520 | 0.648 | 0.994 | 0.789 |
| CEP192_TSPO | 0.984 | 0.500 | 0.640 | 0.934 | 0.882 | 0.788 |
| ASXL2_SERPINB1 | 0.954 | 0.660 | 0.440 | 0.883 | 0.997 | 0.787 |
| BCL11A_BCL6 | 0.998 | 0.560 | 0.520 | 0.964 | 0.888 | 0.786 |
| FBXO11_PGD | 0.962 | 0.660 | 0.440 | 0.974 | 0.885 | 0.784 |
| BCL11A_SERPINB1 | 0.985 | 0.440 | 0.680 | 0.944 | 0.870 | 0.784 |
| BCL11A_ERLIN1 | 0.979 | 0.580 | 0.680 | 0.964 | 0.712 | 0.783 |
| ASXL2_ETV6 | 0.928 | 0.540 | 0.520 | 0.923 | 0.991 | 0.780 |
| ASXL2_RALB | 0.952 | 0.540 | 0.480 | 0.944 | 0.985 | 0.780 |
| USP34_PGD | 0.960 | 0.560 | 0.400 | 0.980 | 1.000 | 0.780 |
| ARID1A_PCBP1 | 0.965 | 0.660 | 0.480 | 0.847 | 0.933 | 0.777 |
| EXOSC2_TSPO | 0.954 | 0.720 | 0.400 | 0.944 | 0.861 | 0.776 |
| CEP192_PRKCD | 0.993 | 0.440 | 0.720 | 0.959 | 0.761 | 0.774 |
| IRF8_SH3GLB1 | 0.985 | 0.460 | 0.680 | 0.791 | 0.933 | 0.770 |
| ARIH2_CD63 | 0.983 | 0.440 | 0.520 | 1.000 | 0.900 | 0.769 |
| ARID1A_RAB7A | 0.962 | 0.680 | 0.440 | 0.934 | 0.827 | 0.769 |
| TTC17_VAMP3 | 0.968 | 0.560 | 0.400 | 0.990 | 0.924 | 0.768 |
| ARID1A_WAS | 0.982 | 0.620 | 0.400 | 0.908 | 0.879 | 0.758 |
| TTC17_WAS | 0.994 | 0.580 | 0.360 | 1.000 | 0.839 | 0.755 |
| HLA-DPA1_POMP | 0.974 | 0.380 | 0.720 | 0.872 | 0.821 | 0.754 |

TABLE 36
TOP PERFORMING (BASED ON AUC) INSIRS DERIVED
BIOMARKERS FOLLOWING A GREEDY SEARCH ON A
COMBINED DATASET
The top derived biomarker was ENTPD1:ARL6IP5 with an AUC of 0.898.
Incremental AUC increases can be made with the addition of further
derived biomarkers as indicated.

| Derived Biomarker | AUC | Increased AUC |
|---|---|---|
| ENTPD1_ARL6IP5 | 0.898 | 0.037 |
| TNFSF8_HEATR1 | 0.935 | 0.013 |
| ADAM19_POLR2A | 0.948 | 0.007 |
| SYNE2_VPS13C | 0.955 | 0.004 |
| TNFSF8_NIP7 | 0.959 | 0.002 |
| CDA_EFHD2 | 0.962 | 0.000 |
| ADAM19_MLLT10 | 0.962 | 0.000 |
| PTGS1 + ENTPD1 | 0.962 | 0.001 |
| ADAM19_EXOC7 | 0.963 | 0.002 |
| CDA_PTGS1 | 0.965 | −0.965 |

TABLE 37
INSIRS NUMERATORS AND DENOMINATORS APPEARING MORE
THAN TWICE IN THE 164 DERIVED BIOMARKERS WITH A MEAN
AUC > 0.82 IN THE VALIDATION DATASETS.
inSIRS numerators and denominators appearing more than once in
derived biomarkers with an AUC > 0.85

| Numerator | # | Denominator | # |
|---|---|---|---|
| TNFSF8 | 90 | MACF1 | 8 |
| ADAM19 | 17 | ARL6IP5 | 6 |
| VNN3 | 12 | TRAPPC2 | 5 |
| RGS2 | 11 | KRIT1 | 3 |
| GAB2 | 8 | RBM26 | 3 |
| STK17B | 4 | SYT11 | 3 |
| ENTPD1 | 3 | YTHDC2 | 3 |
| IGF2R | 3 | CDKN1B | 2 |
| SYNE2 | 3 | CYSLTR1 | 2 |
| CDA | 2 | FCF1 | 2 |
| MXD1 | 2 | LARP1 | 2 |
| | | MLLT10 | 2 |
| | | PHC3 | 2 |
| | | S100PBP | 2 |
| | | THOC2 | 2 |
| | | ZNF507 | 2 |

TABLE 38
TABLE OF INDIVIDUAL PERFORMANCE, IN DESCENDING AUC, OF 164 INSIRS DERIVED BIOMARKERS WITH
AN AVERAGE AUC >0.82 ACROSS EACH OF SIX NON-INFECTIOUS SYSTEMIC INFLAMMATION DATASETS.

| | Children | | | | Acute | Adult | |
|---|---|---|---|---|---|---|---|
| | Sepsis/ | Auto- | | | Respiratory | Sepsis/ | |
| | SIRS | immunity | Trauma | Anaphylaxis | Inflammation | SIRS | |
| Derived Biomarker | GAPPSS | GSE17755 | GSE36809 | GSE47655 | GSE63990 | GSE74224 | MEAN |
| TNFSF8_VEZT | 0.885 | NA | 0.987 | 0.951 | 0.816 | 0.926 | 0.904 |
| TNFSF8_HEATR1 | 0.882 | NA | 0.978 | 0.840 | 0.897 | 0.907 | 0.893 |
| TNFSF8_THOC2 | 0.939 | NA | 0.977 | 0.852 | 0.780 | 0.936 | 0.889 |
| TNFSF8_NIP7 | 0.897 | NA | 0.947 | 0.840 | 0.823 | 0.961 | 0.885 |
| TNFSF8_MLLT10 | 0.859 | NA | 0.966 | 0.901 | 0.819 | 0.905 | 0.882 |
| TNFSF8_EIF5B | 0.900 | NA | 0.994 | 0.926 | 0.766 | 0.873 | 0.882 |
| TNFSF8_LRRC8D | 0.927 | NA | 0.984 | 0.852 | 0.778 | 0.904 | 0.881 |
| TNFSF8_RNMT | 0.906 | NA | 0.994 | 0.914 | 0.741 | 0.889 | 0.879 |
| STK17B_ARL6IP5 | 0.948 | 0.988 | 0.996 | 0.901 | 0.537 | 0.927 | 0.879 |
| ENTPD1_ARL6IP5 | 0.858 | 0.974 | 1.000 | 0.951 | 0.621 | 0.899 | 0.878 |
| TNFSF8_CD84 | 0.885 | NA | 0.982 | 0.951 | 0.789 | 0.841 | 0.878 |
| TNFSF8_PWP1 | 0.861 | NA | 0.996 | 0.889 | 0.773 | 0.910 | 0.877 |
| TNFSF8_IPO7 | 0.879 | NA | 0.994 | 0.901 | 0.720 | 0.936 | 0.876 |
| ADAM19_EXOC7 | 0.942 | NA | 0.987 | 0.790 | 0.805 | 0.902 | 0.875 |
| TNFSF8_ARHGAP5 | 0.891 | NA | 0.989 | 0.975 | 0.643 | 0.909 | 0.874 |
| TNFSF8_RMND1 | 0.898 | NA | 0.983 | 0.877 | 0.775 | 0.877 | 0.874 |
| TNFSF8_IDE | 0.867 | NA | 0.964 | 0.852 | 0.796 | 0.931 | 0.873 |
| TNFSF8_TBCE | 0.900 | NA | 0.974 | 0.864 | 0.784 | 0.877 | 0.873 |
| TNFSF8_G3BP1 | 0.748 | NA | 0.991 | 0.914 | 0.834 | 0.919 | 0.873 |
| TNFSF8_CDK6 | 0.873 | NA | 0.993 | 0.840 | 0.783 | 0.916 | 0.872 |
| TNFSF8_MANEA | 0.885 | NA | 0.963 | 0.877 | 0.716 | 0.944 | 0.870 |
| TNFSF8_CKAP2 | 0.876 | NA | 0.972 | 0.926 | 0.683 | 0.927 | 0.869 |
| TNFSF8_ZNF507 | 0.870 | NA | 0.987 | 0.901 | 0.755 | 0.870 | 0.869 |
| TNFSF8_GGPS1 | 0.912 | NA | 0.954 | 0.827 | 0.797 | 0.892 | 0.868 |
| TNFSF8_XPO4 | 0.885 | NA | 0.985 | 0.877 | 0.717 | 0.924 | 0.867 |
| TNFSF8_PHC3 | 0.845 | NA | 0.983 | 0.864 | 0.823 | 0.863 | 0.867 |
| TNFSF8_ASCC3 | 0.879 | NA | 0.967 | 0.901 | 0.667 | 0.954 | 0.866 |
| TNFSF8_NOL10 | 0.876 | NA | 0.963 | 0.864 | 0.783 | 0.885 | 0.866 |
| TNFSF8_ANK3 | 0.879 | NA | 0.966 | 0.901 | 0.785 | 0.855 | 0.866 |
| TNFSF8_SMC3 | 0.888 | NA | 0.959 | 0.914 | 0.718 | 0.885 | 0.866 |
| TNFSF8_REPS1 | 0.924 | NA | 0.992 | 0.802 | 0.766 | 0.890 | 0.866 |
| TNFSF8_C14orf1 | 0.900 | NA | 0.972 | 0.840 | 0.766 | 0.892 | 0.866 |
| TNFSF8_FUT8 | 0.933 | NA | 0.994 | 0.914 | 0.622 | 0.907 | 0.866 |
| TNFSF8_VPS13A | 0.888 | NA | 0.978 | 0.877 | 0.728 | 0.897 | 0.865 |
| TNFSF8_RAD50 | 0.894 | NA | 0.993 | 0.852 | 0.755 | 0.865 | 0.864 |
| TNFSF8_ESF1 | 0.903 | NA | 0.990 | 0.901 | 0.734 | 0.824 | 0.862 |
| TNFSF8_MRPS10 | 0.880 | NA | 0.946 | 0.852 | 0.738 | 0.929 | 0.862 |
| CDA_EFHD2 | 0.976 | NA | 0.994 | 0.926 | 0.608 | 0.834 | 0.862 |
| TNFSF8_SLC35A3 | 0.861 | NA | 0.982 | 0.889 | 0.761 | 0.851 | 0.862 |
| ADAM19_TMEM87A | 0.942 | NA | 0.999 | 0.864 | 0.657 | 0.878 | 0.861 |
| TNFSF8_LANCL1 | 0.891 | NA | 0.999 | 0.815 | 0.750 | 0.900 | 0.861 |
| ADAM19_ERCC4 | 0.936 | NA | 0.990 | 0.852 | 0.653 | 0.912 | 0.861 |
| TNFSF8_CD28 | 0.942 | NA | 1.000 | 0.840 | 0.692 | 0.870 | 0.860 |
| ADAM19_MLLT10 | 0.939 | NA | 1.000 | 0.926 | 0.647 | 0.828 | 0.860 |
| TNFSF8_IQCB1 | 0.903 | NA | 0.963 | 0.852 | 0.711 | 0.907 | 0.860 |
| TNFSF8_FASTKD2 | 0.891 | NA | 0.995 | 0.877 | 0.680 | 0.897 | 0.859 |
| TNFSF8_RDX | 0.842 | NA | 0.921 | 0.790 | 0.801 | 0.968 | 0.858 |
| TNFSF8_MTO1 | 0.879 | NA | 0.969 | 0.877 | 0.713 | 0.894 | 0.858 |
| IQSEC1_MACF1 | 0.945 | NA | 0.994 | 0.877 | 0.663 | 0.845 | 0.858 |
| TNFSF8_SMC6 | 0.876 | NA | 0.951 | 0.926 | 0.684 | 0.887 | 0.858 |
| TNFSF8_NEK1 | 0.867 | NA | 0.963 | 0.914 | 0.765 | 0.813 | 0.857 |
| TNFSF8_ZNF562 | 0.855 | NA | 0.968 | 0.864 | 0.720 | 0.914 | 0.856 |
| TNFSF8_PEX1 | 0.897 | NA | 0.966 | 0.765 | 0.814 | 0.877 | 0.856 |
| ADAM19_SIDT2 | 0.952 | NA | 0.993 | 0.938 | 0.628 | 0.816 | 0.856 |
| TNFSF8_METTL5 | 0.939 | NA | 0.973 | 0.765 | 0.775 | 0.856 | 0.856 |
| CYP4F3_TRAPPC2 | 0.967 | NA | 0.903 | 0.926 | 0.706 | 0.814 | 0.855 |
| TNFSF8_KRIT1 | 0.864 | NA | 0.935 | 0.901 | 0.721 | 0.895 | 0.855 |
| TNFSF8_YEATS4 | 0.906 | NA | 0.947 | 0.877 | 0.736 | 0.843 | 0.855 |
| TNFSF8_CLUAP1 | 0.902 | NA | 0.980 | 0.877 | 0.672 | 0.885 | 0.854 |
| TNFSF8_LARP4 | 0.876 | NA | 0.979 | 0.753 | 0.767 | 0.939 | 0.854 |
| TNFSF8_SLC35D1 | 0.873 | NA | 0.996 | 0.802 | 0.743 | 0.895 | 0.854 |
| SYNE2_RBM26 | 0.897 | NA | 0.910 | 0.901 | 0.691 | 0.887 | 0.853 |
| TNFSF8_CD40LG | 0.888 | NA | 0.973 | 0.914 | 0.655 | 0.880 | 0.853 |
| VNN3_CYSLTR1 | 0.855 | NA | 0.972 | 0.963 | 0.713 | 0.792 | 0.852 |
| TNFSF8_SYT11 | 0.882 | NA | 0.927 | 0.778 | 0.770 | 0.934 | 0.852 |
| TNFSF8_RIOK2 | 0.888 | NA | 0.972 | 0.802 | 0.731 | 0.904 | 0.852 |
| TNFSF8_BZW2 | 0.918 | NA | 0.996 | 0.778 | 0.701 | 0.914 | 0.852 |
| TNFSF8_LARP1 | 0.830 | NA | 0.982 | 0.840 | 0.719 | 0.916 | 0.852 |
| ADAM19_SYT11 | 0.939 | NA | 1.000 | 0.815 | 0.599 | 0.932 | 0.851 |
| TNFSF8_NCBP1 | 0.877 | NA | 0.915 | 0.778 | 0.785 | 0.936 | 0.851 |
| ADAM19_MACF1 | 0.958 | NA | 1.000 | 0.827 | 0.592 | 0.914 | 0.851 |
| TNFSF8_NOL8 | 0.885 | NA | 0.993 | 0.864 | 0.629 | 0.929 | 0.851 |
| TNFSF8_KIAA0391 | 0.942 | NA | 0.922 | 0.802 | 0.745 | 0.880 | 0.851 |
| TNFSF8_HIBCH | 0.900 | NA | 0.919 | 0.815 | 0.813 | 0.834 | 0.850 |
| TNFSF8_MYO9A | 0.888 | NA | 0.951 | 0.827 | 0.697 | 0.927 | 0.849 |
| EXTL3_CYSLTR1 | 0.876 | NA | 0.951 | 0.889 | 0.784 | 0.780 | 0.849 |
| CLEC4E_ARL6IP5 | 0.800 | 0.977 | 0.998 | 0.938 | 0.511 | 0.904 | 0.849 |
| VNN3_MACF1 | 0.879 | NA | 0.950 | 0.914 | 0.706 | 0.828 | 0.849 |
| ADAM19_MTRR | 0.945 | NA | 0.993 | 0.790 | 0.584 | 0.956 | 0.849 |
| TNFSF8_SUPT7L | 0.891 | NA | 0.960 | 0.790 | 0.728 | 0.905 | 0.849 |
| ADAM19_TFIP11 | 0.958 | NA | 0.928 | 0.901 | 0.603 | 0.883 | 0.849 |
| TNFSF8_ARL6IP5 | 0.839 | NA | 0.967 | 0.852 | 0.681 | 0.932 | 0.848 |
| TNFSF8_ENOSF1 | 0.900 | NA | 0.983 | 0.802 | 0.756 | 0.846 | 0.848 |
| TNFSF8_ADSL | 0.939 | NA | 0.998 | 0.790 | 0.638 | 0.907 | 0.848 |
| TNFSF8_TGS1 | 0.864 | NA | 0.889 | 0.914 | 0.708 | 0.900 | 0.848 |
| GAB2_TRAPPC2 | 0.876 | NA | 0.988 | 0.914 | 0.577 | 0.910 | 0.848 |
| TNFSF8_NR2C1 | 0.924 | NA | 0.988 | 0.753 | 0.713 | 0.900 | 0.847 |
| TNFSF8_ZMYND11 | 0.858 | NA | 0.998 | 0.802 | 0.745 | 0.878 | 0.847 |
| TNFSF8_NGDN | 0.924 | NA | 0.973 | 0.864 | 0.689 | 0.819 | 0.847 |
| TNFSF8_PRKAB2 | 0.888 | NA | 0.981 | 0.778 | 0.737 | 0.890 | 0.847 |
| TNFSF8_MDH1 | 0.933 | NA | 0.980 | 0.802 | 0.626 | 0.931 | 0.847 |
| IGF2R_MACF1 | 0.912 | NA | 0.986 | 0.901 | 0.642 | 0.826 | 0.846 |
| ADAM19_RRAGC | 0.955 | NA | 0.941 | 0.914 | 0.543 | 0.910 | 0.846 |
| STK17B_YTHDC2 | 0.870 | NA | 0.990 | 0.926 | 0.551 | 0.919 | 0.846 |
| TNFSF8_GOLPH3L | 0.903 | NA | 0.991 | 0.840 | 0.625 | 0.910 | 0.846 |
| TNFSF8_BRCC3 | 0.879 | NA | 0.957 | 0.778 | 0.764 | 0.883 | 0.846 |
| TNFSF8_NFX1 | 0.888 | NA | 0.994 | 0.815 | 0.666 | 0.907 | 0.846 |
| VNN3_ATP8A1 | 0.845 | NA | 0.992 | 0.914 | 0.685 | 0.824 | 0.845 |
| TNFSF8_IKBKAP | 0.897 | NA | 0.989 | 0.778 | 0.671 | 0.929 | 0.845 |
| TNFSF8_TRIP11 | 0.864 | NA | 0.901 | 0.889 | 0.788 | 0.816 | 0.845 |
| RGS2_TRAPPC2 | 0.809 | NA | 0.959 | 0.963 | 0.612 | 0.905 | 0.845 |
| TNFSF8_TCF12 | 0.856 | NA | 0.958 | 0.778 | 0.721 | 0.944 | 0.845 |
| TNFSF8_WDR70 | 0.897 | NA | 0.981 | 0.704 | 0.791 | 0.887 | 0.845 |
| TNFSF8_KLHL20 | 0.870 | NA | 0.954 | 0.765 | 0.766 | 0.905 | 0.845 |
| CDA_PTGS1 | 0.939 | 0.770 | 0.983 | 0.951 | 0.546 | 0.912 | 0.845 |
| MXD1_TRAPPC2 | 0.836 | NA | 0.998 | 0.988 | 0.548 | 0.883 | 0.844 |
| RGS2_RBM26 | 0.809 | NA | 0.999 | 0.975 | 0.642 | 0.814 | 0.844 |
| IGF2R_NOTCH2 | 0.924 | NA | 0.962 | 0.963 | 0.551 | 0.863 | 0.844 |
| TNFSF8_HLTF | 0.882 | NA | 0.965 | 0.778 | 0.761 | 0.867 | 0.844 |
| TNFSF8_BCKDHB | 0.873 | NA | 0.919 | 0.815 | 0.797 | 0.858 | 0.844 |
| MXD1_RCBTB2 | 0.852 | NA | 0.979 | 0.963 | 0.581 | 0.885 | 0.844 |
| TNFSF8_AGA | 0.894 | NA | 0.894 | 0.815 | 0.728 | 0.922 | 0.843 |
| TNFSF8_AGPAT5 | 0.876 | NA | 0.999 | 0.815 | 0.671 | 0.892 | 0.843 |
| TNFSF8_TTC27 | 0.891 | NA | 0.997 | 0.802 | 0.658 | 0.902 | 0.842 |
| TNFSF8_TTC17 | 0.815 | NA | 0.918 | 0.802 | 0.769 | 0.938 | 0.842 |
| TNFSF8_S100PBP | 0.885 | NA | 0.971 | 0.889 | 0.605 | 0.900 | 0.842 |
| TNFSF8_PRPF39 | 0.879 | NA | 0.980 | 0.790 | 0.666 | 0.927 | 0.842 |
| TNFSF8_MACF1 | 0.845 | NA | 0.954 | 0.790 | 0.757 | 0.897 | 0.841 |
| ENTPD1_MACF1 | 0.791 | NA | 0.999 | 0.877 | 0.659 | 0.914 | 0.841 |
| MYH9_MACF1 | 0.855 | NA | 0.990 | 0.926 | 0.753 | 0.720 | 0.841 |
| ENTPD1_SYT11 | 0.764 | 0.868 | 0.999 | 0.901 | 0.630 | 0.907 | 0.841 |
| SYNE2_VPS13C | 0.885 | NA | 0.962 | 0.864 | 0.579 | 0.944 | 0.841 |
| VNN3_RAB11FIP2 | 0.852 | NA | 0.965 | 0.938 | 0.646 | 0.831 | 0.840 |
| GAB2_RNF170 | 0.906 | NA | 0.997 | 0.901 | 0.581 | 0.838 | 0.840 |
| ADAM19_PSMD5 | 0.945 | NA | 1.000 | 0.827 | 0.551 | 0.909 | 0.839 |
| ADAM19_DIAPH2 | 0.939 | NA | 0.974 | 0.877 | 0.500 | 0.926 | 0.839 |
| GAB2_FCF1 | 0.900 | NA | 0.980 | 0.889 | 0.566 | 0.880 | 0.838 |
| IGF2R_TCF7L2 | 0.900 | NA | 0.967 | 0.864 | 0.680 | 0.806 | 0.838 |
| VNN3_THOC2 | 0.839 | NA | 0.986 | 0.938 | 0.652 | 0.804 | 0.838 |
| ADAM19_PLCL2 | 0.939 | NA | 0.995 | 0.901 | 0.577 | 0.813 | 0.838 |
| ADAM19_LARP1 | 0.947 | NA | 0.998 | 0.827 | 0.579 | 0.865 | 0.837 |
| RGS2_MACF1 | 0.821 | NA | 0.976 | 0.840 | 0.673 | 0.900 | 0.837 |
| TNFSF8_RFC1 | 0.870 | NA | 0.967 | 0.840 | 0.673 | 0.867 | 0.837 |
| VNN3_CDKN1B | 0.861 | NA | 0.967 | 0.951 | 0.678 | 0.764 | 0.837 |
| ADAM19_POLR2A | 0.970 | NA | 0.996 | 0.778 | 0.576 | 0.899 | 0.837 |
| HEBP2_ARL6IP5 | 0.800 | 0.974 | 0.993 | 0.914 | 0.544 | 0.828 | 0.836 |
| VNN3_TIA1 | 0.852 | NA | 0.986 | 0.951 | 0.654 | 0.764 | 0.836 |
| RGS2_ATXN3 | 0.809 | NA | 0.993 | 1.000 | 0.624 | 0.780 | 0.835 |
| RGS2_CLOCK | 0.809 | NA | 0.997 | 0.951 | 0.572 | 0.875 | 0.835 |
| TNFSF8_EFTUD1 | 0.882 | NA | 0.986 | 0.753 | 0.685 | 0.902 | 0.835 |
| GAB2_KLHL24 | 0.891 | NA | 0.923 | 0.926 | 0.688 | 0.764 | 0.835 |
| VNN3_YTHDC2 | 0.858 | NA | 0.972 | 0.938 | 0.660 | 0.774 | 0.834 |
| VNN3_KRIT1 | 0.855 | NA | 0.971 | 0.951 | 0.657 | 0.769 | 0.834 |
| RGS2_S100PBP | 0.809 | NA | 0.992 | 0.963 | 0.542 | 0.883 | 0.834 |
| VNN3_TRAPPC2 | 0.848 | NA | 0.949 | 0.951 | 0.645 | 0.802 | 0.833 |
| GAB2_BTN2A1 | 0.915 | NA | 0.965 | 0.889 | 0.548 | 0.875 | 0.833 |
| ADAM19_HRH4 | 0.939 | NA | 0.983 | 0.926 | 0.608 | 0.740 | 0.833 |
| GAB2_ADRBK2 | 0.903 | NA | 0.995 | 0.889 | 0.517 | 0.887 | 0.832 |
| KCMF1_ARL6IP5 | 0.858 | 0.974 | 0.999 | 0.914 | 0.589 | 0.694 | 0.832 |
| VNN3_RBM26 | 0.842 | NA | 0.993 | 0.938 | 0.679 | 0.736 | 0.832 |
| ADAM19_SLCO3A1 | 0.945 | NA | 0.965 | 0.827 | 0.499 | 0.951 | 0.831 |
| STK17B_RABGAP1L | 0.892 | NA | 0.988 | 0.901 | 0.598 | 0.802 | 0.831 |
| GAB2_PRUNE | 0.906 | NA | 0.965 | 0.901 | 0.540 | 0.865 | 0.830 |
| RGS2_ZNF507 | 0.809 | NA | 0.998 | 0.938 | 0.602 | 0.818 | 0.829 |
| RGS2_ARHGEF6 | 0.809 | NA | 0.987 | 0.975 | 0.501 | 0.895 | 0.829 |
| RGS2_PHC3 | 0.809 | NA | 0.996 | 0.938 | 0.626 | 0.797 | 0.828 |
| GAB2_CREB1 | 0.891 | NA | 0.995 | 0.926 | 0.570 | 0.784 | 0.828 |
| VNN3_VPS13B | 0.845 | NA | 0.928 | 0.938 | 0.648 | 0.804 | 0.828 |
| PELI1_CDKN1B | 0.906 | NA | 0.923 | 0.963 | 0.490 | 0.880 | 0.827 |
| RGS2_YTHDC2 | 0.809 | NA | 0.980 | 0.864 | 0.628 | 0.865 | 0.825 |
| STK17B_TLK1 | 0.879 | NA | 0.972 | 0.901 | 0.473 | 0.909 | 0.823 |
| RGS2_FCF1 | 0.809 | NA | 0.968 | 0.951 | 0.576 | 0.826 | 0.823 |
| SYNE2_KRIT1 | 0.900 | NA | 0.800 | 0.926 | 0.650 | 0.855 | 0.822 |
| HAL_CPA3 | 0.835 | NA | 0.923 | 0.963 | 0.540 | 0.860 | 0.820 |

TABLE 39
INTERPRETATION OF RESULTS OBTAINED WHEN USING
A COMBINATION OF BASIRS AND BACTERIAL DETECTION

| | Bacterial Pathogen Antigen | |
|---|---|---|
| Host Immune Response | Positive | Negative |
| Positive | Confirmed BaSIRS | Organism did not grow? |
| | | Organism not present? |
| Negative | Contaminant? | Confirmed inSIRS |
| | Commensal? | |

TABLE 40
INTERPRETATION OF RESULTS OBTAINED WHEN USING
A COMBINATION OF VASIRS AND VIRUS DETECTION

| Host Immune | Viral Pathogen Antigen | |
|---|---|---|
| Response | Positive | Negative |
| Positive | Confirmed VaSIRS | Assay not sensitive enough? |
| | | Organism not present? |
| | | Not enough sample taken? |
| | | Wrong assay performed? |
| | | Antibodies not yet produced? |
| Negative | Commensal? | Confirmed inSIRS |
| | Residual antibody? | |

TABLE 41
INTERPRETATION OF RESULTS OBTAINED WHEN USING
A COMBINATION OF PASIRS AND PROTOZOAN DETECTION

| Host Immune | Protozoal Pathogen Antigen | |
|---|---|---|
| Response | Positive | Negative |
| Positive | Confirmed PaSIRS | Assay not sensitive enough? |
| | | Organism not present? |
| | | Not enough sample taken? |
| | | Wrong assay performed? |
| | | Antibodies not yet produced? |
| Negative | Commensal? | Confirmed inSIRS |
| | Residual antibody? | |

What is claimed is:

1. A method for determining an indicator used in assessing a likelihood of a subject having a presence, absence or degree of BaSIRS or VaSIRS, the method comprising: (1) determining a plurality of host response specific biomarker values including a plurality of BaSIRS biomarker values and a plurality of VaSIRS biomarker values, the plurality of BaSIRS biomarker values being indicative of values measured for a corresponding plurality of BaSIRS biomarkers in a sample taken from the subject, the plurality of VaSIRS biomarker values being indicative of values measured for a corresponding plurality of VaSIRS biomarkers in the sample; (2) determining a plurality of host response specific derived biomarker values including at least one BaSIRS derived biomarker value and at least one VaSIRS derived biomarker value, each derived BaSIRS biomarker value being determined using at least a subset of the plurality of BaSIRS biomarker values, and being indicative of a ratio of levels of a corresponding at least a subset of the plurality of BaSIRS biomarkers, and each derived VaSIRS biomarker value being determined using at least a subset of the plurality of VaSIRS biomarker values, and being indicative of a ratio of levels of a corresponding at least a subset of the plurality of VaSIRS biomarkers; and (3) determining the indicator using the plurality of host response specific derived biomarker values, wherein the at least a subset of BaSIRS biomarkers forms a BaSIRS derived biomarker combination which is not a derived biomarker combination for VaSIRS, PaSIRS or InSIRS, and wherein the at least a subset of VaSIRS biomarkers forms a VaSIRS derived biomarker combination which is not a derived biomarker combination for BaSIRS, PaSIRS or InSIRS.

2. The method of claim 1, wherein the BaSIRS derived biomarker combination and the VaSIRS derived biomarker combination are not derived biomarker combinations for any one or more inflammatory conditions selected from autoimmunity, asthma, stress, anaphylaxis, trauma and obesity. Alternatively, or in addition, the derived BaSIRS biomarkers and derived VaSIRS biomarkers are not derived biomarkers for any one or more of age, gender and race.

3. The method of claim 1 or claim 2, further comprising: (a) determining a plurality of pathogen specific biomarker values including at least one bacterial biomarker value and at least one viral biomarker value, the least one bacterial biomarker value being indicative of a value measured for a corresponding bacterial biomarker in the sample, the least one viral biomarker value being indicative of a value measured for a corresponding viral biomarker in the sample; and (b) determining the indicator using the host response specific derived biomarker values in combination with the pathogen specific biomarker values.

4. The method of any one of claims 1 to 3, wherein each BaSIRS derived biomarker value is determined using a pair of the BaSIRS biomarker values, and is indicative of a ratio of levels of a corresponding pair of BaSIRS biomarkers. Alternatively, or in addition, each VaSIRS derived biomarker value is determined using a pair of the VaSIRS biomarker values, and is indicative of a ratio of levels of a corresponding pair of VaSIRS biomarkers.

5. The method of any one of claims 1 to 4, wherein the plurality of host response specific biomarker values further includes a plurality of PaSIRS biomarker values, the plurality of PaSIRS biomarker values being indicative of values measured for a corresponding plurality of PaSIRS biomarkers in the sample, and the plurality of host response specific derived biomarker values further includes at least one PaSIRS derived biomarker value, and the methods further comprise: determining each PaSIRS derived biomarker value using at least a subset of the plurality of PaSIRS biomarker values, the PaSIRS derived biomarker value being indicative of a ratio of levels of a corresponding at least a subset of the plurality of PaSIRS biomarkers; and determining the indicator using the plurality of host response specific derived biomarker values, wherein the at least a subset of PaSIRS biomarkers forms a PaSIRS derived biomarker combination which is not a derived biomarker combination for BaSIRS, VaSIRS or InSIRS.

6. The method of any one of claims 1 to 5, wherein each PaSIRS derived biomarker value is determined using a pair of the PaSIRS biomarker values, and is indicative of a ratio of levels of a corresponding pair of PaSIRS biomarkers.

7. A method for determining an indicator used in assessing a likelihood of a subject having a presence, absence or degree of BaSIRS, VaSIRS or PaSIRS, the method comprising: (1) determining a plurality of host response specific biomarker values including a plurality of BaSIRS biomarker values, a plurality of VaSIRS biomarker values, and a plurality of PaSIRS biomarker values, the plurality of BaSIRS biomarker values being indicative of values measured for a corresponding plurality of BaSIRS biomarkers in a sample taken from the subject, the plurality of VaSIRS biomarker values being indicative of values measured for a corresponding plurality of VaSIRS biomarkers in the sample, the plurality of PaSIRS biomarker values being indicative of values measured for a corresponding plurality of PaSIRS biomarkers in the sample; (2) determining a plurality of host response specific derived biomarker values including at least one BaSIRS derived biomarker value, at least one VaSIRS derived biomarker value, and at least one PaSIRS derived biomarker value, each derived BaSIRS biomarker value being determined using at least a subset of the plurality of BaSIRS biomarker values, and being indicative of a ratio of levels of a corresponding at least a subset of the plurality of BaSIRS biomarkers, each derived VaSIRS biomarker value being determined using at least a subset of the plurality of VaSIRS biomarker values, and being indicative of a ratio of levels of a corresponding at least a subset of the plurality of VaSIRS biomarkers, and each derived PaSIRS biomarker value being determined using at least a subset of the plurality of PaSIRS biomarker values, and being indicative of a ratio of levels of a corresponding at least a subset of the plurality of PaSIRS biomarkers; and (3) determining the indicator using the plurality of host response specific derived biomarker values, wherein the at least a subset of BaSIRS biomarkers forms a BaSIRS derived biomarker combination which is not a derived biomarker combination for VaSIRS, PaSIRS or InSIRS, wherein the at least a subset of VaSIRS biomarkers forms a VaSIRS derived biomarker combination which is not a derived biomarker combination for BaSIRS, PaSIRS or InSIRS, and wherein the at least a subset of PaSIRS biomarkers forms a PaSIRS derived biomarker combination which is not a derived biomarker combination for BaSIRS, VaSIRS or InSIRS.

8. The method of any one of claims 1 to 7, further comprising: (a) determining a plurality of pathogen specific biomarker values including at least one bacterial biomarker value, at least one viral biomarker value and at least one protozoal biomarker value, the at least one bacterial biomarker value being indicative of a value measured for a corresponding bacterial biomarker in the sample, the least one viral biomarker value being indicative of a value measured for a corresponding viral biomarker in the sample, and the least one protozoal biomarker value being indicative of a value measured for a corresponding protozoal biomarker in the sample; and (b) determining the indicator using the host response specific derived biomarker values in combination with the pathogen specific biomarker values.

9. The method of any one of claims 1 to 8, wherein the plurality of host response specific biomarker values further includes a plurality of InSIRS biomarker values, the plurality of InSIRS biomarker values being indicative of values measured for a corresponding plurality of InSIRS biomarkers in the sample, and the plurality of host response specific derived biomarker values further includes at least one InSIRS derived biomarker value, and the methods further comprise: determining each InSIRS derived biomarker value using at least a subset of the plurality of InSIRS biomarker values, the InSIRS derived biomarker value being indicative of a ratio of levels of a corresponding at least a subset of the plurality of InSIRS biomarkers; and determining the indicator using the plurality of host response specific derived biomarker values, wherein the at least a subset of InSIRS biomarkers forms a InSIRS derived biomarker combination which is not a derived marker combination for BaSIRS, VaSIRS or PaSIRS.

10. A method for determining an indicator used in assessing a likelihood of a subject having a presence, absence or degree of BaSIRS, VaSIRS or InSIRS, the method comprising: (1) determining a plurality of host response specific biomarker values including a plurality of BaSIRS biomarker values, a plurality of VaSIRS biomarker values, and a plurality of InSIRS biomarker values, the plurality of BaSIRS biomarker values being indicative of values measured for a corresponding plurality of BaSIRS biomarkers in a sample taken from the subject, the plurality of VaSIRS biomarker values being indicative of values measured for a corresponding plurality of VaSIRS biomarkers in the sample, the plurality of InSIRS biomarker values being indicative of values measured for a corresponding plurality of InSIRS biomarkers in the sample; (2) determining a plurality of host response specific derived biomarker values including at least one BaSIRS derived biomarker value, at least one VaSIRS derived biomarker value, and at least one InSIRS derived biomarker value, each derived BaSIRS biomarker value being determined using at least a subset of the plurality of BaSIRS biomarker values, and being indicative of a ratio of levels of a corresponding at least a subset of the plurality of BaSIRS biomarkers, each derived VaSIRS biomarker value being determined using at least a subset of the plurality of VaSIRS biomarker values, and being indicative of a ratio of levels of a corresponding at least a subset of the plurality of VaSIRS biomarkers, and each derived InSIRS biomarker value being determined using at least a subset of the plurality of InSIRS biomarker values, and being indicative of a ratio of levels of a corresponding at least a subset of the plurality of InSIRS biomarkers; and (3) determining the indicator using the plurality of host response specific derived biomarker values, wherein the at least a subset of BaSIRS biomarkers forms a BaSIRS derived biomarker combination which is not a derived biomarker combination for VaSIRS, PaSIRS or InSIRS, wherein the at least a subset of VaSIRS biomarkers forms a VaSIRS derived biomarker combination which is not a derived biomarker combination for BaSIRS, PaSIRS or InSIRS, and wherein the at least a subset of InSIRS biomarkers forms an InSIRS derived biomarker combination which is not a derived biomarker combination for BaSIRS, VaSIRS or PaSIRS.

11. A method for determining an indicator used in assessing a likelihood of a subject having a presence, absence or degree of BaSIRS, VaSIRS, PaSIRS or InSIRS, the method comprising: (1) determining a plurality of host response specific biomarker values including a plurality of BaSIRS biomarker values, a plurality of VaSIRS biomarker values, a plurality of PaSIRS biomarker values, and a plurality of InSIRS biomarker values, the plurality of BaSIRS biomarker values being indicative of values measured for a corresponding plurality of BaSIRS biomarkers in a sample taken from the subject, the plurality of VaSIRS biomarker values being indicative of values measured for a corresponding plurality of VaSIRS biomarkers in the sample, the plurality of PaSIRS biomarker values being indicative of values measured for a corresponding plurality of PaSIRS biomarkers in the sample, the plurality of InSIRS biomarker values being indicative of values measured for a corresponding plurality of InSIRS biomarkers in the sample; (2) determining a plurality of host response specific derived biomarker values including at least one BaSIRS derived biomarker value, at least one VaSIRS derived biomarker value, at least one PaSIRS derived biomarker value, and at least one InSIRS derived biomarker value, each derived BaSIRS biomarker value being determined using at least a subset of the plurality of BaSIRS biomarker values, and being indicative of a ratio of levels of a corresponding at least a subset of the plurality of BaSIRS biomarkers, each derived VaSIRS biomarker value being determined using at least a subset of the plurality of VaSIRS biomarker values, and being indicative of a ratio of levels of a corresponding at least a subset of the plurality of VaSIRS biomarkers, each derived PaSIRS biomarker value being determined using at least a subset of the plurality of PaSIRS biomarker values, and being indicative of a ratio of levels of a corresponding at least a subset of the plurality of PaSIRS biomarkers, and each derived InSIRS biomarker value being determined using at least a subset of the plurality of InSIRS biomarker values, and being indicative of a ratio of levels of a corresponding at least a subset of the plurality of InSIRS biomarkers; and (3) determining the indicator using the plurality of host response specific derived biomarker values, wherein the at least a subset of BaSIRS biomarkers forms a BaSIRS derived biomarker combination which is not a derived biomarker combination for VaSIRS, PaSIRS or InSIRS, wherein the at least a subset of VaSIRS biomarkers forms a VaSIRS derived biomarker combination which is not a derived biomarker combination for BaSIRS, PaSIRS or InSIRS, wherein the at least a subset of PaSIRS biomarkers forms a PaSIRS derived biomarker combination which is not a derived biomarker combination for BaSIRS, VaSIRS or InSIRS, and wherein the at least a subset of InSIRS biomarkers forms an InSIRS derived biomarker combination which is not a derived biomarker combination for BaSIRS, VaSIRS or PaSIRS.

12. The method of any one of claims 1 to 11, wherein the indicator is determined by combining a plurality (e.g., 2, 3, 4, 5, 6, 7, 8, etc.) of derived biomarker values.

13. The method of claim 12, comprising combining the derived biomarker values using a combining function, wherein the combining function is at least one of: an additive model; a linear model; a support vector machine; a neural network model; a random forest model; a regression model; a genetic algorithm; an annealing algorithm; a weighted sum; a nearest neighbor model; and a probabilistic model.

14. The method of any one of claims 1 to 13, wherein individual BaSIRS derived biomarker combinations are selected from TABLE A.
TABLE A
BaSIRS Derived Biomarkers

| | | | |
|---|---|---|---|
| PDGFC:KLRF1 | PDGFC:CCNK | GALNT2:KLRD1 | GAS7:CAMK1D |
| TMEM165:PARP8 | CR1:ADAM19 | KIAA0101:IL2RB | MGAM:MME |
| ITGA7:KLRF1 | ITGA7:CCNK | CR1:HAL | GAS7:GAB2 |
| CR1:GAB2 | PCOLCE2:PRSS23 | PDGFC:RFC1 | PDGFC:INPP5D |
| PCOLCE2:KLRF1 | TMEM165:PRPF38B | ENTPD7:KLRF1 | ST3GAL2:PRKD2 |
| ITGA7:INPP5D | PDGFC:PHF3 | PDGFC:GRK5 | HK3:INPP5D |
| GALNT2:CCNK | GAS7:NLRP1 | PCOLCE2:PYHIN1 | ENTPD7:KLRD1 |
| PDGFC:KLRD1 | PCOLCE2:KLRD1 | GAS7:PRKDC | PDGFC:SIDT1 |
| PDGFC:SPIN1 | COX15:UTRN | MCTP1:PARP8 | TSPO:CAMK1D |
| PCOLCE2:YPEL1 | SMPDL3A:QRICH1 | TSPO:HCLS1 | OPLAH:POGZ |
| PDGFC:SYTL2 | PDGFC:LPIN2 | TSPO:CASS4 | ALPL:RNASE6 |
| PDGFC:TGFBR3 | TSPO:NLRP1 | GAS7:RBM23 | RAB32:NLRP1 |
| IGFBP7:KLRF1 | PCOLCE2:NMUR1 | GAS7:EPHB4 | TLR5:SEMA4D |
| PCOLCE2:RUNX2 | FAM129A:GAB2 | PDGFC:RBM15 | IMPDH1:NLRP1 |
| SMPDL3A:KLRD1 | ALPL:NLRP1 | ADM:CLEC7A | ALPL:CAMK1D |
| GALNT2:KLRF1 | TSPO:ZFP36L2 | PDGFC:LEPROTL1 | TSPO:NFIC |
| PDGFC:YPEL1 | ALPL:ZFP36L2 | PDGFC:NPAT | GAS7:HAL |
| HK3:DENND3 | PCOLCE2:FOXJ3 | TSPO:PLA2G7 | PDGFC:NCOA6 |
| PDGFC:CBLL1 | PDGFC:KIAA0355 | GALNT2:IK | PDGFC:PIK3C2A |
| OPLAH:KLRD1 | PDGFC:KIAA0907 | CD82:JARID2 | TSPO:ADAM19 |
| OPLAH:ZHX2 | GAS7:DOCK5 | PDGFC:ICK | CD82:NOV |
| PDGFC:RYK | CD82:CNNM3 | GALNT2:SAP130 | PDGFC:PDS5B |
| PDGFC:IKZF5 | GAS7:EXTL3 | PDGFC:FBXO28 | FIG4:INPP5D |
| GALNT2:INPP5D | TSPO:RNASE6 | TSPO:GAB2 | TSPO:NOV |
| PDGFC:GCC2 | ALPL:MME | COX15:INPP5D | |
| PDGFC:MBIP | HK3:TLE3 | ITGA7:LAG3 | |

15. The method of any one of claims 1 to 14, wherein a single BaSIRS derived biomarker combination (e.g., any one from TABLE A) is used for determining the indicator.

16. The method of any one of claims 1 to 14, wherein two BaSIRS derived biomarker combinations (e.g., any two from TABLE A) are used for determining the indicator.

17. The method of any one of claims 1 to 14, wherein three BaSIRS derived biomarker combinations (e.g., any three from TABLE A) are used for determining the indicator.

18. The method of any one of claims 1 to 14, wherein four BaSIRS derived biomarker combinations (e.g., any four from TABLE A) are used for determining the indicator.

19. The method of claim 15, comprising: (a) determining a single BaSIRS derived biomarker value using a pair of BaSIRS biomarker values, the single BaSIRS derived biomarker value being indicative of a ratio of levels of first and second BaSIRS biomarkers; and (b) determining the indicator using the single derived BaSIRS biomarker value.

20. The method of claim 16, comprising: (a) determining a first BaSIRS derived biomarker value using a first pair of BaSIRS biomarker values, the first BaSIRS derived biomarker value being indicative of a ratio of levels of first and second BaSIRS biomarkers; (b) determining a second BaSIRS derived biomarker value using a second pair of BaSIRS biomarker values, the second BaSIRS derived biomarker value being indicative of a ratio of levels of third and fourth BaSIRS biomarkers; and (c) determining the indicator by combining the first and second derived BaSIRS biomarker values, using for example a combining function as disclosed herein.

21. The method of claim 17, comprising: (a) determining a first BaSIRS derived biomarker value using a first pair of BaSIRS biomarker values, the first BaSIRS derived biomarker value being indicative of a ratio of levels of first and second BaSIRS biomarkers; (b) determining a second BaSIRS derived biomarker value using a second pair of BaSIRS biomarker values, the second BaSIRS derived biomarker value being indicative of a ratio of levels of third and fourth BaSIRS biomarkers; (c) determining a third BaSIRS derived biomarker value using a third pair of BaSIRS biomarker values, the third BaSIRS derived biomarker value being indicative of a ratio of levels of fifth and fourth BaSIRS biomarkers; and (d) determining the indicator by combining the first and sixth derived BaSIRS biomarker values, using for example a combining function as disclosed herein.

22. The method of any one of claims 1 to 21, wherein individual BaSIRS derived biomarker combinations are selected from TSPO:HCLS1, OPLAH:ZHX2, TSPO:RNASE6; GAS7:CAMK1D, ST3GAL2:PRKD2, PCOLCE2:NMUR1 and CR1:HAL.

23. The method of any one of claims 1 to 21, wherein individual BaSIRS derived biomarker combinations are selected from OPLAH:ZHX2 and TSPO:HCLS1.

24. The method of any one of claims 1 to 23, wherein the bacterium associated with the BaSIRS is selected from any Gram positive or Gram negative bacterial species which is capable of inducing at least one of the clinical signs of SIRS.

25. The method of any one of claims 1 to 13, wherein individual VaSIRS derived biomarker combinations are selected from TABLE B.
TABLE B
VaSIRS Derived Biomarker

| | | | |
|---|---|---|---|
| IFI6:IL16 | OASL:SP3 | IFI6:ABLIM1 | OASL:SMAD4 |
| OASL:NR3C1 | OASL:ABLIM1 | OAS2:FAIM3 | OASL:ST3GAL1 |
| OASL:EMR2 | OASL:AOAH | OASL:ARHGAP25 | OASL:ZNF292 |
| OASL:SORL1 | OASL:MBP | OASL:GNA12 | IFI44:IL4R |
| OASL:SERTAD2 | OASL:NLRP1 | OASL:NUMB | OASL:HPCAL1 |
| OASL:LPAR2 | OASL:PBX3 | OASL:CREBBP | OASL:IGSF6 |
| OASL:ITGAX | OASL:PTPN6 | OASL:PINK1 | OASL:MTMR3 |
| OASL:TGFBR2 | OASL:RYBP | OASL:PITPNA | OASL:PHF20 |
| OASL:KIAA0247 | OASL:IL13RA1 | OASL:SEMA4D | OASL:PPARD |
| OASL:ARHGAP26 | OASL:LCP2 | OASL:TGFBI | OASL:PPP4R1 |
| OASL:LYN | OASL:LRP10 | OASL:APLP2 | OASL:RBMS1 |
| OASL:PCBP2 | OASL:SYPL1 | OASL:CCNG2 | OASL:RHOG |
| OASL:TOPORS | OASL:VAMP3 | OASL:MKRN1 | OASL:TIAM1 |
| EIF2AK2:IL16 | IFI44:LTB | OASL:RGS14 | USP18:IL16 |
| OASL:NCOA1 | OASL:ARHGEF2 | OASL:LYST | OASL:CBX7 |
| OASL:PTGER4 | OASL:CTDSP2 | OASL:TNRC6B | OASL:RAF1 |
| OASL:TLR2 | OASL:LST1 | OASL:TYROBP | OASL:SERINC5 |
| OASL:PACSIN2 | OASL:MAPK1 | OASL:WDR37 | OASL:UBQLN2 |
| OASL:LILRA2 | OASL:N4BP1 | OASL:WDR47 | OASL:XPO6 |
| OASL:PTPRE | OASL:STAT5B | UBE2L6:IL16 | OASL:ATP6V1B2 |
| OASL:RPS6KA1 | IFI44:ABLIM1 | OASL:BTG1 | OASL:CSF2RB |
| OASL:CASC3 | IFI44:IL6ST | OASL:CD93 | OASL:GYPC |
| OASL:VEZF1 | OASL:BACH1 | OASL:DCP2 | OASL:IL4R |
| OASL:CRLF3 | OASL:KLF7 | OASL:FYB | OASL:MMP25 |
| OASL:NDEL1 | OASL:PRMT2 | OASL:MAML1 | OASL:PSEN1 |
| OASL:RASSF2 | OASL:HCK | OASL:SNRK | OASL:SH2B3 |
| OASL:TLE4 | OASL:ITPKB | OASL:USP4 | OASL:STAT5A |
| OASL:CD97 | OASL:MAP4K4 | OASL:YTHDF3 | ISG15:IL16 |
| OASL:CEP68 | OASL:PPM1F | OASL:CEP170 | MX1:LEF1 |
| OASL:RXRA | OASL:RAB14 | OASL:PLEKHO2 | OASL:CAMK2G |
| OASL:ETS2 | OASL:ST13 | OASL:KBTBD2 | OASL:PSAP |
| OASL:POLB | OASL:TFEB | OASL:PHC2 | OASL:STX3 |
| OASL:STK38L | OASL:ZFYVE16 | OASL:PUM2 | OASL:TNK2 |
| OASL:TFE3 | EIF2AK2:SATB1 | OASL:SSFA2 | EIF2AK2:ZNF274 |
| OASL:ICAM3 | OASL:ABAT | IFI44:MYC | OASL:ACAA1 |
| OASL:ITGB2 | OASL:ABI1 | OASL:ABHD2 | OASL:CHD3 |
| OASL:PISD | OASL:ACVR1B | OASL:CYLD | OASL:FRY |
| OASL:PLXNC1 | OASL:GPSM3 | OASL:MAST3 | OASL:GRB2 |
| OASL:SNX27 | OASL:MPPE1 | OASL:UBN1 | OASL:MAP3K11 |
| OASL:TNIP1 | OASL:PTEN | IFI6:IL6ST | OASL:NEK7 |
| OASL:ZMIZ1 | OASL:SEC62 | IFIH1:TGFBR2 | OASL:PPP2R5A |
| OASL:FOXO3 | IFI6:MYC | OASL:CNPY3 | USP18:ST13 |
| OASL:IL10RB | IFI6:PCF11 | OASL:KIAA0232 | XAF1:LEF1 |
| OASL:MAP3K5 | OASL:AIF1 | USP18:CHMP7 | OASL:CASP8 |
| OASL:POLD4 | OASL:CSNK1D | USP18:NECAP2 | OASL:PCF11 |
| OASL:ARAP1 | OASL:GABARAP | OASL:CAP1 | OASL:PRKCD |
| OASL:CTBP2 | OASL:HAL | OASL:HPS1 | OASL:PSTPIP1 |
| OASL:DGKA | OASL:LAPTM5 | OASL:IL1RAP | OASL:SLCO3A1 |
| OASL:NFYA | OASL:XPC | OASL:MEF2A | OASL:ZDHHC17 |
| OASL:PCNX | USP18:NFKB1 | OASL:RNF19B | USP18:FOXO1 |
| OASL:PFDN5 | OASL:ACAP2 | OASL:TMEM127 | OASL:ASAP1 |
| OASL:R3HDM2 | OASL:CLEC4A | USP18:IL27RA | OASL:BAZ2B |
| OASL:STX6 | OASL:HIP1 | OASL:CDIPT | OASL:FAM65B |
| EIF2AK2:SYPL1 | OASL:PIAS1 | OASL:CREB1 | OASL:HHEX |
| ISG15:ABLIM1 | OASL:PPP3R1 | OASL:GPS2 | OASL:MAX |
| OASL:FOXJ2 | OASL:RALB | OASL:NDE1 | OASL:PHF2 |
| OASL:IQSEC1 | OASL:RGS19 | OASL:RAB11FIP1 | OASL:RNF130 |
| OASL:LRMP | OASL:TRIOBP | USP18:ABLIM1 | OASL:SOS2 |
| OASL:NAB1 | EIF2AK2:PDE3B | EIF2AK2:TNRC6B | OASL:STAM2 |
| OASL:RAB31 | OASL:NCOA4 | OASL:FAM134A | OASL:ZFC3H1 |
| OASL:WASF2 | OASL:RARA | OASL:FCGRT | IFI44:CYLD |
| OASL:ZNF274 | OASL:RPS6KA3 | OASL:LPIN2 | IFIH1:CRLF3 |
| OAS2:LEF1 | OASL:SIRPA | OASL:PECAM1 | OASL:BANP |
| OASL:BRD1 | OASL:TLE3 | OASL:WBP2 | OASL:CCND3 |
| OASL:GNAQ | OASL:TNFRSF1A | OASL:ZNF148 | OASL:DGCR2 |
| OASL:GSK3B | DDX60:TGFBR2 | OASL:RTN3 | OASL:USP15 |
| OASL:IL6R | OASL:FLOT2 | OASL:TYK2 | USP18:EIF3H |
| OASL:MAPK14 | OASL:FNBP1 | USP18:LTB | OASL:LAT2 |
| USP18:TGFBR2 | OASL:MAP3K3 | DHX58:IL16 | OASL:ZYX |
| ISG15:LTB | OASL:STX10 | ISG15:IL4R | USP18:CAMK1D |
| OASL:INPP5D | OASL:ZDHHC18 | OASL:BRD4 | ZBP1:NDE1 |
| OASL:MED13 | OASL:ZNF143 | OASL:CCNT2 | |
| OASL:MORC3 | TAP1:TGFBR2 | OASL:FGR | |
| OASL:PTAFR | OAS2:ABLIM1 | OASL:ITSN2 | |
| OASL:RBM23 | OASL:ARRB2 | OASL:LYL1 | |
| OASL:SNN | OASL:IKBKB | OASL:PHF3 | |

26. The method of any one of claims 1 to 25, wherein a single VaSIRS derived biomarker combination (e.g., any one from TABLE B) is used for determining the indicator.

27. The method of any one of claims 1 to 25, wherein two VaSIRS derived biomarker combinations (e.g., any two from TABLE B) are used for determining the indicator.

28. The method of any one of claims 1 to 25, wherein three VaSIRS derived biomarker combinations (e.g., any three from TABLE B) are used for determining the indicator.

29. The method of any one of claims 1 to 25, wherein four VaSIRS derived biomarker combinations (e.g., any four from TABLE B) are used for determining the indicator.

30. The method of claim 26, comprising: (a) determining a single VaSIRS derived biomarker value using a pair of VaSIRS biomarker values, the single VaSIRS derived biomarker value being indicative of a ratio of levels of first and second VaSIRS biomarkers; and (b) determining the indicator using the single derived VaSIRS biomarker value.

31. The method of claim 27, comprising: (a) determining a first VaSIRS derived biomarker value using a first pair of VaSIRS biomarker values, the first VaSIRS derived biomarker value being indicative of a ratio of levels of first and second VaSIRS biomarkers; (b) determining a second VaSIRS derived biomarker value using a second pair of VaSIRS biomarker values, the second VaSIRS derived biomarker value being indicative of a ratio of levels of third and fourth VaSIRS biomarkers; and (c) determining the indicator by combining the first and second derived VaSIRS biomarker values, using for example a combining function as disclosed herein.

32. The method of claim 28, comprising: (a) determining a first VaSIRS derived biomarker value using a first pair of VaSIRS biomarker values, the first VaSIRS derived biomarker value being indicative of a ratio of levels of first and second VaSIRS biomarkers; (b) determining a second VaSIRS derived biomarker value using a second pair of VaSIRS biomarker values, the second VaSIRS derived biomarker value being indicative of a ratio of levels of third and fourth VaSIRS biomarkers; (c) determining a third VaSIRS derived biomarker value using a third pair of VaSIRS biomarker values, the third VaSIRS derived biomarker value being indicative of a ratio of levels of fifth and fourth VaSIRS biomarkers; and (d) determining the indicator by combining the first and sixth derived VaSIRS biomarker values, using for example a combining function as disclosed herein.

33. The method of any one of claims 1 to 32, wherein individual VaSIRS derived biomarker combinations are selected from ISG15:IL16, OASL:ADGRE5, TAP1:TGFBR2, IFIH1:CRLF3, IFI44:IL4R, EIF2AK2:SYPL1, OAS2:LEF1, STAT1:PCBP2 and IFI6:IL6ST.

34. The method of any one of claims 1 to 32, wherein individual VaSIRS derived biomarker combinations are selected from ISG15:IL16 and OASL:ADGRE5.

35. The method of any one of claims 1 to 34, wherein the virus associated with the VaSIRS is suitably selected from any one of Baltimore virus classification Groups I, II, III, IV, V, VI and VII, which is capable of inducing at least one of the clinical signs of SIRS.

36. The method of any one of claims 5 to 9 and 11 to 35, wherein individual PaSIRS derived biomarker combinations are selected from TABLE C.
TABLE C
PaSIRS Derived Biomarker

| | | | |
|---|---|---|---|
| RPL9:WARS | SUCLG2:CEBPB | TTC17:ATOX1 | NOSIP:WARS |
| RPL9:CSTB | EXOSC10:G6PD | CSNK1G2:G6PD | RPS4X:UPP1 |
| NUP160:WARS | CEP192:WARS | SETX:CEBPB | CNOT7:CEBPB |
| IMP3:ATOX1 | NUP160:CD63 | ARHGAP17:CEBPB | ARHGAP17:WARS |
| RPS4X:WARS | TMEM50B:WARS | ZMYND11:WARS | UFM1:WARS |
| TCF4:CEBPB | EXOSC10:LDHA | IMP3:UPP1 | PREPL:SQRDL |
| IMP3:LAP3 | ARID1A:CSTB | EXOSC10:IRF1 | IMP3:TAP1 |
| EXOSC10:WARS | SUCLG2:WARS | UFM1:CEBPB | ARID1A:PCMT1 |
| TTC17:WARS | ARID1A:CEBPB | ARID1A:LDHA | SUCLG2:SQRDL |
| TCF4:WARS | FBXO11:TANK | RPL9:ATOX1 | RPL22:SH3GLB1 |
| METAP1:WARS | SUCLG2:SH3GLB1 | TTC17:GNG5 | BCL11A:WARS |
| FNTA:POMP | TTC17:G6PD | EXOSC10:POMP | CNOT7:WARS |
| TCF4:TANK | IMP3:PCMT1 | ARID1A:ATOX1 | ZBED5:TCIRG1 |
| TOP2B:CEBPB | ARID1A:LAP3 | RPL9:SH3GLB1 | EXOSC10:SQRDL |
| AHCTF1:CEBPB | IMP3:SQRDL | LY9:CEBPB | AHCTF1:GNG5 |
| RPS4X:MYD88 | TCF4:ATOX1 | RPS14:WARS | ZMYND11:FCER1G |
| IMP3:CEBPB | IMP3:SH3GLB1 | FNTA:SQRDL | TOP2B:ENO1 |
| RPL9:CEBPB | EXOSC10:MYD88 | APEX1:CD63 | IMP3:IRF1 |
| RPS4X:CEBPB | LY9:WARS | SETX:WARS | CEP192:TAP1 |
| TTC17:CEBPB | IMP3:CSTB | IMP3:TNIP1 | RPL9:MYD88 |
| PREPL:WARS | RPL15:CEBPB | FNTA:CD63 | RPL22:GNG5 |
| TCF4:LAP3 | ARHGAP17:ATOX1 | TTC17:TCIRG1 | FNTA:MYD88 |
| ZBED5:WARS | TTC17:MYD88 | EXOSC10:SH3GLB1 | TCF4:GNG5 |
| TCF4:POMP | EXOSC10:TCIRG1 | RPS4X:FCER1G | EXOSC10:TANK |
| NUP160:SQRDL | ZMYND11:CEBPB | RPS4X:PGD | MLLT10:WARS |
| TRIT1:WARS | CEP192:TANK | CAMK2G:CEBPB | TTC17:POMP |
| ZBED5:CEBPB | IMP3:UBE2L6 | ZMYND11:G6PD | TCF4:MYD88 |
| IMP3:WARS | RPS4X:CD63 | FNTA:CEBPB | IMP3:MYD88 |
| RPS4X:SQRDL | RPL9:CD63 | ZMYND11:CD63 | TOP2B:CD63 |
| NUP160:POMP | ARID1A:UBE2L6 | TCF4:RALB | CEP192:RALB |
| EXOSC10:LAP3 | TCF4:UBE2L6 | ARHGAP17:LAP3 | NUP160:PGD |
| RPS4X:GNG5 | ARID1A:WARS | IMP3:CD63 | RPL9:SQRDL |
| TOP2B:WARS | CAMK2G:G6PD | ZMYND11:C3AR1 | CEP192:PCMT1 |
| RPL9:POMP | RPS4X:SH3GLB1 | AHCTF1:WARS | TCF4:SQRDL |
| EXOSC10:ATOX1 | RPL9:TANK | RPS4X:ENO1 | RPL9:GNG5 |
| TTC17:TANK | IMP3:TANK | CEP192:PLSCR1 | EXOSC10:CD63 |
| EXOSC10:CEBPB | ZBED5:SH3GLB1 | EXOSC9:POMP | TCF4:SH3GLB1 |
| NOSIP:CEBPB | TMEM50B:CEBPB | FNTA:GNG5 | ADSL:WARS |
| RPL22:CEBPB | RPS4X:POMP | CEP192:IRF1 | TTC17:SH3GLB1 |
| TTC17:ATP2A2 | TOP2B:POMP | CEP192:CEBPB | ARID1A:SQRDL |
| SEH1L:WARS | METAP1:POMP | ZMYND11:CSTB | ARID1A:G6PD |
| EXOSC10:UBE2L6 | EXOSC10:CSTB | FNTA:SH3GLB1 | AHCTF1:TANK |
| TTC17:LAP3 | ZNF266:CEBPB | ARID1A:TAP1 | EXOSC2:CEBPB |
| RPS4X:SERPINB1 | IMP3:G6PD | TTC17:TIMP2 | NOSIP:TCIRG1 |
| FBXO11:RALB | CEP192:POMP | TTC17:SQRDL | RPL9:FCER1G |
| TMEM50B:SQRDL | TMEM50B:CD63 | ARID1A:CD63 | ARID1A:TRPC4AP |
| CSNK1G2:CEBPB | ZMYND11:ENO1 | FNTA:LAP3 | ARID1A:SH3GLB1 |
| RPL15:SH3GLB1 | CEP192:LAP3 | BCL11A:LAP3 | CEP192:RAB27A |
| BCL11A:G6PD | RPL9:UPP1 | IMP3:FCER1G | EXOSC10:FCER1G |
| ZBED5:SQRDL | TCF4:SERPINB1 | CEP192:TNIP1 | SETX:SQRDL |
| ARID1A:SERPINB1 | AHCTF1:PLAUR | ZMYND11:SQRDL | CEP192:MYD88 |
| RPS14:SH3GLB1 | RPL22:WARS | ZMYND11:GNG5 | ARID1A:BCL6 |
| EXOSC10:TAP1 | EXOSC2:POMP | ARID1A:SLAMF7 | EXOSC2:CD63 |
| BCL11A:CEBPB | ZMYND11:SH3GLB1 | ARID1A:TCIRG1 | AHCTF1:UPP1 |
| ADSL:ATOX1 | RPS14:CD63 | ARID1A:TNIP1 | IMP3:RALB |
| TCF4:FCER1G | CAMK2G:SQRDL | ZMYND11:PGD | ADK:SH3GLB1 |
| LY9:SH3GLB1 | ARIH2:CEBPB | CSNK1G2:TCIRG1 | SUCLG2:CD63 |
| IMP3:GNG5 | ARID1A:NFIL3 | TTC17:CD63 | FNTA:WARS |
| SERTAD2:CEBPB | IMP3:POMP | NUP160:RTN4 | EXOSC10:TUBA1B |
| AHCTF1:MYD88 | EXOSC10:ENO1 | RPL15:SQRDL | IMP3:PCBP1 |
| ARID1A:ENO1 | PREPL:SH3GLB1 | TTC17:UPP1 | ARID1A:GRINA |
| EXOSC10:UPP1 | TTC17:BCL6 | CAMK2G:FCER1G | TTC17:PGD |
| CEP192:CSTB | ZMYND11:POMP | CEP192:TCIRG1 | ARID1A:TANK |
| LY9:SQRDL | IMP3:RIT1 | IRF8:CEBPB | CSNK1G2:FLII |
| LY9:TNIP1 | CAMK2G:CD63 | CEP192:G6PD | CEP192:STAT3 |
| CNOT7:G6PD | IL10RA:CEBPB | FBXO11:UPP1 | AHCTF1:SH3GLB1 |
| ARID1A:PLSCR1 | FNTA:TCIRG1 | ARIH2:TCIRG1 | TTC17:SERPINB1 |
| CEP192:ATOX1 | CAMK2G:TCIRG1 | PCID2:WARS | EXOSC2:UPP1 |
| IMP3:ENO1 | EXOSC10:PCMT1 | CAMK2G:PGD | IMP3:TSPO |
| ARID1A:IRF1 | RPS14:SQRDL | EXOSC10:FLII | BCL11A:TNIP1 |
| EXOSC10:GNG5 | IMP3:PGD | RPL15:CD63 | ADSL:ENO1 |
| LY9:ATOX1 | ZBED5:TNIP1 | RPL22:CD63 | NOSIP:SQRDL |
| FBXO11:CEBPB | CHN2:WARS | CNOT7:SQRDL | SERBP1:SH3GLB1 |
| RPL9:SLAMF7 | IMP3:TCIRG1 | FBXO11:SQRDL | ARID1A:NFKBIA |
| RPL9:TNIP1 | AHCTF1:SQRDL | TCF4:UPP1 | RPL9:ENO1 |
| PREPL:CD63 | CLIP4:WARS | PCID2:CEBPB | ARID1A:RAB27A |
| ARHGAP17:SQRDL | NOSIP:POMP | CNOT7:CSTB | RPL15:WARS |
| ZBED5:POMP | RPL22:SQRDL | ARID1A:PGD | BCL11A:CSTB |
| RPS4X:TSPO | IMP3:VAMP3 | ARID1A:STAT3 | |

37. The method of any one of claims 5 to 9 and 11 to 36, wherein a single PaSIRS derived biomarker combination (e.g., any one from TABLE C) is used for determining the indicator.

38. The method of any one of claims 5 to 9 and 11 to 36, wherein two PaSIRS derived biomarker combinations (e.g., any two from TABLE C) are used for determining the indicator.

39. The method of any one of claims 5 to 9 and 11 to 36, wherein three PaSIRS derived biomarker combinations (e.g., any three from TABLE C) are used for determining the indicator.

40. The method of any one of claims 5 to 9 and 11 to 36, wherein four PaSIRS derived biomarker combinations (e.g., any four from TABLE C) are used for determining the indicator.

41. The method of claim 37, comprising: (a) determining a single PaSIRS derived biomarker value using a pair of PaSIRS biomarker values, the single PaSIRS derived biomarker value being indicative of a ratio of levels of first and second PaSIRS biomarkers; and (b) determining the indicator using the single derived PaSIRS biomarker value.

42. The method of claim 38, comprising: (a) determining a first PaSIRS derived biomarker value using a first pair of PaSIRS biomarker values, the first PaSIRS derived biomarker value being indicative of a ratio of levels of first and second PaSIRS biomarkers; (b) determining a second PaSIRS derived biomarker value using a second pair of PaSIRS biomarker values, the second PaSIRS derived biomarker value being indicative of a ratio of levels of third and fourth PaSIRS biomarkers; and (c) determining the indicator by combining the first and second derived PaSIRS biomarker values, using for example a combining function as disclosed herein.

43. The method of claim 39, comprising: (a) determining a first PaSIRS derived biomarker value using a first pair of PaSIRS biomarker values, the first PaSIRS derived biomarker value being indicative of a ratio of levels of first and second PaSIRS biomarkers; (b) determining a second PaSIRS derived biomarker value using a second pair of PaSIRS biomarker values, the second PaSIRS derived biomarker value being indicative of a ratio of levels of third and fourth PaSIRS biomarkers; (c) determining a third PaSIRS derived biomarker value using a third pair of PaSIRS biomarker values, the third PaSIRS derived biomarker value being indicative of a ratio of levels of fifth and fourth PaSIRS biomarkers; and (d) determining the indicator by combining the first and sixth derived PaSIRS biomarker values, using for example a combining function as disclosed herein.

44. The method of any one of claims 5 to 9 and 11 to 43, wherein individual PaSIRS derived biomarker combinations are suitably selected from TTC17:G6PD, HERC6:LAP3 and NUP160:TPP1.

45. The method of any one of claims 5 to 9 and 11 to 43, wherein the protozoan associated with the PaSIRS is selected from any of the following protozoal genera, which are capable of inducing at least one of the clinical signs of SIRS; for example, Toxoplasma, Babesia, Plasmodium, Trypanosoma, Giardia, Entamoeba, Cryptosporidium, Balantidium and Leishmania.

46. The method of any one of claims 9 to 45, wherein individual InSIRS derived biomarker combinations are selected from TABLE D.
TABLE D
InSIRS Derived Biomarker

| | | | |
|---|---|---|---|
| TNFSF8:VEZT | TNFSF8:NIP7 | TNFSF8:LRRC8D | ENTPD1:ARL6IP5 |
| TNFSF8:HEATR1 | TNFSF8:MLLT10 | TNFSF8:RNMT | TNFSF8:CD84 |
| TNFSF8:THOC2 | TNFSF8:EIF5B | STK17B:ARL6IP5 | TNFSF8:PWP1 |
| TNFSF8:IPO7 | TNFSF8:ANK3 | TNFSF8:IQCB1 | TNFSF8:SLC35D1 |
| ADAM19:EXOC7 | TNFSF8:SMC3 | TNFSF8:FASTKD2 | SYNE2:RBM26 |
| TNFSF8:ARHGAP5 | TNFSF8:REPS1 | TNFSF8:RDX | TNFSF8:CD40LG |
| TNFSF8:RMND1 | TNFSF8:C14orf1 | TNFSF8:MTO1 | VNN3:CYSLTR1 |
| TNFSF8:IDE | TNFSF8:FUT8 | IQSEC1:MACF1 | TNFSF8:SYT11 |
| TNFSF8:TBCE | TNFSF8:VPS13A | TNFSF8:SMC6 | TNFSF8:RIOK2 |
| TNFSF8:G3BP1 | TNFSF8:RAD50 | TNFSF8:NEK1 | TNFSF8:BZW2 |
| TNFSF8:CDK6 | TNFSF8:ESF1 | TNFSF8:ZNF562 | TNFSF8:LARP1 |
| TNFSF8:MANEA | TNFSF8:MRPS10 | TNFSF8:PEX1 | ADAM19:SYT11 |
| TNFSF8:CKAP2 | CDA:EFHD2 | ADAM19:SIDT2 | TNFSF8:NCBP1 |
| TNFSF8:ZNF507 | TNFSF8:SLC35A3 | TNFSF8:METTL5 | ADAM19:MACF1 |
| TNFSF8:GGPS1 | ADAM19:TMEM87A | CYP4F3:TRAPPC2 | TNFSF8:NOL8 |
| TNFSF8:XPO4 | TNFSF8:LANCL1 | TNFSF8:KRIT1 | TNFSF8:KIAA0391 |
| TNFSF8:PHC3 | ADAM19:ERCC4 | TNFSF8:YEATS4 | |
| TNFSF8:ASCC3 | TNFSF8:CD28 | TNFSF8:CLUAP1 | |
| TNFSF8:NOL10 | ADAM19:MLLT10 | TNFSF8:LARP4 | |

47. The method of any one of claims 9 to 46, wherein a single InSIRS derived biomarker combination (e.g., any one from TABLE D) is used for determining the indicator.

48. The method of any one of claims 9 to 46, wherein two InSIRS derived biomarker combinations (e.g., any two from TABLE D) are used for determining the indicator.

49. The method of any one of claims 9 to 46, wherein three InSIRS derived biomarker combinations (e.g., any three from TABLE D) are used for determining the indicator.

50. The method of any one of claims 9 to 46, wherein four InSIRS derived biomarker combinations (e.g., any four from TABLE D) are used for determining the indicator.

51. The method of claim 47, comprising: (a) determining a single InSIRS derived biomarker value using a pair of InSIRS biomarker values, the single InSIRS derived biomarker value being indicative of a ratio of levels of first and second InSIRS biomarkers; and (b) determining the indicator using the single derived InSIRS biomarker value.

52. The method of claim 48, comprising: (a) determining a first InSIRS derived biomarker value using a first pair of InSIRS biomarker values, the first InSIRS derived biomarker value being indicative of a ratio of levels of first and second InSIRS biomarkers; (b) determining a second InSIRS derived biomarker value using a second pair of InSIRS biomarker values, the second InSIRS derived biomarker value being indicative of a ratio of levels of third and fourth InSIRS biomarkers; and (c) determining the indicator by combining the first and second derived InSIRS biomarker values, using for example a combining function as disclosed herein.

53. The method of claim 49, comprising: (a) determining a first InSIRS derived biomarker value using a first pair of InSIRS biomarker values, the first InSIRS derived biomarker value being indicative of a ratio of levels of first and second InSIRS biomarkers; (b) determining a second InSIRS derived biomarker value using a second pair of InSIRS biomarker values, the second InSIRS derived biomarker value being indicative of a ratio of levels of third and fourth InSIRS biomarkers; (c) determining a third InSIRS derived biomarker value using a third pair of InSIRS biomarker values, the third InSIRS derived biomarker value being indicative of a ratio of levels of fifth and fourth InSIRS biomarkers; and (d) determining the indicator by combining the first and sixth derived InSIRS biomarker values, using for example a combining function as disclosed herein.

54. The method of any one of claims 9 to 54, wherein individual InSIRS derived biomarker combinations are suitably selected from ENTPD1:ARL6IP5, TNFSF8:HEATR1, ADAM19:POLR2A, SYNE2:VPS13C, TNFSF8:NIP7, CDA:EFHD2, ADAM19:MLLT10, PTGS1:ENTPD1, ADAM19:EXOC7 and CDA:PTGS1.

55. The method of any one of claims 9 to 54, wherein individual InSIRS derived biomarker combinations are suitably selected from ENTPD1:ARL6IP5 and TNFSF8:HEATR1.

56. An apparatus for determining an indicator used in assessing a likelihood of a subject having a presence, absence or degree of BaSIRS or VaSIRS. This apparatus generally comprises at least one electronic processing device that:
determines a plurality of host response specific biomarker values including a plurality of BaSIRS biomarker values and a plurality of VaSIRS biomarker values, the plurality of BaSIRS biomarker values being indicative of values measured for a corresponding plurality of BaSIRS biomarkers in a sample taken from the subject, the plurality of VaSIRS biomarker values being indicative of values measured for a corresponding plurality of VaSIRS biomarkers in the sample;
determines a plurality of host response specific derived biomarker values including at least one BaSIRS derived biomarker value and at least one VaSIRS derived biomarker value, each derived BaSIRS biomarker value being determined using at least a subset of the plurality of BaSIRS biomarker values, and being indicative of a ratio of levels of a corresponding at least a subset of the plurality of BaSIRS biomarkers, and each derived VaSIRS biomarker value being determined using at least a subset of the plurality of VaSIRS biomarker values, and being indicative of a ratio of levels of a corresponding at least a subset of the plurality of VaSIRS biomarkers; and
determines the indicator using the plurality of host response specific derived biomarker values, wherein the at least a subset of BaSIRS biomarkers forms a BaSIRS derived biomarker combination which is not a derived biomarker combination for VaSIRS, PaSIRS or InSIRS, and wherein the at least a subset of VaSIRS biomarkers forms a VaSIRS derived biomarker combination which is not a derived biomarker combination for BaSIRS, PaSIRS or InSIRS.

57. The apparatus of claim 56, wherein the at least one processing device:
(a) determines a plurality of pathogen specific biomarker values including at least one bacterial biomarker value and at least one viral biomarker value, the least one bacterial biomarker value being indicative of a value measured for a corresponding bacterial biomarker in the sample, the least one viral biomarker value being indicative of a value measured for a corresponding viral biomarker in the sample; and
(b) determines the indicator using the host response specific derived biomarker values in combination with the pathogen specific biomarker values.

58. The apparatus of claim 56 or claim 57, wherein the plurality of host response specific biomarker values determined by the least one electronic processing device further include a plurality of PaSIRS biomarker values, the plurality of PaSIRS biomarker values being indicative of values measured for a corresponding plurality of PaSIRS biomarkers in the sample, and the plurality of host response specific derived biomarker values further includes at least one PaSIRS derived biomarker value, and the least one electronic processing device further:
determines each PaSIRS derived biomarker value using at least a subset of the plurality of PaSIRS biomarker values, the PaSIRS derived biomarker value being indicative of a ratio of levels of a corresponding at least a subset of the plurality of PaSIRS biomarkers; and
determines the indicator using the plurality of host response specific derived biomarker values, wherein the at least a subset of PaSIRS biomarkers forms a PaSIRS derived biomarker combination which is not a derived biomarker combination for BaSIRS, VaSIRS or InSIRS.

59. The apparatus of any one of claims 56 to 58, wherein the least one electronic processing device:
(a) determines a plurality of pathogen specific biomarker values including at least one bacterial biomarker value, at least one viral biomarker value and at least one protozoal biomarker value, the at least one bacterial biomarker value being indicative of a value measured for a corresponding bacterial biomarker in the sample, the least one viral biomarker value being indicative of a value measured for a corresponding viral biomarker in the sample, and the least one protozoal biomarker value being indicative of a value measured for a corresponding protozoal biomarker in the sample; and
(b) determines the indicator using the host response specific derived biomarker values in combination with the pathogen specific biomarker values.

60. The apparatus of any one of claims 56 to 59, wherein the plurality of host response specific biomarker values determined by the least one electronic processing device further include a plurality of InSIRS biomarker values, the plurality of InSIRS biomarker values being indicative of values measured for a corresponding plurality of InSIRS biomarkers in the sample, and the plurality of host response specific derived biomarker values further includes at least one InSIRS derived biomarker value, and the least one electronic processing device further:
determines each InSIRS derived biomarker value using at least a subset of the plurality of InSIRS biomarker values, the InSIRS derived biomarker value being indicative of a ratio of levels of a corresponding at least a subset of the plurality of InSIRS biomarkers; and
determines the indicator using the plurality of host response specific derived biomarker values, wherein the at least a subset of InSIRS biomarkers forms a InSIRS derived biomarker combination which is not a derived biomarker combination for BaSIRS, VaSIRS or PaSIRS.

61. A composition for determining an indicator used in assessing a likelihood of a subject having a presence, absence or degree of BaSIRS or VaSIRS, the composition comprising: (1) a pair of BaSIRS biomarker cDNAs, and for each BaSIRS biomarker cDNA at least one oligonucleotide primer that hybridizes to the BaSIRS biomarker cDNA, and/or at least one oligonucleotide probe that hybridizes to the BaSIRS biomarker cDNA, wherein the at least one oligonucleotide primer and/or the at least one oligonucleotide probe comprises a heterologous label, and (2) a pair of VaSIRS biomarker cDNAs, and for each VaSIRS biomarker cDNA at least one oligonucleotide primer that hybridizes to the VaSIRS biomarker cDNA, and/or at least one oligonucleotide probe that hybridizes to the VaSIRS biomarker cDNA, wherein the at least one oligonucleotide primer and/or the at least one oligonucleotide probe comprises a heterologous label, wherein the pair of BaSIRS biomarker cDNAs forms a BaSIRS derived biomarker combination which is not a derived biomarker combination for VaSIRS, PaSIRS or InSIRS, wherein the pair of VaSIRS biomarker cDNAs forms a VaSIRS derived biomarker combination which is not a derived biomarker combination for BaSIRS, PaSIRS or InSIRS, wherein the BaSIRS derived biomarker combination is selected from the BaSIRS derived biomarker combinations set out in TABLE A, and wherein the VaSIRS derived biomarker combination is selected from the VaSIRS derived biomarker combinations set out in TABLE B.

62. The composition of claim 61, further comprising: (a) a pair of PaSIRS biomarker cDNAs, and for each PaSIRS biomarker cDNA at least one oligonucleotide primer that hybridizes to the PaSIRS biomarker cDNA, and/or at least one oligonucleotide probe that hybridizes to the PaSIRS biomarker cDNA, wherein the at least one oligonucleotide primer and/or the at least one oligonucleotide probe comprises a heterologous label, wherein the pair of PaSIRS biomarker cDNAs forms a PaSIRS derived biomarker combination which is not a derived biomarker combination for BaSIRS, VaSIRS or InSIRS, and wherein the PaSIRS derived biomarker combination is selected from the PaSIRS derived biomarker combinations set out in TABLE C.

63. The composition of claim 61 or claim 62, further comprising: (b) a pair of InSIRS biomarker cDNAs, and for each InSIRS biomarker cDNA at least one oligonucleotide primer that hybridizes to the InSIRS biomarker cDNA, and/or at least one oligonucleotide probe that hybridizes to the InSIRS biomarker cDNA, wherein the at least one oligonucleotide primer and/or the at least one oligonucleotide probe comprises a heterologous label, wherein the pair of InSIRS biomarker cDNAs forms an InSIRS derived biomarker combination which is not a derived biomarker combination for BaSIRS, VaSIRS or PaSIRS, and wherein the InSIRS derived biomarker combination is selected from the InSIRS derived biomarker combinations set out in TABLE D.

64. The composition of any one of claims 61 to 63, further comprising a DNA polymerase.

65. The composition of claim 64, wherein the DNA polymerase is a thermostable DNA polymerase.

66. The composition of any one of claims 61 to 65, comprising for each cDNA a pair of forward and reverse oligonucleotide primers that permit nucleic acid amplification of at least a portion of the cDNA to produce an amplicon.

67. The composition of claim 66, further comprising for each cDNA an oligonucleotide probe that comprises a heterologous label and hybridizes to the amplicon.

68. The composition of any one of claims 61 to 67, wherein the components of an individual composition are comprised in a mixture.

69. The composition of any one of claims 61 to 68, comprising a population of cDNAs corresponding to mRNA derived from a cell or cell population from a patient sample.

70. The composition of claim 69, wherein the population of cDNAs represents whole leukocyte cDNA (e.g., whole peripheral blood leukocyte cDNA) with a cDNA expression profile characteristic of a subject with a SIRS condition selected from BaSIRS, VaSIRS, PaSIRS and InSIRS, wherein the cDNA expression profile comprises at least one pair of biomarkers (e.g., 1, 2, 3, 4, 5, 6, 7, 8, 9, 10, 11, 12, 13, 14, 15, 16, 17, 18, 19, 20, 30, 40, 50 or more pairs of biomarkers), wherein a respective pair of biomarkers comprises a first biomarker and a second biomarker, wherein the first biomarker is expressed at a higher level in leukocytes (e.g., whole peripheral blood leukocytes) from a subject with the SIRS condition than in leukocytes (e.g., whole peripheral blood leukocytes) from a healthy subject or from a subject without the SIRS condition (e.g., the first biomarker is expressed in leukocytes from a subject with the SIRS condition at a level that is at least 110%, 120%, 130%, 140%, 150%, 160%, 170%, 180%, 190%, 200%, 250%, 300%, 350%, 400%, 450%, 500%, 600%, 700%, 800%, 900%, 1000%, 2000%, 3000%, 4000%, or 5000% of the level of the first biomarker in leukocytes from a healthy subject or from a subject without the SIRS condition), wherein the second biomarker is expressed at about the same or at a lower level in leukocytes (e.g., whole peripheral blood leukocytes) from a subject with the SIRS condition than in leukocytes (e.g., whole peripheral blood leukocytes) from a healthy subject or from a subject without the SIRS condition (e.g., the second biomarker is expressed in leukocytes from a subject with the SIRS condition at a level that is no more than 105%, 104%, 103%, 102%, 100%, 99%, 98%, 97%, 96%, 95%, 90%, 85%, 80%, 75%, 70%, 65%, 60%, 55%, 50%, 45%, 40%, 35%, 30%, 25%, 20%, 15%, 10%, 5%, 1%, 0.5%, 0.1%, 0.05%, 0.01%, 0.005%, 0.001% of the level of the second biomarker in leukocytes from a healthy subject or from a subject without the SIRS condition) and wherein the first biomarker is a first mentioned or ‘numerator’ biomarker of a respective pair of biomarkers in any one of TABLES A, B, C or D, and the second biomarker represents a second mentioned or ‘denominator’ biomarker of the respective pair of biomarkers.

71. The composition of claim 69, wherein the sample is a body fluid, including blood, urine, plasma, serum, urine, secretion or excretion.

72. The composition of claim 69, wherein the cell population is from blood, suitably peripheral blood.

73. The composition of claim 69, wherein the sample comprises blood, suitably peripheral blood.

74. The composition of any one of claims 69 to 73, wherein the cell or cell population is a cell or cell population of the immune system, suitably a leukocyte or leukocyte population.

75. The composition of any one of claims 61 to 74, further comprising a pathogen nucleic acid and at least one oligonucleotide primer that hybridizes to the pathogen nucleic acid, and/or at least one oligonucleotide probe that hybridizes to the pathogen nucleic acid, wherein the at least one oligonucleotide primer and/or the at least one oligonucleotide probe comprises a heterologous label.

76. The composition of claim 75, wherein the pathogen from which the pathogen nucleic acid is selected is from a bacterium, a virus and a protozoan.

77. The composition of claim 76, wherein the pathogen nucleic acid is derived from a patient sample, suitably a body fluid.

78. The composition of claim 77, wherein the body fluid is selected from blood, urine, plasma, serum, urine, secretion and excretion.

79. The composition of claim 77, wherein the sample comprises blood, suitably peripheral blood.

80. A kit for determining an indicator used in assessing a likelihood of a subject having a presence, absence or degree of BaSIRS or VaSIRS, the kit comprising: (1) for each of a pair of BaSIRS biomarker cDNAs at least one oligonucleotide primer and/or at least one oligonucleotide probe that hybridizes to the BaSIRS biomarker cDNA, wherein the at least one oligonucleotide primer and/or the at least one oligonucleotide probe comprises a heterologous label; and (2) for each of a pair of VaSIRS biomarker cDNA at least one oligonucleotide primer and/or at least one oligonucleotide probe that hybridizes to the VaSIRS biomarker cDNA, wherein the at least one oligonucleotide primer and/or the at least one oligonucleotide probe comprise(s) a heterologous label, wherein the pair of BaSIRS biomarker cDNAs forms a BaSIRS derived biomarker combination which is not a derived biomarker combination for VaSIRS, PaSIRS or InSIRS, wherein the pair of VaSIRS biomarker cDNAs forms a VaSIRS derived biomarker combination which is not a derived biomarker combination for BaSIRS, PaSIRS or InSIRS, wherein the BaSIRS derived biomarker combination is selected from the BaSIRS derived biomarker combinations set out in TABLE A, and wherein the VaSIRS derived biomarker combination is selected from the VaSIRS derived biomarker combinations set out in TABLE B.

81. The kit of claim 80, further comprising: (a) for each of a pair of PaSIRS biomarker cDNAs at least one oligonucleotide primer and/or at least one oligonucleotide probe that hybridizes to the PaSIRS biomarker cDNA, wherein the at least one oligonucleotide primer and/or the at least one oligonucleotide probe comprises a heterologous label, wherein the at least one oligonucleotide primer and/or the at least one oligonucleotide probe comprises a heterologous label, wherein the pair of PaSIRS biomarker cDNAs forms a PaSIRS derived biomarker combination which is not a derived biomarker combination for BaSIRS, VaSIRS or InSIRS, and wherein the PaSIRS derived biomarker combination is selected from the PaSIRS derived biomarker combinations set out in TABLE C.

82. The kit of claim 80 or claim 81, further comprising: (b) for each of a pair of InSIRS biomarker cDNAs at least one oligonucleotide primer and/or at least one oligonucleotide probe that hybridizes to the InSIRS biomarker cDNA, wherein the at least one oligonucleotide primer and/or the at least one oligonucleotide probe comprises a heterologous label, wherein the pair of InSIRS biomarker cDNAs forms an InSIRS derived biomarker combination which is not a derived biomarker combination for BaSIRS, VaSIRS or PaSIRS, and wherein the InSIRS derived biomarker combination is selected from the InSIRS derived biomarker combinations set out in TABLE D.

83. The kit of any one of claims 80 to 82, further comprising: at least one oligonucleotide primer that hybridizes to a pathogen nucleic acid, and/or at least one oligonucleotide probe that hybridizes to the pathogen nucleic acid, wherein the at least one oligonucleotide primer and/or the at least one oligonucleotide probe comprises a heterologous label.

84. The kit of any one of claims 80 to 83, further comprising: a DNA polymerase.

85. The kit of claim 84, wherein the DNA polymerase is a thermostable DNA polymerase.

86. The kit of any one of claims 80 to 85, further comprising: for each cDNA a pair of forward and reverse oligonucleotide primers that permit nucleic acid amplification of at least a portion of the cDNA to produce an amplicon.

87. The kit of any one of claims 80 to 86, further comprising: for each cDNA an oligonucleotide probe that comprises a heterologous label and hybridizes to the amplicon.

88. The kit of any one of claims 80 to 87, wherein the components of the kit when used to determine the indicator are combined to form a mixture.

89. The kit of any one of claims 80 to 88, further comprising: one or more reagents for preparing mRNA from a cell or cell population from a patient sample (e.g., a body fluid such as blood, urine, plasma, serum, urine, secretion or excretion).

90. The kit of any one of claims 80 to 89, further comprising: a reagent for preparing cDNA from the mRNA.

91. A method for treating a subject with a SIRS condition selected from BaSIRS and VaSIRS and optionally one of PaSIRS or InSIRS, ther method comprising: exposing the subject to a treatment regimen for treating the SIRS condition based on an indicator obtained from an indicator-determining method, wherein the indicator is indicative of the presence, absence or degree of the SIRS condition in the subject, and wherein the indicator-determining method is as defined in any one of claims 1 to 55.

92. The method of claim 91, further comprising: taking a sample from the subject and determining an indicator indicative of the likelihood of the presence, absence or degree of the SIRS condition using the indicator-determining method.

93. The method of claim 91 or claim 92, further comprising: sending a sample taken from the subject to a laboratory at which the indicator is determined according to the indicator-determining method.

94. The method of claim 93, further comprising: receiving the indicator from the laboratory.

95. A method for managing a subject with a specific SIRS condition selected from BaSIRS and VaSIRS and optionally one of PaSIRS or InSIRS, ther method comprising: exposing the subject to a treatment regimen for the specific SIRS condition and avoiding exposing the subject to a treatment regimen for a SIRS condition other than the specific SIRS condition, based on an indicator obtained from an indicator-determining method, wherein the indicator is indicative of the presence, absence or degree of the SIRS condition in the subject, and wherein the indicator-determining method is an indicator-determining method as defined in any one of claims 1 to 55.

96. The method of claim 95, further comprising: taking a sample from the subject and determining an indicator indicative of the likelihood of the presence, absence or degree of the SIRS condition using the indicator-determining method.

97. The method of claim 95 or claim 96, further comprising: sending a sample taken from the subject to a laboratory at which the indicator is determined according to the indicator-determining method.

98. The method of claim 97, further comprising: receiving the indicator from the laboratory.

99. A method of monitoring the efficacy of a treatment regimen in a subject with a SIRS condition selected from BaSIRS and VaSIRS and optionally one of PaSIRS or InSIRS, wherein the treatment regimen is monitored for efficacy towards a desired health state (e.g., absence of the SIRS condition), the method comprising: (1) obtaining a biomarker profile of a sample taken from the subject after treatment of the subject with the treatment regimen, wherein the sample biomarker profile comprises (a) for each of a plurality of derived biomarkers as defined in any one of claims 1 to 55 a plurality of host response specific derived biomarker values, and optionally (b) if the SIRS condition is an infection positive SIRS condition (“IpSIRS”), a pathogen specific biomarker value as defined in claim 3 or claim 8 for a pathogen biomarker associated with the SIRS condition; and (2) comparing the sample biomarker profile to a reference biomarker profile that is correlated with a presence, absence or degree of the SIRS condition to thereby determine whether the treatment regimen is effective for changing the health status of the subject to the desired health state.

100. A method of monitoring the efficacy of a treatment regimen in a subject towards a desired health state (e.g., absence of BaSIRS, VaSIRS, PaSIRS, or InSIRS), the method comprising: (1) determining an indicator according to an indicator-determining method as broadly described above and elsewhere herein based on a sample taken from the subject after treatment of the subject with the treatment regimen; and (2) assessing the likelihood of the subject having a presence, absence or degree of a SIRS condition selected from BaSIRS and VaSIRS and optionally one of PaSIRS or InSIRS using the indicator to thereby determine whether the treatment regimen is effective for changing the health status of the subject to the desired health state.

101. The method of claim 100, wherein the indicator is determined using a plurality of host response specific derived biomarker values.

102. The method of claim 100, wherein the indicator is determined using a plurality of host response specific derived biomarker values and a plurality of pathogen specific biomarker values.

103. A method of correlating a biomarker profile with an effective treatment regimen for a SIRS condition selected from BaSIRS and VaSIRS and optionally one of PaSIRS or InSIRS, the method comprising: (1) determining a biomarker profile of a sample taken from a subject with the SIRS condition and for whom an effective treatment has been identified, wherein the biomarker profile comprises: (a) for each of a plurality of derived biomarkers as defined in any one of claims 1 to 55 a plurality of host response specific derived biomarker values, and optionally (b) if the SIRS condition is an IpSIRS, a pathogen specific biomarker value as defined in claim 3 or claim 8 for a pathogen biomarker associated with the SIRS condition; and (2) correlating the biomarker profile so determined with an effective treatment regimen for the SIRS condition.

104. A method of determining whether a treatment regimen is effective for treating a subject with a SIRS condition selected from BaSIRS and VaSIRS and optionally one of PaSIRS or InSIRS, the method comprising: (1) determining a post-treatment biomarker profile of a sample taken from the subject after treatment with a treatment regimen, wherein the biomarker profile comprises: (a) for each of a plurality of derived biomarkers as defined in any one of claims 1 to 55 a plurality of host response specific derived biomarker values, and optionally (b) if the SIRS condition is an IpSIRS, a pathogen specific biomarker value as defined in claim 3 or claim 8 for a pathogen biomarker associated with the SIRS condition; and (2) determining a post-treatment indicator using the post-treatment biomarker profile, wherein the post-treatment indicator is at least partially indicative of the presence, absence or degree of the SIRS condition, wherein the post-treatment indicator indicates whether the treatment regimen is effective for treating the SIRS condition in the subject on the basis that post-treatment indicator indicates the presence of a healthy condition or the presence of the SIRS condition of a lower degree relative to the degree of the SIRS condition in the subject before treatment with the treatment regimen.

105. A method of correlating a biomarker profile with a positive or negative response to a treatment regimen for treating a SIRS condition selected from BaSIRS and VaSIRS and optionally one of PaSIRS or InSIRS, the method comprising: (1) determining a biomarker profile of a sample taken from a subject with the SIRS condition following commencement of the treatment regimen, wherein the reference biomarker profile comprises: (a) for each of a plurality of derived biomarkers as defined in any one of claims 1 to 55 a plurality of host response specific derived biomarker values, and optionally (b) if the SIRS condition is an IpSIRS, a pathogen specific biomarker value as defined in claim 3 or claim 8 for a pathogen biomarker associated with the SIRS condition; and (2) correlating the sample biomarker profile with a positive or negative response to the treatment regimen.

106. A method of determining a positive or negative response to a treatment regimen by a subject with a SIRS condition selected from BaSIRS and VaSIRS and optionally one of PaSIRS or InSIRS, the method comprising: (1) correlating a reference biomarker profile with a positive or negative response to the treatment regimen, wherein the biomarker profile comprises: (a) for each of a plurality of derived biomarkers as defined in any one of claims 1 to 55 a plurality of host response specific derived biomarker values, and optionally (b) if the SIRS condition is an IpSIRS, a pathogen specific biomarker value as defined in claim 3 or claim 8 for a pathogen biomarker associated with the SIRS condition; (2) detecting a biomarker profile of a sample taken from the subject, wherein the sample biomarker profile comprises (i) a plurality of host response specific derived biomarker values for each of the plurality of derived biomarkers in the reference biomarker profile, and optionally (ii) a pathogen specific biomarker value for the pathogen biomarker in the reference biomarker profile, wherein the sample biomarker profile indicates whether the subject is responding positively or negatively to the treatment regimen.

107. A method of determining a positive or negative response to a treatment regimen by a subject with a SIRS condition selected from BaSIRS and VaSIRS and optionally one of PaSIRS or InSIRS, the method comprising: (1) obtaining a biomarker profile of a sample taken from the subject following commencement of the treatment regimen, wherein the biomarker profile comprises: (a) for each of a plurality of derived biomarkers as defined in any one of claims 1 to 55 a plurality of host response specific derived biomarker values, and optionally (b) if the SIRS condition is an IpSIRS, a pathogen specific biomarker value as defined in claim 3 or claim 8 for a pathogen biomarker associated with the SIRS condition, wherein the sample biomarker profile is correlated with a positive or negative response to the treatment regimen; and (2) and determining whether the subject is responding positively or negatively to the treatment regimen.

108. Use of the indicator-determining methods as defined in any one of claims 1 to 55 in methods for correlating a biomarker profile with an effective treatment regimen for a SIRS condition selected from BaSIRS and VaSIRS and optionally one of PaSIRS or InSIRS, or for determining whether a treatment regimen is effective for treating a subject with the SIRS condition, or for correlating a biomarker profile with a positive or negative response to a treatment regimen, or for determining a positive or negative response to a treatment regimen by a subject with the SIRS condition.
